SEQUENCE LISTING (124 sequences)

SEQ ID NO: 1 - protein, 261 aa, Haemophilus ducreyi
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFNSSSSPPERRVYSWMVVGDFNRAPANLEVALRQEPAVSENTIIIAPTEPTHRSGNILDYAILHDAHLPRREQARERIGASLMLNQLRSQITSDHFPVSFVRDR

SEQ ID NO: 2 - protein, 256 aa, Haemophilus parasuis
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKR

SEQ ID NO: 3 - protein, 251 aa, Escherichia coli
DLTDFRVATWNLQGASATTESKWNINVRQLISGENAVDILAVQEAGSPPSTAVDTGTLIPSPGIPVRELIWNLSTNSRPQQVYIYFSAVDALGGRVNLALVSNRRADEVFVLSPVRQGGRPLLGIRIGNDAFFTAHAIAMRNNDAPALVEEVYNFFRDSRDPVHQALNWMILGDFNREPADLEMNLTVPVRRASEIISPAAATQTSQRTLDYAVAGNSVAFRPSPLQAGIVYGARRTQISSDHFPVGVSRR

SEQ ID NO: 4 - protein, 261 aa, Aggregatibacter actinomycetemcomitans
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFTSSPSSPERRGYSWMVVGDFNRAPVNLEAALRQEPAVSENTIIIAPTEPTHRSGNILDYAILHDAHLPRREQARERIGASLMLNQLRSQITSDHFPVSFVRDR

SEQ ID NO: 5 - protein, 257 aa, Salmonella enterica
ALTDYKVATWNLQGSSTRSENKWNVNVRQLVSGAGAVDILMVQEAGRPPASAVDTGRIINSPGIPVRELTWNLGSNSRPQQVFIYFSQLDVFAGRVNLAIVSHRRADEVIVLPPPSTASRPIMGIRIGSDAFFTIHALANRGVDAPAVVNSVFEFFRNSTRPDMQATNWMIAGDFNRNPDNLRMAIETPVRNNTVILAPSDPTQRSGGILDYAVVGNAIAFIPPVLRAGLLFGERATQISSDHYPVGIFLPPPGEPR

SEQ ID NO: 6 - protein, 4 aa, Synthetic construct
RVRR

SEQ ID NO: 7 - protein, 16 aa, synthetic construct
CAGNRVRRSVGSSLSC

SEQ ID NO: 8 - protein, 15 aa, Synthetic construct
AGNRVRRSVGSSLSG

SEQ ID NO: 9 - protein, 15 aa, Synthetic construct
CKGGASATFGVASLC

SEQ ID NO: 10 - protein, 185 aa, Corynebacterium diphtheriae
INLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKT

SEQ ID NO: 11 - protein, 127 aa, Homo sapiens [sequence omitted]

SEQ ID NO: 12 - protein, 133 aa, Homo sapiens
APMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 13 - protein, 113 aa, Homo sapiens
SWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQ

SEQ ID NO: 14 - protein, 133 aa, Synthetic construct
APMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLWTLENAQAQQTTLSLAIF

SEQ ID NO: 15 - protein, 113 aa, Synthetic construct
SWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLWTLENAQAQ

SEQ ID NO: 16 - protein, 247 aa, Synthetic construct
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGAPQSGSEGASQGAGQPGGDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKR

SEQ ID NO: 17 - protein, 7 aa, Synthetic construct
GSGGGGS

SEQ ID NO: 18 - protein, 6 aa, Synthetic construct
GGEASG

SEQ ID NO: 19 - protein, 4 aa, Synthetic construct
HGQG

SEQ ID NO: 20 - protein, 6 aa, Synthetic construct
GGEGSG

SEQ ID NO: 21 - protein, 7 aa, Synthetic construct
GGEASGG

SEQ ID NO: 22 - protein, 16 aa, Synthetic construct
HGGEGQGEPGAQSAGG

SEQ ID NO: 23 - protein, 19 aa, Synthetic construct
GAPQSGSEGASQGAGQPGG

SEQ ID NO: 24 - protein, 9 aa, Synthetic construct
GGGGEGGGG

SEQ ID NO: 25 - protein, 10 aa, Synthetic construct
HGGGGEGGGG

SEQ ID NO: 26 - protein, 15 aa, Synthetic construct
GGEGQGEPGAQSAGG

SEQ ID NO: 27 - protein, 15 aa, Synthetic construct
GGGGQGGGGEGGGSG

SEQ ID NO: 28 - protein, 16 aa, Synthetic construct
HGGGGQGGGGEGGGSG

SEQ ID NO: 29 - protein, 5 aa, Synthetic construct
GGGGS

SEQ ID NO: 30 - protein, 14 aa, Synthetic construct
GGGGSGGGGSGGGG

SEQ ID NO: 31 - protein, 599 aa, Synthetic construct
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFNSSSSPPERRVYSWMVVGDFNRAPANLEVALRQEPAVSENTIIIAPTEPTHRSGNILDYAILHDAHLPRREQARERIGASLMLNQLRSQITSDHFPVSFVRDRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGQGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 32 - protein, 605 aa, Synthetic construct
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFNSSSSPPERRVYSWMVVGDFNRAPANLEVALRQEPAVSENTIIIAPTEPTHRSGNILDYAILHDAHLPRREQARERIGASLMLNQLRSQITSDHFPVSFVRDRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGGGGEGGGGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 33 - protein, 614 aa, Synthetic construct
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFNSSSSPPERRVYSWMVVGDFNRAPANLEVALRQEPAVSENTIIIAPTEPTHRSGNILDYAILHDAHLPRREQARERIGASLMLNQLRSQITSDHFPVSFVRDRGGEGQGEPGAQSAGGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGGGGEGGGGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 34 - protein, 599 aa, Synthetic construct
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFTSSPSSPERRGYSWMVVGDFNRAPVNLEAALRQEPAVSENTIIIAPTEPTHRSGNILDYAILHDAHLPRREQARERIGASLMLNQLRSQITSDHFPVSFVRDRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGQGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 35 - protein, 589 aa, Synthetic construct
DLTDFRVATWNLQGASATTESKWNINVRQLISGENAVDILAVQEAGSPPSTAVDTGTLIPSPGIPVRELIWNLSTNSRPQQVYIYFSAVDALGGRVNLALVSNRRADEVFVLSPVRQGGRPLLGIRIGNDAFFTAHAIAMRNNDAPALVEEVYNFFRDSRDPVHQALNWMILGDFNREPADLEMNLTVPVRRASEIISPAAATQTSQRTLDYAVAGNSVAFRPSPLQAGIVYGARRTQISSDHFPVGVSRRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGQGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 36 - protein, 594 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGQGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 37 - protein, 595 aa, Synthetic construct
ALTDYKVATWNLQGSSTRSENKWNVNVRQLVSGAGAVDILMVQEAGRPPASAVDTGRIINSPGIPVRELTWNLGSNSRPQQVFIYFSQLDVFAGRVNLAIVSHRRADEVIVLPPPSTASRPIMGIRIGSDAFFTIHALANRGVDAPAVVNSVFEFFRNSTRPDMQATNWMIAGDFNRNPDNLRMAIETPVRNNTVILAPSDPTQRSGGILDYAVVGNAIAFIPPVLRAGLLFGERATQISSDHYPVGIFLPPPGEPRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGQGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 38 - protein, 719 aa, Synthetic construct
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFNSSSSPPERRVYSWMVVGDFNRAPANLEVALRQEPAVSENTIIIAPTEPTHRSGNILDYAILHDAHLPRREQARERIGASLMLNQLRSQITSDHFPVSFVRDRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGQGEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGAPQSGSEGASQGAGQPGGDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKR

SEQ ID NO: 39 - protein, 605 aa, Synthetic construct
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFNSSSSPPERRVYSWMVVGDFNRAPANLEVALRQEPAVSENTIIIAPTEPTHRSGNILDYAILHDAHLPRREQARERIGASLMLNQLRSQITSDHFPVSFVRDRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGQGAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLWTLENAQAQQTTLSLAIF

SEQ ID NO: 40 - protein, 605 aa, Synthetic construct
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFNSSSSPPERRVYSWMVVGDFNRAPANLEVALRQEPAVSENTIIIAPTEPTHRSGNILDYAILHDAHLPRREQARERIGASLMLNQLRSQITSDHFPVSFVRDRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGQGAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 41 - protein, 597 aa, Synthetic construct
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFNSSSSPPERRVYSWMVVGDFNRAPANLEVALRQEPAVSENTIIIAPTEPTHRSGNILDYAILHDAHLPRREQARERIGASLMLNQLRSQITSDHFPVSFVRDRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGGGGQGGGGEGGGSGSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLWTLENAQAQ

SEQ ID NO: 42 - protein, 721 aa, Synthetic construct
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFNSSSSPPERRVYSWMVVGDFNRAPANLEVALRQEPAVSENTIIIAPTEPTHRSGNILDYAILHDAHLPRREQARERIGASLMLNQLRSQITSDHFPVSFVRDRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGQGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGEGGGGSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLWTLENAQAQ

SEQ ID NO: 43 - protein, 721 aa, Synthetic construct
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFNSSSSPPERRVYSWMVVGDFNRAPANLEVALRQEPAVSENTIIIAPTEPTHRSGNILDYAILHDAHLPRREQARERIGASLMLNQLRSQITSDHFPVSFVRDRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGQGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGEGGGGSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQ

SEQ ID NO: 44 - protein, 733 aa, Synthetic construct
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFNSSSSPPERRVYSWMVVGDFNRAPANLEVALRQEPAVSENTIIIAPTEPTHRSGNILDYAILHDAHLPRREQARERIGASLMLNQLRSQITSDHFPVSFVRDRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGGGGEGGGGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGQGGGGEGGGSGSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQ

SEQ ID NO: 45 - protein, 733 aa, Synthetic construct
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFNSSSSPPERRVYSWMVVGDFNRAPANLEVALRQEPAVSENTIIIAPTEPTHRSGNILDYAILHDAHLPRREQARERIGASLMLNQLRSQITSDHFPVSFVRDRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGGGGEGGGGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGQGGGGEGGGSGSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLWTLENAQAQ

SEQ ID NO: 46 - protein, 721 aa, Synthetic construct
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFTSSPSSPERRGYSWMVVGDFNRAPVNLEAALRQEPAVSENTIIIAPTEPTHRSGNILDYAILHDAHLPRREQARERIGASLMLNQLRSQITSDHFPVSFVRDRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGQGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGEGGGGSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLWTLENAQAQ

SEQ ID NO: 47 - protein, 733 aa, Synthetic construct
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFTSSPSSPERRGYSWMVVGDFNRAPVNLEAALRQEPAVSENTIIIAPTEPTHRSGNILDYAILHDAHLPRREQARERIGASLMLNQLRSQITSDHFPVSFVRDRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGGGGEGGGGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGQGGGGEGGGSGSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLWTLENAQAQ

SEQ ID NO: 48 - protein, 711 aa, Synthetic construct
DLTDFRVATWNLQGASATTESKWNINVRQLISGENAVDILAVQEAGSPPSTAVDTGTLIPSPGIPVRELIWNLSTNSRPQQVYIYFSAVDALGGRVNLALVSNRRADEVFVLSPVRQGGRPLLGIRIGNDAFFTAHAIAMRNNDAPALVEEVYNFFRDSRDPVHQALNWMILGDFNREPADLEMNLTVPVRRASEIISPAAATQTSQRTLDYAVAGNSVAFRPSPLQAGIVYGARRTQISSDHFPVGVSRRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGQGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGEGGGGSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLWTLENAQAQ

SEQ ID NO: 49 - protein, 723 aa, Synthetic construct
DLTDFRVATWNLQGASATTESKWNINVRQLISGENAVDILAVQEAGSPPSTAVDTGTLIPSPGIPVRELIWNLSTNSRPQQVYIYFSAVDALGGRVNLALVSNRRADEVFVLSPVRQGGRPLLGIRIGNDAFFTAHAIAMRNNDAPALVEEVYNFFRDSRDPVHQALNWMILGDFNREPADLEMNLTVPVRRASEIISPAAATQTSQRTLDYAVAGNSVAFRPSPLQAGIVYGARRTQISSDHFPVGVSRRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGGGGEGGGGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGQGGGGEGGGSGSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLWTLENAQAQ

SEQ ID NO: 50 - protein, 716 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGQGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGEGGGGSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLWTLENAQAQ

SEQ ID NO: 51 - protein, 728 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGGGGEGGGGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGQGGGGEGGGSGSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLWTLENAQAQ

SEQ ID NO: 52 - protein, 599 aa, Synthetic construct
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFNSSSSPPERRVYSWMVVGDFNRAPANLEVALRQEPAVSENTIIIAPTEPTHRSGNILDYAILHDAHLPRREQARERIGASLMLNQLRSQITSDHFPVSFVRDRGGEASGGCKGGASATFGVASLCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGQGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 53 - protein, 605 aa, Synthetic construct
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFNSSSSPPERRVYSWMVVGDFNRAPANLEVALRQEPAVSENTIIIAPTEPTHRSGNILDYAILHDAHLPRREQARERIGASLMLNQLRSQITSDHFPVSFVRDRGGEASGCASAGQGGGGEGSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGGGGEGGGGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 54 - protein, 409 aa, Synthetic construct
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFNSSSSPPERRVYSWMVVGDFNRAPANLEVALRQEPAVSENTIIIAPTEPTHRSGNILDYAILHDAHLPRREQARERIGASLMLNQLRSQITSDHFPVSFVRDRGGEASGAGNRVRRSVGSSLSGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 55 - protein, 403 aa, Synthetic construct
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFNSSSSPPERRVYSWMVVGDFNRAPANLEVALRQEPAVSENTIIIAPTEPTHRSGNILDYAILHDAHLPRREQARERIGASLMLNQLRSQITSDHFPVSFVRDRGGGGEGGSGGGQSGGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 56 - protein, 629 aa, Synthetic construct
MSENQSSEGHHHHHHSSEVLFQGPNLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFNSSSSPPERRVYSWMVVGDFNRAPANLEVALRQEPAVSENTIIIAPTEPTHRSGNILDYAILHDAHLPRREQARERIGASLMLNQLRSQITSDHFPVSFVRDRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGGGGEGGGGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 57 - protein, 607 aa, Synthetic construct
GPNLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFNSSSSPPERRVYSWMVVGDFNRAPANLEVALRQEPAVSENTIIIAPTEPTHRSGNILDYAILHDAHLPRREQARERIGASLMLNQLRSQITSDHFPVSFVRDRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGGGGEGGGGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 58 - protein, 5 aa, Synthetic construct
GGSGG

SEQ ID NO: 59 - protein, 21 aa, Synthetic construct
AEAAAKAPAPQPAPAEAAAKA

SEQ ID NO: 60 - protein, 18 aa, Synthetic construct
GPAPEPAPAPQPAPAPAG

SEQ ID NO: 61 - protein, 743 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRAEAAAKAPAPQPAPAEAAAKACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGGGGEGGGGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGQGGGGEGGGSGSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLWTLENAQAQ

SEQ ID NO: 62 - protein, 738 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRAEAAAKAPAPQPAPAEAAAKACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPGAEGAGQAGGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGPAPEPAPAPQPAPAPAGSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLWTLENAQAQ

SEQ ID NO: 63 - protein, 177 aa, Corynebacterium diphtheriae
INLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRP

SEQ ID NO: 64 - protein, 16 aa, Synthetic construct
CPGNRVRRSVGSSLSC

SEQ ID NO: 65 - protein, 10 aa, Synthetic construct
GAEGAGQAGG

SEQ ID NO: 66 - protein, 40 aa, Synthetic construct
GQAGGPSGSGGSEPGQSGGQQAEAAAKEAAAKGPGSQGGA

SEQ ID NO: 67 - protein, 15 aa, Synthetic construct
APAPAPAPAPAPAPA

SEQ ID NO: 68 - protein, 18 aa, Synthetic construct
GPAPEPAPAPQPAPAPAG

SEQ ID NO: 69 - protein, 20 aa, Synthetic construct
GAEAAAKEAQAKEAQAAKAG

SEQ ID NO: 70 - protein, 23 aa, Synthetic construct
GAEAAAKAPAPQPAPAEAAAKAG

SEQ ID NO: 71 - protein, 19 aa, Synthetic construct
GAPQAAPEGAPQAPAPEAG

SEQ ID NO: 72 - protein, 18 aa, Synthetic construct
LETQYKETQQKEHQLFEV

SEQ ID NO: 73 - protein, 21 aa, Synthetic construct
AEAAAKAPAPQPAPAEAAAKA

SEQ ID NO: 74 - protein, 8 aa, Synthetic construct
PAPQPHPA

SEQ ID NO: 75 - protein, 19 aa, Synthetic construct
GAPQSGSEGASQGAGQPGG

SEQ ID NO: 76 - protein, 20 aa, Synthetic construct
GGAQPSEAQATESQGGQPGG

SEQ ID NO: 77 - protein, 54 aa, Synthetic construct
APGEAAPAEATAKQAAQEAVEKAPQAEQEAKHQAQEAVEKAEPAEKAAKHAGQG

SEQ ID NO: 78 - protein, 70 aa, Synthetic construct
APGEAAPAEATAKQAAQEAVEKAPAPQPAPAEQEAKHQAQEAVEKAPQPEPAPAEKAAKHAGQGQAPGEA

SEQ ID NO: 79 - protein, 252 aa, Synthetic construct
QSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVLGAPQSGSEGASQGAGQPGGEVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSS

SEQ ID NO: 80 - protein, 725 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTGAEGAGQAGGQSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVLGAPQSGSEGASQGAGQPGGEVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSS

SEQ ID NO: 81 - protein, 254 aa, Synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGAPQSGSEGASQGAGQPGGQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 82 - protein, 727 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTGAEGAGQAGGDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGAPQSGSEGASQGAGQPGGQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 83 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 84 - protein, 613 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRAEAAAKAPAPQPAPAEAAAKACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPGAEGAGQAGGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 85 - protein, 129 aa, Synthetic construct [sequence omitted]

SEQ ID NO: 86 - protein, 623 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRAEAAAKAPAPQPAPAEAAAKACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPGAEGAGQAGGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGEGSGWSHPQFEK

SEQ ID NO: 87 - protein, 762 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRAEAAAKAPAPQPAPAEAAAKACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPGAEGAGQAGGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGAQPSEAQATESQGGQPGGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 88 - protein, 766 aa, Synthetic construct
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFNSSSSPPERRVYSWMVVGDFNRAPANLEVALRQEPAVSENTIIIAPTEPTHRSGNILDYAILHDAHLPRREQARERIGASLMLNQLRSQITSDHFPVSFVRDRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGGGGQGGGGEGGGSGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGAQPSEAQATESQGGQPGGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 89 - protein, 613 aa, Synthetic construct
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFNSSSSPPERRVYSWMVVGDFNRAPANLEVALRQEPAVSENTIIIAPTEPTHRSGNILDYAILHDAHLPRREQARERIGASLMLNQLRSQITSDHFPVSFVRDRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGGGGQGGGGEGGGSGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 90 - protein, 770 aa, Synthetic construct
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFNSSSSPPERRVYSWMVVGDFNRAPANLEVALRQEPAVSENTIIIAPTEPTHRSGNILDYAILHDAHLPRREQARERIGASLMLNQLRSQITSDHFPVSFVRDRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGGGGQGGGGEGGGSGKQHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSQEGGAQPSEAQATESQGGQPGGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 91 - protein, 622 aa, Synthetic construct
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFNSSSSPPERRVYSWMVVGDFNRAPANLEVALRQEPAVSENTIIIAPTEPTHRSGNILDYAILHDAHLPRREQARERIGASLMLNQLRSQITSDHFPVSFVRDRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGGAQPSEAQATESQGGQPGGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 92 - protein, 250 aa, Synthetic construct
DIVMTQSPDSLAVSLGERATINCKSSQSLLNSGNLKNYLTWYQQKPGQPPKLLIYWASTRKSGVPDRFSGSGSGTDFTLTISSLQAEDVAVYYCQNDYSYPLTFGGGTKVEIKGAPQSGSEGASQGAGQPGGQVQLVQSGAEVKKPGASVKVSCKASGYTFTGYNMNWVRQAPGQGLEWMGNIDPYYGGTSYNQKFKGRVTMTIDKSTSTVYMELSSLRSEDTAVYYCARMYHGNAFDYWGQGTTVTVSS

SEQ ID NO: 93 - protein, 728 aa, Synthetic construct
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFNSSSSPPERRVYSWMVVGDFNRAPANLEVALRQEPAVSENTIIIAPTEPTHRSGNILDYAILHDAHLPRREQARERIGASLMLNQLRSQITSDHFPVSFVRDRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGGGGEGGGPDIVMTQSPDSLAVSLGERATINCKSSQSLLNSGNLKNYLTWYQQKPGQPPKLLIYWASTRKSGVPDRFSGSGSGTDFTLTISSLQAEDVAVYYCQNDYSYPLTFGGGTKVEIKGAPQSGSEGASQGAGQPGGQVQLVQSGAEVKKPGASVKVSCKASGYTFTGYNMNWVRQAPGQGLEWMGNIDPYYGGTSYNQKFKGRVTMTIDKSTSTVYMELSSLRSEDTAVYYCARMYHGNAFDYWGQGTTVTVSS

SEQ ID NO: 94 - protein, 250 aa, Synthetic construct
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSGAPQSGSEGASQGAGQPGGDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 95 - protein, 728 aa, Synthetic construct
NLSDFKVATWNLQGSSAVNESKWNINVRQLLSGEQGADILMVQEAGSLPSSAVRTSRVIQHGGTPIEEYTWNLGTRSRPNMVYIYYSRLDVGANRVNLAIVSRRQADEAFIVHSDSSVLQSRPAVGIRIGTDVFFTVHALATGGSDAVSLIRNIFTTFNSSSSPPERRVYSWMVVGDFNRAPANLEVALRQEPAVSENTIIIAPTEPTHRSGNILDYAILHDAHLPRREQARERIGASLMLNQLRSQITSDHFPVSFVRDRGGEASGCAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGGGGEGGGPQVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSGAPQSGSEGASQGAGQPGGDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 96 - protein, 730 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRAEAAAKAPAPQPAPAEAAAKACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPGAEGAGQAGGDIVMTQSPDSLAVSLGERATINCKSSQSLLNSGNLKNYLTWYQQKPGQPPKLLIYWASTRKSGVPDRFSGSGSGTDFTLTISSLQAEDVAVYYCQNDYSYPLTFGGGTKVEIKGAPQSGSEGASQGAGQPGGQVQLVQSGAEVKKPGASVKVSCKASGYTFTGYNMNWVRQAPGQGLEWMGNIDPYYGGTSYNQKFKGRVTMTIDKSTSTVYMELSSLRSEDTAVYYCARMYHGNAFDYWGQGTTVTVSS

SEQ ID NO: 97 - protein, 730 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRAEAAAKAPAPQPAPAEAAAKACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPGAEGAGQAGGDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKGAPQSGSEGASQGAGQPGGQVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSS

SEQ ID NO: 98 - protein, 58 aa, Synthetic construct
VDNKFNKEMRNAYWEIALLPNLNNQQKRAFIRSLYDDPSQSANLLAEAKKLNDAQAPK

SEQ ID NO: 99 - protein, 538 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRAEAAAKAPAPQPAPAEAAAKACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPGAEGAGQAGGVDNKFNKEMRNAYWEIALLPNLNNQQKRAFIRSLYDDPSQSANLLAEAKKLNDAQAPK

SEQ ID NO: 100 - protein, 126 aa, Synthetic construct
GSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 101 - protein, 606 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRAEAAAKAPAPQPAPAEAAAKACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPGAEGAGQAGGGSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 102 - protein, 161 aa, Synthetic construct
GSDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTPFDLAIDNGNEDIAEVLQKAAKLN

SEQ ID NO: 103 - protein, 641 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRAEAAAKAPAPQPAPAEAAAKACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPGAEGAGQAGGGSDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTPFDLAIDNGNEDIAEVLQKAAKLN

SEQ ID NO: 104 - protein, 250 aa, Synthetic construct
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSGAPQSGSEGASQGAGQPGGDIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVL

SEQ ID NO: 105 - protein, 730 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRAEAAAKAPAPQPAPAEAAAKACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPGAEGAGQAGGQVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSGAPQSGSEGASQGAGQPGGDIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVL

SEQ ID NO: 106 - protein, 246 aa, Synthetic construct
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKGAPQSGSEGASQGAGQPGGQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 107 - protein, 726 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRAEAAAKAPAPQPAPAEAAAKACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPGAEGAGQAGGDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKGAPQSGSEGASQGAGQPGGQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 108 - protein, 245 aa, Synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGAPQSGSEGASQGAGQPGGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 109 - protein, 725 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRAEAAAKAPAPQPAPAEAAAKACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPGAEGAGQAGGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGAPQSGSEGASQGAGQPGGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 110 - protein, 130 aa, Synthetic construct
QVQLQESGGGLVQPGGSLRLSCAASGRTFSSYAMGWFRQAPGKQREFVAAIRWSGGYTYYTDSVKGRFTISRDNAKTTVYLQMNSLKPEDTAVYYCAATYLSSDYSRYALPQRPLDYDYWGQGTQVTVSS

SEQ ID NO: 111 - protein, 610 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRAEAAAKAPAPQPAPAEAAAKACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPGAEGAGQAGGQVQLQESGGGLVQPGGSLRLSCAASGRTFSSYAMGWFRQAPGKQREFVAAIRWSGGYTYYTDSVKGRFTISRDNAKTTVYLQMNSLKPEDTAVYYCAATYLSSDYSRYALPQRPLDYDYWGQGTQVTVSS

SEQ ID NO: 112 - protein, 125 aa, Synthetic construct
QVQLQESGGGSVQAGGSLKLSCAASGRSFSTYAMGWFRQAPGQDREFVATISWTDSTDYADSVKGRFTISRDNAKNTGYLQMNSLKPEDTAVYYCAADRWASSRRNVDYDYWGQGTQVTVSSHGS

SEQ ID NO: 113 - protein, 605 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRAEAAAKAPAPQPAPAEAAAKACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPGAEGAGQAGGQVQLQESGGGSVQAGGSLKLSCAASGRSFSTYAMGWFRQAPGQDREFVATISWTDSTDYADSVKGRFTISRDNAKNTGYLQMNSLKPEDTAVYYCAADRWASSRRNVDYDYWGQGTQVTVSSHGS

SEQ ID NO: 114 - protein, 247 aa, Synthetic construct
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSGAPQSGSEGASQGAGQPGGDIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIK

SEQ ID NO: 115 - protein, 727 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRAEAAAKAPAPQPAPAEAAAKACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPGAEGAGQAGGQVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSGAPQSGSEGASQGAGQPGGDIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIK

SEQ ID NO: 116 - protein, 774 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRAEAAAKAPAPQPAPAEAAAKACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPGAEGAGQAGGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEAPGEAAPAEATAKQAAQEAVEKAPQAEQEAKHQAQEAVEKAEPAEKAAKHAGQGSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLWTLENAQAQ

SEQ ID NO: 117 - protein, 787 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRAPGEAAPAEATAKQAAQEAVEKAPAPQPAPAEQEAKHQAQEAVEKAPQPEPAPAEKAAKHAGQGQAPGEACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPGAEGAGQAGGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGPAPEPAPAPQPAPAPAGSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLWTLENAQAQ

SEQ ID NO: 118 - protein, 246 aa, Synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGAPQSGSEGASQGAGQPGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 119 - protein, 726 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRAEAAAKAPAPQPAPAEAAAKACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPGAEGAGQAGGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGAPQSGSEGASQGAGQPGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 120 - protein, 745 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRAEAAAKAPAPQPAPAEAAAKACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPGAEGAGQAGGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGSGGGGSGGGSAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQ

SEQ ID NO: 121 - protein, 745 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRAEAAAKAPAPQPAPAEAAAKACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPGAEGAGQAGGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGSGGGGSGGGSAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLWTLENAQAQ

SEQ ID NO: 122 - protein, 734 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRAEAAAKAPAPQPAPAEAAAKACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPGAEGAGQAGGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGSGGGGSGGGSSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQ

SEQ ID NO: 123 - protein, 734 aa, Synthetic construct
NLENYTVATWNLQGSSAINESKWNINVRQLLTGPQAAGILMVQEAGSLPSTAVHTRRMVQPEGVGFPIDEYVWNLGTRSRPNNVYIYYSRLDVGANRVNLAIIARRMASEVFVINSSSSVLTSRPAIGIRIDDDAFFSIHALSSGGADSLSLIQNIHTFFNTEGRRHINWMAVGDFNRAPGRMQEALDSEPGLRNATLIVAPTEPTHRSGGVLDYAVLHNANQTTQNTTVSASIMFNQMRSQITSDHFPVSFVKKRAEAAAKAPAPQPAPAEAAAKACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPGAEGAGQAGGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGSGGGGSGGGSSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLWTLENAQAQ

SEQ ID NO: 124 - protein, 14 aa, Synthetic construct
GGGGSGGGGSGGGS

(19) United States
(12) Patent Application Publication
(10) Pub. No.: US 2026/0297152 A1
(43) Pub. Date: Oct. 1, 2026

(54) METHODS AND COMPOSITIONS FOR TREATING CANCER

(71) Applicant: Omeros Corporation, Seattle, WA (US)

(72) Inventors: Ariel B. Abramov, Seattle, WA (US); George A. Gaitanaris, Seattle, WA (US); Marc A. Gavin, Seattle, WA (US); Heidi LeBlanc, Seattle, WA (US); Zachary Matthew James, Kent, WA (US); Jason O'Neill, Lake Forest Park, WA (US); Peter K. Nollert von Specht, Bainbridge Island, WA (US); Mohammed Oukka, Newcastle, WA (US); Munehisa Yabuki, Seattle, WA (US); Ami Yamamoto, Seattle, WA (US)

(21) Appl. No.: 19/633,095

(22) Filed: Mar. 30, 2026

Related U.S. Application Data
(60) Provisional application No. 63/780,759, filed on Mar. 31, 2025.
(60) Provisional application No. 63/943,809, filed on Dec. 18, 2025.

(51) Int. Cl. C07K 14/55; A61K 38/00; A61P 35/00; C07K 14/535; C07K 14/54; C07K 16/18; C07K 16/28; C07K 16/30; C07K 16/32; C12N 9/22

(52) U.S. Cl. CPC C07K 14/55; A61P 35/00; C07K 14/535; C07K 14/5403; C07K 14/5406; C07K 16/18; C07K 16/28; C07K 16/2803; C07K 16/2863; C07K 16/2878; C07K 16/30; C07K 16/303; C07K 16/32; C12N 9/22; A61K 38/00; C07K 2319/50; C07K 2319/55

(57) ABSTRACT

Provided herein are compositions comprising a toxin-derived domain, a translocation domain derived from diphtheria toxin domain B (DTB), and one or more targeting moieties that bind a cancer-related surface antigen or receptor. The toxin domain has DNase activity and is linked to the translocation domain by a protease-cleavable linker. Also provided are methods of using the compositions for the treatment of cancer.

### CROSS REFERENCE TO RELATED APPLICATIONS

This application claims the benefit of U.S. Provisional Application No. 63/780,759, filed Mar. 31, 2025, and claims the benefit of U.S. Provisional Application No. 63/943,809, filed Dec. 18, 2025, the disclosures of both of which are hereby incorporated by reference in their entirety.

### FIELD OF THE INVENTION

The present invention relates to the field of medicine and biotechnology; specifically, to compositions useful for treating cancer and methods of use thereof.

### STATEMENT REGARDING SEQUENCE LISTING

The sequence listing associated with this application is provided in XML format in lieu of a paper copy and is hereby incorporated by reference in its entirety into the specification. The name of the XML file containing the sequence listing is: CT_1_0358_US_SequenceListing_20260324. The XML file is 11,259,138 bytes; was created on Mar. 24, 2026; and is being submitted electronically via the Patent Center with the filing of the specification.

### BACKGROUND

An estimated 1.9 million new cancer cases are diagnosed annually in the U.S. and approximately 16.9 million Americans are living with a history of cancer. The incidence of cancer is expected to increase in upcoming years as the population ages, further augmenting the impact of this condition and the related financial burden.

Cancer arises from mutations that cause defects in the cellular mechanisms that normally control cell growth and preserve genome integrity. Currently, cancer treatment varies based on the type, stage, genetic profile, and patient's overall health. The main treatment strategies include surgery, chemotherapy, radiation therapy, targeted therapy, immunotherapy, hormonal therapy, and stem cell transplantation. Many cancers require a multimodal approach for better outcomes.

Significant progress has been made within oncology through targeted therapies, advanced biologics (e.g., antibody-drug conjugates and radio ligands), and immuno-therapies (e.g., checkpoint inhibitors and chimeric antigen receptor (CAR) T-cell therapies). While these therapies have shown some efficacy, they also carry significant toxicity which impacts the patient's quality of life, especially for older patients and patients with associated co-morbidities. Chemotherapy targets rapidly dividing cells, which includes healthy cells as well as cancer cells, especially in the bone marrow, digestive tract, hair follicles, and reproductive system. The side effects of chemotherapy due to killing of healthy cells vary depending on the type, dose, and duration of chemotherapy. In particular, chemotherapy damages blood-forming cells in the bone marrow, leading to neutropenia (low white blood cell count), which results in increased risk of infections, anemia (low red blood cell count), which results in fatigue, weakness, and shortness of breath, and thrombocytopenia (low platelet count), which results in increased risk of bleeding. Some chemotherapies (e.g., alkylating agents, etoposide) increase the risk of secondary leukemias such as acute myeloid leukemia (AML) and myelodysplastic syndromes (MDS), while alkylating agents like cyclophosphamide and ifosfamide can cause permanent infertility. Chemotherapies can also cause undesirable hormonal changes in both men and women.

An attempt at improved treatment has been made using antibody-drug conjugates (ADCs), which combine a chemotherapy agent with an antibody that binds a cancer-related antigen in a single molecule. ADCs have shown some promise, but have some significant challenges as well. For example, the chemotherapy agent may be cleaved from the antibody before the antibody binds to the target antigen, thereby releasing the chemotherapy agent into the patient's bloodstream. This causes a high level of toxicity that limits the amount of ADC that can be administered. Because the chemotherapy agent itself is typically not specific to a particular cell type, the therapeutic window for ADCs, i.e., the difference between the efficacious dose and the toxic dose, is very narrow. In the case of some attempts to develop ADCs, the efficacious dose overlaps with the toxic dose, such that there is no dose that is both safe and efficacious.

There is a need for improved treatments that can precisely target actively dividing cancer cells without affecting normal cells, even actively dividing normal cells. Such a treatment would significantly limit side effects and provide opportunities for more efficacious and lasting treatments.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Provided herein are molecules comprising a toxin domain, an intracellular translocation domain, and one or more targeting moieties that bind a cancer-related surface antigen or receptor, wherein the toxin domain is linked to the translocation domain by a protease-cleavable linker, wherein the translocation domain is capable of translocating the toxin domain from within the endosome, across the endosomal membrane, and into the cytosol of a cell, and wherein the toxin domain has DNase activity.

In some embodiments, the toxin domain is derived from cytolethal distending toxin (CDT). In some embodiments, the CDT-derived domain is derived from a CdtB domain. In some embodiments, the CDT-derived domain is derived from a CDT from a Haemophilus species, an Escherichia species, an Aggregatibacter species, or a Salmonella species. In some embodiments, the CDT-derived domain is derived from a CDT from Haemophilus ducreyi, Haemophilus parasuis, Escherichia coli, Aggregatibacter actinomycetemcomitans, or Salmonella enterica.

In some embodiments, the translocation domain is derived from diphtheria toxin domain B (DTB).

In some embodiments, the molecules provided herein comprise one or more targeting moieties that allow the toxic effects of the molecule to be targeted to specific cell types. In some embodiments, the targeted cell type is cancer cells. The targeted cells may be hematological cancer cells or non-hematological cancer cells.

In some embodiments, the molecules provided herein comprise multiple domains, any of which may be connected by a linker. In some embodiments, the linker is protease-cleavable. In some embodiments, the linker is non-cleavable. In some embodiments, the molecules provided herein comprise a toxin domain linked to a translocation domain by a protease-cleavable linker. In some embodiments, the molecules provided herein comprise a CDT-derived toxin domain linked to a DTB-derived translocation domain by a furin protease-cleavable linker, further linked to one or more targeting moieties.

In some embodiments, the molecules provided herein may be used for the treatment of cancer.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a diagram of the structure of a molecule of one embodiment of the present disclosure. In this embodiment, the molecule comprises a toxin domain, linked by a furin-cleavable linker to a translocation domain, which is then linked to a targeting moiety.

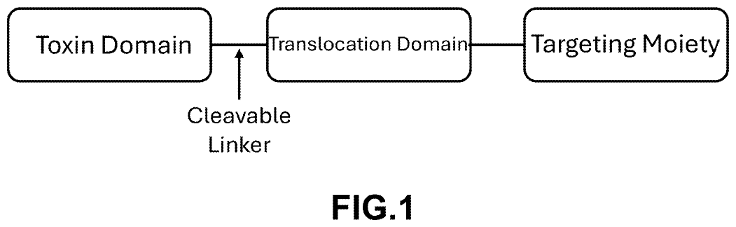

FIG. 2 is a diagram showing how the molecule of one embodiment of the present disclosure is hypothesized to kill a targeted cell. Without wishing to be bound by theory, it is hypothesized that the molecule binds to a cell surface antigen or receptor (A) via a targeting moiety and is internalized by endocytosis (B). At endosomal pH, the translocation domain inserts in the membrane and translocates the toxin domain out of the endosome. The linkage between the translocation domain and the toxin domain is cleaved (C). The toxin domain enters the nucleus and damages the cellular DNA (D), resulting in apoptosis and cell death.

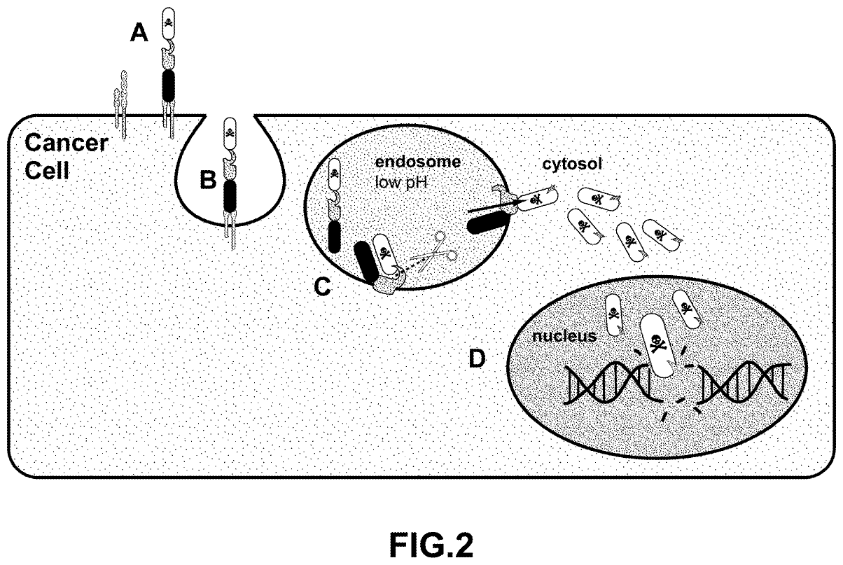

FIG. 3 shows the results of an in vitro cytotoxicity assay for one molecule of the present invention, with or without an N-terminal tag. Molecule VCID0719 (SEQ ID NO:56) comprises, from N-terminus to C-terminus, a CDT-derived domain, a furin-cleavable linker, a DTB-derived translocation domain, and a GM-CSF-derived targeting moiety. This molecule also includes an N-terminal tag for production purposes. A variant was also prepared in which the N-terminal tag was cleaved from the purified molecule (VCID0719 N-terminal tag removed; SEQ ID NO:57). For the cytotoxicity assay, briefly, AML cell lines U-937 or TF-1a were exposed to different concentrations of VCID0719 either with or without the N-terminal tag. After five days, cell viability of each well was determined by CellTiter-Glo®. When the N-terminal tag was removed after purification, cell killing activity increased in both U-937 cells (top panel) and TF-1a cells (bottom panel).

FIG. 4 shows the results of an in vitro cytotoxicity assay for one molecule of the present invention. Molecule VCID0230 (SEQ ID NO:31) comprises, from N-terminus to C-terminus, a CDT-derived domain, furin-cleavable linker, a DTB-derived translocation domain, and a GM-CSF-derived targeting moiety. Briefly, cells from AML cancer cell lines U-937, TF-1a, MV4-11, or MOLM-13 were exposed to different concentrations of VCID0230 in 96-well plates. After five days, cell viability of each well was determined by CellTiter-Glo®. Each cell line tested showed greater than 50% killing at one or more of the concentrations tested.

FIG. 5 shows the results of an in vitro cytotoxicity assay for one molecule of the present invention. Molecule VCID0904 (SEQ ID NO:42) comprises, from N-terminus to C-terminus, a CDT-derived domain, a furin-cleavable linker, a DTB-derived translocation domain, a GM-CSF-derived targeting moiety, and an IL3-derived targeting moiety. Briefly, cells from AML cancer cell lines U-937, TF-1a, MV4-11, or MOLM-13 were exposed to different concentrations of VCID0904 in 96-well plates. After five days, cell viability of each well was determined by CellTiter-Glo®. Each cell line tested showed greater than 50% killing at one or more of the concentrations tested.

FIG. 6 shows the results of an in vitro cytotoxicity assay for one molecule of the present invention. Molecule VCID0231 (SEQ ID NO:38) comprises, from N-terminus to C-terminus, a CDT-derived domain, a furin-cleavable linker, a DTB-derived translocation domain, and an anti-CD33-derived targeting moiety. Briefly, cells from AML cancer cell lines U-937, TF-1a, or MOLM-13 were exposed to different concentrations of VCID0231 in 96-well plates. After five days, cell viability of each well was determined by CellTiter-Glo®. Each cell line tested showed greater than 50% killing at one or more of the concentrations tested.

FIG. 7 shows the results of an in vivo study using a human tumor xenograft model in mice. Molecule VCID0230 (SEQ ID NO:31) comprises, from N-terminus to C-terminus, a CDT-derived domain, a furin-cleavable linker, a DTB-derived translocation domain, and a GM-CSF-derived targeting moiety. Briefly, luciferase-expressing cells from human AML cancer cell line U-937 were infused into NSG mice by tail vein injection to create a disseminated in vivo tumor model in mice. Beginning five days after cell infusion, mice were treated with different doses of VCID0230 daily for five days. Tumor burden was monitored through in vivo imaging (IVIS) of the luciferase signal. Average tumor burden for the treated mice was notably lower than for untreated mice at all doses tested.

FIG. 8 shows the results of an in vivo study using a human tumor xenograft model in mice. Experimental details are the same as described for FIG. 7, except that mice were dosed at 50 μg/day, which is approximately 2.5 mg/kg. Average tumor burden for the VCID0230-treated mice was notably lower than for vehicle-treated mice.

FIG. 9 shows the results of an in vivo study using a human tumor xenograft model in mice. Experimental details are the same as described for FIG. 7, except that the cells infused were luciferase-expressing cells from AML cancer cell line TF-1a and mice were dosed at 50 μg/day, which is approximately 2.5 mg/kg. Average tumor burden for the VCID0230-treated mice was notably lower than for vehicle-treated mice.

FIG. 10 shows the results of an in vivo study using a human tumor xenograft model in mice. Experimental details are the same as described for FIG. 7, except that the cells infused were luciferase-expressing cells from cancer cell line MOLM-13 and treatment began one day after cell infusion. Average tumor burden for the VCID0230-treated mice was notably lower than for vehicle-treated mice.

FIG. 11 shows the results of an in vivo study using a human tumor xenograft model in mice. Experimental details are the same as described for FIG. 7, except that the cells infused were luciferase-expressing cells from cancer cell line MOLM-13, treatment began one day after cell infusion and mice were dosed at 200 μg/day, which is approximately 10 mg/kg. Average tumor burden for the VCID0230-treated mice was notably lower than for vehicle-treated mice.

FIG. 12 shows the survival time plot for the experiment described in FIG. 11. Compared to vehicle-treated mice, median survival of VCID0230-treated mice increased from 19 days to 25 days post cell infusion.

FIG. 13 shows the results of an in vivo study using a human tumor xenograft model in mice. Experimental details are the same as described for FIG. 7, except that the cells infused were luciferase-expressing cells from cancer cell line MV4-11 and treatment began ten days after cell infusion. Average tumor burden for the VCID0230-treated mice was notably lower than for vehicle-treated mice.

FIG. 14 shows the results of an in vivo study using a human tumor xenograft model in mice. Molecule VCID0904 (SEQ ID NO:42) comprises, from N-terminus to C-terminus, a CDT-derived domain, a furin-cleavable linker, a DTB-derived translocation domain, a GM-CSF-derived targeting moiety, and an IL3-derived targeting moiety. Briefly, luciferase-expressing cells from AML cancer cell line U-937 were infused into NSG mice by tail vein injection to create a disseminated in vivo tumor model in mice. Beginning five days after cell infusion, mice were treated with 50 μg of VCID0904 (approximately 2.5 mg/kg) daily for five days. Tumor burden was monitored through in vivo imaging as previously described. Average tumor burden for the VCID0904-treated mice was notably lower than for vehicle-treated mice.

FIG. 15 shows the results of an in vivo study using a human tumor xenograft model in mice. Experimental details are the same as described for FIG. 14, except that the cells infused were luciferase-expressing cells from cancer cell line MOLM-13, and mice were treated with different doses beginning one day after cell infusion. Average tumor burden for the VCID0904-treated mice was notably lower than for vehicle-treated mice.

FIG. 16 shows the results of an in vivo study using a human tumor xenograft model in mice. Experimental details are the same as described for FIG. 14, except that the cells infused were luciferase-expressing cells from cancer cell line MOLM-13 and VCID0904 was administered at a dosage of 200 μg (approximately 10 mg/kg) beginning one day after cell infusion. Average tumor burden for the VCID0904-treated mice was notably lower than for vehicle-treated mice.

FIG. 17 shows the survival time plot for the experiment described in FIG. 16. Compared to vehicle-treated mice, median survival of VCID0904-treated mice increased from 19 days to greater than 40 days post cell infusion.

FIG. 18 shows the results of an in vivo study using a human tumor xenograft model in mice. Experimental details are the same as described for FIG. 14, except that the cells infused were luciferase-expressing cells from cancer AML cell line MV4-11 and mice were treated with different doses beginning ten days after cell infusion. Average tumor burden for the VCID0904-treated mice was notably lower than for vehicle-treated mice.

FIG. 19 shows the results of an in vivo study using a human tumor xenograft model in mice. Molecule VCID0231 (SEQ ID NO:38) comprises, from N-terminus to C-terminus, a CDT-derived domain, a furin-cleavable linker, a DTB-derived translocation domain, and an anti-CD33-derived targeting moiety. Briefly, luciferase-expressing cells from human AML cancer cell line U-937 were infused into NSG mice by tail vein injection to create a disseminated in vivo tumor model in mice. Beginning five days after cell infusion, mice were treated with 100 μg of VCID0231 (approximately 5 mg/kg) daily for five days. Tumor burden was monitored through in vivo imaging as previously described. Average tumor burden for the VCID0231-treated mice was notably lower than for vehicle-treated mice.

FIG. 20 shows the results of an in vitro cytotoxicity assay for one molecule of the present invention as compared to a control molecule. Molecule VCID0230 (SEQ ID NO:31) comprises, from N-terminus to C-terminus, a CDT-derived domain, a furin-cleavable linker, a DTB-derived translocation domain, and a GM-CSF-derived targeting moiety. Molecule VCID0539 (SEQ ID NO:52) is similar to VCID0230, but incorporates a linker having reduced furin cleavage between the CDT-derived domain and the DTB-derived translocation domain. Briefly, cells from AML cancer cell line U-937 (top) or TF-1a (bottom) were exposed to different concentrations of VCID0230 or VCID0539 in 96-well plates. After five days, relative viability of cells in each well was determined by CellTiter-Glo®. VCID0539 showed markedly less cell killing activity than VCID0230.

FIG. 21 shows the results of an in vitro cytotoxicity assay for one molecule of the present invention as compared to a control molecule. Molecule VCID0230 (SEQ ID NO:31) comprises, from N-terminus to C-terminus, a CDT-derived domain, a furin-cleavable linker, a DTB-derived translocation domain, and a GM-CSF-derived targeting moiety. Molecule VCID1172 (SEQ ID NO:54) is similar to VCID0230, but does not include a DTB-derived translocation domain and lacks the cysteines bounding the furin domain. Briefly, cells from cancer cell line U-937 (top) or MOLM-13 (bottom) were exposed to different concentrations of VCID0230 or VCID1172 in 96-well plates. After five days, relative viability of cells in each well was determined. VCID1172 showed markedly less cell killing activity than VCID0230.

FIG. 22 shows the results of an in vitro cytotoxicity assay for one molecule of the present invention as compared to a control molecule. Molecule VCID0230 (SEQ ID NO:31) comprises, from N-terminus to C-terminus, a CDT-derived domain, a furin-cleavable linker, a DTB-derived translocation domain, and a GM-CSF-derived targeting moiety. Molecule VCID1170 (SEQ ID NO:55) is similar to VCID0230, but does not include either a furin-cleavable linker or a DTB-derived translocation domain. Briefly, cells from cancer cell line U-937 (top) or MOLM-13 (bottom) were exposed to different concentrations of VCID0230 or VCID1170 in 96-well plates. After five days, relative viability of cells in each well was determined. VCID1170 showed markedly less cell killing activity than VCID0230.

FIG. 23 shows the results of an in vitro cytotoxicity assay for one molecule of the present invention. Molecule VCID1726 (SEQ ID NO:61) comprises, from N-terminus to C-terminus, a CDT-derived domain, a furin-cleavable linker, a DTB-derived translocation domain, a GM-CSF-derived targeting moiety, and an IL3-derived targeting moiety. Briefly, cells from cancer cell lines U-937, TF-1a, MOLM-13, and MV4-11 were exposed to different concentrations of VCID1726 in 96-well plates. After five days, relative viability of cells in each well was determined. IC₅₀ values for each cell line were calculated from non-linear fitted data. VCID1726 showed killing of all tested cell lines with IC₅₀ values in the picomolar to sub-picomolar range.

FIG. 24 shows the results of an in vitro cytotoxicity assay for one molecule of the present invention. Molecule VCID1728 (SEQ ID NO:62) comprises, from N-terminus to C-terminus, a CDT-derived domain, a furin-cleavable linker, a DTB-derived translocation domain, a GM-CSF-derived targeting moiety, and an IL3-derived targeting moiety. Briefly, cells from cancer cell lines U-937, TF-1a, MOLM-13, and MV4-11 were exposed to different concentrations of VCID1728 in 96-well plates. After five days, relative viability of cells in each well was determined. IC₅₀ values for each cell line were calculated from non-linear fitted data. VCID1728 showed killing of all tested cell lines with IC₅₀ values in the picomolar to sub-picomolar range.

FIG. 25 shows the results of an in vivo study using a human tumor xenograft model in mice. Molecule VCID1726 (SEQ ID NO:61) comprises, from N-terminus to C-terminus, a CDT-derived domain, a furin-cleavable linker, a DTB-derived translocation domain, a GM-CSF-derived targeting moiety, and an IL3-derived targeting moiety. Briefly, luciferase-expressing cells from AML cancer cell line MOLM-13 were infused into NSG mice to create a mouse tumor model. Beginning three days after cell infusion, mice were treated with 0.025 mg/kg of VCID1726 daily for five days. The top panel shows average tumor burden as monitored through in vivo imaging, as previously described. Tumor burden is well controlled in VCID1726-treated mice as compared to vehicle-treated mice. The bottom panel shows survival of VCID1726-treated mice as compared to vehicle-treated mice. The VCID1726-treated mice showed a higher probability of survival over an extended period as compared to vehicle-treated mice.

FIG. 26 shows the results of an in vivo study using a human tumor xenograft model in mice. Molecule VCID1728 (SEQ ID NO:62) comprises, from N-terminus to C-terminus, a CDT-derived domain, a furin-cleavable linker, a DTB-derived translocation domain, a GM-CSF-derived targeting moiety, and an IL3-derived targeting moiety. Briefly, luciferase-expressing cells from AML cancer cell line MOLM-13 were infused into NSG mice to create a mouse tumor model. Beginning three days after cell infusion, mice were treated with 0.025 mg/kg of VCID1728 daily for five days. The top panel shows average tumor burden as monitored through in vivo imaging, as previously described. Tumor burden is well controlled in VCID1728-treated mice as compared to vehicle-treated mice. The bottom panel shows survival of VCID1728-treated mice as compared to vehicle-treated mice. The VCID1728-treated mice showed 100% survival over an extended period (longer than 50 days) as compared to vehicle-treated mice, which did not survive past 20 days.

FIG. 27 shows the results of an in vitro cytotoxicity assay for one molecule of the present invention. Molecule VCID1694 (SEQ ID NO:80) comprises, from N-terminus to C-terminus, a CDT-derived domain, a furin-cleavable linker, a DTB-derived translocation domain, and an anti-BCMA-derived targeting moiety. Briefly, cells from the Ramos cancer cell line or the RPMI-8226 cancer cell line were exposed to different concentrations of VCID1694 in 96-well plates. After five days, relative viability of cells in each well was determined. IC₅₀ values were calculated from non-linear fitted data.

FIG. 28 shows the results of an in vivo study using a human tumor xenograft model in mice. Molecule VCID1694 (SEQ ID NO:80) comprises, from N-terminus to C-terminus, a CDT-derived domain, a furin-cleavable linker, a DTB-derived translocation domain, and an anti-BCMA-derived targeting moiety. Briefly, luciferase-expressing Ramos cells were infused into NSG mice to create a mouse disseminated in vivo tumor model. in mice. Beginning five days after cell infusion, mice were treated with 1 mg/kg or 5 mg/kg of VCID1694 daily for five days. The top panel shows average tumor burden as monitored through in vivo imaging, as previously described. Tumor burden is reduced in VCID1694-treated mice as compared to vehicle-treated mice. The bottom panel shows survival of VCID1694-treated mice as compared to vehicle-treated mice. The VCID1694-treated mice showed longer survival as compared to vehicle-treated mice.

FIG. 29 shows the results of an in vitro cytotoxicity assay for one molecule of the present invention. Molecule VCID1700 (SEQ ID NO:82) comprises, from N-terminus to C-terminus, a CDT-derived domain, a furin-cleavable linker, a DTB-derived translocation domain, and an anti-CD19-derived targeting moiety. Briefly, cells from the Ramos or Daudi cancer cell lines were exposed to different concentrations of VCID1700 in 96-well plates. After five days, relative viability of cells in each well was determined. IC₅₀ values for each cell line were calculated from non-linear fitted data.

FIG. 30 shows the results of an in vivo study using a human tumor xenograft model in mice. Molecule VCID1700 (SEQ ID NO:82) comprises, from N-terminus to C-terminus, a CDT-derived domain, a furin-cleavable linker, a DTB-derived translocation domain, and an anti-BCMA-derived targeting moiety. Briefly, luciferase-expressing Ramos cells were infused into NSG mice to create a mouse disseminated in vivo tumor model. Beginning five days after cell infusion, mice were treated with 1 mg/kg or 5 mg/kg of VCID1694VCID1700 daily for five days. The top panel shows average tumor burden as monitored through in vivo imaging, as previously described. Tumor burden is well controlled in VCID1700-treated mice as compared to vehicle-treated mice. The bottom panel shows survival of VCID1700-treated mice as compared to vehicle-treated mice. The VCID1700-treated mice showed 100% survival over an extended period (longer than 50 days) as compared to vehicle-treated mice, which did not survive past 28 days.

FIG. 31 shows the results of an in vitro cytotoxicity assay for three molecules of the present invention. Molecule VCID1572 (SEQ ID NO:88) comprises, from N-terminus to C-terminus, a CDT-derived domain, a furin-cleavable linker, a DTB-derived translocation domain, an IL4-derived targeting moiety, and an IL2-derived targeting moiety. Molecule VCID1573 (SEQ ID NO:89) comprises, from N-terminus to C-terminus, a CDT-derived domain, a furin-cleavable linker, a DTB-derived translocation domain, and an IL4-derived targeting moiety. Molecule VCID1219 (SEQ ID NO:91) comprises, from N-terminus to C-terminus, a CDT-derived domain, a furin-cleavable linker, a DTB-derived translocation domain, and an IL2-derived targeting moiety. Briefly, cells from the SU-DHL-1 cancer cell line were exposed to different concentrations of one of the tested molecules in 96-well plates. After five days, relative viability of cells in each well was determined. IC₅₀ values for each molecule were calculated from non-linear fitted data.

FIG. 32 shows the results of an in vitro cytotoxicity assay for two molecules of the present invention. Molecule VCID1800 (SEQ ID NO:87) comprises, from N-terminus to C-terminus, a CDT-derived domain, a furin-cleavable linker, a DTB-derived translocation domain, an IL4-derived targeting moiety, and an IL2-derived targeting moiety. Molecule VCID1801 (SEQ ID NO:86) comprises, from N-terminus to C-terminus, a CDT-derived domain, a furin-cleavable linker, a DTB-derived translocation domain, and an IL4-derived targeting moiety. Briefly, cells from the Ramos or Daudi cancer cell lines were exposed to different concentrations of VCID1800 or VCID1801 in 96-well plates. After five days, relative viability of cells in each well was determined. IC₅₀ values for each cell line were calculated from non-linear fitted data.

FIG. 33 shows the results of an in vitro cytotoxicity assay for one molecule of the present invention. Molecule VCID1418 (SEQ ID NO:93) comprises, from N-terminus to C-terminus, a CDT-derived domain, a furin-cleavable linker, a DTB-derived translocation domain, and an anti-CLDN18.2-derived targeting moiety. Briefly, cells from a HEK293 cancer cell line engineered to express CLDN18.2 were exposed to different concentrations of VCID1418 in 96-well plates. Plates were immediately placed inside an imaging incubator for live cell imaging. Images were taken every 12 hours and analyzed for % confluence. Top panel shows % confluence at tested timepoints. Bottom panel shows dose curve at 72 hours. IC₅₀ values were calculated from non-linear fitted data.

FIG. 34 shows the results of an in vitro cytotoxicity assay for one molecule of the present invention. Molecule VCID1419 (SEQ ID NO:93) comprises, from N-terminus to C-terminus, a CDT-derived domain, a furin-cleavable linker, a DTB-derived translocation domain, and an anti-CLDN18.2-derived targeting moiety. Briefly, cells from a HEK293 cancer cell line engineered to express CLDN18.2 were exposed to different concentrations of VCID1419 in 96-well plates. Plates were immediately placed inside an imaging incubator for live cell imaging. Images were taken every 12 hours and analyzed for % confluence. Top panel shows % confluence at tested timepoints. Bottom panel shows dose curve at 72 hours. IC₅₀ values were calculated from non-linear fitted data.

FIG. 35 shows the results of an in vitro cytotoxicity assay for one molecule of the present invention. Molecule VCID1793 (SEQ ID NO:96) comprises, from N-terminus to C-terminus, a CDT-derived domain, a furin-cleavable linker, a DTB-derived translocation domain, and an anti-CLDN18.2-derived targeting moiety. Briefly, cells from a HEK293 cancer cell line engineered to express CLDN18.2 were exposed to different concentrations of VCID1793 in 96-well plates. Plates were immediately placed inside an imaging incubator for live cell imaging. Images were taken every 12 hours and analyzed for % confluence. Top panel shows % confluence at tested timepoints. Bottom panel shows dose curve at 72 hours. IC₅₀ values were calculated from non-linear fitted data.

FIG. 36 shows the results of an in vitro cytotoxicity assay for one molecule of the present invention. Molecule VCID1794 (SEQ ID NO:97) comprises, from N-terminus to C-terminus, a CDT-derived domain, a furin-cleavable linker, a DTB-derived translocation domain, and an anti-CLDN18.2-derived targeting moiety. Briefly, cells from a HEK293 cancer cell line engineered to express CLDN18.2 were exposed to different concentrations of VCID1794 in 96-well plates. Plates were immediately placed inside an imaging incubator for live cell imaging. Images were taken every 12 hours and analyzed for % confluence. Top panel shows % confluence at tested timepoints. Bottom panel shows dose curve at 72 hours. IC₅₀ values were calculated from non-linear fitted data.

FIG. 37 shows the results of an in vitro cytotoxicity assay for one molecule of the present invention. Molecule VCID1896 (SEQ ID NO:99) comprises, from N-terminus to C-terminus, a CDT-derived domain, a furin-cleavable linker, a DTB-derived translocation domain, and an anti-HER2-derived targeting moiety. Briefly, cells from a HEK293 cancer cell line engineered to express HER2 were exposed to different concentrations of VCID1896 in 96-well plates. Plates were immediately placed inside an imaging incubator for live cell imaging. Images were taken every 12 hours and analyzed for % confluence. Top panel shows % confluence at tested timepoints. Bottom panel shows dose curve at 72 hours. IC₅₀ values were calculated from non-linear fitted data.

FIG. 38 shows the results of an in vitro cytotoxicity assay for one molecule of the present invention. Molecule VCID1898 (SEQ ID NO:101) comprises, from N-terminus to C-terminus, a CDT-derived domain, a furin-cleavable linker, a DTB-derived translocation domain, and an anti-HER2-derived targeting moiety. Briefly, cells from a HEK293 cancer cell line engineered to express HER2 were exposed to different concentrations of VCID1898 in 96-well plates. Plates were immediately placed inside an imaging incubator for live cell imaging. Images were taken every 12 hours and analyzed for % confluence. Top panel shows % confluence at tested timepoints. Bottom panel shows dose curve at 72 hours. IC₅₀ values were calculated from non-linear fitted data.

FIG. 39 shows the results of an in vitro cytotoxicity assay for one molecule of the present invention. Molecule VCID1899 (SEQ ID NO:103) comprises, from N-terminus to C-terminus, a CDT-derived domain, a furin-cleavable linker, a DTB-derived translocation domain, and an anti-HER2-derived targeting moiety. Briefly, cells from a HEK293 cancer cell line engineered to express HER2 were exposed to different concentrations of VCID1899 in 96-well plates. Plates were immediately placed inside an imaging incubator for live cell imaging. Images were taken every 12 hours and analyzed for % confluence. Top panel shows % confluence at tested timepoints. Bottom panel shows dose curve at 72 hours. IC₅₀ values were calculated from non-linear fitted data.

FIG. 40 shows the results of an in vitro cytotoxicity assay for one molecule of the present invention. Molecule VCID1862 (SEQ ID NO:105) comprises, from N-terminus to C-terminus, a CDT-derived domain, a furin-cleavable linker, a DTB-derived translocation domain, and an anti-MSLN-derived targeting moiety. Briefly, cells from a HEK293 cancer cell line engineered to express MSLN were exposed to different concentrations of VCID1862 in 96-well plates. Plates were immediately placed inside an imaging incubator for live cell imaging. Images were taken every 12 hours and analyzed for % confluence. Top panel shows % confluence at tested timepoints. Bottom panel shows dose curve at 72 hours. IC₅₀ values were calculated from non-linear fitted data.

FIG. 41 shows the results of an in vitro cytotoxicity assay for one molecule of the present invention. Molecule VCID1861 (SEQ ID NO:107) comprises, from N-terminus to C-terminus, a CDT-derived domain, a furin-cleavable linker, a DTB-derived translocation domain, and an anti-GPC3-derived targeting moiety. Briefly, cells from a HEK293 cancer cell line engineered to express GPC3 were exposed to different concentrations of VCID1861 in 96-well plates. Plates were immediately placed inside an imaging incubator for live cell imaging. Images were taken every 12 hours and analyzed for % confluence. Top panel shows % confluence at tested timepoints. Bottom panel shows dose curve at 72 hours. IC₅₀ values were calculated from non-linear fitted data.

FIG. 42 shows the results of an in vitro cytotoxicity assay for one molecule of the present invention. Molecule VCID1795 (SEQ ID NO:109) comprises, from N-terminus to C-terminus, a CDT-derived domain, a furin-cleavable linker, a DTB-derived translocation domain, and an anti-EGFR-derived targeting moiety. Briefly, cells from a HEK293 cancer cell line engineered to express EGFR were exposed to different concentrations of VCID1795 in 96-well plates. Plates were immediately placed inside an imaging incubator for live cell imaging. Images were taken every 12 hours and analyzed for % confluence. Top panel shows % confluence at tested timepoints. Bottom panel shows dose curve at 72 hours. IC₅₀ values were calculated from non-linear fitted data.

FIG. 43 shows the results of an in vitro cytotoxicity assay for one molecule of the present invention. Molecule VCID1906 (SEQ ID NO:111) comprises, from N-terminus to C-terminus, a CDT-derived domain, a furin-cleavable linker, a DTB-derived translocation domain, and an anti-EGFR-derived targeting moiety. Briefly, cells from a HEK293 cancer cell line engineered to express EGFR were exposed to different concentrations of VCID1906 in 96-well plates. Plates were immediately placed inside an imaging incubator for live cell imaging. Images were taken every 12 hours and analyzed for % confluence. Top panel shows % confluence at tested timepoints. Bottom panel shows dose curve at 72 hours. IC₅₀ values were calculated from non-linear fitted data.

FIG. 44 shows the results of an in vitro cytotoxicity assay for one molecule of the present invention. Molecule VCID1907 (SEQ ID NO:113) comprises, from N-terminus to C-terminus, a CDT-derived domain, a furin-cleavable linker, a DTB-derived translocation domain, and an anti-EGFR-derived targeting moiety. Briefly, cells from a HEK293 cancer cell line engineered to express EGFR were exposed to different concentrations of VCID1907 in 96-well plates. Plates were immediately placed inside an imaging incubator for live cell imaging. Images were taken every 12 hours and analyzed for % confluence. Top panel shows % confluence at tested timepoints. Bottom panel shows dose curve at 72 hours. IC₅₀ values were calculated from non-linear fitted data.

FIG. 45 shows the results of an in vitro cytotoxicity assay for one molecule of the present invention. Molecule VCID1880 (SEQ ID NO:115) comprises, from N-terminus to C-terminus, a CDT-derived domain, a furin-cleavable linker, a DTB-derived translocation domain, and an anti-TROP2-derived targeting moiety. Briefly, cells from a HEK293 cancer cell line engineered to express TROP2 were exposed to different concentrations of VCID1880 in 96-well plates. Plates were immediately placed inside an imaging incubator for live cell imaging. Images were taken every 12 hours and analyzed for % confluence. Top panel shows % confluence at tested timepoints. Bottom panel shows dose curve at 72 hours. IC₅₀ values were calculated from non-linear fitted data.

FIG. 46 shows the results of an in vivo study using a human tumor xenograft model in mice. Molecule VCID1793 (SEQ ID NO:96) comprises, from N-terminus to C-terminus, a CDT-derived domain, a furin-cleavable linker, a DTB-derived translocation domain, and an anti-CLDN18.2-derived targeting moiety. Briefly, HEK293 cells engineered to express CLDN18.2 were transplanted subcutaneously into NSG mice to create a solid tumor in vivo model. Beginning three days after cell transplantation, mice were treated with 5 mg/kg of VCID1793 daily for two weeks with a 5 days on, two days off cycle. Tumor size was monitored twice weekly by caliper measurements. Estimated tumor volume was calculated by V=0.5×L×W², where V is estimated tumor volume, L is longest tumor dimension, and W is dimension perpendicular to L. Tumor burden is well controlled in VCID1793-treated mice as compared to vehicle-treated mice.

FIG. 47 shows the results of an in vivo study using a human tumor xenograft model in mice. Molecule VCID1797 (SEQ ID NO:119) comprises, from N-terminus to C-terminus, a CDT-derived domain, a furin-cleavable linker, a DTB-derived translocation domain, and an anti-HER2-derived targeting moiety. Briefly, HEK293 cells engineered to express HER2 were transplanted subcutaneously into NSG mice to create a solid tumor in vivo model. Beginning three days after cell transplantation, mice were treated with 5 mg/kg of VCID1797 daily for two weeks with a 5 days on, two days off cycle. Tumor size was monitored twice weekly by caliper measurements. Estimated tumor volume was calculated by V=0.5×L×W², where V is estimated tumor volume, L is longest tumor dimension, and W is dimension perpendicular to L. Tumor burden is well controlled in VCID1797-treated mice as compared to vehicle-treated mice.

FIG. 48 shows the results of an in vivo study using a human tumor xenograft model in mice. Molecule VCID1896 (SEQ ID NO:99) comprises, from N-terminus to C-terminus, a CDT-derived domain, a furin-cleavable linker, a DTB-derived translocation domain, and an anti-HER2-derived targeting moiety. Molecule VCID1898 (SEQ ID NO:101) comprises, from N-terminus to C-terminus, a CDT-derived domain, a furin-cleavable linker, a DTB-derived translocation domain, and an anti-HER2-derived targeting moiety. Briefly, HEK293 cells engineered to express HER2 were transplanted subcutaneously into NSG mice to create a solid tumor in vivo model. Beginning three days after cell transplantation, mice were treated with 5 mg/kg of VCID1896 or VCID1898 daily for two weeks with a 5 days on, two days off cycle. Tumor size was monitored twice weekly by caliper measurements. Estimated tumor volume was calculated by V=0.5×L×W², where V is estimated tumor volume, L is longest tumor dimension, and W is dimension perpendicular to L. Tumor burden is well controlled in VCID1896-treated and VCID1898-treated mice as compared to vehicle-treated mice.

FIG. 49 shows the results of an in vivo study using a human tumor xenograft model in mice. Molecule VCID1862 (SEQ ID NO:105) comprises, from N-terminus to C-terminus, a CDT-derived domain, a furin-cleavable linker, a DTB-derived translocation domain, and an anti-MSLN-derived targeting moiety. Briefly, HEK293 cells engineered to express MSLN were transplanted subcutaneously into NSG mice to create a solid tumor in vivo model. Beginning three days after cell transplantation, mice were treated with 5 mg/kg of VCID1892 daily for two weeks with a 5 days on, two days off cycle. Tumor size was monitored twice weekly by caliper measurements. Estimated tumor volume was calculated by V=0.5×L×W², where V is estimated tumor volume, L is longest tumor dimension, and W is dimension perpendicular to L. Tumor burden is well controlled in VCID1892-treated mice as compared to vehicle-treated mice.

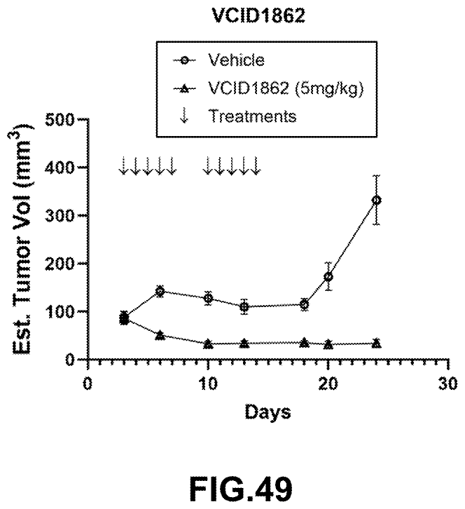

FIG. 50 shows the results of an in vitro cytotoxicity assay for five molecules of the present invention using a cell line that does not express the target antigen. Molecule VCID1793 (SEQ ID NO:96) comprises, from N-terminus to C-terminus, a CDT-derived domain, a furin-cleavable linker, a DTB-derived translocation domain, and an anti-CLDN18.2-derived targeting moiety. Molecule VCID1797 (SEQ ID NO: 119) comprises, from N-terminus to C-terminus, a CDT-derived domain, a furin-cleavable linker, a DTB-derived translocation domain, and an anti-HER2derived targeting moiety. Molecule VCID1896 (SEQ ID NO:99) comprises, from N-terminus to C-terminus, a CDT-derived domain, a furin-cleavable linker, a DTB-derived translocation domain, and an anti-HER2-derived targeting moiety. Molecule VCID1862 (SEQ ID NO:105) comprises, from N-terminus to C-terminus, a CDT-derived domain, a furin-cleavable linker, a DTB-derived translocation domain, and an anti-MSLN-derived targeting moiety. Molecule VCID1861 (SEQ ID NO: 107) comprises, from N-terminus to C-terminus, a CDT-derived domain, a furin-cleavable linker, a DTB-derived translocation domain, and an anti-GPC3-derived targeting moiety. Briefly, cells from a HEK293 cancer cell line were exposed to different concentrations of each of the drugs shown in 96-well plates. Plates were immediately placed inside an imaging incubator for live cell imaging. Images were taken every 12 hours and analyzed for % confluence. Graph shows dose curve at 72 hours. IC₅₀ values were calculated from non-linear fitted data.

FIG. 51 shows the results of an in vitro cytotoxicity assay for five molecules of the present invention. Molecule VCID1950 (SEQ ID NO: 120) comprises from N-terminus to C-terminus, a CDT-derived domain, a furin-cleavable linker, a DTB-derived translocation domain, a GM-CSF-derived targeting moiety, and an IL3-derived targeting moiety. Molecule VCID1951 (SEQ ID NO:121) comprises from N-terminus to C-terminus, a CDT-derived domain, a furin-cleavable linker, a DTB-derived translocation domain, a GM-CSF-derived targeting moiety, and an IL3-derived targeting moiety. Molecule VCID1952 (SEQ ID NO: 122) comprises from N-terminus to C-terminus, a CDT-derived domain, a furin-cleavable linker, a DTB-derived translocation domain, a GM-CSF-derived targeting moiety, and an IL3-derived targeting moiety. Molecule VCID1953 (SEQ ID NO: 123) comprises from N-terminus to C-terminus, a CDT-derived domain, a furin-cleavable linker, a DTB-derived translocation domain, a GM-CSF-derived targeting moiety, and an IL3-derived targeting moiety. Molecule VCID1728 (SEQ ID NO:62) comprises, from N-terminus to C-terminus, a CDT-derived domain, a furin-cleavable linker, a DTB-derived translocation domain, a GM-CSF-derived targeting moiety, and an IL3-derived targeting moiety. Briefly, cells from either the MOLM-13 cancer cell line or the MV4-11 cancer cell line were exposed to different concentrations of one of the tested molecules in 96-well plates. After five days, relative viability of cells in each well was determined. Results for MOLM-13 cells are shown in the top panel. Results for MV4-11 cells are shown in the bottom panel. IC₅₀ values for each molecule were calculated from non-linear fitted data.

FIG. 52 shows the results of an in vitro cytotoxicity assay for one molecule of the present invention. Molecule VCID1728 (SEQ ID NO:62) comprises, from N-terminus to C-terminus, a CDT-derived domain, a furin-cleavable linker, a DTB-derived translocation domain, a GM-CSF-derived targeting moiety, and an IL3-derived targeting moiety. Briefly, cells from cancer cell lines U-937, TF-1a, MOLM-13, and MV4-11 were exposed to different concentrations of VCID1728 in 96-well plates. After five days, relative viability of cells in each well was determined. IC₅₀ values for each cell line were calculated from non-linear fitted data. VCID1728 showed killing of all tested cell lines with IC₅₀ values in the picomolar to sub-picomolar range.

### DETAILED DESCRIPTION

### I. Definitions

Unless specifically defined herein, all terms used herein have the same meaning as would be understood by those of ordinary skill in the art of the present invention. The following definitions are provided in order to provide clarity with respect to the terms as they are used in the specification and claims to describe the present invention. Additional definitions are set forth throughout this disclosure.

In the present descriptions, any concentration range, percentage range, ratio range, or integer range is to be understood to include the value of any integer within the recited range and, when appropriate, fractions thereof (such as one tenth and one hundredth of an integer), unless otherwise indicated or evident from the context. Any number range recited herein relating to any physical feature, such as polymer subunits, size, or thickness, is to be understood to include any integer within the recited range and, when appropriate, fractions thereof, unless otherwise indicated or evident from the context. As used herein, the term “about” is meant to specify that the range or value provided may vary by ±10% of the indicated range or value, unless otherwise indicated.

It should be understood that the terms “a”, “an”, and “the” as used herein refer to one or more of the referenced components. The use of the alternative (e.g., “or”) should be understood to mean either one, both, or any combination of the alternatives. As used herein, the terms “include”, “have”, and “comprise” are used synonymously, which terms and variants thereof are intended to be construed as non-limiting.

“Optional” or “optionally” means that the subsequently described element, component, event, or circumstance may or may not occur, and that the description includes instances in which the element component, event, or circumstance occurs and instances in which it does not. It should be understood that the individual constructs or groups of constructs derived from the various combinations of the structures and subunits described herein are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain) or a protein “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, the terms “treat”, “treatment”, or “ameliorate” refer to medical management of a disease, disorder, or condition of a subject. In general, an appropriate dose or treatment regimen comprising a molecule or composition of the present disclosure is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay or prevention of disease progression; remission; survival; prolonged survival; or any combination thereof.

A “therapeutically effective amount” or “effective amount” of a molecule, polynucleotide, vector, host cell, or composition of this disclosure refers to an amount of the composition or molecule sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient, administered alone, a therapeutically effective amount refers to the effects of that ingredient or a cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially, sequentially, or simultaneously.

As used herein, “a subject” includes all mammals, including without limitation humans, non-human primates, dogs, cats, horses, sheep, goats, cows, rabbits, pigs, and rodents. A subject may be male or female, and can be any suitable age, including infant, juvenile, adolescent, adult, and geriatric subjects.

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

In the broadest sense, the naturally occurring amino acids can be divided into groups based upon the chemical characteristic of the side chain of the respective amino acids. By “hydrophobic” amino acid is meant either Ile, Leu, Met, Phe, Trp, Tyr, Val, Ala, Cys or Pro. By “hydrophilic” amino acid is meant either Gly, Asn, Gln, Ser, Thr, Asp, Glu, Lys, Arg or His.

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

As used herein, “protein” or “peptide” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid, and non-naturally occurring amino acid polymers. Variants of proteins, peptides, and polypeptides of this disclosure are also contemplated. In certain embodiments, variant proteins, peptides, and polypeptides comprise or consist of an amino acid sequence that is at least 70%, 75%, 80%, 85%, 90%, 91%, 92%, 93%, 94%, 95%, 96%, 97%, 98%, 99%, or 99.9% identical to an amino acid sequence of a defined or reference amino acid sequence as described herein.

“Nucleic acid molecule” or “oligonucleotide” or “polynucleotide” or “polynucleic acid” refers to an oligomeric or polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), which includes, for example, mRNA, microRNA, siRNA, viral genomic RNA, and synthetic RNA, and polydeoxyribonucleic acid (DNA), which includes, for example, cDNA, genomic DNA, and synthetic DNA. Both RNA and DNA may be single or double stranded. If single-stranded, the nucleic acid molecule may be the coding strand or non-coding (anti-sense) strand. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

Variants of nucleic acid molecules of this disclosure are also contemplated. Variant nucleic acid molecules are at least 70%, 75%, 80%, 85%, 90%, and are preferably 95%, 96%, 97%, 98%, 99%, or 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a binding domain thereof having a functionality described herein, such as binding a target molecule.

“Percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX), or Megalign (DNASTAR) software. The mathematical algorithm used in the BLAST programs can be found in Altschul et al., Nucleic Acids Res. 25:3389-3402, 1997. Appropriate parameters for measuring alignment, including any algorithms needed to achieve maximal alignment over the full-length of the sequences being compared can be determined by known methods.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such a nucleic acid could be part of a vector and/or such a nucleic acid or polypeptide could be part of a composition (e.g., a cell lysate), and still be isolated in that such vector or composition is not part of the natural environment for the nucleic acid or polypeptide. “Isolated” can, in some embodiments, also describe a protein, polypeptide, polynucleotide, vector, host cell, or composition, that is outside of a human body.

The term “gene” means the segment of DNA or RNA involved in producing a polypeptide chain; in certain contexts, it includes regions preceding and following the coding region (e.g., 5′ untranslated region (UTR) and 3′ UTR) as well as intervening sequences (introns) between individual coding segments (exons).

A “functional variant” refers to a polypeptide or polynucleotide that is structurally similar or substantially structurally similar to a parent or reference compound of this disclosure, but differs slightly in composition (e.g., one or more base, atom or functional group is different, added, or removed), such that the polypeptide or encoded polypeptide is capable of performing at least one function of the parent polypeptide with at least 50% efficiency, preferably at least 55%, 60%, 65%, 70%, 75%, 80%, 85%, 90%, 95%, 96%, 97%, 98%, 99%, 99.9%, 100% level of activity of the parent polypeptide, or a level of activity greater than that of the parent polypeptide. In other words, a functional variant of a polypeptide or encoded polypeptide of this disclosure has “similar binding,” “similar affinity” or “similar activity” when the functional variant displays an improvement in performance, or no more than a 50% reduction in performance, in a selected assay as compared to the parent or reference polypeptide, such as an assay for measuring enzymatic activity or binding affinity.

As used herein, a “functional portion” or “functional fragment” refers to a polypeptide or polynucleotide that comprises only a domain, portion or fragment of a parent or reference compound, and the polypeptide or encoded polypeptide retains at least 50% activity associated with the domain, portion or fragment of the parent or reference compound, preferably at least 55%, 60%, 65%, 70%, 75%, 80%, 85%, 90%, 95%, 96%, 97%, 98%, 99%, 99.9%, 100% level of activity of the parent polypeptide, or a level of activity greater than that of the parent polypeptide, or provides a biological benefit (e.g., effector function). A “functional portion” or “functional fragment” of a polypeptide or encoded polypeptide of this disclosure has “similar binding” or “similar activity” when the functional portion or fragment displays an improvement in performance, or no more than a 50% reduction in performance, in a selected assay as compared to the parent or reference polypeptide (preferably no more than 20% or 10% reduction, or no more than a log difference as compared to the parent or reference with regard to affinity).

As used herein, the term “engineered,” “recombinant,” or “non-natural” refers to an organism, microorganism, cell, protein, polypeptide, nucleic acid molecule, or vector that includes at least one genetic alteration or has been modified by introduction of an exogenous or heterologous nucleic acid molecule, wherein such alterations or modifications are introduced by genetic engineering (i.e., human intervention). Genetic alterations include, for example, modifications introducing expressible nucleic acid molecules encoding functional RNA, proteins, fusion proteins or enzymes, or other nucleic acid molecule additions, deletions, substitutions, or other functional disruption of a cell's genetic material. Additional modifications include, for example, non-coding regulatory regions in which the modifications alter expression of a polynucleotide, gene, or operon.

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

In certain embodiments, a nucleic acid molecule or portion thereof native to a host cell will be considered heterologous to the host cell if it has been altered or mutated, or a nucleic acid molecule native to a host cell may be considered heterologous if it has been altered with a heterologous expression control sequence or has been altered with an endogenous expression control sequence not normally associated with the nucleic acid molecule native to a host cell. In addition, the term “heterologous” can refer to a biological activity that is different, altered, or not endogenous to a host cell. As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding an antibody or antigen-binding fragment (or other polypeptide), or any combination thereof.

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, posttranslational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a protein (e.g., a heavy chain of an antibody), or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of different encoding nucleic acid molecules or the number of different protein activities, not the number of separate nucleic acid molecules introduced into a host cell.

The term “construct” refers to any polynucleotide that contains a recombinant nucleic acid molecule (or, when the context clearly indicates, a fusion protein of the present disclosure). A (polynucleotide) construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, an RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g., Geurts et al., Mol. Ther. 8:108, 2003: Mites et al., Nat. Genet. 41:753, 2009). Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, “expression vector” or “vector” refers to a DNA construct containing a nucleic acid molecule that is operably linked to a suitable control sequence capable of effecting the expression of the nucleic acid molecule in a suitable host. Such control sequences typically include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself or deliver the polynucleotide contained in the vector into the genome without the vector sequence. In the present specification, “plasmid,” “expression plasmid,” “virus,” and “vector” are often used interchangeably.

The term “introduced” in the context of inserting a nucleic acid molecule into a cell, means “transfection”, “transformation,” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

In certain embodiments, polynucleotides of the present disclosure may be operatively linked to certain elements of a vector. For example, polynucleotide sequences that are needed to affect the expression and processing of coding sequences to which they are ligated may be operatively linked. Expression control sequences may include appropriate transcription initiation, termination, promoter, and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest may also be considered operatively linked.

In certain embodiments, the vector comprises a plasmid vector or a viral vector (e.g., a lentiviral vector or a γ-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox, and canarypox). Other viruses include, for example, Norwalk virus, togavirus, flavivirus, reoviruses, papovavirus, hepadnavirus, and hepatitis virus. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996). Methods of using retroviral and lentiviral viral vectors and packaging cells for transducing mammalian host cells with viral particles containing transgenes are known in the art and have been previous described, for example, in: U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors that can be used with the compositions and methods of this disclosure include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as Sleeping Beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

Plasmid vectors, including DNA-based plasmid vectors for expression of one or more proteins in vitro or for direct administration to a subject, are also known in the art. Such vectors may comprise a bacterial origin of replication, a viral origin of replication, genes encoding components required for plasmid replication, and/or one or more selection markers, and may also contain additional sequences allowing for bicistronic or multicistronic expression.

As used herein, the term “host” refers to a cell or microorganism targeted for genetic modification with a heterologous nucleic acid molecule to produce a polypeptide of interest (e.g., an antibody of the present disclosure).

A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

“Antigen”, as used herein, refers to an immunogenic molecule that provokes an immune response. This immune response may involve antibody production, activation of specific immunologically competent cells, activation of complement, antibody dependent cytotoxicity, or any combination thereof. An antigen (immunogenic molecule) may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, stool samples, cells, biological fluids, or combinations thereof. Antigens can be produced by cells that have been modified or genetically engineered to express an antigen. Antigens can also be present in or on an infectious agent, such as present in a virion, or expressed or presented on the surface of a cell infected by infectious agent. In the context of antigens used for targeting certain cell types, the term “antigen” includes any molecule that is present on the surface of the targeted cell and that may be selectively bound by a targeting moiety, regardless of whether the molecule is immunogenic.

The term “epitope” or “antigenic epitope” includes any molecule, structure, amino acid sequence, or protein determinant that is recognized and specifically bound by a cognate binding molecule, such as an immunoglobulin, or other binding molecule, domain, or protein. Epitopic determinants generally contain chemically active surface groupings of molecules, such as amino acids or sugar side chains, and can have specific three-dimensional structural characteristics, as well as specific charge characteristics. Where an antigen is or comprises a peptide or protein, the epitope can be comprised of consecutive amino acids (e.g., a linear epitope), or can be comprised of amino acids from different parts or regions of the protein that are brought into proximity by protein folding (e.g., a discontinuous or conformational epitope), or non-contiguous amino acids that are in close proximity irrespective of protein folding.

The term “antibody” refers to an immunoglobulin molecule consisting of one or more polypeptides that specifically binds an antigen through at least one epitope recognition site. For example, the term “antibody” encompasses an intact antibody comprising at least two heavy chains and two light chains connected by disulfide bonds, as well as any antigen-binding portion or fragment of an intact antibody that has or retains the ability to bind to the antigen target molecule recognized by the intact antibody, such as an scFv, Fab, or Fab′2 fragment. The term also encompasses full-length or fragments of antibodies of any class or sub-class, including IgG and sub-classes thereof (such as IgG1, IgG2, IgG3, and IgG4), IgM, IgE, IgA, and IgD.

The term “antibody” is used herein in the broadest sense, encompassing antibodies and antibody fragments thereof, derived from any antibody producing mammal (e.g., mouse, rat, rabbit, and primate including human), or from a hybridoma, phage selection, recombinant expression or transgenic animals (or other methods of producing antibodies or antibody fragments). It is not intended that the term “antibody” be limited as regards to the source of the antibody or manner in which it is made (e.g., by hybridoma, phage selection, recombinant expression, transgenic animal, peptide synthesis, etc.). Exemplary antibodies include polyclonal, monoclonal and recombinant antibodies; multispecific antibodies (e.g., bispecific antibodies); humanized antibodies; fully human antibodies, murine antibodies; chimeric, mouse human, mouse primate, primate human monoclonal antibodies; and anti-idiotype antibodies, and may be any intact molecule or fragment thereof. As used herein, the term “antibody” encompasses not only intact polyclonal or monoclonal antibodies, but also fragments thereof (such as dAb, Fab, Fab′, F(ab′)2, Fv), single chain (ScFv), synthetic variants thereof, naturally occurring variants, fusion proteins comprising an antibody portion with an antigen-binding fragment of the required specificity, humanized antibodies, chimeric antibodies, and any other modified configuration of the immunoglobulin molecule that comprises an antigen-binding site or fragment (epitope recognition site) of the required specificity. The term encompasses genetically engineered and-or otherwise modified forms of immunoglobulins such as intrabodies, peptibodies, diabodies, triabodies, tetrabodies, tandem di-scFv, tandem tri-scFv, and the like, including antigen-binding fragments thereof.

The terms “VH” and “VL” refer to the variable binding regions from an antibody heavy chain and an antibody light chain, respectively. A VL may be a kappa class chain or a lambda class chain. The variable binding regions comprise discrete, well-defined sub-regions known as complementarity determining regions (CDRs) and framework regions (FRs). The CDRs are located within a hypervariable region (HVR) of the antibody and refer to sequences of amino acids within antibody variable regions which, in general, together confer the antigen specificity and/or binding affinity of the antibody. Consecutive CDRs (i.e., CDR1 and CDR2, and CDR2 and CDR3) are separated from one another in primary structure by a framework region.

As used herein, a “chimeric antibody” is a recombinant protein that contains the variable domains and complementarity determining regions derived from a non-human species (e.g., rodent) antibody, while the remainder of the antibody molecule is derived from a human antibody. A chimeric antibody may be comprised of an antigen-binding fragment of one antibody operably linked or otherwise fused to a heterologous Fc portion of a different antibody. For example, a mouse-human chimeric antibody may comprise an antigen-binding fragment of a mouse antibody fused to an Fc portion derived from a human antibody. The heterologous Fc domain may be from a different Ig class from the parent antibody, including IgA (including subclasses IgA1 and IgA2), IgD, IgE, IgG (including subclasses IgG1, IgG2, IgG3 and IgG4) and IgM.

As used herein, a “humanized antibody” is a molecule, generally prepared using recombinant techniques, having an antigen-binding site derived from an immunoglobulin from a non-human species and the remaining immunoglobulin structure of the molecule based upon the structure and/or sequence of a human immunoglobulin. A humanized antibody differs from a chimeric antibody in that typically only the CDRs from the non-human species are used, grafted onto appropriate framework regions in a human variable domain. Antigen binding sites may be wild type or may be modified by one or more amino acid substitutions. Humanized antibodies may preserve all CDR sequences (for example, a humanized mouse antibody which contains all six CDRs from the mouse antibodies). Alternatively, humanized antibodies may have one or more CDRs (one, two, three, four, five, six) which are altered with respect to the original antibody, which are also termed one or more CDRs “derived from” one or more CDRs from the original antibody.

As used herein, the term “antibody fragment” refers to a portion derived from or related to a full-length antibody, generally including the antigen binding or variable region thereof. Illustrative examples of antibody fragments include Fab, Fab′, F(ab)2, F(ab′)2 and Fv fragments, scFv fragments, diabodies, linear antibodies, single chain antibody molecules and multispecific antibodies formed from antibody fragments.

As used herein, the term “antigen-binding fragment” refers to a polypeptide fragment that contains at least one CDR of an immunoglobulin heavy and/or light chains and that specifically binds to the antigen to which the antibody was raised. An antigen-binding fragment may comprise 1, 2, 3, 4, 5, or all 6 CDRs of a VH and VL sequence from an antibody.

A “Fab” (fragment antigen binding) is the part of an antibody that binds to antigens and includes the variable region and CH1 of the heavy chain linked to the light chain via an inter-chain disulfide bond. Each Fab fragment is monovalent with respect to antigen binding, i.e., it has a single antigen-binding site. Pepsin treatment of an antibody yields a single large F(ab′)2 fragment that roughly corresponds to two disulfide-linked Fab fragments having divalent antigen-binding activity and is still capable of cross-linking antigen. Both the Fab and F(ab′)2 are examples of “antigen-binding fragments.” Fab′ fragments differ from Fab fragments by having a few additional residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments are often produced as pairs of Fab′ fragments that have hinge cysteines between them. Other chemical couplings of antibody fragments are also known.

Fab fragments may be joined, e.g., by a peptide linker, to form a single chain Fab, also referred to herein as “scFab.” In such a case, an inter-chain disulfide bond that is present in a native Fab may not be present, and the linker serves in full or in part to link or connect the Fab fragments in a single polypeptide chain. A heavy-chain derived Fab fragment (e.g., comprising, consisting of, or consisting essentially of VH+CH1, or “Fd”) and a light chain-derived Fab fragment (e.g., comprising, consisting of, or consisting essentially of VL+CL) may be linked in any arrangement to form a scFab. For example, a scFab may be arranged, in N-terminal to C-terminal direction, according to (heavy chain Fab fragment-linker-light chain Fab fragment) or (light chain Fab fragment-linker-heavy chain Fab fragment).

“Fv” is a small antibody fragment that contains a complete antigen-recognition and antigen-binding site. This fragment generally consists of a dimer of one heavy- and one light-chain variable region domain in tight, non-covalent association. However, even a single variable domain (or half of an Fv comprising only three CDRs specific for an antigen) has the ability to recognize and bind antigen, although typically at a lower affinity than the entire binding site.

“Single-chain Fv” also abbreviated as “sFv” or “scFv”, are antibody fragments that comprise the VH and VL antibody domains connected into a single polypeptide chain. The scFv polypeptide may comprise a polypeptide linker disposed between and linking the VH and VL domains that enables the scFv to retain or form the desired structure for antigen binding, although a linker is not always required. Such a peptide linker can be incorporated into a fusion polypeptide using standard techniques well known in the art. Additionally, or alternatively, Fv can have a disulfide bond formed between and stabilizing the VH and the VL. For a review of scFv, see Pluckthun in The Pharmacology of Monoclonal Antibodies, vol. 113, Rosenburg and Moore eds., Springer-Verlag, New York, pp. 269-315 (1994). An antibody or antigen-binding fragment may comprise a scFv comprising a VH domain, a VL domain, and a peptide linker linking the VH domain to the VL domain. A scFv may comprise a VH domain linked to a VL domain by a peptide linker, which can be in a VH-linker-VL orientation or in a VL-linker-VH orientation. Any scFv may be engineered so that the C-terminal end of the VL domain is linked by a short peptide sequence to the N-terminal end of the VH domain, or vice versa (i.e., (N)VL(C)-linker-(N)VH(C) or (N)VH(C)-linker-(N)VL(C)). Alternatively, in some a linker may be linked to an N-terminal portion or end of the VH domain, the VL domain, or both.

Peptide linker sequences for use in scFv or in other fusion proteins, such as the molecules described herein, may be chosen, for example, based on: (1) their ability to adopt a flexible extended conformation; (2) their inability or lack of ability to adopt a secondary structure that could interact with functional epitopes on the first and second polypeptides and/or on a target molecule; and/or (3) the lack or relative lack of hydrophobic or charged residues that might react with the polypeptides and/or target molecule. Other considerations regarding linker design (e.g., length) can include the conformation or range of conformations in which the VH and VL can form a functional antigen-binding site. Unstructured, rigid, or structured recombinant linkers allow for a fusion between any two proteins in a manner that allows for the desired properties of the fusion to be retained. The desired effect may be to minimize steric hinderance within the fusion, allowing maximal contextual function of the protein fusion elements. A variety of linkers with different properties are known in the art. Unstructured recombinant linkers are known in the art to be generally 1-100 amino acids in length and to generally contain polar, neutral, hydrophobic, or charged amino acids such as glycine, alanine, valine, proline, serine, glutamate, glutamine, lysine, arginine, aspartate, asparagine, and/or histidine, in any combination. Hydrophobic amino acids such as leucine, isoleucine, tryptophan, cysteine, tyrosine, and phenylalanine may be used to add structural rigidity to a linker. In certain embodiments, peptide linker sequences contain, for example, Gly, Asn and Ser residues. Other near neutral amino acids, such as Thr and Ala, may also be included in a linker sequence. Other amino acid sequences which may be usefully employed as linker include those disclosed in Maratea et al., Gene 40:39 46(1985); Murphy et al., Proc. Natl. Acad. Sci. USA 83:8258 8262 (1986); U.S. Pat. No. 4,935,233, and 4,751,180. Other illustrative and non-limiting examples of linkers may include, for example, a single glycine, or glycine repeated two to five times or more, the pentamer Gly-Gly-Gly-Gly-Ser (GGGGS; SEQ ID NO:29) when present in a single iteration or repeated one to five times or more, and may begin or end in a partial iteration, such as, for example, GGGGSGGGGSGGGG (SEQ ID NO:30), and the pentamer Gly-Gly-Ser-Gly-Gly (GGSGG; SEQ ID NO:58) when present in a single iteration or repeated one to five times or more. Any suitable linker may be used, and in general can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or 100 amino acids in length, or less than about 200 amino acids in length, and will preferably provide the appropriate amount of flexibility for conformational movement between two regions, domains, motifs, fragments, or modules connected by the linker, and will preferably be biologically inert and/or have a low risk of immunogenicity in a human.

Antibodies may be monospecific (e.g., binding to a single epitope) or multispecific (e.g., binding to multiple epitopes and/or target molecules). A bispecific or multispecific antibody or antigen-binding fragment may comprise one, two, or more antigen-binding domains (e.g., a VH and a VL). Two or more binding domains may be present that bind to the same or different epitopes, and a bispecific or multispecific antibody or antigen-binding fragment can comprise two or more binding domains, that bind to different antigens or pathogens altogether.

Antibodies and antigen-binding fragments may be constructed in various formats. Exemplary antibody formats disclosed in Spiess et al., Mol. Immunol. 67(2):95 (2015), and in Brinkmann and Kontermann, mAbs 9(2):182-212 (2017), which formats and methods of making the same are incorporated herein by reference and include, for example, Bispecific T cell Engagers (BiTEs), DARTs, Knobs-Into-Holes (KIH) assemblies, scFv-CH3-KIH assemblies, KIH Common Light-Chain antibodies, TandAbs, Triple Bodies, TriBi Minibodies, Fab-scFv, scFv-CH-CL-scFv, F(ab′)2-scFv2, tetravalent HCabs, Intrabodies, CrossMabs, Dual Action Fabs (DAFs) (two-in-one or four-in-one), DutaMabs, DT-IgG, Charge Pairs, Fab-arm Exchange, SEEDbodies, Triomabs, LUZ-Y assemblies, Fcabs, κλ-bodies, orthogonal Fabs, DVD-Igs (e.g., U.S. Pat. No. 8,258,268, which formats are incorporated herein by reference in their entirety), IgG(H)-scFv, scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, Zybody, and DVI-IgG (four-in-one), as well as so-called FIT-Ig (e.g., PCT 5 Publication No. WO 2015/103072, which formats are incorporated herein by reference in their entirety), so-called WuxiBody formats (e.g., PCT Publication No. WO 2019/057122, which formats are incorporated herein by reference in their entirety), and so-called In-Elbow-Insert Ig formats (IEI-Ig; e.g., PCT Publication Nos. WO 2019/024979 and WO 2019/025391, which formats are incorporated herein by reference in their entirety).

An antibody or antigen-binding fragment may comprise two or more VH domains, two or more VL domains, or both (i.e., two or more VH domains and two or more VL domains). An antigen-binding fragment may comprise the format (N-terminal to C-terminal direction) VH-linker-VL-linker-VH-linker-VL, wherein the two VH sequences can be the same or different and the two VL sequences can be the same or different. Such linked scFvs can include any combination of VH and VL domains arranged to bind to a given target, and in formats comprising two or more VH and/or two or more VL, one, two, or more different epitopes or antigens may be bound. It will be appreciated that formats incorporating multiple antigen-binding domains may include VH and/or VL sequences in any combination or orientation. For example, the antigen-binding fragment can comprise the format VL-linker-VH-linker-VL-linker-VH, VH-linker-VL-linker-VL-linker-VH, or VL-linker-VH-linker-VH-linker-VL.

As used herein, the modifier “monoclonal” indicates the character of the antibody as being obtained from a substantially homogenous population of antibodies, and is not intended to be limited as regards the source of the antibody or the manner in which it is made (e.g., by hybridoma, phage selection, recombinant expression, transgenic animals, etc.). The term “monoclonal antibody” encompasses not only intact monoclonal antibodies and full-length monoclonal antibodies, but also fragments thereof (such as Fab, Fab′, F(ab′)2, Fv), single chain (ScFv), variants thereof, fusion proteins comprising an antigen-binding portion, humanized monoclonal antibodies, chimeric monoclonal antibodies, and any other modified configuration of the immunoglobulin molecule that comprises an antigen-binding fragment (epitope recognition site) of the required specificity and the ability to bind to an epitope. Monoclonal antibodies can be obtained using any technique that provides for the production of antibody molecules by continuous cell lines in culture, such as the hybridoma method described by Kohler, G., et al., Nature 256:495, 1975, or they may be made by recombinant DNA methods (see, e.g., U.S. Pat. No. 4,816,567 to Cabilly). Monoclonal antibodies may also be isolated from phage antibody libraries using the techniques described in Clackson, T., et al., Nature 352:624 628, 1991, and Marks, J. D., et al., J. Mol. Biol. 222:581 597, 1991. Such antibodies can be of any immunoglobulin class including IgG, IgM, IgE, IgA, IgD and any subclass thereof.

The recognized immunoglobulin polypeptides include the kappa and lambda light chains and the alpha, gamma (IgG1, IgG2, IgG3, IgG4), delta, epsilon and mu heavy chains, or equivalents in other species. Full-length immunoglobulin “light chains” (of about 25 kDa or about 214 amino acids) comprise a variable region of about 110 amino acids at the NH2 terminus and a kappa or lambda constant region at the COOH-terminus. Full-length immunoglobulin “heavy chains” (of about 50 kDa or about 446 amino acids) similarly comprise a variable region (of about 116 amino acids) and one of the aforementioned heavy chain constant regions, e.g., gamma (of about 330 amino acids).

The basic four-chain antibody unit is a heterotetrameric glycoprotein composed of two identical light (L) chains and two identical heavy (H) chains. An IgM antibody differs from this plan in that it consists of five of the basic heterotetramer units along with an additional polypeptide called the J chain, and therefore contains 10 antigen binding sites. Secreted IgA antibodies also differ from the basic structure in that they can polymerize to form polyvalent assemblages comprising two to five of the basic four-chain units along with a J chain. Each L chain is linked to an H chain by one covalent disulfide bond, while the two H chains are linked to each other by one or more by one or more disulfide bonds, depending on the H chain isotype. Each H and L chain also has regularly spaced intrachain disulfide bridges. The pairing of a VH and VL together forms a single antigen-binding site.

Each H chain has, at the N-terminus, a variable domain (VH) followed by three constant domains (CH1, CH2, CH3), in the case of alpha, gamma, and delta chains, or four CH domains (CH1, CH2, CH3, CH4), in the case of mu and epsilon chains.

Each L chain has, at the N-terminus, a variable domain (VL) followed by a constant domain (CL) at its other end. When an L chain and an H chain are paired, the VL is aligned with the VH and the CL is aligned with the first constant domain of the heavy chain (CH1). The L chain from any vertebrate species can be assigned to one of two types, called kappa (κ) and lambda (k), based on the amino acid sequences of their constant domains (CL).

Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated alpha (α), delta (δ), epsilon (ε), gamma (γ) and mu (μ), respectively. The γ and α classes are further divided into subclasses on the basis of minor differences in CH sequence and function, for example, humans express the following subclasses: IgG1, IgG2, IgG3, IgG4, IgA1 and IgA2.

For the structure and properties of the different classes of antibodies, see, e.g., Basic and Clinical Immunology, 8th Edition, Daniel P. Stites, Abba I. Terr and Tristram G. Parslow (eds); Appleton and Lange, Norwalk, Conn., 1994, page 71 and Chapter 6.

The term “variable” refers to that fact that certain segments of the V domains differ extensively in sequence among antibodies. The V domain mediates antigen binding and defines specificity of a particular antibody for its particular antigen. However, the variability is not evenly distributed across the 110 amino acid span of the variable domains. Rather, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat, et al., Sequences of Proteins of Immunological Interest, 5th Ed. Public Health Service, National Institutes of Health, Bethesda, Md (1991)). The constant domains are not involved directly in binding an antibody to an antigen, but exhibit various effector functions.

As used herein, “effector functions” refer to those biological activities attributable to the Fc region of an antibody. Examples of antibody effector functions include participation in antibody-dependent cellular cytotoxicity (ADCC), C1q binding and complement-dependent cytotoxicity, Fc receptor binding, phagocytosis, down-regulation of cell surface receptors, and B cell activation. Modifications such as amino acid substitutions may be made to an Fc domain in order to modify (e.g., enhance or reduce) one or more functions of an Fc-containing polypeptide. Such functions include, for example, Fc receptor binding, antibody half-life modulation, ADCC function, protein A binding, protein G binding, and complement binding. Amino acid modifications that modify Fc functions include, for example, T250Q/M428L, M252Y/S254T/T256E, H433K/N434F, M428L/N434S, E233P/L234V/L235A/G236A/A327G/A330S/P331S, E333A, S239D/A330L/I332E, P257I/Q311, K326W/E333S, S239D/I332E/G236A, N297Q, K322A, S228P, L235E/E318A/K320A/K322A, L234A/L235A, and L234A/L235A/P329G mutations. Other Fc modifications and their effect on Fc function are known in the art.

As used herein, the term “hypervariable region” refers to the amino acid residues of an antibody that are responsible for antigen binding. The hypervariable region contains several “complementarity determining regions” (CDRs). The heavy chain comprises three CDR sequences (CDRH1, CDRH2, and CDRH3) and the light chain comprises three CDR sequences (CDRL1, CDRL2, and CDRL3). A variety of systems exist for identifying and numbering the amino acids that make up the CDRs. For example, the hypervariable region generally comprises CDRs at around about residues 24-34 (L1), 50-56 (L2) and 89-97 (L3) in the light chain variable domain, and at around about 31-35 (H1), 50-65 (H2) and 95-102 (H3) in the heavy chain variable domain when numbering in accordance with the Kabat numbering system as described in Kabat, et al., Sequences of Proteins of Immunological Interest, 5th Ed. Public Health Service, National Institutes of Health, Bethesda, Md (1991); and/or at about residues 24-34 (L1), 50-56 (L2) and 89-97 (L3) in the light chain variable domain, and 26-32 (H1), 52-56 (H2) and 95-102 (H3) in the heavy chain variable domain when numbered in accordance with the Chothia numbering system, as described in Chothia and Lesk, J. Mol. Biol. 196:901-917 (1987); and/or at about residues 27-38 (L1), 56-65 (L2) and 105-117 (L3) in the VL, and 27-38 (H1), 56-65 (H2), and 105-117 (H3) in the VH when numbered in accordance with the IMGT numbering system as described in Lefranc, J. P., et al., Nucleic Acids Res 27:209-212; Ruiz, M., et al., Nucleic Acids Res 28:219-221 (2000). Equivalent residue positions can be annotated and compared for different molecules using Antigen receptor Numbering And Receptor Classification (ANARCI) software tool (2016, Bioinformatics 15:298-300). Accordingly, identification of CDRs of an exemplary variable domain (VH or VL) sequence as provided herein according to one numbering scheme is not exclusive of an antibody comprising CDRs of the same variable domain as determined using a different numbering scheme.

As used herein, “specifically binds” refers to an antibody or antigen-binding fragment that binds to an antigen with a particular affinity, while not significantly associating or uniting with any other molecules or components in a sample. Affinity may be defined as an equilibrium association constant (Ka), calculated as the ratio of kon/koff, with units of 1/M or as an equilibrium dissociation constant Kd), calculated as the ratio of koff/kon with units of M.

In some contexts, antibody and antigen-binding fragments may be described with reference to affinity and/or to avidity for antigen. Unless otherwise indicated, avidity refers to the total binding strength of an antibody or antigen-binding fragment thereof to antigen, and reflects binding affinity, valency of the antibody or antigen-binding fragment (e.g., whether the antibody or antigen-binding fragment comprises one, two, three, four, five, six, seven, eight, nine, ten, or more binding sites), and, for example, whether another agent is present that can affect the binding (e.g., a non-competitive inhibitor of the antibody or antigen-binding fragment).

Each embodiment in this specification is to be applied mutatis mutandis to every other embodiment unless expressly stated otherwise. It is contemplated that any embodiment discussed in this specification can be implemented with respect to any method, kit, reagent, or composition of the invention, and vice versa. Furthermore, compositions of the invention can be used to achieve methods of the invention.

### II. Proteins and Polypeptides

### A. Toxin Domains

The molecules provided herein comprise a toxin domain that has DNase activity. A variety of molecules having DNase activity are known in the art, including mammalian DNase I enzymes, a DNase from Epstein-Barr virus, a variety of bacterial nucleases, and the DNase present in cytolethal distending toxin. (Mori et al. Sci Rep. 2022. 12, 10364; Liu et al. Virology. 1988. 20:247; Lovett S. EcoSal Plus. 2011. 4:10.) DNases have an ability to degrade DNA in vivo, which is useful to cells for some purposes, such as DNA repair and genetic recombination, but can also function as a toxin under certain circumstances. Any appropriate DNase may be the source of a toxin domain for use in the molecules of the present invention. Many DNases have an inherent nuclear localization function. If such a function is not present, as is the case for some bacterial nucleases, a nuclear localization signal may be added to the DNase using recombinant technologies known in the art. (Lu et al. Cell Commun Signal. 2021. 19:60.)

In some embodiments, the toxin domain is derived from cytolethal distending toxin (CDT). CDT is a tripartite genotoxin produced by various Gram-negative pathogens, including, among others, Haemophilus ducreyi, Escherichia coli, Haemophilus parasuis, Aggregatibacter actinomycetemcomitans, and Salmonella enterica. (Lara-Tejero M, Galán J E. Science. 2000. 290:354; Guerra L, et al. Toxins. 2011. 3:172.) The CDT tripartite toxin consists of CdtA and CdtC domains, which facilitate cell surface binding and endocytic uptake, and a CdtB domain, which is a cellular toxin. CdtB contains nuclear localization signals that enable nuclear transport through host import pathways. (McSweeney L A, et al. Cell Microbiol. 2004. 6:447.) Upon nuclear localization, the CdtB domain functions as a DNase I-like enzyme that induces double-strand DNA breaks. This activity triggers the cell's DNA damage response and leads to G2/M cell cycle arrest, resulting in apoptosis or senescence. (Bezine E, et al. Cells. 2014. 3:592.) Several studies have shown that CdtB alone can exhibit toxicity when artificially delivered into the cytosol, indicating that the natural heterotrimeric complex is not a functional requirement for the DNase activity. (Hassane et al. Infect Immun. 2001. 69:5752.) CDTs from different species have the same general structure and function, but varying degrees of sequence homology. For example, the CDT from Haemophilus ducreyi has only 67% sequence identity with the CDTs from Haemophilus parasuis or Aggregatibacter actinomycetemcomitans, only 53% sequence identity with the CDT from Salmonella enterica, and only 50% sequence identity with the CDT from Escherichia coli.

In some embodiments, the molecules provided herein comprise a CDT-derived domain. In some embodiments, the CDT-derived domain is derived from a CdtB domain. The CDT-derived domain may be derived from any known CDT. In some embodiments, the CDT-derived domain is derived from a CDT from a Haemophilus species, an Escherichia species, an Aggregatibacter species, or a Salmonella species. In some embodiments, the CDT-derived domain is derived from a CDT from Haemophilus ducreyi. In some embodiments, the CDT-derived domain is derived from or comprises SEQ ID NO:1. In some embodiments, the CDT-derived domain is derived from a CDT from Haemophilus parasuis. In some embodiments, the CDT-derived domain is derived from or comprises SEQ ID NO:2. In some embodiments, the CDT-derived domain is derived from a CDT from Escherichia coli. In some embodiments, the CDT-derived domain is derived from or comprises SEQ ID NO:3. In some embodiments, the CDT-derived domain is derived from a CDT from Aggregatibacter actinomycetemcomitans. In some embodiments, the CDT-derived domain is derived from or comprises SEQ ID NO:4. In some embodiments, the CDT-derived domain is derived from a CDT from Salmonella enterica. In some embodiments, the CDT-derived domain is derived from or comprises SEQ ID NO:5. In some embodiments, the CDT-derived domain comprises alterations from the naturally occurring sequence that have improved toxin activity or other desirable characteristics in the context of the fusion molecule. Accordingly, in some embodiments, the CDT-derived domain comprises a sequence having 75%, 80%, 85%, 90%, 95%, 97%, or 99% identity with any one of SEQ ID NOs:1-5.

### B. Translocation Domains

The molecules provided herein comprise a translocation domain capable of translocating the toxin domain from within the endosome, across the endosomal membrane, and into the cytosol of a cell. Such domains are found in certain bacterial or viral proteins, and a number of examples are known in the art. Examples of suitable translocation domains include those derived from diphtheria toxin (O'Keefe et al. PNAS. 1992.89:6202), Pseudomonas exotoxin type A (Prior et al. Biochem. 1992. 31:3555), and anthrax toxin (Blanke et al. PNAS. 1996. 93:8437), among others. In some embodiments, the translocation domain is derived from diphtheria toxin (DT). DT is a bacterial toxin produced by Corynebacterium diphtheriae and comprises three functional domains. The A domain (DTA) causes ADP-ribosylation of elongation factor-2, thereby inhibiting protein synthesis. The B domain (DTB) facilitates endosomal escape of the toxin via a pH-dependent membrane insertion functionality. The C-terminal domain binds heparin-binding epidermal growth factor-like growth factor (HB-EGF) to mediate cellular uptake. Activation of DT occurs through furin-dependent proteolytic cleavage at a conserved “RVRR” motif, which separates the DTA and DTB domains while maintaining a disulfide linkage between cysteines on either side of the furin cleavage site that remains intact until endosomal acidification triggers structural changes in DTB, enabling membrane translocation of the DTA domain out of the endosome. The DTB domain alone, when used in combination with a protease cleavage site, is capable of providing pH-dependent translocation of a polypeptide out of the endosome. (Ratts R, et al. J Cell Biol. 2003. 160:1139; Ladokhin A S. Toxins. 2013. 5:1362.)

In some embodiments, the molecules provided herein comprise a DTB-derived translocation domain. Any fragment of DTB that retains translocation functionality is a functional fragment, and may be used as a translocation domain in the molecules of the present disclosure. In some embodiments, the DTB-derived translocation domain comprises alterations from the naturally occurring sequence that have improved functionality in the context of the fusion molecule. In some embodiments, the DTB-derived translocation domain comprises or is derived from SEQ ID NO:10. In some embodiments, the DTB-derived translocation domain comprises an amino acid sequence having 75%, 80%, 85%, 90%, 95%, 97%, or 99% identity with SEQ ID NO:10. In some embodiments, the DTB-derived translocation domain comprises or is derived from SEQ ID NO:63. In some embodiments, the DTB-derived translocation domain comprises an amino acid sequence having 75%, 80%, 85%, 90%, 95%, 97%, or 99% identity with SEQ ID NO:63.

### C. Targeting Moieties

The molecules provided herein comprise one or more targeting moieties that allow the toxic effects of the molecule to be targeted to specific cell types. In some embodiments, the targeted cell type is cancer cells. In some embodiments, the targeted cells are a specific type of cancer. The targeted cells may be hematological cancer cells or they may be non-hematological cancer cells.

The targeting moiety/moieties may be derived from any molecule that binds a molecule present on the surface of a targeted cell. In some embodiments, a targeting moiety is derived from a naturally occurring ligand of the molecule present on the surface of a targeted cell. In some embodiments, a targeting moiety is derived from an artificial, engineered, or synthetic ligand of the molecule present on the surface of a targeted cell. In some embodiments, a targeting moiety is derived from a cytokine, receptor-binding protein or peptide, or other molecule capable of binding a molecule present on the surface of a targeted cell. In some embodiments, a targeting moiety is derived from an antibody that specifically binds a molecule present on the surface of a targeted cell. Molecules present on the surface of a targeted cell may also be referred to herein as antigens, regardless of whether the targeting moiety is derived from an antibody or another ligand of the molecule. In some embodiments, a targeting moiety comprises a ligand of a molecule present on the surface of a targeted cell, or a functional fragment or functional variant of such a ligand. In some embodiments, a targeting moiety comprises a cytokine or receptor-binding protein or peptide or a functional fragment or functional variant thereof that binds a receptor present on the surface of a targeted cell. In some embodiments, a targeting moiety comprises an antibody that selectively binds a molecule present on the surface of a targeted cell, or a functional fragment or functional variant of such an antibody. In some embodiments, a targeting moiety is derived from an intact antibody or an antigen-binding fragment thereof. In some embodiments, a targeting moiety may be derived from a modified or engineered version of an antibody, such as a single chain antibody, nanobody, affibody, DARPin, or the like, or a functional fragment or functional variant thereof.

In some embodiments, the targeting moiety/moieties selectively bind a molecule present on the surface of a targeted cell that is capable of mediating the internalization of the protein comprising the targeting moiety into the target cell. In some embodiments, the binding of a targeting moiety to a molecule present on the surface of a targeted cell results in the receptor-mediated endocytosis of the protein comprising the targeting moiety.

In some embodiments, the targeted cells are hematological cancer cells. Hematological cancers are cancer of the blood, bone marrow, or lymphatic system. There are a large number of different types of hematological cancers, which are classified based on various tissue, system, and/or genetic criteria. Hematological cancers are broadly grouped into leukemias, lymphomas, plasma cell neoplasms, myelodysplastic syndromes (MDS), myeloproliferative neoplasms (MPN), histiocytic and dendritic cell neoplasms, and mast cell neoplasms. Each of these categories includes multiple different types of cancers. For example, leukemias include acute leukemias, such as acute myeloid leukemia (AML), acute promyelocytic leukemia, and acute lymphoblastic leukemia (ALL), chronic leukemias, such as chronic myeloid leukemia (CML), and chronic lymphocytic leukemia (CLL), and other leukemias such as hairy cell leukemia, prolymphocytic leukemia, and large granular lymphocytic leukemia (LGL), which is itself divided into T-cell LGL and NK-cell LGL. Lymphomas include Hodgkin lymphoma and non-Hodgkin lymphoma, with non-Hodgkin lymphoma further divided into B-cell lymphomas and T-cell and/or NK-cell lymphomas. Plasma cell neoplasms include multiple myeloma (MM), monoclonal gammopathy of undetermined significance (MGUS), plasmacytoma, Waldenstrom macroglobulinemia, and light chain (AL) amyloidosis. Myelodysplastic syndromes include a variety of subtypes of disease. Myeloproliferative neoplasms include polycythemia vera (PV), essential thrombocythemia (ET), primary myelofibrosis (PMF), chronic myeloid leukemia (CML), chronic myelomonocytic leukemia (CMML), juvenile myelomonocytic leukemia (JMML), and blastic plasmacytoid dendritic cell neoplasm (BPDCN). Histiocytic and dendritic cell neoplasms include Langerhans cell histiocytosis (LCH), histocytic sarcoma, follicular dendritic cell sarcoma, and interdigitating dendritic cell sarcoma. Mast cell neoplasms include systemic mastocytosis and mast cell leukemia. The targeted cells may be cancer cells of any hematological cancer, including any subtype, variant, or stage of a hematological cancer.

In some embodiments, the molecules provided herein comprise targeting moieties that bind to one or more antigens or receptors present on the surface of hematological cancer cells. A targeting moiety may target any antigen or receptor present on the surface of a hematological cancer cell, preferably an antigen or receptor that is preferentially expressed on the surface of the hematological cancer cells as compared to healthy cells. In some embodiments, the molecules provided herein comprise two or more targeting moieties. These two or more targeting moieties may be the same or they may be different. When the two or more targeting moieties are different, they may bind the same antigen or receptor on the surface of a hematological cancer cell or they may bind different antigens or receptors on the surface of a hematological cancer cell.

In some embodiments, the targeted cells are acute myeloid leukemia (AML) cells. AML is the most fatal form of leukemia and accounts for approximately 80 percent of acute leukemias in adults and one-third of all cancers affecting bone marrow and blood. AML originates from hematopoietic stem/progenitor cells in the bone marrow. AML is an aggressive bone marrow malignancy requiring rapid diagnosis and treatment. Advances in genomics, targeted therapy, and immunotherapy are improving outcomes, but the prognosis for AML patients is still dire.

In AML, genetic mutations lead to blocked differentiation and uncontrolled proliferation of myeloid blasts. The bone marrow becomes crowded with leukemic cells, leading to cytopenias (low blood cell counts). Current treatments for AML consist first of aggressive chemotherapy. The standard treatment is the “7+3” regimen: cytarabine for 7 days with the addition of anthracycline for 3 days. For patients with high-risk AML and mutations in FMS (feline McDonough sarcoma)-like tyrosine kinase 3a (FLT3), midostaurin, a FLT3 inhibitor, is added. This initial chemotherapy is followed by a consolidation therapy consisting of HiDAC (high-dose cytarabine) for younger patients, or venetoclax, a B-cell lymphoma 2 (BCL2) inhibitor, in combination with a demethylating agent such as azacytidine or an allogeneic stem cell transplant (SCT) in high-risk patients. (Adult Acute Myeloid Leukemia Treatment (PDQ)—Health Professional Version. Updated 2025. Accessed Mar. 19, 2026. cancer.gov/types/leukemia/hp/adult-aml-treatment-pdq; Sekeres et al. Blood Adv. 2026. 10:1897.)

Some targeted therapies have been developed in an effort to improve outcomes in certain subsets of patients. For example, venetoclax may be used in combination with hypomethylating agents such as azacitidine or decitabine, particularly in elderly patients or those with co-morbidities. The FLT3 inhibitors midostaurin and gilteritinib may be used during initial chemotherapy or for relapsed AML, respectively, in patients with a FLT3-mutated cancer. Isocitrate dehydrogenase 1/2 (IDH1/IDH2) inhibitors such as ivosidenib and enasidenib may be used in patients with an IDH-mutant AML, and menin inhibitors may be used in patients with lysine methyltransferase 2A (KMT2A) rearranged AML.

Regardless of the treatment, the majority of AML patients will relapse. Even stem cell transplant, which is the only potentially curative therapy, does not have a good prognosis. The overall 5-year survival rate for AML remains below 30% and is even lower for older patients.

One of the reasons AML is so difficult to treat successfully may relate to Leukemia Stem Cells (LSCs), which are a rare population of self-renewing, therapy-resistant cells responsible for AML initiation, progression, and relapse. Unlike bulk leukemia cells, LSCs survive chemotherapy and can repopulate the disease. A therapy that could prevent or reduce LSC-related progression and relapse would significantly improve outcomes for AML patients.

In some embodiments, the molecules provided herein comprise targeting moieties that bind to one or more antigens or receptors present on the surface of AML cells. A targeting moiety may target any antigen or receptor present on the surface of an AML cell, preferably an antigen or receptor that is preferentially expressed on the surface of AML cells as compared to healthy cells. In some embodiments, the molecules provided herein comprise two or more targeting moieties. These two or more targeting moieties may be the same or they may be different. When the two or more targeting moieties are different, they may bind the same antigen or receptor on the surface of an AML cell or they may bind different antigens or receptors on the surface of an AML cell.

In some embodiments, the targeted cells are chronic myelomonocytic leukemia (CMML) cells. CMML is a rare type of cancer that starts in the blood-forming cells of the bone marrow and affects mainly older adults. It is classified as a myelodysplastic/myeloproliferative neoplasm (MDS/MPN), meaning that it has features of both an MDS, where blood cells don't mature properly, and an MPN, where too many blood cells are produced. CMML cells often show hypersensitivity to GM-CSF.

In some embodiments, the molecules provided herein comprise targeting moieties that bind to one or more antigens or receptors present on the surface of CMML cells. A targeting moiety may target any antigen or receptor present on the surface of a CMML cell, preferably an antigen or receptor that is preferentially expressed on the surface of CMML cells as compared to healthy cells. In some embodiments, the molecules provided herein comprise two or more targeting moieties. These two or more targeting moieties may be the same or they may be different. When the two or more targeting moieties are different, they may bind the same antigen or receptor on the surface of a CMML cell or they may bind different antigens or receptors on the surface of a CMML cell.

In some embodiments, the targeted cells are blastic plasmacytoid dendritic cell neoplasm (BPDCN). BPDCN is a rare hematologic cancer of the plasmacytoid dendritic cells, a subset of dendritic cells that is primarily involved in immune regulation. BPDCN cells express CD123, the receptor for IL3, at high levels.

In some embodiments, the molecules provided herein comprise targeting moieties that bind to one or more antigens or receptors present on the surface of BPDCN cells. A targeting moiety may target any antigen or receptor present on the surface of a BPDCN cell, preferably an antigen or receptor that is preferentially expressed on the surface of BPDCN cells as compared to healthy cells. In some embodiments, the molecules provided herein comprise two or more targeting moieties. These two or more targeting moieties may be the same or they may be different. When the two or more targeting moieties are different, they may bind the same antigen or receptor on the surface of a BPDCN cell or they may bind different antigens or receptors on the surface of a BPDCN cell.

In some embodiments, the targeted cells are acute lymphoblastic leukemia (ALL) cells. ALL is a cancer of the blood and bone marrow which results in the overproduction of immature blood cells. ALL can start in either B cells (B-ALL) or T cells (T-ALL). In T-ALL and some cases of B-ALL, particularly in especially high-risk subtypes, CD123 is overexpressed.

In some embodiments, the molecules provided herein comprise targeting moieties that bind to one or more antigens or receptors present on the surface of ALL cells. A targeting moiety may target any antigen or receptor present on the surface of an ALL cell, preferably an antigen or receptor that is preferentially expressed on the surface of ALL cells as compared to healthy cells. In some embodiments, the molecules provided herein comprise two or more targeting moieties. These two or more targeting moieties may be the same or they may be different. When the two or more targeting moieties are different, they may bind the same antigen or receptor on the surface of an ALL cell or they may bind different antigens or receptors on the surface of an ALL cell.

In some embodiments, the targeted cells are juvenile myelomonocytic leukemia (JMML) cells. JMML is a rare pediatric hematological cancer. It is classified as a myelodysplastic/myeloproliferative neoplasm (MDS/MPN), meaning that blood cells don't mature properly and too many blood cells are produced. Nearly all JMML cancers show GM-CSF hypersensitivity, which is used as a diagnostic criterion.

In some embodiments, the molecules provided herein comprise targeting moieties that bind to one or more antigens or receptors present on the surface of JMML cells. A targeting moiety may target any antigen or receptor present on the surface of a JMML cell, preferably an antigen that is preferentially expressed on the surface of JMML cells as compared to healthy cells. In some embodiments, the molecules provided herein comprise two or more targeting moieties. These two or more targeting moieties may be the same or they may be different. When the two or more targeting moieties are different, they may bind the same antigen or receptor on the surface of a JMML cell or they may bind different antigens or receptors on the surface of a JMML cell. In some embodiments, the molecules provided herein comprise a targeting moiety derived from granulocyte-macrophage colony-stimulating factor (GM-CSF). GM-CSF is a cytokine that binds to the CD116/CD131 heterodimeric receptor pair. Any fragment or variant of GM-CSF that has specific binding activity with respect to CD116/CD131 is a functional fragment or variant, and may be used as a targeting moiety in the molecules of the present disclosure. In some embodiments, the GM-CSF-derived targeting moiety comprises alterations from the naturally occurring sequence that have improved binding affinity to the target in the context of the fusion molecule. In some embodiments, the GM-CSF-derived targeting moiety is derived from human GM-CSF. In some embodiments, the GM-CSF-derived targeting moiety comprises SEQ ID NO:11. In some embodiments, the GM-CSF-derived targeting moiety comprises an amino acid sequence having 75%, 80%, 85%, 90%, 95%, 97%, or 99% identity with SEQ ID NO:11.

In some embodiments, the molecules provided herein comprise a targeting moiety derived from interleukin 3 (IL3). IL3 is a cytokine that binds to the CD123/CD131 heterodimeric receptor pair. Any fragment or variant of IL3 that has specific binding activity with respect to CD123/CD131 is a functional fragment or variant, and may be used as a targeting moiety in the molecules of the present disclosure. In some embodiments, the IL3-derived targeting moiety comprises alterations from the naturally occurring sequence that have improved binding affinity to the target in the context of the fusion molecule. In some embodiments, the IL3-derived targeting moiety is derived from human IL3. In some embodiments, the IL3-derived targeting moiety comprises SEQ ID NO:12. In some embodiment, the IL3-derived targeting moiety comprises SEQ ID NO:13. In some embodiments, the IL3-derived targeting moiety comprises SEQ ID NO:14. In some embodiments, the IL3-derived targeting moiety comprises SEQ ID NO:15. In some embodiments, the IL3-derived targeting moiety comprises an amino acid sequence having 75%, 80%, 85%, 90%, 95%, 97%, or 99% identity with any one of SEQ ID NOs:12-15.

Some cancers may not express GM-CSF receptors and/or IL3 receptors at high levels. Therefore, for certain cancers, it may be appropriate to select an alternative targeting domain. In some embodiments, the molecule provided comprises a targeting moiety selected to target a cancer cell surface antigen or receptor that is present on the surface of the selected hematological cancer cells.

In some embodiments, the molecules provided herein comprise a targeting moiety derived from an anti-CD33 antibody. CD33 is expressed on myeloid cells and is known to be overexpressed in the majority of AML cancers. Any antibody that specifically binds CD33 may be used. For example, one antibody known to bind CD33 is gemtuzumab (National Cancer Institute (NCI) code C171025). Any fragment or variant of an anti-CD33 antibody that has specific binding activity with respect to CD33 is a functional fragment or variant, and may be used as a targeting moiety in the molecules of the present disclosure. In some embodiments, the anti-CD33-derived targeting moiety comprises the 3 CDR sequences from the light chain variable region and the 3 CDR sequences from the heavy chain variable region of an anti-CD33 antibody. In some embodiments, the anti-CD33-derived targeting moiety comprises alterations from the naturally occurring sequence that have improved binding affinity to the target in the context of the fusion molecule. In some embodiments, the anti-CD33-derived targeting moiety is derived from gemtuzumab. In some embodiments, the anti-CD33-derived targeting moiety is derived from a modified version of gemtuzumab, such as an scFv format of gemtuzumab. In some embodiments, the anti-CD33-derived targeting moiety comprises SEQ ID NO:16. In some embodiments, the anti-CD33-derived targeting moiety comprises an amino acid sequence having 75%, 80%, 85%, 90%, 95%, 97%, or 99% identity with SEQ ID NO:16.

In some embodiments, the targeting moiety is derived from Interleukin-2 (IL2). Adult T cell leukemia/lymphoma (ATLL), Hodgkin lymphoma, cutaneous T cell lymphoma (CTCL), T cell large granular lymphocytic leukemia (T-LGL), and T cell acute lymphoblastic leukemia (T-ALL) are all known to express CD25, a receptor for IL2, at high levels. Any fragment or variant of IL2 that has specific binding activity with respect to CD25 is a functional fragment or variant, and may be used as a targeting moiety in the molecules of the present disclosure. In some embodiments, the IL2-derived targeting moiety comprises alterations from the naturally occurring sequence that have improved binding affinity to the target in the context of the fusion molecule. In some embodiments, the IL2-derived targeting moiety is derived from human IL2. In some embodiments, the IL2-derived targeting moiety comprises SEQ ID NO:83. In some embodiments, the IL2-derived targeting moiety comprises an amino acid sequence having 75%, 80%, 85%, 90%, 95%, 97%, or 99% identity with any one of SEQ ID NOs:83.

In some embodiments, the targeting moiety is derived from Interleukin-4 (IL4). The key receptor for IL4, CD124, is implicated in a variety of hematological cancers. Chronic lymphocytic leukemia (CLL), non-Hodgkin lymphomas, Hodgkin lymphomas, and multiple myeloma (MM) are known to express CD124 at high levels. Any fragment or variant of IL4 that has specific binding activity with respect to CD124 is a functional fragment or variant, and may be used as a targeting moiety in the molecules of the present disclosure. In some embodiments, the IL4-derived targeting moiety comprises alterations from the naturally occurring sequence that have improved binding affinity to the target in the context of the fusion molecule. In some embodiments, the IL4-derived targeting moiety is derived from human IL4. In some embodiments, the IL4-derived targeting moiety comprises SEQ ID NO:85. In some embodiments, the IL4-derived targeting moiety comprises an amino acid sequence having 75%, 80%, 85%, 90%, 95%, 97%, or 99% identity with any one of SEQ ID NOs:85.

In some embodiments, the targeting moiety is derived from an anti-B-cell maturation antigen (BCMA) antibody. BCMA is known to be highly expressed on the surface of multiple myeloma (MM) cells, a type of hematological cancer. Any antibody that specifically binds BCMA may be used. For example, several anti-BCMA antibodies are described in U.S. Pat. No. 9,243,058. Any fragment or variant of an anti-BCMA antibody that has specific binding activity with respect to BCMA is a functional fragment or variant, and may be used as a targeting moiety in the molecules of the present disclosure. In some embodiments, the anti-BCMA-derived targeting moiety comprises the 3 CDR sequences from the light chain variable region and the 3 CDR sequences from the heavy chain variable region of an anti-BCMA antibody. In some embodiments, the anti-BCMA-derived targeting moiety comprises alterations from the naturally occurring sequence that have improved binding affinity to the target in the context of the fusion molecule. In some embodiments, the anti-BCMA-derived targeting moiety is derived from a modified version of an anti-BCMA antibody, such as an scFv format. In some embodiments, the anti-BCMA-derived targeting moiety comprises SEQ ID NO:79. In some embodiments, the anti-BCMA-derived targeting moiety comprises an amino acid sequence having 75%, 80%, 85%, 90%, 95%, 97%, or 99% identity with SEQ ID NO:79.

In some embodiments, the targeting moiety is derived from an anti-CD19 antibody. CD19 is expressed on B-cells and contributes to regulation of B-cell activation and differentiation. It is known to be highly expressed on the surface of B-cell lymphomas and leukemias. Any antibody that specifically binds CD19 may be used. For example, an anti-CD19 antibody is described in U.S. Pat. No. 7,635,472. Any fragment or variant of an anti-CD19 antibody that has specific binding activity with respect to CD19 is a functional fragment or variant, and may be used as a targeting moiety in the molecules of the present disclosure. In some embodiments, the anti-CD19-derived targeting moiety comprises the 3 CDR sequences from the light chain variable region and the 3 CDR sequences from the heavy chain variable region of an anti-CD19 antibody. In some embodiments, the anti-CD19-derived targeting moiety comprises alterations from the naturally occurring sequence that have improved binding affinity to the target in the context of the fusion molecule. In some embodiments, the anti-CD19-derived targeting moiety is derived from blinitumumab. In some embodiments, the anti-CD19-derived targeting moiety comprises SEQ ID NO:81. In some embodiments, the anti-CD19-derived targeting moiety comprises an amino acid sequence having 75%, 80%, 85%, 90%, 95%, 97%, or 99% identity with SEQ ID NO:81.

In some embodiments, the targeted cells are non-hematological cancer cells. Non-hematological cancers may also be referred to as solid tumors, solid tumor cancers, or solid tissue cancers. Non-hematological cancers can be difficult to treat because the cancer cells are often clustered, resulting in treatment challenges arising from the conditions in the tumor microenvironment and the difficulty of getting drug to penetrate to cells interior to the tumor.

Non-hematological cancers are classified based on various criteria, including tissue, organ, system, histological, and genetic factors. Non-hematological cancers include carcinomas, sarcomas, melanomas, neuroendocrine tumors, central nervous system tumors, germ cell tumors, and embryonal tumors. Non-hematological cancers may be identified by their organ/system of origin. For example, non-hematological cancers include thoracic cancers, including lung cancer (both small cell lung cancer and non-small cell lung cancer) and mesothelioma, gastrointestinal cancer, including pancreatic, colorectal, gastric, gastroesophageal, hepatic, and biliary tract cancers, genitourinary cancer, including prostate, renal, bladder/urothelial, and testicular cancers, gynecologic cancer, including ovarian, cervical, and endometrial/uterine cancers, breast cancers, head and neck cancers, esophageal cancers, thyroid cancers, and skin cancers, along with cancers arising from any other bodily organs. Other types of non-hematological cancers include sarcomas, including soft tissue sarcoma, osteosarcoma, and chondrosarcoma, melanomas, including cutaneous melanoma and uveal melanoma, central nervous system tumors, including glioblastoma, astrocytoma, and medulloblastoma, neuroendocrine tumors, including carcinoid tumors and pancreatic neuroendocrine tumors, and germ cell tumors, including testicular germ cell tumors and ovarian germ cell tumors. Non-hematological cancers categorized by histology include adenocarcinomas, squamous cell carcinomas, small cell carcinomas, and large cell carcinomas. The targeted cells may be cancer cells of any non-hematological cancer, including any subtype, variant, or stage of a non-hematological cancer.

In some embodiments, the molecules provided herein comprise targeting moieties that bind to one or more antigens or receptors present on the surface of non-hematological cancer cells. A targeting moiety may target any antigen or receptor present on the surface of a non-hematological cancer cell, preferably an antigen or receptor that is preferentially expressed on the surface of the non-hematological cancer cells as compared to healthy cells. In some embodiments, the molecules provided herein comprise two or more targeting moieties. These two or more targeting moieties may be the same or they may be different. When the two or more targeting moieties are different, they may bind the same antigen or receptor on the surface of a non-hematological cancer cell or they may bind different antigens or receptors on the surface of a non-hematological cancer cell.

In some embodiments, the targeting moiety is derived from an anti-claudin18.2 (CLDN18.2) antibody. CLDN18.2 is known to be highly expressed on the surface of certain gastric, pancreatic and gastroesophageal cancers. Any antibody that specifically binds CLDN18.2 may be used. For example, known anti-CLDN18.2 antibodies include osemitamab (NCI code C173726) and zolbetuximab (NCI code C85475). Any fragment or variant of an anti-CLDN18.2 antibody that has specific binding activity with respect to CLDN18.2 is a functional fragment or variant, and may be used as a targeting moiety in the molecules of the present disclosure. In some embodiments, the anti-CLDN18.2-derived targeting moiety comprises the 3 CDR sequences from the light chain variable region and the 3 CDR sequences from the heavy chain variable region of an anti-CLDN18.2 antibody. In some embodiments, the anti-CLDN18.2-derived targeting moiety comprises alterations from the naturally occurring sequence that have improved binding affinity to the target in the context of the fusion molecule. In some embodiments, the anti-CLDN18.2-derived targeting moiety is derived from a modified version of an anti-CLDN18.2 antibody, such as an scFv format. In some embodiments, the anti-CLDN18.2-derived targeting moiety is derived from osemitamab. In some embodiments, the anti-CLDN18.2-derived antibody is derived from zolbetuximab. In some embodiments, the anti-CLDN18.2-derived targeting moiety comprises SEQ ID NO:92. In some embodiments, the anti-CLDN18.2-derived targeting moiety comprises SEQ ID NO:94. In some embodiments, the anti-CLDN18.2-derived targeting moiety comprises an amino acid sequence having 75%, 80%, 85%, 90%, 95%, 97%, or 99% identity with SEQ ID NO:92 or SEQ ID NO:94.

In some embodiments, the targeting moiety is derived from an anti-human epidermal growth factor receptor 2 (HER2) antibody. HER2 is known to be highly expressed on the surface of certain lung and breast cancers. Any antibody that specifically binds HER2 may be used. For example, known anti-HER2 antibodies include trastuzumab (NCI code C1647), and those described in Eigenbrot, C. et al. PNAS. 2010. 107:15039. Any fragment or variant of an anti-HER2 antibody that has specific binding activity with respect to HER2 is a functional fragment or variant, and may be used as a targeting moiety in the molecules of the present disclosure. In some embodiments, the anti-HER2-derived targeting moiety comprises the 3 CDR sequences from the light chain variable region and the 3 CDR sequences from the heavy chain variable region of an anti-HER2 antibody. In some embodiments, the anti-HER2-derived targeting moiety comprises alterations from the naturally occurring sequence that have improved binding affinity to the target in the context of the fusion molecule. In some embodiments, the anti-HER2-derived targeting moiety is derived from a modified version of an anti-HER2 antibody, such as an affibody or DARPin format. In some embodiments, the anti-HER2-derived targeting moiety comprises SEQ ID NO:98. In some embodiments, the anti-HER2-derived targeting moiety comprises SEQ ID NO: 100. In some embodiments, the anti-HER2-derived targeting moiety comprises SEQ ID NO: 102. In some embodiments, the anti-HER2-derived targeting moiety comprises SEQ ID NO: 118. In some embodiments, the anti-HER2-derived targeting moiety comprises an amino acid sequence having 75%, 80%, 85%, 90%, 95%, 97%, or 99% identity with any one of SEQ ID NOs:98, 100, 102, or 118.

In some embodiments, the targeting moiety is derived from an anti-mesothelin (MSLN) antibody. MSLN is known to be highly expressed on the surface of a variety of cancers, including mesotheliomas, ovarian, pancreatic, and lung cancers. Any antibody that specifically binds MSLN may be used. For example, one known anti-MSLN antibody is anetumab (NCI code C156400). Any fragment or variant of an anti-MSLN antibody that has specific binding activity with respect to MSLN is a functional fragment or variant, and may be used as a targeting moiety in the molecules of the present disclosure. In some embodiments, the anti-MSLN-derived targeting moiety comprises the 3 CDR sequences from the light chain variable region and the 3 CDR sequences from the heavy chain variable region of an anti-MSLN antibody. In some embodiments, the anti-MSLN-derived targeting moiety comprises alterations from the naturally occurring sequence that have improved binding affinity to the target in the context of the fusion molecule. In some embodiments, the anti-MSLN-derived targeting moiety is derived from a modified version of an anti-MSLN antibody, such as an scFv format. In some embodiments, the anti-MSLN-derived targeting moiety comprises SEQ ID NO: 104. In some embodiments, the anti-MSLN-derived targeting moiety comprises an amino acid sequence having 75%, 80%, 85%, 90%, 95%, 97%, or 99% identity with SEQ ID NO: 104.

In some embodiments, the targeting moiety is derived from an anti-glypican-3 (GPC3) antibody. GPC3 is an oncofetal protein known to be highly expressed on the surface of liver cancers. Any antibody that specifically binds GPC3 may be used. For example, one known anti-GPC3 antibody is codrituzumab (NCI code C80043). Any fragment or variant of an anti-GPC3 antibody that has specific binding activity with respect to GPC3 is a functional fragment or variant, and may be used as a targeting moiety in the molecules of the present disclosure. In some embodiments, the anti-GPC3-derived targeting moiety comprises the 3 CDR sequences from the light chain variable region and the 3 CDR sequences from the heavy chain variable region of an anti-GPC3 antibody. In some embodiments, the anti-GPC3-derived targeting moiety comprises alterations from the naturally occurring sequence that have improved binding affinity to the target in the context of the fusion molecule. In some embodiments, the anti-GPC3-derived targeting moiety is derived from a modified version of an anti-GPC3 antibody, such as an scFv format. In some embodiments, the anti-GPC3-derived targeting moiety comprises SEQ ID NO:106. In some embodiments, the anti-GPC3-derived targeting moiety comprises an amino acid sequence having 75%, 80%, 85%, 90%, 95%, 97%, or 99% identity with SEQ ID NO: 106.

In some embodiments, the targeting moiety is derived from an anti-epidermal growth factor receptor (EGFR) antibody. EGFR is known to be overexpressed on the surface of non-small cell lung cancers, glioblastoma, squamous cell carcinoma, and colorectal cancer. Any antibody that specifically binds EGFR may be used. For example, known anti-EGFR antibodies include cetuximab (NCI code C1723) and those described in Zeronian, M. et al. BMC Mol and Cell Biol. 2022. 23:12. Any fragment or variant of an anti-EGFR antibody that has specific binding activity with respect to EGFR is a functional fragment or variant, and may be used as a targeting moiety in the molecules of the present disclosure. In some embodiments, the anti-EGFR-derived targeting moiety comprises the 3 CDR sequences from the light chain variable region and the 3 CDR sequences from the heavy chain variable region of an anti-EGFR antibody. In some embodiments, the anti-EGFR-derived targeting moiety comprises alterations from the naturally occurring sequence that have improved binding affinity to the target in the context of the fusion molecule. In some embodiments, the anti-EGFR-derived targeting moiety is derived from a modified version of an anti-EGFR antibody, such as an scFv format or a nanobody. In some embodiments, the anti-EGFR-derived targeting moiety comprises SEQ ID NO: 108. In some embodiments, the anti-EGFR-derived targeting moiety comprises SEQ ID NO: 110. In some embodiments, the anti-EGFR-derived targeting moiety comprises SEQ ID NO: 112. In some embodiments, the anti-EGFR-derived targeting moiety comprises an amino acid sequence having 75%, 80%, 85%, 90%, 95%, 97%, or 99% identity with any one of SEQ ID NOs:108, 110, or 112.

In some embodiments, the targeting moiety is derived from an anti-trophoblast cell surface antigen 2 (TROP2) antibody. TROP2 is known to be overexpressed on the surface of certain epithelial cancers, including breast, lung, pancreatic, and gastric cancers. Any antibody that specifically binds TROP2 may be used. For example, one known anti-TROP2 antibody is datopotamab (NCI code C174638). Any fragment or variant of an anti-TROP2 antibody that has specific binding activity with respect to TROP2 is a functional fragment or variant, and may be used as a targeting moiety in the molecules of the present disclosure. In some embodiments, the anti-TROP2-derived targeting moiety comprises the 3 CDR sequences from the light chain variable region and the 3 CDR sequences from the heavy chain variable region of an anti-TROP2 antibody. In some embodiments, the anti-TROP2-derived targeting moiety comprises alterations from the naturally occurring sequence that have improved binding affinity to the target in the context of the fusion molecule. In some embodiments, the anti-TROP2-derived targeting moiety is derived from a modified version of an anti-TROP2 antibody, such as an scFv format. In some embodiments, the anti-TROP2-derived targeting moiety comprises SEQ ID NO: 114. In some embodiments, the anti-TROP2-derived targeting moiety comprises an amino acid sequence having 75%, 80%, 85%, 90%, 95%, 97%, or 99% identity with SEQ ID NO: 114.

In some embodiments, the molecules provided herein comprise more than one targeting moiety. As discussed above, where two or more targeting moieties are present in the molecule, they may be the same or different, and they may bind to the same target or to different targets. In some embodiments, the molecules provided herein comprise two targeting moieties. Any two relevant targeting moieties may be selected for combination. In some embodiments, the molecules provided herein comprise a GM-CSF-derived targeting moiety and an IL3-derived targeting moiety. In some embodiments, the molecules provided herein comprise an IL2-derived targeting moiety and an IL4-derived targeting moiety. Where two or more targeting moieties are used, they may be connected by any suitable linker.

### D. Linkers

In some embodiments, the molecules provided herein comprise linkers. Linkers are used to provide a physical connection between subunits of a protein or polypeptide. In addition, linkers may serve other functions. In some cases, linkers should be flexible and have a relative lack of hydrophobic or charged residues in order to avoid interacting with the linked polypeptides in a manner that alters their conformation or activity. In other cases, a more rigid linker should be used to prevent undesired domain interactions or improve stability. In some cases, the number of amino acids that make up the linker may be increased or decreased to provide greater or lesser structural flexibility between the linked polypeptides. In some cases, linkers provide an additional specific function, such as providing a cleavage site. In some embodiments, the molecules provided herein comprise multiple domains, any of which may be connected by a linker.

In some embodiments, the linker is protease-cleavable. Without wishing to be bound by theory, protease cleavage of the linker between the toxin domain and the translocation domain may facilitate release of the toxin into the cytosol. The protease-cleavable linker may be cleaved by a protease present in the circulation or in the tumor microenvironment, such as matrix associated proteases or fibroblast activation protein, by a cell membrane proximal protease, such as angiotensin-converting enzyme, by a protease resident in the endosome, such as furin, cathepsins, or legumains, or by a protease resident in the cytosol, such as caspases or calpains.

In some embodiments, the linker is furin-cleavable. Furin is a protease present in many organisms that cleaves at specific furin cleavage sites within a target protein. Furin cleavage sites typically include the sequence “RXRR”, or +Arg/Lys-X-Lys/Arg-Arg/Lys”, where “X” can be any amino acid. There are many known variants of the furin cleavage site, and one skilled in the art can identify additional potential furin cleavage sites. Tools exist to help identify potential protease cleavage sites, including ProP-1.0, which can be found on the website: services.healthtech.dtu.dk/services/ProP-1.0. In some embodiments, the linker is non-cleavable. In some embodiments, the linker incorporates bounding cysteines, i.e., a cysteine on either side of the cleavage site, which may form a disulfide bond. Without wishing to be bound by theory, such bounding cysteines may assist in keeping the cleaved protein fragments together until released by the pH changes that occur in the endosome. In some embodiments, the molecules provided herein comprise multiple linkers, each independently selected to be protease-cleavable or non-cleavable. In some embodiments, the molecules provided herein comprise a CDT-derived domain linked to a DTB-derived translocation domain by a protease-cleavable linker. In some embodiments, the protease-cleavable linker is a furin-cleavable linker. In some embodiments, the furin-cleavable linker comprises SEQ ID NO:6, SEQ ID NO:7, or SEQ ID NO:8. In some embodiments, the molecules provided herein comprise one or more non-cleavable linkers between any two pairs of domains. In some embodiments, the non-cleavable linkers are independently selected from any of SEQ ID NOs:17-30, 58-60, and 65-78, or from the amino acid sequences “GP”, “KQ”, “QE”, or “GQG”.

### E. Molecules for Targeted Cancer Treatment

In some embodiments, the molecules provided herein comprise a toxin domain, a DTB-derived translocation domain, and one or more targeting moieties, wherein the toxin domain and the DTB-derived translocation domain are linked by a protease-cleavable linker and wherein the toxin domain has DNase activity. In some embodiments, the toxin domain is derived from cytolethal distending toxin (CDT). In some embodiments, the protease-cleavable linker is a furin-cleavable linker. In some embodiments, the furin-cleavable linker is a cysteine-bounded furin-cleavable linker.

Without wishing to be bound by theory, this structure may allow for targeted binding and internalization of the molecule facilitated by the targeting moiety or moieties, followed by cleavage and endosomal escape of the toxin domain by action of the DTB-derived domain and the protease-cleavable linker, after which the toxin domain causes the death of the targeted cell. When a furin-cleavable linker is used, the presence of the cysteines on either side of the furin-cleavable linker is believed to hold the toxin domain together with the DTB-derived domain after the linker is cleaved, permitting the DTB-derived domain to translocate the toxin domain out of the endosome.

This approach has multiple advantages over existing toxin-based cancer therapies. For example, the use of selective receptor-mediated uptake enhances tumor selectivity and minimizes off-target toxicity. The use of a cysteine-bounded furin-cleavable linker and translocation system further reduces the risk of non-specific effects from the toxin by ensuring that the toxic domain is preferentially released within the target cells. Additionally, the use of a toxin domain that is derived from only the CdtB domain, and does not require the presence of the CdtA or CdtC components, has several benefits. Not only does this reduce complexity in manufacturing and decrease the risk of adverse immune reactions to the molecule, but it also removes the native ability for CTD to enter a cell, thereby ensuring that the toxin only affects the cells targeted by the targeting moieties. Together, these factors result in an increase in both safety and efficacy as compared to conventional chemotherapies or antibody-drug conjugates.

In some embodiments, the molecules of the present disclosure comprise a CDT-derived domain, furin-cleavable linker, a DTB-derived translocation domain, and a GM-CSF-derived targeting moiety. In some embodiments, the molecule comprises any one of SEQ ID NOs:31-37 or SEQ ID NO:57. In some embodiments, the molecule comprises a functional variant of any one of SEQ ID NOs:31-37 or SEQ ID NO:57. In some embodiments, the molecule comprises an amino acid sequence having 75%, 80%, 85%, 90%, 95%, 97%, or 99% identity with any one of SEQ ID NOs:31-37 or SEQ ID NO:57.

In some embodiments, the molecules of the present disclosure comprise a CDT-derived domain, a furin-cleavable linker, a DTB-derived translocation domain, and an IL3-derived targeting moiety. In some embodiments, the molecule comprises any one of SEQ ID NOs:39-41. In some embodiments, the molecule comprises a functional variant of any one of SEQ ID NOs:39-41. In some embodiments, the molecule comprises an amino acid sequence having 75%, 80%, 85%, 90%, 95%, 97%, or 99% identity with any one of SEQ ID NOs:39-41.

In some embodiments, the molecules of the present disclosure comprise a CDT-derived domain, a furin-cleavable linker, a DTB-derived translocation domain, and an anti-CD33-derived targeting moiety. In some embodiments, the molecule comprises SEQ ID NO:38. In some embodiments, the molecule comprises a functional variant of SEQ ID NO:38. In some embodiments, the molecule comprises an amino acid sequence having 75%, 80%, 85%, 90%, 95%, 97%, or 99% identity with SEQ ID NO:38.

In some embodiments, the molecules of the present disclosure comprise a CDT-derived domain, a furin-cleavable linker, a DTB-derived translocation domain, a GM-CSF-derived targeting moiety, and an IL3-derived targeting moiety. In some embodiments, the molecule comprises any one of SEQ ID NOs:42-51 and 61-62. In some embodiments, the molecule comprises a functional variant of any one of SEQ ID NOs:42-51 and 61-62. In some embodiments, the molecule comprises an amino acid sequence having 75%, 80%, 85%, 90%, 95%, 97%, or 99% identity with any one of SEQ ID NOs:42-51 and 61-62.

In some embodiments, the molecules of the present disclosure comprise a CDT-derived domain, a furin-cleavable linker, a DTB-derived translocation domain, and an anti-BCMA-derived targeting moiety. In some embodiments, the molecule comprises SEQ ID NO:80 In some embodiments, the molecule comprises a functional variant of SEQ ID NO:80. In some embodiments, the molecule comprises an amino acid sequence having 75%, 80%, 85%, 90%, 95%, 97%, or 99% identity with SEQ ID NO:80.

In some embodiments, the molecules of the present disclosure comprise a CDT-derived domain, a furin-cleavable linker, a DTB-derived translocation domain, and an anti-CD19-derived targeting moiety. In some embodiments, the molecule comprises SEQ ID NO:82. In some embodiments, the molecule comprises a functional variant of SEQ ID NO:82. In some embodiments, the molecule comprises an amino acid sequence having 75%, 80%, 85%, 90%, 95%, 97%, or 99% identity with SEQ ID NO:82.

In some embodiments, the molecules of the present disclosure comprise a CDT-derived domain, a furin-cleavable linker, a DTB-derived translocation domain, and an IL2-derived targeting moiety. In some embodiments, the molecule comprises SEQ ID NO:84 or 91. In some embodiments, the molecule comprises a functional variant of SEQ ID NO:84 or 91. In some embodiments, the molecule comprises an amino acid sequence having 75%, 80%, 85%, 90%, 95%, 97%, or 99% identity with SEQ ID NO:84 or 91.

In some embodiments, the molecules of the present disclosure comprise a CDT-derived domain, a furin-cleavable linker, a DTB-derived translocation domain, and an IL4-derived targeting moiety. In some embodiments, the molecule comprises SEQ ID NO:86 or 89. In some embodiments, the molecule comprises a functional variant of SEQ ID NO:86 or 89. In some embodiments, the molecule comprises an amino acid sequence having 75%, 80%, 85%, 90%, 95%, 97%, or 99% identity with SEQ ID NO:86 or 89.

In some embodiments, the molecules of the present disclosure comprise a CDT-derived domain, a furin-cleavable linker, a DTB-derived translocation domain, and IL2-derived targeting moiety and an IL4-derived targeting moiety. In some embodiments, the molecule comprises any one of SEQ ID NOs:87, 88 and 90. In some embodiments, the molecule comprises a functional variant of any one of SEQ ID NOs:87, 88, and 90. In some embodiments, the molecule comprises an amino acid sequence having 75%, 80%, 85%, 90%, 95%, 97%, or 99% identity with any one of SEQ ID NOs:87, 88, and 90.

In some embodiments, the molecules of the present disclosure comprise a CDT-derived domain, a furin-cleavable linker, a DTB-derived translocation domain, and an anti-CLDN18.2-derived targeting moiety. In some embodiments, the molecule comprises any one of SEQ ID NOs:93 and 95-97. In some embodiments, the molecule comprises a functional variant of any one of SEQ ID NOs:93 and 95-97. In some embodiments, the molecule comprises an amino acid sequence having 75%, 80%, 85%, 90%, 95%, 97%, or 99% identity with SEQ ID NOs:93 and 95-97.

In some embodiments, the molecules of the present disclosure comprise a CDT-derived domain, a furin-cleavable linker, a DTB-derived translocation domain, and an anti-HER2-derived targeting moiety. In some embodiments, the molecule comprises any one of SEQ ID NOs:99, 101, 103, and 119. In some embodiments, the molecule comprises a functional variant of any one of SEQ ID NOs:99, 101, 103, and 119. In some embodiments, the molecule comprises an amino acid sequence having 75%, 80%, 85%, 90%, 95%, 97%, or 99% identity with any one of SEQ ID NOs:99, 101, 103, and 119.

In some embodiments, the molecules of the present disclosure comprise a CDT-derived domain, a furin-cleavable linker, a DTB-derived translocation domain, and an anti-MSLN-derived targeting moiety. In some embodiments, the molecule comprises SEQ ID NO: 105. In some embodiments, the molecule comprises a functional variant of SEQ ID NO:105. In some embodiments, the molecule comprises an amino acid sequence having 75%, 80%, 85%, 90%, 95%, 97%, or 99% identity with SEQ ID NO:105.

In some embodiments, the molecules of the present disclosure comprise a CDT-derived domain, a furin-cleavable linker, a DTB-derived translocation domain, and an anti-GPC3-derived targeting moiety. In some embodiments, the molecule comprises SEQ ID NO: 107. In some embodiments, the molecule comprises a functional variant of SEQ ID NO:107. In some embodiments, the molecule comprises an amino acid sequence having 75%, 80%, 85%, 90%, 95%, 97%, or 99% identity with SEQ ID NO:107.

In some embodiments, the molecules of the present disclosure comprise a CDT-derived domain, a furin-cleavable linker, a DTB-derived translocation domain, and an anti-EGFR-derived targeting moiety. In some embodiments, the molecule comprises any one of SEQ ID NOs:109, 111, and 113. In some embodiments, the molecule comprises a functional variant of any one of SEQ ID NOs:109, 111, and 113. In some embodiments, the molecule comprises an amino acid sequence having 75%, 80%, 85%, 90%, 95%, 97%, or 99% identity with any one of SEQ ID NOs:109, 111, and 113.

In some embodiments, the molecules of the present disclosure comprise a CDT-derived domain, a furin-cleavable linker, a DTB-derived translocation domain, and an anti-TROP2-derived targeting moiety. In some embodiments, the molecule comprises SEQ ID NO:115. In some embodiments, the molecule comprises a functional variant of SEQ ID NO:115. In some embodiments, the molecule comprises an amino acid sequence having 75%, 80%, 85%, 90%, 95%, 97%, or 99% identity with SEQ ID NO: 115.

It will be apparent that additional molecules that comprise a toxin domain, a DTB-derived translocation domain, and one or more targeting moieties, wherein the toxin domain and the DTB-derived translocation domain are linked by a protease-cleavable linker may be generated on the basis of the present disclosure by various arrangements of the described components. Such molecules are also explicitly contemplated as being within the scope of this disclosure.

The molecules of the present disclosure have been found to be effective for targeted killing of both hematological and non-hematological cancer cells using in vitro and in vivo testing, as described in the Examples. Efficacy has been demonstrated for multiple cancer-derived cell lines, suggesting that the molecules of the present disclosure will provide an effective treatment for patients suffering from cancer, regardless of the specific mutations present. Further, the molecules of the present disclosure incorporate multiple safety features, as described above, to minimize side effects, allowing for better treatment of all patients, including those who are elderly or have co-morbidities.

### III. Polynucleotides, Vectors, and Host Cells

Further provided herein are isolated polynucleotides that encode any of the presently disclosed molecules or a portion thereof (e.g., fusion protein or individual domains thereof). In certain embodiments, the polynucleotide is codon-optimized for expression in a host cell. Once a coding sequence is known or identified, codon optimization can be performed using known techniques and tools, such as the GenScript® OptimumGene™ tool or the ThermoFisher Scientific® GeneArt GeneOptimizer™. Codon-optimized sequences include sequences that are partially codon optimized, having one or more codons optimized for expression in the host cell, and those that are fully codon-optimized. It will also be appreciated that polynucleotides encoding the molecules of the present disclosure or a portion thereof may possess different nucleotide sequences while still encoding the same protein due to the degeneracy of the genetic code, splicing, etc.

In certain embodiments, a polynucleotide encoding the molecules of the present disclosure or a portion thereof may be comprised in a polynucleotide that includes other sequences and/or features. For example, a polynucleotide may include one or more sequences useful for control or expression of the encoding proteins, such as promoter sequence(s), polyadenylation sequence(s), sequence(s) encoding signal peptides, etc. The polynucleotide may comprise deoxyribonucleic acid (DNA) or ribonucleic acid (RNA).

Also provided are vectors comprising or containing a polynucleotide that encodes any of the presently disclosed molecules or a portion thereof. Any appropriate vector may be used, including viral vectors and plasmid vectors.

In a further aspect, the present disclosure also provides a host cell comprising a polynucleotide or vector disclosed herein. Any appropriate cell into which such a polynucleotide or vector may be introduced may be used. Examples of such cells include eukaryotic cells, including yeast cells, animal cells, insect cells, mammalian cells, and plant cells, and prokaryotic cells, including bacterial cells such as E. coli. In some embodiments, the host cell is a mammalian cell. In some embodiments, the host cell is an immortalized mammalian cell line. Cells appropriate for use in producing and expressing polynucleotides and vectors are known in the art.

In some embodiments, the cell may be transfected with a polynucleotide or vector disclosed herein. The term “transfection” encompasses any method known to one of skill in the art for introducing nucleic acid molecules into cells. Such methods include, for example, electroporation, lipofection, nanoparticle-based transfection, virus-based transfection, etc. Host cells may be transfected stably or transiently.

In some embodiments, the host cell expresses a molecule provided herein, or a portion thereof, encoded by the polynucleotide or vector. Such expression may include post-translational modifications such as removal of signal sequence, glycosylation, and other such modifications. In a related aspect, the present disclosure provides methods for producing the provided molecules or portions thereof, which methods comprise culturing a host cell for a sufficient time under conditions allowing for expression of the molecules and isolating the molecules. Methods useful for isolating and purifying recombinantly produced proteins include, for example, obtaining supernatant from suitable host cells that secrete the proteins into culture medium, concentrating the medium, and purifying the protein by passing the concentrate through a suitable purification matrix or series of matrices. Methods for purification of proteins are well known in the art.

### IV. Pharmaceutical Compositions

Also provided herein are compositions that comprise a therapeutic agent selected from any one or more of the presently disclosed molecules, polynucleotides, vectors, or host cells, singly or in any combination, and may also include other selected therapeutic agents. Such compositions may further comprise one or more pharmaceutically acceptable carriers, excipients, or diluents.

A pharmaceutically acceptable carrier is non-toxic, biocompatible and is selected so as not to detrimentally affect the biological activity of the therapeutic agent (and any other therapeutic agents combined therewith). Examples of pharmaceutically acceptable carriers for peptides are described in U.S. Pat. No. 5,211,657 to Yamada. The therapeutic agents described herein may be formulated into preparations in solid, semi solid, gel, liquid or gaseous forms such as tablets, capsules, powders, granules, ointments, solutions, depositories, inhalants and injections allowing for oral, parenteral or surgical administration. Local administration of the compositions by coating medical devices and the like is also contemplated.

Suitable carriers for parenteral delivery via injectable, infusion or irrigation and topical delivery include distilled water, physiological phosphate buffered saline, normal or lactated Ringer's solutions, dextrose solution, Hank's solution, or propanediol. In addition, sterile, fixed oils may be employed as a solvent or suspending medium. For this purpose, any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting example, microparticles, microspheres, nanospheres or nanoparticles composed of proteins, liposomes, carbohydrates, synthetic organic compounds, inorganic compounds, polymeric or copolymeric hydrogels and polymeric micelles. Suitable hydrogel and micelle delivery systems include the PEO:PHB:PEO copolymers and copolymer/cyclodextrin complexes disclosed in WO 2004/009664 A2 and the PEO and PEO/cyclodextrin complexes disclosed in U.S. Patent Application Publication No. 2002/0019369 A1. Such hydrogels may be injected locally at the site of intended action, or subcutaneously or intramuscularly to form a sustained release depot.

Compositions of the present invention may be formulated for delivery by any appropriate method including, without limitation, oral, topical, transdermal, sublingual, buccal, subcutaneously, intra-muscularly, intravenously, intra-arterially or as an inhalant.

The compositions of the present invention may also include biocompatible excipients, such as dispersing or wetting agents, suspending agents, diluents, buffers, penetration enhancers, emulsifiers, binders, thickeners, flavoring agents (for oral administration).

Pharmaceutical compositions according to certain embodiments of the present invention are formulated so as to allow the active ingredients contained therein to be bioavailable upon administration of the composition to a patient. Compositions that will be administered to a subject may take the form of one or more dosage units, and a container of a herein described therapeutic agent may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain an effective amount of therapeutic agent or composition of the present disclosure, for treatment of a disease or condition of interest in accordance with teachings herein.

A composition may be in the form of a solid or liquid. In some embodiments, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert fillers or diluents such as sucrose, corn starch, or cellulose. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the composition is in the form of a capsule, for example, a gelatin capsule, it may contain, in addition to materials of the above type, a liquid carrier such as polyethylene glycol or oil.

The composition may be in the form of a liquid, for example, an elixir, syrup, solution, emulsion or suspension. The liquid may be for oral administration or for delivery by injection, as two examples. When intended for oral administration, preferred compositions contain, in addition to the present compounds, one or more of a sweetening agent, preservative, dye/colorant and flavor enhancer. In a composition intended to be administered by injection, one or more of a surfactant, preservative, wetting agent, dispersing agent, suspending agent, buffer, stabilizer and isotonic agent may be included.

Liquid pharmaceutical compositions, whether they be solutions, suspensions or other like form, may include one or more of the following excipients: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred excipient. An injectable pharmaceutical composition is preferably sterile.

A liquid composition intended for either parenteral or oral administration should contain an amount of a therapeutic agent as described herein such that a suitable dosage will be obtained. The term “parenteral” includes subcutaneous, intravenous, intramuscular, intrasternal, or intra-arterial injection or infusion. Typically, the therapeutic agent is at least 0.01% of the composition. When intended for oral administration, this amount may be varied to be between about 0.1% and about 70% of the weight of the composition. Certain oral pharmaceutical compositions contain between about 4% and about 75% therapeutic agent.

The composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

A composition may include various materials which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The composition in solid or liquid form may include an agent that binds to the therapeutic agent(s) of the disclosure and thereby assists in the delivery of the compound. Suitable agents that may act in this capacity include one or more proteins or a liposome.

The composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi-phasic, or tri-phasic system in order to deliver the active ingredient(s). Delivery of the aerosol includes the necessary container, activators, valves, sub-containers, and the like, which together may form a kit. One of ordinary skill in the art, without undue experimentation, may determine preferred aerosols.

It will be understood that compositions of the present disclosure also encompass carrier molecules for polynucleotides, as described herein (e.g., lipid nanoparticles, nanoscale delivery platforms, and the like).

The pharmaceutical compositions may be prepared by methodology well known in the pharmaceutical art. For example, a composition intended to be administered by injection can be prepared by combining a composition that comprises therapeutic agent as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the composition so as to facilitate dissolution or homogeneous suspension in the aqueous delivery system.

### V. Methods and Uses

Provided herein are methods for use of the molecules, polynucleotides, vectors, host cells, or compositions of the present disclosure for the treatment of cancer. In some embodiments, the cancer is a hematological cancer. In some embodiments, the cancer is a leukemia, lymphoma, plasma cell neoplasm, MDS, MPN, histiocytic or dendritic cell neoplasm, or a mast cell neoplasm. In some embodiments, the cancer is an acute leukemia, a chronic leukemia, or another leukemia. In some embodiments, the cancer is acute myeloid leukemia (AMIL), acute promyelocytic leukemia, acute lymphoblastic leukemia (ALL), chronic myeloid leukemia (CML), chronic lymphocytic leukemia (CLL), chronic myelomonocytic leukemia (CMML), juvenile myelomonocytic leukemia (JMML), hairy cell leukemia, prolymphocytic leukemia, large granular lymphocytic leukemia (LGL) including T-cell LGL and NK-cell LGL, Hodgkin lymphoma, non-Hodgkin lymphoma including B-cell lymphoma and T-cell/NK-cell lymphoma, cutaneous T cell lymphoma (CTCL), adult T cell leukemia/lymphoma (ATLL), multiple myeloma (MM), monoclonal gammopathy of undetermined significance (MGUS), plasmacytoma, Waldenstrom macroglobulinemia, light chain (AL) amyloidosis, polycythemia vera (PV), essential thrombocythemia (ET), primary myelofibrosis (PMF), Langerhans cell histiocytosis (LCH), histiocytic sarcoma, follicular dendritic cell sarcoma, interdigitating dendritic cell sarcoma, systemic mastocytosis, mast cell leukemia, or blastic plasmacytoid dendritic cell neoplasm (BPDCN). In some embodiments, the cancer is a non-hematological cancer. In some embodiments, the cancer is a carcinoma, a sarcoma, a melanoma, a neuroendocrine tumor, a central nervous system tumor, a germ cell tumor, or an embryonal tumor. In some embodiments, the cancer is a thoracic cancer, such as a lung cancer (including small cell lung cancer and non-small cell lung cancer) or a mesothelioma; a gastrointestinal cancer, such as a pancreatic, colorectal, gastric, gastroesophageal, hepatic, or biliary tract cancer; a genitourinary cancer, such as a prostate, renal, bladder/urothelial, or testicular cancer; a gynecologic cancer, such as an ovarian, cervical, endometrial, or uterine cancer; a breast cancer; a head and neck cancer; an esophageal cancer; a thyroid cancer; a skin cancer; or another epithelial cancer. In some embodiments, the cancer is a sarcoma, such as a soft tissue sarcoma, osteosarcoma, or chondrosarcoma; a melanoma, such as a cutaneous melanoma or an uveal melanoma; a central nervous system tumor, such as a glioblastoma, astrocytoma, or medulloblastoma; a neuroendocrine tumor, such as a carcinoid tumor or a pancreatic neuroendocrine tumor; or a germ cell tumor such as a testicular germ cell tumor or an ovarian germ cell tumor. In some embodiments, the cancer is an adenocarcinoma, squamous cell carcinoma, small cell carcinoma, or large cell carcinoma.

Also provided herein are methods of treating cancer in a subject comprising administering a therapeutically effective amount of a molecule or composition of the present disclosure to the subject. In some embodiments, the molecule comprises one or more targeting moieties that specifically bind to a cancer-related surface antigen or receptor and is administered to a subject suffering from cancer.

In some embodiments, the molecule comprises one or more targeting moieties that specifically bind to a surface antigen or receptor present on the surface of a hematological cancer cell and is administered to a subject suffering from a hematological cancer. In some embodiments, the hematological cancer cell is a leukemia, lymphoma, plasma cell neoplasm, myelodysplastic syndrome (MDS), myeloproliferative neoplasm (MPN), histiocytic or dendritic cell neoplasm, or a mast cell neoplasm cancer cell and the subject is suffering from a leukemia, lymphoma, plasma cell neoplasm, myelodysplastic syndrome (MDS), myeloproliferative neoplasm (MPN), histiocytic or dendritic cell neoplasm, or a mast cell neoplasm cancer, respectively. In some embodiments, the hematological cancer cell is an acute myeloid leukemia (AMIL), acute promyelocytic leukemia, acute lymphoblastic leukemia (ALL), chronic myeloid leukemia (CML), chronic lymphocytic leukemia (CLL), chronic myelomonocytic leukemia (CMML), juvenile myelomonocytic leukemia (JMML), hairy cell leukemia, prolymphocytic leukemia, large granular lymphocytic leukemia (LGL) including T-cell LGL and NK-cell LGL, Hodgkin lymphoma, non-Hodgkin lymphoma including B-cell lymphoma and T-cell/NK-cell lymphoma, cutaneous T cell lymphoma (CTCL), adult T cell leukemia/lymphoma (ATLL), multiple myeloma (MM), monoclonal gammopathy of undetermined significance (MGUS), plasmacytoma, Waldenstrom macroglobulinemia, light chain (AL) amyloidosis, polycythemia vera (PV), essential thrombocythemia (ET), primary myelofibrosis (PMF), Langerhans cell histiocytosis (LCH), histiocytic sarcoma, follicular dendritic cell sarcoma, interdigitating dendritic cell sarcoma, systemic mastocytosis, mast cell leukemia, or blastic plasmacytoid dendritic cell neoplasm (BPDCN) cancer cell and the subject is suffering from an acute myeloid leukemia (AMIL), acute promyelocytic leukemia, acute lymphoblastic leukemia (ALL), chronic myeloid leukemia (CML), chronic lymphocytic leukemia (CLL), chronic myelomonocytic leukemia (CMML), juvenile myelomonocytic leukemia (JMML), hairy cell leukemia, prolymphocytic leukemia, large granular lymphocytic leukemia (LGL) including T-cell LGL and NK-cell LGL, Hodgkin lymphoma, non-Hodgkin lymphoma including B-cell lymphoma and T-cell/NK-cell lymphoma, cutaneous T cell lymphoma (CTCL), adult T cell leukemia/lymphoma (ATLL), multiple myeloma (MM), monoclonal gammopathy of undetermined significance (MGUS), plasmacytoma, Waldenstrom macroglobulinemia, light chain (AL) amyloidosis, polycythemia vera (PV), essential thrombocythemia (ET), primary myelofibrosis (PMF), Langerhans cell histiocytosis (LCH), histiocytic sarcoma, follicular dendritic cell sarcoma, interdigitating dendritic cell sarcoma, systemic mastocytosis, mast cell leukemia, or blastic plasmacytoid dendritic cell neoplasm (BPDCN) cancer, respectively.

In some embodiments, the molecule comprises one or more targeting moieties that specifically bind to an AML-related surface antigen or receptor and is administered to a subject suffering from AML. In some embodiments, the molecule comprises a GM-CSF-derived targeting moiety and is administered to a subject suffering from AML, CMML, BPDCN, ALL, or JMML. In some embodiments, the molecule comprises an IL-3-derived targeting moiety and is administered to a subject suffering from AML, CMML, BPDCN, ALL, or JMML. In some embodiments, the molecule comprises an anti-CD33-derived targeting moiety and is administered to a subject suffering from AML. In some embodiments, the molecule comprises a GM-CSF-derived targeting moiety and an IL-3-derived targeting moiety and is administered to a subject suffering from AML, CMML, BPDCN, ALL, or JMML. In some embodiments, the molecule comprises an IL-2-derived targeting moiety and is administered to a subject suffering from ATLL, Hodgkin lymphoma, CTCL, T-LGL, or T-ALL. In some embodiments, the molecule comprises an IL4-derived targeting moiety and is administered to a subject suffering from CLL, non-Hodgkin lymphoma, Hodgkin lymphoma, or MM. In some embodiments, the molecule comprises an anti-BCMA-derived targeting moiety and is administered to a subject suffering from MM. In some embodiments, the molecule comprises an anti-CD19-derived targeting moiety and is administered to a subject suffering from B-cell leukemia or lymphoma.

In some embodiments, the molecule comprises one or more targeting moieties that specifically bind to a surface antigen or receptor present on the surface of a non-hematological cancer cell and is administered to a subject suffering from a non-hematological cancer. In some embodiments, the non-hematological cancer cell is a carcinoma, a sarcoma, a melanoma, a neuroendocrine tumor, a central nervous system tumor, a germ cell tumor, or an embryonal tumor cancer cell and the subject is suffering from a carcinoma, a sarcoma, a melanoma, a neuroendocrine tumor, a central nervous system tumor, a germ cell tumor, or an embryonal tumor cancer, respectively. In some embodiments, the non-hematological cancer cell is a thoracic cancer, such as a lung cancer (including small cell lung cancer and non-small cell lung cancer) or a mesothelioma; a gastrointestinal cancer, such as a pancreatic, colorectal, gastric, gastroesophageal, hepatic, or biliary tract cancer; a genitourinary cancer, such as a prostate, renal, bladder/urothelial, or testicular cancer; a gynecologic cancer, such as an ovarian, cervical, endometrial, or uterine cancer; a breast cancer; a head and neck cancer; an esophageal cancer; a thyroid cancer; a skin cancer; or another epithelial cancer cell and the subject is suffering from a thoracic cancer, such as a lung cancer (including small cell lung cancer and non-small cell lung cancer) or a mesothelioma; a gastrointestinal cancer, such as a pancreatic, colorectal, gastric, gastroesophageal, hepatic, or biliary tract cancer; a genitourinary cancer, such as a prostate, renal, bladder/urothelial, or testicular cancer; a gynecologic cancer, such as an ovarian, cervical, endometrial, or uterine cancer; a breast cancer; a head and neck cancer; an esophageal cancer; a thyroid cancer; a skin cancer; or another epithelial cancer, respectively. In some embodiments, the non-hematological cancer cell is a sarcoma, such as a soft tissue sarcoma, osteosarcoma, or chondrosarcoma; a melanoma, such as a cutaneous melanoma or an uveal melanoma; a central nervous system tumor, such as a glioblastoma, astrocytoma, or medulloblastoma; a neuroendocrine tumor, such as a carcinoid tumor or a pancreatic neuroendocrine tumor; or a germ cell tumor such as a testicular germ cell tumor or an ovarian germ cell tumor cell and the subject is suffering from a sarcoma, such as a soft tissue sarcoma, osteosarcoma, or chondrosarcoma; a melanoma, such as a cutaneous melanoma or an uveal melanoma; a central nervous system tumor, such as a glioblastoma, astrocytoma, or medulloblastoma; a neuroendocrine tumor, such as a carcinoid tumor or a pancreatic neuroendocrine tumor; or a germ cell tumor such as a testicular germ cell tumor or an ovarian germ cell tumor cancer, respectively. In some embodiments, the non-hematological cancer cell is an adenocarcinoma, squamous cell carcinoma, small cell carcinoma, or large cell carcinoma cancer cell and the subject is suffering from an adenocarcinoma, squamous cell carcinoma, small cell carcinoma, or large cell carcinoma, respectively.

In some embodiments, the molecule comprises an anti-CLDN18.2-derived targeting moiety and is administered to a subject suffering from gastric, pancreatic, or gastroesophageal cancer. In some embodiments, the molecule comprises an anti-HER2-derived targeting moiety and is administered to a subject suffering from breast cancer or lung cancer. In some embodiments, the molecule comprises an anti-MSLN-derived targeting moiety and is administered to a subject suffering from mesothelioma, ovarian cancer, pancreatic cancer, or lung cancer. In some embodiments, the molecule comprises an anti-GPC3-derived targeting moiety and is administered to a subject suffering from liver cancer. In some embodiments, the molecule comprises an anti-EGFR-derived targeting moiety and is administered to a subject suffering from non-small cell lung cancer, glioblastoma, squamous cell carcinoma, or colorectal cancer. In some embodiments, the molecule comprises an anti-TROP2-derived targeting moiety and is administered to a subject suffering from an epithelial cancer, such as breast, lung, pancreatic, or gastric cancer.

Further provided herein is the use of a molecule, polynucleotide, vector, host cell, or composition of the present disclosure for the treatment of cancer in a subject. In some embodiments, the molecule comprises one or more targeting moieties that specifically bind to a cancer-related surface antigen or receptor and is used for treatment of a subject suffering from cancer. In some embodiments, the molecule comprises one or more targeting moieties that specifically bind to a surface antigen or receptor present on a hematological cancer cell and is used for treatment of a subject suffering from a hematological cancer. In some embodiments, the molecule comprises one or more targeting moieties that specifically bind to a surface antigen or receptor present on a non-hematological cancer cell and is used for treatment of a subject suffering from a non-hematological cancer. In some embodiments, the molecule comprises one or more targeting moieties that specifically bind to an AML-related surface antigen or receptor and is used for treatment of a subject suffering from AML. In some embodiments, the molecule comprises a GM-CSF-derived targeting moiety and is used for treatment of a subject suffering from AML, CMML, BPDCN, ALL, or JMML. In some embodiments, the molecule comprises an IL3-derived targeting moiety and is used for treatment of a subject suffering from AML, CMML, BPDCN, ALL, or JMML. In some embodiments, the molecule comprises an anti-CD33-derived targeting moiety and is used for treatment of a subject suffering from AIL. In some embodiments, the molecule comprises a GM-CSF-derived targeting moiety and an IL3-derived targeting moiety and is used for treatment of a subject suffering from AML, CMML, BPDCN, ALL, or JMML. In some embodiments, the molecule comprises an IL-2-derived targeting moiety and is used for treatment of a subject suffering from ATLL, Hodgkin lymphoma, CTCL, T-LGL, or T-ALL. In some embodiments, the molecule comprises an IL-4-derived targeting moiety and is used for treatment of a subject suffering from CLL, non-Hodgkin lymphoma, Hodgkin lymphoma, or MM. In some embodiments, the molecule comprises an anti-BCMA-derived targeting moiety and is used for treatment of a subject suffering from MM. In some embodiments, the molecule comprises an anti-CD19-derived targeting moiety and is used for treatment of a subject suffering from B-cell leukemia or lymphoma. In some embodiments, the molecule comprises an anti-CLDN18.2-derived targeting moiety and is used for treatment of a subject suffering from gastric, pancreatic, or gastroesophageal cancer. In some embodiments, the molecule comprises an anti-HER2-derived targeting moiety and is used for treatment of a subject suffering from breast cancer or lung cancer. In some embodiments, the molecule comprises an anti-MSLN-derived targeting moiety and is used for treatment of a subject suffering from mesothelioma, ovarian cancer, pancreatic cancer, or lung cancer. In some embodiments, the molecule comprises an anti-GPC3-derived targeting moiety and is used for treatment of a subject suffering from liver cancer. In some embodiments, the molecule comprises an anti-EGFR-derived targeting moiety and is used for treatment of a subject suffering from non-small cell lung cancer, glioblastoma, squamous cell carcinoma, or colorectal cancer. In some embodiments, the molecule comprises an anti-TROP2-derived targeting moiety and is used for treatment of a subject suffering from an epithelial cancer, such as breast, lung, pancreatic, or gastric cancer.

Additionally provided herein are a molecule, polynucleotide, vector, host cell, or composition of the present disclosure for use in the manufacture of a medicament for treating cancer. In some embodiments, the molecule comprises one or more targeting moieties that specifically bind to a cancer-related surface antigen or receptor and is used for manufacture of a medicament for treating cancer. In some embodiments, the molecule comprises one or more targeting moieties that specifically bind to a surface antigen or receptor present on a hematological cancer cell and is used for manufacture of a medicament for treating a hematological cancer. In some embodiments, the molecule comprises one or more targeting moieties that specifically bind to a surface antigen or receptor present on a non-hematological cancer cell and is used for manufacture of a medicament for treating a non-hematological cancer. In some embodiments, the molecule comprises one or more targeting moieties that specifically bind to an AML-related surface antigen or receptor and is used for manufacture of a medicament for treating AML. In some embodiments, the molecule comprises a GM-CSF-derived targeting moiety and is used for manufacture of a medicament for treating AML, CMML, BPDCN, ALL, or JMML. In some embodiments, the molecule comprises an IL3-derived targeting moiety and is used for manufacture of a medicament for treating AML, CMML, BPDCN, ALL, or JMML. In some embodiments, the molecule comprises an anti-CD33-derived targeting moiety and is used for manufacture of a medicament for treating AML. In some embodiments, the molecule comprises a GM-CSF-derived targeting moiety and an IL3-derived targeting moiety and is used for manufacture of a medicament for treating AML, CMML, BPDCN, ALL, or JMML. In some embodiments, the molecule comprises an IL2-derived targeting moiety and is used in the manufacture of a medicament for treating ATLL, Hodgkin lymphoma, CTCL, T-LGL, or T-ALL. In some embodiments, the molecule comprises an IL4-derived targeting moiety and is used in the manufacture of a medicament for treating CLL, non-Hodgkin lymphoma, Hodgkin lymphoma, or MM. In some embodiments, the molecule comprises an anti-BCMA-derived targeting moiety and is used for manufacture of a medicament for treating MM. In some embodiments, the molecule comprises an anti-CD19-derived targeting moiety and is used for manufacture of a medicament for treating B-cell leukemia or lymphoma. In some embodiments, the molecule comprises an anti-CLDN18.2-derived targeting moiety and is used for manufacture of a medicament for treating gastric, pancreatic, or gastroesophageal cancer. In some embodiments, the molecule comprises an anti-HER2-derived targeting moiety and is used for manufacture of a medicament for treating breast cancer or lung cancer. In some embodiments, the molecule comprises an anti-MSLN-derived targeting moiety and is used for manufacture of a medicament for treating mesothelioma, ovarian cancer, pancreatic cancer, or lung cancer. In some embodiments, the molecule comprises an anti-GPC3-derived targeting moiety and is used for manufacture of a medicament for treating liver cancer. In some embodiments, the molecule comprises an anti-EGFR-derived targeting moiety and is used for manufacture of a medicament for treating non-small cell lung cancer, glioblastoma, squamous cell carcinoma, or colorectal cancer. In some embodiments, the molecule comprises an anti-TROP2-derived targeting moiety and is used for manufacture of a medicament for treating an epithelial cancer, such as breast, lung, pancreatic, or gastric cancer.

Administration of the molecules or compositions of the present disclosure may be by any appropriate route, including oral, topical, transdermal, sublingual, buccal, subcutaneously, intra-muscularly, intravenously, intra-arterially or as an inhalant. The molecules or compositions of the present disclosure are administered in a therapeutically effective amount, which amount will vary depending upon a variety of factors including the specific molecules employed, the metabolic stability and length of action of the molecules, the age, sex, body weight, general health, and diet of the subject, the mode and time of administration, the rate of excretion, any additional therapeutic agents administered to the subject in the same time frame, the severity of the particular disorder or disease, and the genetic and epigenetic makeup of the subject. In certain embodiments, the molecules or compositions may be administered to the subject 1, 2, 3, 4, 5, 6, 7, 8, 9, or 10 times, or more. Successive administration may be carried out at any interval, including about 6, about 12, about 24, about 36, about 48, about 72, about 96, or about 108 hours apart, or more.

In some embodiments, the molecules, polynucleotides, vectors, host cells, or compositions of the present disclosure are used in combination with other therapeutic agents. For example, the molecules of the present disclosure may be used in combination with cancer therapeutics, including conventional chemotherapy drugs, antibody drug conjugates, or any other agent used for cancer therapy. Such combination therapy may include administration of a single pharmaceutical dosage formulation that contains molecules or compositions of the present disclosure together with one or more additional therapeutic agents, or the molecules or compositions of the present disclosure and the additional therapeutic agents may each be administered as a separate dosage formulation. Where separate dosage formulations are used, the molecules or compositions of the present disclosure and the additional therapeutic agents may be administered at essentially the same time, i.e., concurrently, or at separate times, i.e., sequentially in any order. In some embodiments, a combination therapy may comprise administration of the two or more different molecules of the present disclosure, or two or more compositions each comprising a different molecule of the present disclosure.

### VI. Sequences

The sequences referred to within the present specification are summarized in Table 1.

TABLE 1

| SEQ ID | | |
|---|---|---|
| NO. | Description | Sequence |
| 1 | CdtB from | NLSDFKVATWNLQGSSAVNESKWNINVRQLLSGEQGADI |
| | Haemophilus | LMVQEAGSLPSSAVRTSRVIQHGGTPIEEYTWNLGTRSRP |
| | ducreyi | NMVYIYYSRLDVGANRVNLAIVSRRQADEAFIVHSDSSV |
| | | LQSRPAVGIRIGTDVFFTVHALATGGSDAVSLIRNIFTTFN |
| | | SSSSPPERRVYSWMVVGDFNRAPANLEVALRQEPAVSEN |
| | | TIIIAPTEPTHRSGNILDYAILHDAHLPRREQARERIGASLM |
| | | LNQLRSQITSDHFPVSFVRDR |
| 2 | CdtB from | NLENYTVATWNLQGSSAINESKWNINVRQLLTGPQAAGI |
| | Haemophilus | LMVQEAGSLPSTAVHTRRMVQPEGVGFPIDEYVWNLGT |
| | parasuis | RSRPNNVYIYYSRLDVGANRVNLAIIARRMASEVFVINSS |
| | | SSVLTSRPAIGIRIDDDAFFSIHALSSGGADSLSLIQNIHTFF |
| | | NTEGRRHINWMAVGDFNRAPGRMQEALDSEPGLRNATL |
| | | IVAPTEPTHRSGGVLDYAVLHNANQTTQNTTVSASIMEN |
| | | QMRSQITSDHFPVSFVKKR |
| 3 | CdtB from | DLTDFRVATWNLQGASATTESKWNINVRQLISGENAVDI |
| | Escherichia coli | LAVQEAGSPPSTAVDTGTLIPSPGIPVRELIWNLSTNSRPQ |
| | | QVYIYFSAVDALGGRVNLALVSNRRADEVFVLSPVRQGG |
| | | RPLLGIRIGNDAFFTAHAIAMRNNDAPALVEEVYNFFRDS |
| | | RDPVHQALNWMILGDFNREPADLEMNLTVPVRRASEIISP |
| | | AAATQTSQRTLDYAVAGNSVAFRPSPLQAGIVYGARRTQ |
| | | ISSDHFPVGVSRR |
| 4 | CdtB from | NLSDFKVATWNLQGSSAVNESKWNINVRQLLSGEQGADI |
| | Aggregatibacter | LMVQEAGSLPSSAVRTSRVIQHGGTPIEEYTWNLGTRSRP |
| | actinomycet- | NMVYIYYSRLDVGANRVNLAIVSRRQADEAFIVHSDSSV |
| | emcomitans | LQSRPAVGIRIGTDVFFTVHALATGGSDAVSLIRNIFTTFT |
| | | SSPSSPERRGYSWMVVGDFNRAPVNLEAALRQEPAVSEN |
| | | TIIIAPTEPTHRSGNILDYAILHDAHLPRREQARERIGASLM |
| | | LNQLRSQITSDHFPVSFVRDR |
| 5 | CdtB from | ALTDYKVATWNLQGSSTRSENKWNVNVRQLVSGAGAV |
| | Salmonella | DILMVQEAGRPPASAVDTGRIINSPGIPVRELTWNLGSNS |
| | enterica | RPQQVFIYFSQLDVFAGRVNLAIVSHRRADEVIVLPPPSTA |
| | | SRPIMGIRIGSDAFFTIHALANRGVDAPAVVNSVFEFFRNS |
| | | TRPDMQATNWMIAGDFNRNPDNLRMAIETPVRNNTVILA |
| | | PSDPTQRSGGILDYAVVGNAIAFIPPVLRAGLLFGERATQI |
| | | SSDHYPVGIFLPPPGEPR |
| 6 | Furin-cleavable | RVRR |
| | linker | |
| 7 | Cysteine- | CAGNRVRRSVGSSLSC |
| | bounded furin- | |
| | cleavable linker | |
| 8 | Non-cysteine- | AGNRVRRSVGSSLSG |
| | bounded furin- | |
| | cleavable linker | |
| 9 | Cysteine- | CKGGASATFGVASLC |
| | bounded non- | |
| | cleavable linker | |
| 10 | DTB | INLDWDVIRDKTKTKIESLKEHGPIKNKMSESPNKTVSEE |
| | translocation | KAKQYLEEFHQTALEHPELSELKTVTGTNPVFAGANYAA |
| | domain | WAVNVAQVIDSETADNLEKTTAALSILPGIGSVMGIADG |
| | | AVHHNTEEIVAQSIALSSLMVAQAIPLVGELVDIGFAAYN |
| | | FVESIINLFQVVHNSYNRPAYSPGHKT |
| 11 | Human GM- | APARSPSPSTQPWEHVNAIQEARRLLNLSRDTAAEMNET |
| | CSF | VEVISEMFDLQEPTCLQTRLELYKQGLRGSLTKLKGPLTM |
| | | MASHYKQHCPPTPETSCATQIITFESFKENLKDFLLVIPFD |
| | | CWEPVQE |
| 12 | Human IL3 | APMTQTTPLKTSWVNCSNMIDEIITHLKQPPLPLLDENNL |
| | | NGEDQDILMENNLRRPNLEAFNRAVKSLQNASAIESILKN |
| | | LLPCLPLATAAPTRHPIHIKDGDWNEFRRKLTFYLKTLEN |
| | | AQAQQTTLSLAIF |
| 13 | Human IL3 | SWVNCSNMIDEIITHLKQPPLPLLDFNNLNGEDQDILMEN |
| | fragment (31- | NLRRPNLEAFNRAVKSLQNASAIESILKNLLPCLPLATAAP |
| | 143) | TRHPIHIKDGDWNEFRRKLTFYLKTLENAQAQ |
| 14 | Human IL3 | APMTQTTPLKTSWVNCSNMIDEIITHLKQPPLPLLDENNL |
| | (K135W | NGEDQDILMENNLRRPNLEAFNRAVKSLQNASAIESILKN |
| | mutant) | LLPCLPLATAAPTRHPIHIKDGDWNEFRRKLTFYLWTLEN |
| | | AQAQQTTLSLAIF |
| 15 | Human IL3 | SWVNCSNMIDEIITHLKQPPLPLLDFNNLNGEDQDILMEN |
| | fragment (31- | NLRRPNLEAFNRAVKSLQNASAIESILKNLLPCLPLATAAP |
| | 143, K135W | TRHPIHIKDGDWNEFRRKLTFYLWTLENAQAQ |
| | mutant) | |
| 16 | Anti-CD33 | EVQLVQSGAEVKKPGSSVKVSCKASGYTITDSNIHWVRQ |
| | (gemtuzumab, | APGQSLEWIGYIYPYNGGTDYNQKFKNRATLTVDNPTNT |
| | scFv format) | AYMELSSLRSEDTAFYYCVNGNPWLAYWGQGTLVTVSS |
| | | GAPQSGSEGASQGAGQPGGDIQLTQSPSTLSASVGDRVTI |
| | | TCRASESLDNYGIRFLTWFQQKPGKAPKLLMYAASNQGS |
| | | GVPSRFSGSGSGTEFTLTISSLQPDDFATYYCQQTKEVPW |
| | | SFGQGTKVEVKR |
| 17 | Linker | GSGGGGS |
| 18 | Linker | GGEASG |
| 19 | Linker | HGQG |
| 20 | Linker | GGEGSG |
| 21 | Linker | GGEASGG |
| 22 | Linker | HGGEGQGEPGAQSAGG |
| 23 | Linker | GAPQSGSEGASQGAGQPGG |
| 24 | Linker | GGGGEGGGG |
| 25 | Linker | HGGGGEGGGG |
| 26 | Linker | GGEGQGEPGAQSAGG |
| 27 | Linker | GGGGQGGGGEGGGSG |
| 28 | Linker | HGGGGQGGGGEGGGSG |
| 29 | Linker | GGGGS |
| 30 | Linker | GGGGSGGGGSGGGG |
| 31 | VCID0230 | NLSDFKVATWNLQGSSAVNESKWNINVRQLLSGEQGADI |
| | (CDT-furin site- | LMVQEAGSLPSSAVRTSRVIQHGGTPIEEYTWNLGTRSRP |
| | DTB-GMCSF) | NMVYIYYSRLDVGANRVNLAIVSRRQADEAFIVHSDSSV |
| | | LQSRPAVGIRIGTDVFFTVHALATGGSDAVSLIRNIFTTFN |
| | | SSSSPPERRVYSWMVVGDENRAPANLEVALRQEPAVSEN |
| | | TIIIAPTEPTHRSGNILDYAILHDAHLPRREQARERIGASLM |
| | | LNQLRSQITSDHFPVSFVRDRGGEASGCAGNRVRRSVGSS |
| | | LSCINLDWDVIRDKTKTKIESLKEHGPIKNKMSESPNKTV |
| | | SEEKAKQYLEEFHQTALEHPELSELKTVTGTNPVFAGAN |
| | | YAAWAVNVAQVIDSETADNLEKTTAALSILPGIGSVMGI |
| | | ADGAVHHNTEEIVAQSIALSSLMVAQAIPLVGELVDIGFA |
| | | AYNFVESIINLFQVVHNSYNRPAYSPGHKTHGQGAPARSP |
| | | SPSTQPWEHVNAIQEARRLLNLSRDTAAEMNETVEVISE |
| | | MFDLQEPTCLQTRLELYKQGLRGSLTKLKGPLTMMASH |
| | | YKQHCPPTPETSCATQIITFESFKENLKDFLLVIPFDCWEP |
| | | VQE |
| 32 | VCID1341 | NLSDFKVATWNLQGSSAVNESKWNINVRQLLSGEQGADI |
| | (CDT-furin site- | LMVQEAGSLPSSAVRTSRVIQHGGTPIEEYTWNLGTRSRP |
| | DTB-linker- | NMVYIYYSRLDVGANRVNLAIVSRRQADEAFIVHSDSSV |
| | GMCSF) | LQSRPAVGIRIGTDVFFTVHALATGGSDAVSLIRNIFTTFN |
| | | SSSSPPERRVYSWMVVGDFNRAPANLEVALRQEPAVSEN |
| | | TIIIAPTEPTHRSGNILDYAILHDAHLPRREQARERIGASLM |
| | | LNQLRSQITSDHFPVSFVRDRGGEASGCAGNRVRRSVGSS |
| | | LSCINLDWDVIRDKTKTKIESLKEHGPIKNKMSESPNKTV |
| | | SEEKAKQYLEEFHQTALEHPELSELKTVTGTNPVFAGAN |
| | | YAAWAVNVAQVIDSETADNLEKTTAALSILPGIGSVMGI |
| | | ADGAVHHNTEEIVAQSIALSSLMVAQAIPLVGELVDIGFA |
| | | AYNFVESIINLFQVVHNSYNRPAYSPGHKTHGGGGEGGG |
| | | GAPARSPSPSTQPWEHVNAIQEARRLLNLSRDTAAEMNE |
| | | TVEVISEMFDLQEPTCLQTRLELYKQGLRGSLTKLKGPLT |
| | | MMASHYKQHCPPTPETSCATQIITFESFKENLKDFLLVIPF |
| | | DCWEPVQE |
| 33 | VCID1342 | NLSDFKVATWNLQGSSAVNESKWNINVRQLLSGEQGADI |
| | (CDT-linker- | LMVQEAGSLPSSAVRTSRVIQHGGTPIEEYTWNLGTRSRP |
| | furin site-linker- | NMVYIYYSRLDVGANRVNLAIVSRRQADEAFIVHSDSSV |
| | GMCSF) | LQSRPAVGIRIGTDVFFTVHALATGGSDAVSLIRNIFTTFN |
| | | SSSSPPERRVYSWMVVGDFNRAPANLEVALRQEPAVSEN |
| | | TIIIAPTEPTHRSGNILDYAILHDAHLPRREQARERIGASLM |
| | | LNQLRSQITSDHFPVSFVRDRGGEGQGEPGAQSAGGCAG |
| | | NRVRRSVGSSLSCINLDWDVIRDKTKTKIESLKEHGPIKN |
| | | KMSESPNKTVSEEKAKQYLEEFHQTALEHPELSELKTVT |
| | | GTNPVFAGANYAAWAVNVAQVIDSETADNLEKTTAALSI |
| | | LPGIGSVMGIADGAVHHNTEEIVAQSIALSSLMVAQAIPL |
| | | VGELVDIGFAAYNFVESIINLFQVVHNSYNRPAYSPGHKT |
| | | HGGGGEGGGGAPARSPSPSTQPWEHVNAIQEARRLLNLS |
| | | RDTAAEMNETVEVISEMFDLQEPTCLQTRLELYKQGLRG |
| | | SLTKLKGPLTMMASHYKQHCPPTPETSCATQIITFESFKEN |
| | | LKDFLLVIPFDCWEPVQE |
| 34 | VCID1225 | NLSDFKVATWNLQGSSAVNESKWNINVRQLLSGEQGADI |
| | (AaCDT-furin | LMVQEAGSLPSSAVRTSRVIQHGGTPIEEYTWNLGTRSRP |
| | site-DTB- | NMVYIYYSRLDVGANRVNLAIVSRRQADEAFIVHSDSSV |
| | GMCSF) | LQSRPAVGIRIGTDVFFTVHALATGGSDAVSLIRNIFTTFT |
| | | SSPSSPERRGYSWMVVGDFNRAPVNLEAALRQEPAVSEN |
| | | TIIIAPTEPTHRSGNILDYAILHDAHLPRREQARERIGASLM |
| | | LNQLRSQITSDHFPVSFVRDRGGEASGCAGNRVRRSVGSS |
| | | LSCINLDWDVIRDKTKTKIESLKEHGPIKNKMSESPNKTV |
| | | SEEKAKQYLEEFHQTALEHPELSELKTVTGTNPVFAGAN |
| | | YAAWAVNVAQVIDSETADNLEKTTAALSILPGIGSVMGI |
| | | ADGAVHHNTEEIVAQSIALSSLMVAQAIPLVGELVDIGFA |
| | | AYNFVESIINLFQVVHNSYNRPAYSPGHKTHGQGAPARSP |
| | | SPSTQPWEHVNAIQEARRLLNLSRDTAAEMNETVEVISE |
| | | MFDLQEPTCLQTRLELYKQGLRGSLTKLKGPLTMMASH |
| | | YKQHCPPTPETSCATQIITFESFKENLKDFLLVIPFDCWEP |
| | | VQE |
| 35 | VCID1228 | DLTDFRVATWNLQGASATTESKWNINVRQLISGENAVDI |
| | (EcCDT-furin | LAVQEAGSPPSTAVDTGTLIPSPGIPVRELIWNLSTNSRPQ |
| | site-DTB- | QVYIYFSAVDALGGRVNLALVSNRRADEVFVLSPVRQGG |
| | GMCSF) | RPLLGIRIGNDAFFTAHAIAMRNNDAPALVEEVYNFFRDS |
| | | RDPVHQALNWMILGDFNREPADLEMNLTVPVRRASEIISP |
| | | AAATQTSQRTLDYAVAGNSVAFRPSPLQAGIVYGARRTQ |
| | | ISSDHFPVGVSRRGGEASGCAGNRVRRSVGSSLSCINLDW |
| | | DVIRDKTKTKIESLKEHGPIKNKMSESPNKTVSEEKAKQY |
| | | LEEFHQTALEHPELSELKTVTGTNPVFAGANYAAWAVN |
| | | VAQVIDSETADNLEKTTAALSILPGIGSVMGIADGAVHHN |
| | | TEEIVAQSIALSSLMVAQAIPLVGELVDIGFAAYNFVESIIN |
| | | LFQVVHNSYNRPAYSPGHKTHGQGAPARSPSPSTQPWEH |
| | | VNAIQEARRLLNLSRDTAAEMNETVEVISEMFDLQEPTCL |
| | | QTRLELYKQGLRGSLTKLKGPLTMMASHYKQHCPPTPET |
| | | SCATQIITFESFKENLKDFLLVIPFDCWEPVQE |
| 36 | VCID1230 | NLENYTVATWNLQGSSAINESKWNINVRQLLTGPQAAGI |
| | (HpCDT-furin | LMVQEAGSLPSTAVHTRRMVQPEGVGFPIDEYVWNLGT |
| | site-DTB- | RSRPNNVYIYYSRLDVGANRVNLAIIARRMASEVFVINSS |
| | GMCSF) | SSVLTSRPAIGIRIDDDAFFSIHALSSGGADSLSLIQNIHTFF |
| | | NTEGRRHINWMAVGDFNRAPGRMQEALDSEPGLRNATL |
| | | IVAPTEPTHRSGGVLDYAVLHNANQTTQNTTVSASIMEN |
| | | QMRSQITSDHFPVSFVKKRGGEASGCAGNRVRRSVGSSL |
| | | SCINLDWDVIRDKTKTKIESLKEHGPIKNKMSESPNKTVS |
| | | EEKAKQYLEEFHQTALEHPELSELKTVTGTNPVFAGANY |
| | | AAWAVNVAQVIDSETADNLEKTTAALSILPGIGSVMGIA |
| | | DGAVHHNTEEIVAQSIALSSLMVAQAIPLVGELVDIGFAA |
| | | YNFVESIINLFQVVHNSYNRPAYSPGHKTHGQGAPARSPS |
| | | PSTQPWEHVNAIQEARRLLNLSRDTAAEMNETVEVISEM |
| | | FDLQEPTCLQTRLELYKQGLRGSLTKLKGPLTMMASHYK |
| | | QHCPPTPETSCATQIITFESFKENLKDFLLVIPFDCWEPVQE |
| 37 | VCID1232 | ALTDYKVATWNLQGSSTRSENKWNVNVRQLVSGAGAV |
| | (SeCDT-furin | DILMVQEAGRPPASAVDTGRIINSPGIPVRELTWNLGSNS |
| | site-DTB- | RPQQVFIYFSQLDVFAGRVNLAIVSHRRADEVIVLPPPSTA |
| | GMCSF) | SRPIMGIRIGSDAFFTIHALANRGVDAPAVVNSVFEFFRNS |
| | | TRPDMQATNWMIAGDFNRNPDNLRMAIETPVRNNTVILA |
| | | PSDPTQRSGGILDYAVVGNAIAFIPPVLRAGLLFGERATQI |
| | | SSDHYPVGIFLPPPGEPRGGEASGCAGNRVRRSVGSSLSCI |
| | | NLDWDVIRDKTKTKIESLKEHGPIKNKMSESPNKTVSEEK |
| | | AKQYLEEFHQTALEHPELSELKTVTGTNPVFAGANYAA |
| | | WAVNVAQVIDSETADNLEKTTAALSILPGIGSVMGIADG |
| | | AVHHNTEEIVAQSIALSSLMVAQAIPLVGELVDIGFAAYN |
| | | FVESIINLFQVVHNSYNRPAYSPGHKTHGQGAPARSPSPS |
| | | TQPWEHVNAIQEARRLLNLSRDTAAEMNETVEVISEMFD |
| | | LQEPTCLQTRLELYKQGLRGSLTKLKGPLTMMASHYKQ |
| | | HCPPTPETSCATQIITFESFKENLKDFLLVIPFDCWEPVQE |
| 38 | VCID0231 | NLSDFKVATWNLQGSSAVNESKWNINVRQLLSGEQGADI |
| | (CDT-furin site- | LMVQEAGSLPSSAVRTSRVIQHGGTPIEEYTWNLGTRSRP |
| | DTB-anti- | NMVYIYYSRLDVGANRVNLAIVSRRQADEAFIVHSDSSV |
| | CD33) | LQSRPAVGIRIGTDVFFTVHALATGGSDAVSLIRNIFTTFN |
| | | SSSSPPERRVYSWMVVGDFNRAPANLEVALRQEPAVSEN |
| | | TIIIAPTEPTHRSGNILDYAILHDAHLPRREQARERIGASLM |
| | | LNQLRSQITSDHFPVSFVRDRGGEASGCAGNRVRRSVGSS |
| | | LSCINLDWDVIRDKTKTKIESLKEHGPIKNKMSESPNKTV |
| | | SEEKAKQYLEEFHQTALEHPELSELKTVTGTNPVFAGAN |
| | | YAAWAVNVAQVIDSETADNLEKTTAALSILPGIGSVMGI |
| | | ADGAVHHNTEEIVAQSIALSSLMVAQAIPLVGELVDIGFA |
| | | AYNFVESIINLFQVVHNSYNRPAYSPGHKTHGQGEVQLV |
| | | QSGAEVKKPGSSVKVSCKASGYTITDSNIHWVRQAPGQS |
| | | LEWIGYIYPYNGGTDYNQKFKNRATLTVDNPTNTAYME |
| | | LSSLRSEDTAFYYCVNGNPWLAYWGQGTLVTVSSGAPQ |
| | | SGSEGASQGAGQPGGDIQLTQSPSTLSASVGDRVTITCRA |
| | | SESLDNYGIRFLTWFQQKPGKAPKLLMYAASNQGSGVPS |
| | | RFSGSGSGTEFTLTISSLQPDDFATYYCQQTKEVPWSFGQ |
| | | GTKVEVKR |
| 39 | VCID0253 | NLSDFKVATWNLQGSSAVNESKWNINVRQLLSGEQGADI |
| | (CDT-furin site- | LMVQEAGSLPSSAVRTSRVIQHGGTPIEEYTWNLGTRSRP |
| | DTB- | NMVYIYYSRLDVGANRVNLAIVSRRQADEAFIVHSDSSV |
| | IL3(K135W)) | LQSRPAVGIRIGTDVFFTVHALATGGSDAVSLIRNIFTTFN |
| | | SSSSPPERRVYSWMVVGDFNRAPANLEVALRQEPAVSEN |
| | | TIIIAPTEPTHRSGNILDYAILHDAHLPRREQARERIGASLM |
| | | LNQLRSQITSDHFPVSFVRDRGGEASGCAGNRVRRSVGSS |
| | | LSCINLDWDVIRDKTKTKIESLKEHGPIKNKMSESPNKTV |
| | | SEEKAKQYLEEFHQTALEHPELSELKTVTGTNPVFAGAN |
| | | YAAWAVNVAQVIDSETADNLEKTTAALSILPGIGSVMGI |
| | | ADGAVHHNTEEIVAQSIALSSLMVAQAIPLVGELVDIGFA |
| | | AYNFVESIINLFQVVHNSYNRPAYSPGHKTHGQGAPMTQ |
| | | TTPLKTSWVNCSNMIDEIITHLKQPPLPLLDENNLNGEDQ |
| | | DILMENNLRRPNLEAFNRAVKSLQNASAIESILKNLLPCLP |
| | | LATAAPTRHPIHIKDGDWNEFRRKLTFYLWTLENAQAQQ |
| | | TTLSLAIF |
| 40 | VCID0488 | NLSDFKVATWNLQGSSAVNESKWNINVRQLLSGEQGADI |
| | (CDT-furin site- | LMVQEAGSLPSSAVRTSRVIQHGGTPIEEYTWNLGTRSRP |
| | DTB-IL3) | NMVYIYYSRLDVGANRVNLAIVSRRQADEAFIVHSDSSV |
| | | LQSRPAVGIRIGTDVFFTVHALATGGSDAVSLIRNIFTTFN |
| | | SSSSPPERRVYSWMVVGDFNRAPANLEVALRQEPAVSEN |
| | | TIIIAPTEPTHRSGNILDYAILHDAHLPRREQARERIGASLM |
| | | LNQLRSQITSDHFPVSFVRDRGGEASGCAGNRVRRSVGSS |
| | | LSCINLDWDVIRDKTKTKIESLKEHGPIKNKMSESPNKTV |
| | | SEEKAKQYLEEFHQTALEHPELSELKTVTGTNPVFAGAN |
| | | YAAWAVNVAQVIDSETADNLEKTTAALSILPGIGSVMGI |
| | | ADGAVHHNTEEIVAQSIALSSLMVAQAIPLVGELVDIGFA |
| | | AYNFVESIINLFQVVHNSYNRPAYSPGHKTHGQGAPMTQ |
| | | TTPLKTSWVNCSNMIDEIITHLKQPPLPLLDENNLNGEDQ |
| | | DILMENNLRRPNLEAFNRAVKSLQNASAIESILKNLLPCLP |
| | | LATAAPTRHPIHIKDGDWNEFRRKLTFYLKTLENAQAQQ |
| | | TTLSLAIF |
| 41 | VCID1312 | NLSDFKVATWNLQGSSAVNESKWNINVRQLLSGEQGADI |
| | (CDT-furin site- | LMVQEAGSLPSSAVRTSRVIQHGGTPIEEYTWNLGTRSRP |
| | DTB-linker- | NMVYIYYSRLDVGANRVNLAIVSRRQADEAFIVHSDSSV |
| | IL3(31-143, | LQSRPAVGIRIGTDVFFTVHALATGGSDAVSLIRNIFTTFN |
| | K135W)) | SSSSPPERRVYSWMVVGDFNRAPANLEVALRQEPAVSEN |
| | | TIIIAPTEPTHRSGNILDYAILHDAHLPRREQARERIGASLM |
| | | LNQLRSQITSDHFPVSFVRDRGGEASGCAGNRVRRSVGSS |
| | | LSCINLDWDVIRDKTKTKIESLKEHGPIKNKMSESPNKTV |
| | | SEEKAKQYLEEFHQTALEHPELSELKTVTGTNPVFAGAN |
| | | YAAWAVNVAQVIDSETADNLEKTTAALSILPGIGSVMGI |
| | | ADGAVHHNTEEIVAQSIALSSLMVAQAIPLVGELVDIGFA |
| | | AYNFVESIINLFQVVHNSYNRPAYSPGHKTHGGGGQGGG |
| | | GEGGGSGSWVNCSNMIDEIITHLKQPPLPLLDENNLNGED |
| | | QDILMENNLRRPNLEAFNRAVKSLQNASAIESILKNLLPC |
| | | LPLATAAPTRHPIHIKDGDWNEFRRKLTFYLWTLENAQA |
| | | Q |
| 42 | VCID0904 | NLSDFKVATWNLQGSSAVNESKWNINVRQLLSGEQGADI |
| | (CDT-furin site- | LMVQEAGSLPSSAVRTSRVIQHGGTPIEEYTWNLGTRSRP |
| | DTB-GMCSF- | NMVYIYYSRLDVGANRVNLAIVSRRQADEAFIVHSDSSV |
| | IL3(31-143, | LQSRPAVGIRIGTDVFFTVHALATGGSDAVSLIRNIFTTFN |
| | K135W)) | SSSSPPERRVYSWMVVGDFNRAPANLEVALRQEPAVSEN |
| | | TIIIAPTEPTHRSGNILDYAILHDAHLPRREQARERIGASLM |
| | | LNQLRSQITSDHFPVSFVRDRGGEASGCAGNRVRRSVGSS |
| | | LSCINLDWDVIRDKTKTKIESLKEHGPIKNKMSESPNKTV |
| | | SEEKAKQYLEEFHQTALEHPELSELKTVTGTNPVFAGAN |
| | | YAAWAVNVAQVIDSETADNLEKTTAALSILPGIGSVMGI |
| | | ADGAVHHNTEEIVAQSIALSSLMVAQAIPLVGELVDIGFA |
| | | AYNFVESIINLFQVVHNSYNRPAYSPGHKTHGQGAPARSP |
| | | SPSTQPWEHVNAIQEARRLLNLSRDTAAEMNETVEVISE |
| | | MFDLQEPTCLQTRLELYKQGLRGSLTKLKGPLTMMASH |
| | | YKQHCPPTPETSCATQIITFESFKENLKDFLLVIPFDCWEP |
| | | VQEGGGGEGGGGSWVNCSNMIDEIITHLKQPPLPLLDFN |
| | | NLNGEDQDILMENNLRRPNLEAFNRAVKSLQNASAIESIL |
| | | KNLLPCLPLATAAPTRHPIHIKDGDWNEFRRKLTFYLWTL |
| | | ENAQAQ |
| 43 | VCID1300 | NLSDFKVATWNLQGSSAVNESKWNINVRQLLSGEQGADI |
| | (CDT-furin site- | LMVQEAGSLPSSAVRTSRVIQHGGTPIEEYTWNLGTRSRP |
| | DTB-GMCSF- | NMVYIYYSRLDVGANRVNLAIVSRRQADEAFIVHSDSSV |
| | IL3(31-143)) | LQSRPAVGIRIGTDVFFTVHALATGGSDAVSLIRNIFTTFN |
| | | SSSSPPERRVYSWMVVGDFNRAPANLEVALRQEPAVSEN |
| | | TIIIAPTEPTHRSGNILDYAILHDAHLPRREQARERIGASLM |
| | | LNQLRSQITSDHFPVSFVRDRGGEASGCAGNRVRRSVGSS |
| | | LSCINLDWDVIRDKTKTKIESLKEHGPIKNKMSESPNKTV |
| | | SEEKAKQYLEEFHQTALEHPELSELKTVTGTNPVFAGAN |
| | | YAAWAVNVAQVIDSETADNLEKTTAALSILPGIGSVMGI |
| | | ADGAVHHNTEEIVAQSIALSSLMVAQAIPLVGELVDIGFA |
| | | AYNFVESIINLFQVVHNSYNRPAYSPGHKTHGQGAPARSP |
| | | SPSTQPWEHVNAIQEARRLLNLSRDTAAEMNETVEVISE |
| | | MFDLQEPTCLQTRLELYKQGLRGSLTKLKGPLTMMASH |
| | | YKQHCPPTPETSCATQIITFESFKENLKDFLLVIPFDCWEP |
| | | VQEGGGGEGGGGSWVNCSNMIDEIITHLKQPPLPLLDFN |
| | | NLNGEDQDILMENNLRRPNLEAFNRAVKSLQNASAIESIL |
| | | KNLLPCLPLATAAPTRHPIHIKDGDWNEFRRKLTFYLKTL |
| | | ENAQAQ |
| 44 | VCID1302 | NLSDFKVATWNLQGSSAVNESKWNINVRQLLSGEQGADI |
| | (CDT-furin site- | LMVQEAGSLPSSAVRTSRVIQHGGTPIEEYTWNLGTRSRP |
| | DTB-linker- | NMVYIYYSRLDVGANRVNLAIVSRRQADEAFIVHSDSSV |
| | GMCSF-linker- | LQSRPAVGIRIGTDVFFTVHALATGGSDAVSLIRNIFTTFN |
| | IL3(31-143)) | SSSSPPERRVYSWMVVGDFNRAPANLEVALRQEPAVSEN |
| | | TIIIAPTEPTHRSGNILDYAILHDAHLPRREQARERIGASLM |
| | | LNQLRSQITSDHFPVSFVRDRGGEASGCAGNRVRRSVGSS |
| | | LSCINLDWDVIRDKTKTKIESLKEHGPIKNKMSESPNKTV |
| | | SEEKAKQYLEEFHQTALEHPELSELKTVTGTNPVFAGAN |
| | | YAAWAVNVAQVIDSETADNLEKTTAALSILPGIGSVMGI |
| | | ADGAVHHNTEEIVAQSIALSSLMVAQAIPLVGELVDIGFA |
| | | AYNFVESIINLFQVVHNSYNRPAYSPGHKTHGGGGEGGG |
| | | GAPARSPSPSTQPWEHVNAIQEARRLLNLSRDTAAEMNE |
| | | TVEVISEMFDLQEPTCLQTRLELYKQGLRGSLTKLKGPLT |
| | | MMASHYKQHCPPTPETSCATQIITFESFKENLKDFLLVIPF |
| | | DCWEPVQEGGGGQGGGGEGGGSGSWVNCSNMIDEIITH |
| | | LKQPPLPLLDFNNLNGEDQDILMENNLRRPNLEAFNRAV |
| | | KSLQNASAIESILKNLLPCLPLATAAPTRHPIHIKDGDWNE |
| | | FRRKLTFYLKTLENAQAQ |
| 45 | VCID1303 | NLSDFKVATWNLQGSSAVNESKWNINVRQLLSGEQGADI |
| | (CDT-furin site- | LMVQEAGSLPSSAVRTSRVIQHGGTPIEEYTWNLGTRSRP |
| | DTB-linker- | NMVYIYYSRLDVGANRVNLAIVSRRQADEAFIVHSDSSV |
| | GMCSF-linker- | LQSRPAVGIRIGTDVFFTVHALATGGSDAVSLIRNIFTTFN |
| | IL3(31-143, | SSSSPPERRVYSWMVVGDFNRAPANLEVALRQEPAVSEN |
| | K135W)) | TIIIAPTEPTHRSGNILDYAILHDAHLPRREQARERIGASLM |
| | | LNQLRSQITSDHFPVSFVRDRGGEASGCAGNRVRRSVGSS |
| | | LSCINLDWDVIRDKTKTKIESLKEHGPIKNKMSESPNKTV |
| | | SEEKAKQYLEEFHQTALEHPELSELKTVTGTNPVFAGAN |
| | | YAAWAVNVAQVIDSETADNLEKTTAALSILPGIGSVMGI |
| | | ADGAVHHNTEEIVAQSIALSSLMVAQAIPLVGELVDIGFA |
| | | AYNFVESIINLFQVVHNSYNRPAYSPGHKTHGGGGEGGG |
| | | GAPARSPSPSTQPWEHVNAIQEARRLLNLSRDTAAEMNE |
| | | TVEVISEMFDLQEPTCLQTRLELYKQGLRGSLTKLKGPLT |
| | | MMASHYKQHCPPTPETSCATQIITFESFKENLKDFLLVIPF |
| | | DCWEPVQEGGGGQGGGGEGGGSGSWVNCSNMIDEIITH |
| | | LKQPPLPLLDFNNLNGEDQDILMENNLRRPNLEAFNRAV |
| | | KSLQNASAIESILKNLLPCLPLATAAPTRHPIHIKDGDWNE |
| | | FRRKLTFYLWTLENAQAQ |
| 46 | VCID1333 | NLSDFKVATWNLQGSSAVNESKWNINVRQLLSGEQGADI |
| | (AaCDT-furin | LMVQEAGSLPSSAVRTSRVIQHGGTPIEEYTWNLGTRSRP |
| | site-DTB- | NMVYIYYSRLDVGANRVNLAIVSRRQADEAFIVHSDSSV |
| | GMCSF- | LQSRPAVGIRIGTDVFFTVHALATGGSDAVSLIRNIFTTFT |
| | IL3(31-143, | SSPSSPERRGYSWMVVGDFNRAPVNLEAALRQEPAVSEN |
| | K135W)) | TIIIAPTEPTHRSGNILDYAILHDAHLPRREQARERIGASLM |
| | | LNQLRSQITSDHFPVSFVRDRGGEASGCAGNRVRRSVGSS |
| | | LSCINLDWDVIRDKTKTKIESLKEHGPIKNKMSESPNKTV |
| | | SEEKAKQYLEEFHQTALEHPELSELKTVTGTNPVFAGAN |
| | | YAAWAVNVAQVIDSETADNLEKTTAALSILPGIGSVMGI |
| | | ADGAVHHNTEEIVAQSIALSSLMVAQAIPLVGELVDIGFA |
| | | AYNFVESIINLFQVVHNSYNRPAYSPGHKTHGQGAPARSP |
| | | SPSTQPWEHVNAIQEARRLLNLSRDTAAEMNETVEVISE |
| | | MFDLQEPTCLQTRLELYKQGLRGSLTKLKGPLTMMASH |
| | | YKQHCPPTPETSCATQIITFESFKENLKDFLLVIPFDCWEP |
| | | VQEGGGGEGGGGSWVNCSNMIDEIITHLKQPPLPLLDEN |
| | | NLNGEDQDILMENNLRRPNLEAFNRAVKSLQNASAIESIL |
| | | KNLLPCLPLATAAPTRHPIHIKDGDWNEFRRKLTFYLWTL |
| | | ENAQAQ |
| 47 | VCID1334 | NLSDFKVATWNLQGSSAVNESKWNINVRQLLSGEQGADI |
| | (AaCDT-furin | LMVQEAGSLPSSAVRTSRVIQHGGTPIEEYTWNLGTRSRP |
| | site-DTB- | NMVYIYYSRLDVGANRVNLAIVSRRQADEAFIVHSDSSV |
| | linker-GMCSF- | LQSRPAVGIRIGTDVFFTVHALATGGSDAVSLIRNIFTTFT |
| | linker-IL3(31- | SSPSSPERRGYSWMVVGDFNRAPVNLEAALRQEPAVSEN |
| | 143, K135W)) | TIIIAPTEPTHRSGNILDYAILHDAHLPRREQARERIGASLM |
| | | LNQLRSQITSDHFPVSFVRDRGGEASGCAGNRVRRSVGSS |
| | | LSCINLDWDVIRDKTKTKIESLKEHGPIKNKMSESPNKTV |
| | | SEEKAKQYLEEFHQTALEHPELSELKTVTGTNPVFAGAN |
| | | YAAWAVNVAQVIDSETADNLEKTTAALSILPGIGSVMGI |
| | | ADGAVHHNTEEIVAQSIALSSLMVAQAIPLVGELVDIGFA |
| | | AYNFVESIINLFQVVHNSYNRPAYSPGHKTHGGGGEGGG |
| | | GAPARSPSPSTQPWEHVNAIQEARRLLNLSRDTAAEMNE |
| | | TVEVISEMFDLQEPTCLQTRLELYKQGLRGSLTKLKGPLT |
| | | MMASHYKQHCPPTPETSCATQIITFESFKENLKDFLLVIPF |
| | | DCWEPVQEGGGGQGGGGEGGGSGSWVNCSNMIDEIITH |
| | | LKQPPLPLLDFNNLNGEDQDILMENNLRRPNLEAFNRAV |
| | | KSLQNASAIESILKNLLPCLPLATAAPTRHPIHIKDGDWNE |
| | | FRRKLTFYLWTLENAQAQ |
| 48 | VCID1337 | DLTDFRVATWNLQGASATTESKWNINVRQLISGENAVDI |
| | (EcCDT-furin | LAVQEAGSPPSTAVDTGTLIPSPGIPVRELIWNLSTNSRPQ |
| | site-DTB- | QVYIYFSAVDALGGRVNLALVSNRRADEVFVLSPVRQGG |
| | GMCSF- | RPLLGIRIGNDAFFTAHAIAMRNNDAPALVEEVYNFFRDS |
| | IL3(31-143, | RDPVHQALNWMILGDFNREPADLEMNLTVPVRRASEIISP |
| | K135W)) | AAATQTSQRTLDYAVAGNSVAFRPSPLQAGIVYGARRTQ |
| | | ISSDHFPVGVSRRGGEASGCAGNRVRRSVGSSLSCINLDW |
| | | DVIRDKTKTKIESLKEHGPIKNKMSESPNKTVSEEKAKQY |
| | | LEEFHQTALEHPELSELKTVTGTNPVFAGANYAAWAVN |
| | | VAQVIDSETADNLEKTTAALSILPGIGSVMGIADGAVHHN |
| | | TEEIVAQSIALSSLMVAQAIPLVGELVDIGFAAYNFVESIIN |
| | | LFQVVHNSYNRPAYSPGHKTHGQGAPARSPSPSTQPWEH |
| | | VNAIQEARRLLNLSRDTAAEMNETVEVISEMFDLQEPTCL |
| | | QTRLELYKQGLRGSLTKLKGPLTMMASHYKQHCPPTPET |
| | | SCATQIITFESFKENLKDFLLVIPFDCWEPVQEGGGGEGG |
| | | GGSWVNCSNMIDEIITHLKQPPLPLLDFNNLNGEDQDILM |
| | | ENNLRRPNLEAFNRAVKSLQNASAIESILKNLLPCLPLAT |
| | | AAPTRHPIHIKDGDWNEFRRKLTFYLWTLENAQAQ |
| 49 | VCID1338 | DLTDFRVATWNLQGASATTESKWNINVRQLISGENAVDI |
| | (EcCDT-furin | LAVQEAGSPPSTAVDTGTLIPSPGIPVRELIWNLSTNSRPQ |
| | site-DTB- | QVYIYFSAVDALGGRVNLALVSNRRADEVFVLSPVRQGG |
| | linker-GMCSF- | RPLLGIRIGNDAFFTAHAIAMRNNDAPALVEEVYNFFRDS |
| | linker-IL3(31- | RDPVHQALNWMILGDFNREPADLEMNLTVPVRRASEIISP |
| | 143, K135W)) | AAATQTSQRTLDYAVAGNSVAFRPSPLQAGIVYGARRTQ |
| | | ISSDHFPVGVSRRGGEASGCAGNRVRRSVGSSLSCINLDW |
| | | DVIRDKTKTKIESLKEHGPIKNKMSESPNKTVSEEKAKQY |
| | | LEEFHQTALEHPELSELKTVTGTNPVFAGANYAAWAVN |
| | | VAQVIDSETADNLEKTTAALSILPGIGSVMGIADGAVHHN |
| | | TEEIVAQSIALSSLMVAQAIPLVGELVDIGFAAYNFVESIIN |
| | | LFQVVHNSYNRPAYSPGHKTHGGGGEGGGGAPARSPSPS |
| | | TQPWEHVNAIQEARRLLNLSRDTAAEMNETVEVISEMFD |
| | | LQEPTCLQTRLELYKQGLRGSLTKLKGPLTMMASHYKQ |
| | | HCPPTPETSCATQIITFESFKENLKDFLLVIPFDCWEPVQEG |
| | | GGGQGGGGEGGGSGSWVNCSNMIDEIITHLKQPPLPLLD |
| | | FNNLNGEDQDILMENNLRRPNLEAFNRAVKSLQNASAIE |
| | | SILKNLLPCLPLATAAPTRHPIHIKDGDWNEFRRKLTFYL |
| | | WTLENAQAQ |
| 50 | VCID1339 | NLENYTVATWNLQGSSAINESKWNINVRQLLTGPQAAGI |
| | (HpCDT-furin | LMVQEAGSLPSTAVHTRRMVQPEGVGFPIDEYVWNLGT |
| | site-DTB- | RSRPNNVYIYYSRLDVGANRVNLAIIARRMASEVFVINSS |
| | GMCSF- | SSVLTSRPAIGIRIDDDAFFSIHALSSGGADSLSLIQNIHTFF |
| | IL3(31-143, | NTEGRRHINWMAVGDFNRAPGRMQEALDSEPGLRNATL |
| | K135W)) | IVAPTEPTHRSGGVLDYAVLHNANQTTQNTTVSASIMEN |
| | | QMRSQITSDHFPVSFVKKRGGEASGCAGNRVRRSVGSSL |
| | | SCINLDWDVIRDKTKTKIESLKEHGPIKNKMSESPNKTVS |
| | | EEKAKQYLEEFHQTALEHPELSELKTVTGTNPVFAGANY |
| | | AAWAVNVAQVIDSETADNLEKTTAALSILPGIGSVMGIA |
| | | DGAVHHNTEEIVAQSIALSSLMVAQAIPLVGELVDIGFAA |
| | | YNFVESIINLFQVVHNSYNRPAYSPGHKTHGQGAPARSPS |
| | | PSTQPWEHVNAIQEARRLLNLSRDTAAEMNETVEVISEM |
| | | FDLQEPTCLQTRLELYKQGLRGSLTKLKGPLTMMASHYK |
| | | QHCPPTPETSCATQIITFESFKENLKDFLLVIPFDCWEPVQE |
| | | GGGGEGGGGSWVNCSNMIDEIITHLKQPPLPLLDFNNLN |
| | | GEDQDILMENNLRRPNLEAFNRAVKSLQNASAIESILKNL |
| | | LPCLPLATAAPTRHPIHIKDGDWNEFRRKLTFYLWTLENA |
| | | QAQ |
| 51 | VCID1340 | NLENYTVATWNLQGSSAINESKWNINVRQLLTGPQAAGI |
| | (HpCDT-furin | LMVQEAGSLPSTAVHTRRMVQPEGVGFPIDEYVWNLGT |
| | site-DTB- | RSRPNNVYIYYSRLDVGANRVNLAIIARRMASEVFVINSS |
| | linker-GMCSF- | SSVLTSRPAIGIRIDDDAFFSIHALSSGGADSLSLIQNIHTFF |
| | linker-IL3(31- | NTEGRRHINWMAVGDFNRAPGRMQEALDSEPGLRNATL |
| | 143, K135W)) | IVAPTEPTHRSGGVLDYAVLHNANQTTQNTTVSASIMEN |
| | | QMRSQITSDHFPVSFVKKRGGEASGCAGNRVRRSVGSSL |
| | | SCINLDWDVIRDKTKTKIESLKEHGPIKNKMSESPNKTVS |
| | | EEKAKQYLEEFHQTALEHPELSELKTVTGTNPVFAGANY |
| | | AAWAVNVAQVIDSETADNLEKTTAALSILPGIGSVMGIA |
| | | DGAVHHNTEEIVAQSIALSSLMVAQAIPLVGELVDIGFAA |
| | | YNFVESIINLFQVVHNSYNRPAYSPGHKTHGGGGEGGGG |
| | | APARSPSPSTQPWEHVNAIQEARRLLNLSRDTAAEMNET |
| | | VEVISEMFDLQEPTCLQTRLELYKQGLRGSLTKLKGPLTM |
| | | MASHYKQHCPPTPETSCATQIITFESFKENLKDFLLVIPFD |
| | | CWEPVQEGGGGQGGGGEGGGSGSWVNCSNMIDEIITHL |
| | | KQPPLPLLDFNNLNGEDQDILMENNLRRPNLEAFNRAVK |
| | | SLQNASAIESILKNLLPCLPLATAAPTRHPIHIKDGDWNEF |
| | | RRKLTFYLWTLENAQAQ |
| 52 | VCID0539 | NLSDFKVATWNLQGSSAVNESKWNINVRQLLSGEQGADI |
| | (CDT-reduced | LMVQEAGSLPSSAVRTSRVIQHGGTPIEEYTWNLGTRSRP |
| | cleavage linker- | NMVYIYYSRLDVGANRVNLAIVSRRQADEAFIVHSDSSV |
| | DTB-GMCSF) | LQSRPAVGIRIGTDVFFTVHALATGGSDAVSLIRNIFTTFN |
| | | SSSSPPERRVYSWMVVGDFNRAPANLEVALRQEPAVSEN |
| | | TIIIAPTEPTHRSGNILDYAILHDAHLPRREQARERIGASLM |
| | | LNQLRSQITSDHFPVSFVRDRGGEASGGCKGGASATFGV |
| | | ASLCINLDWDVIRDKTKTKIESLKEHGPIKNKMSESPNKT |
| | | VSEEKAKQYLEEFHQTALEHPELSELKTVTGTNPVFAGA |
| | | NYAAWAVNVAQVIDSETADNLEKTTAALSILPGIGSVMG |
| | | IADGAVHHNTEEIVAQSIALSSLMVAQAIPLVGELVDIGF |
| | | AAYNFVESIINLFQVVHNSYNRPAYSPGHKTHGQGAPAR |
| | | SPSPSTQPWEHVNAIQEARRLLNLSRDTAAEMNETVEVIS |
| | | EMFDLQEPTCLQTRLELYKQGLRGSLTKLKGPLTMMASH |
| | | YKQHCPPTPETSCATQIITFESFKENLKDFLLVIPFDCWEP |
| | | VQE |
| 53 | VCID1411 | NLSDFKVATWNLQGSSAVNESKWNINVRQLLSGEQGADI |
| | (CDT-DTB- | LMVQEAGSLPSSAVRTSRVIQHGGTPIEEYTWNLGTRSRP |
| | linker-GMCSF) | NMVYIYYSRLDVGANRVNLAIVSRRQADEAFIVHSDSSV |
| | | LQSRPAVGIRIGTDVFFTVHALATGGSDAVSLIRNIFTTFN |
| | | SSSSPPERRVYSWMVVGDFNRAPANLEVALRQEPAVSEN |
| | | TIIIAPTEPTHRSGNILDYAILHDAHLPRREQARERIGASLM |
| | | LNQLRSQITSDHFPVSFVRDRGGEASGCASAGQGGGGEG |
| | | SLSCINLDWDVIRDKTKTKIESLKEHGPIKNKMSESPNKT |
| | | VSEEKAKQYLEEFHQTALEHPELSELKTVTGTNPVFAGA |
| | | NYAAWAVNVAQVIDSETADNLEKTTAALSILPGIGSVMG |
| | | IADGAVHHNTEEIVAQSIALSSLMVAQAIPLVGELVDIGF |
| | | AAYNFVESIINLFQVVHNSYNRPAYSPGHKTHGGGGEGG |
| | | GGAPARSPSPSTQPWEHVNAIQEARRLLNLSRDTAAEMN |
| | | ETVEVISEMFDLQEPTCLQTRLELYKQGLRGSLTKLKGPL |
| | | TMMASHYKQHCPPTPETSCATQIITFESFKENLKDFLLVIP |
| | | FDCWEPVQE |
| 54 | VCID1172 | NLSDFKVATWNLQGSSAVNESKWNINVRQLLSGEQGADI |
| | (CDT-furin site- | LMVQEAGSLPSSAVRTSRVIQHGGTPIEEYTWNLGTRSRP |
| | GMCSF) | NMVYIYYSRLDVGANRVNLAIVSRRQADEAFIVHSDSSV |
| | | LQSRPAVGIRIGTDVFFTVHALATGGSDAVSLIRNIFTTFN |
| | | SSSSPPERRVYSWMVVGDFNRAPANLEVALRQEPAVSEN |
| | | TIIIAPTEPTHRSGNILDYAILHDAHLPRREQARERIGASLM |
| | | LNQLRSQITSDHFPVSFVRDRGGEASGAGNRVRRSVGSSL |
| | | SGAPARSPSPSTQPWEHVNAIQEARRLLNLSRDTAAEMN |
| | | ETVEVISEMFDLQEPTCLQTRLELYKQGLRGSLTKLKGPL |
| | | TMMASHYKQHCPPTPETSCATQIITFESFKENLKDFLLVIP |
| | | FDCWEPVQE |
| 55 | VCID1170 | NLSDFKVATWNLQGSSAVNESKWNINVRQLLSGEQGADI |
| | (CDT-linker- | LMVQEAGSLPSSAVRTSRVIQHGGTPIEEYTWNLGTRSRP |
| | GMCSF) | NMVYIYYSRLDVGANRVNLAIVSRRQADEAFIVHSDSSV |
| | | LQSRPAVGIRIGTDVFFTVHALATGGSDAVSLIRNIFTTFN |
| | | SSSSPPERRVYSWMVVGDFNRAPANLEVALRQEPAVSEN |
| | | TIIIAPTEPTHRSGNILDYAILHDAHLPRREQARERIGASLM |
| | | LNQLRSQITSDHFPVSFVRDRGGGGEGGSGGGQSGGAPA |
| | | RSPSPSTQPWEHVNAIQEARRLLNLSRDTAAEMNETVEVI |
| | | SEMFDLQEPTCLQTRLELYKQGLRGSLTKLKGPLTMMAS |
| | | HYKQHCPPTPETSCATQIITFESFKENLKDFLLVIPFDCWE |
| | | PVQE |
| 56 | VCID0719 (tag- | MSENQSSEGHHHHHHSSEVLFQGPNLSDFKVATWNLQG |
| | CDT-furin site- | SSAVNESKWNINVRQLLSGEQGADILMVQEAGSLPSSAV |
| | DTB-GMCSF) | RTSRVIQHGGTPIEEYTWNLGTRSRPNMVYIYYSRLDVG |
| | | ANRVNLAIVSRRQADEAFIVHSDSSVLQSRPAVGIRIGTD |
| | | VFFTVHALATGGSDAVSLIRNIFTTFNSSSSPPERRVYSW |
| | | MVVGDFNRAPANLEVALRQEPAVSENTIIIAPTEPTHRSG |
| | | NILDYAILHDAHLPRREQARERIGASLMLNQLRSQITSDH |
| | | FPVSFVRDRGGEASGCAGNRVRRSVGSSLSCINLDWDVIR |
| | | DKTKTKIESLKEHGPIKNKMSESPNKTVSEEKAKQYLEEF |
| | | HQTALEHPELSELKTVTGTNPVFAGANYAAWAVNVAQV |
| | | IDSETADNLEKTTAALSILPGIGSVMGIADGAVHHNTEEIV |
| | | AQSIALSSLMVAQAIPLVGELVDIGFAAYNFVESIINLFQV |
| | | VHNSYNRPAYSPGHKTHGGGGEGGGGAPARSPSPSTQP |
| | | WEHVNAIQEARRLLNLSRDTAAEMNETVEVISEMFDLQE |
| | | PTCLQTRLELYKQGLRGSLTKLKGPLTMMASHYKQHCPP |
| | | TPETSCATQIITFESFKENLKDFLLVIPFDCWEPVQE |
| 57 | VCID0719 N- | GPNLSDFKVATWNLQGSSAVNESKWNINVRQLLSGEQG |
| | terminal tag | ADILMVQEAGSLPSSAVRTSRVIQHGGTPIEEYTWNLGTR |
| | removed (CDT- | SRPNMVYIYYSRLDVGANRVNLAIVSRRQADEAFIVHSD |
| | furin site-DTB- | SSVLQSRPAVGIRIGTDVFFTVHALATGGSDAVSLIRNIFT |
| | GMCSF) | TFNSSSSPPERRVYSWMVVGDFNRAPANLEVALRQEPAV |
| | | SENTIIIAPTEPTHRSGNILDYAILHDAHLPRREQARERIGA |
| | | SLMLNQLRSQITSDHFPVSFVRDRGGEASGCAGNRVRRS |
| | | VGSSLSCINLDWDVIRDKTKTKIESLKEHGPIKNKMSESP |
| | | NKTVSEEKAKQYLEEFHQTALEHPELSELKTVTGTNPVF |
| | | AGANYAAWAVNVAQVIDSETADNLEKTTAALSILPGIGS |
| | | VMGIADGAVHHNTEEIVAQSIALSSLMVAQAIPLVGELV |
| | | DIGFAAYNFVESIINLFQVVHNSYNRPAYSPGHKTHGGGG |
| | | EGGGGAPARSPSPSTQPWEHVNAIQEARRLLNLSRDTAA |
| | | EMNETVEVISEMFDLQEPTCLQTRLELYKQGLRGSLTKL |
| | | KGPLTMMASHYKQHCPPTPETSCATQIITFESFKENLKDF |
| | | LLVIPFDCWEPVQE |
| 58 | Linker | GGSGG |
| 59 | Linker | AEAAAKAPAPQPAPAEAAAKA |
| 60 | Linker | GPAPEPAPAPQPAPAPAG |
| 61 | VCID1726 | NLENYTVATWNLQGSSAINESKWNINVRQLLTGPQAAGI |
| | (HpCDT-furin | LMVQEAGSLPSTAVHTRRMVQPEGVGFPIDEYVWNLGT |
| | site-DTB- | RSRPNNVYIYYSRLDVGANRVNLAIIARRMASEVFVINSS |
| | GMCSF- | SSVLTSRPAIGIRIDDDAFFSIHALSSGGADSLSLIQNIHTFF |
| | IL3(31-143, | NTEGRRHINWMAVGDFNRAPGRMQEALDSEPGLRNATL |
| | K135W) | IVAPTEPTHRSGGVLDYAVLHNANQTTQNTTVSASIMFN |
| | | QMRSQITSDHFPVSFVKKRAEAAAKAPAPQPAPAEAAAK |
| | | ACAGNRVRRSVGSSLSCINLDWDVIRDKTKTKIESLKEHG |
| | | PIKNKMSESPNKTVSEEKAKQYLEEFHQTALEHPELSELK |
| | | TVTGTNPVFAGANYAAWAVNVAQVIDSETADNLEKTTA |
| | | ALSILPGIGSVMGIADGAVHHNTEEIVAQSIALSSLMVAQ |
| | | AIPLVGELVDIGFAAYNFVESIINLFQVVHNSYNRPAYSPG |
| | | HKTHGGGGEGGGGAPARSPSPSTQPWEHVNAIQEARRLL |
| | | NLSRDTAAEMNETVEVISEMFDLQEPTCLQTRLELYKQG |
| | | LRGSLTKLKGPLTMMASHYKQHCPPTPETSCATQIITFESF |
| | | KENLKDFLLVIPFDCWEPVQEGGGGQGGGGEGGGSGSW |
| | | VNCSNMIDEIITHLKQPPLPLLDFNNLNGEDQDILMENNL |
| | | RRPNLEAFNRAVKSLQNASAIESILKNLLPCLPLATAAPTR |
| | | HPIHIKDGDWNEFRRKLTFYLWTLENAQAQ |
| 62 | VCID1728 | NLENYTVATWNLQGSSAINESKWNINVRQLLTGPQAAGI |
| | (HpCDT-furin | LMVQEAGSLPSTAVHTRRMVQPEGVGFPIDEYVWNLGT |
| | site-DTB- | RSRPNNVYIYYSRLDVGANRVNLAIIARRMASEVFVINSS |
| | GMCSF- | SSVLTSRPAIGIRIDDDAFFSIHALSSGGADSLSLIQNIHTFF |
| | IL3(31-143, | NTEGRRHINWMAVGDFNRAPGRMQEALDSEPGLRNATL |
| | K135W) | IVAPTEPTHRSGGVLDYAVLHNANQTTQNTTVSASIMEN |
| | | QMRSQITSDHFPVSFVKKRAEAAAKAPAPQPAPAEAAAK |
| | | ACAGNRVRRSVGSSLSCINLDWDVIRDKTKTKIESLKEHG |
| | | PIKNKMSESPNKTVSEEKAKQYLEEFHQTALEHPELSELK |
| | | TVTGTNPVFAGANYAAWAVNVAQVIDSETADNLEKTTA |
| | | ALSILPGIGSVMGIADGAVHHNTEEIVAQSIALSSLMVAQ |
| | | AIPLVGELVDIGFAAYNFVESIINLFQVVHNSYNRPGAEG |
| | | AGQAGGAPARSPSPSTQPWEHVNAIQEARRLLNLSRDTA |
| | | AEMNETVEVISEMFDLQEPTCLQTRLELYKQGLRGSLTK |
| | | LKGPLTMMASHYKQHCPPTPETSCATQIITFESFKENLKD |
| | | FLLVIPFDCWEPVQEGPAPEPAPAPQPAPAPAGSWVNCSN |
| | | MIDEIITHLKQPPLPLLDFNNLNGEDQDILMENNLRRPNLE |
| | | AFNRAVKSLQNASAIESILKNLLPCLPLATAAPTRHPIHIK |
| | | DGDWNEFRRKLTFYLWTLENAQAQ |
| 63 | DTB(403) | INLDWDVIRDKTKTKIESLKEHGPIKNKMSESPNKTVSEE |
| | | KAKQYLEEFHQTALEHPELSELKTVTGTNPVFAGANYAA |
| | | WAVNVAQVIDSETADNLEKTTAALSILPGIGSVMGIADG |
| | | AVHHNTEEIVAQSIALSSLMVAQAIPLVGELVDIGFAAYN |
| | | FVESIINLFQVVHNSYNRP |
| 64 | Cysteine- | CPGNRVRRSVGSSLSC |
| | bounded furin- | |
| | cleavable linker | |
| 65 | Linker | GAEGAGQAGG |
| 66 | Linker | GQAGGPSGSGGSEPGQSGGQQAEAAAKEAAAKGPGSQG |
| | | GA |
| 67 | Linker | APAPAPAPAPAPAPA |
| 68 | Linker | GPAPEPAPAPQPAPAPAG |
| 69 | Linker | GAEAAAKEAQAKEAQAAKAG |
| 70 | Linker | GAEAAAKAPAPQPAPAEAAAKAG |
| 71 | Linker | GAPQAAPEGAPQAPAPEAG |
| 72 | Linker | LETQYKETQQKEHQLFEV |
| 73 | Linker | AEAAAKAPAPQPAPAEAAAKA |
| 74 | Linker | PAPQPHPA |
| 75 | Linker | GAPQSGSEGASQGAGQPGG |
| 76 | Linker | GGAQPSEAQATESQGGQPGG |
| 77 | Linker | APGEAAPAEATAKQAAQEAVEKAPQAEQEAKHQAQEAV |
| | | EKAEPAEKAAKHAGQG |
| 78 | Linker | APGEAAPAEATAKQAAQEAVEKAPAPQPAPAEQEAKHQ |
| | | AQEAVEKAPQPEPAPAEKAAKHAGQGQAPGEA |
| 79 | Anti-BCMA | QSVLTQPPSASGTPGQRVTISCSGSSSNIGSNTVNWYQQL |
| | single chain | PGTAPKLLIFNYHQRPSGVPDRFSGSKSGSSASLAISGLQS |
| | antibody (VL- | EDEADYYCAAWDDSLNGWVFGGGTKLTVLGAPQSGSE |
| | linker-VH) | GASQGAGQPGGEVQLVESGGGLVKPGGSLRLSCAASGFT |
| | | FGDYALSWFRQAPGKGLEWVGVSRSKAYGGTTDYAASV |
| | | KGRFTISRDDSKSTAYLQMNSLKTEDTAVYYCASSGYSS |
| | | GWTPFDYWGQGTLVTVSS |
| 80 | VCID1694 | NLENYTVATWNLQGSSAINESKWNINVRQLLTGPQAAGI |
| | (HpCDT-furin- | LMVQEAGSLPSTAVHTRRMVQPEGVGFPIDEYVWNLGT |
| | DTB-linker- | RSRPNNVYIYYSRLDVGANRVNLAIIARRMASEVFVINSS |
| | antiBCMAsc) | SSVLTSRPAIGIRIDDDAFFSIHALSSGGADSLSLIQNIHTFF |
| | | NTEGRRHINWMAVGDFNRAPGRMQEALDSEPGLRNATL |
| | | IVAPTEPTHRSGGVLDYAVLHNANQTTQNTTVSASIMEN |
| | | QMRSQITSDHFPVSFVKKRGGEASGCAGNRVRRSVGSSL |
| | | SCINLDWDVIRDKTKTKIESLKEHGPIKNKMSESPNKTVS |
| | | EEKAKQYLEEFHQTALEHPELSELKTVTGTNPVFAGANY |
| | | AAWAVNVAQVIDSETADNLEKTTAALSILPGIGSVMGIA |
| | | DGAVHHNTEEIVAQSIALSSLMVAQAIPLVGELVDIGFAA |
| | | YNFVESIINLFQVVHNSYNRPAYSPGHKTGAEGAGQAGG |
| | | QSVLTQPPSASGTPGQRVTISCSGSSSNIGSNTVNWYQQL |
| | | PGTAPKLLIFNYHQRPSGVPDRFSGSKSGSSASLAISGLQS |
| | | EDEADYYCAAWDDSLNGWVFGGGTKLTVLGAPQSGSE |
| | | GASQGAGQPGGEVQLVESGGGLVKPGGSLRLSCAASGFT |
| | | FGDYALSWFRQAPGKGLEWVGVSRSKAYGGTTDYAASV |
| | | KGRFTISRDDSKSTAYLQMNSLKTEDTAVYYCASSGYSS |
| | | GWTPFDYWGQGTLVTVSS |
| 81 | Anti-CD19 | DIQLTQSPASLAVSLGQRATISCKASQSVDYDGDSYLNW |
| | single chain | YQQIPGQPPKLLIYDASNLVSGIPPRFSGSGSGTDFTLNIHP |
| | antibody (VL- | VEKVDAATYHCQQSTEDPWTFGGGTKLEIKGAPQSGSEG |
| | linker-VH) | ASQGAGQPGGQVQLQQSGAELVRPGSSVKISCKASGYAF |
| | | SSYWMNWVKQRPGQGLEWIGQIWPGDGDTNYNGKFKG |
| | | KATLTADESSSTAYMQLSSLASEDSAVYFCARRETTTVG |
| | | RYYYAMDYWGQGTTVTVSS |
| 82 | VCID1700 | NLENYTVATWNLQGSSAINESKWNINVRQLLTGPQAAGI |
| | (HpCDT-furin- | LMVQEAGSLPSTAVHTRRMVQPEGVGFPIDEYVWNLGT |
| | DTB-linker- | RSRPNNVYIYYSRLDVGANRVNLAIIARRMASEVFVINSS |
| | antiCD19sc) | SSVLTSRPAIGIRIDDDAFFSIHALSSGGADSLSLIQNIHTFF |
| | | NTEGRRHINWMAVGDFNRAPGRMQEALDSEPGLRNATL |
| | | IVAPTEPTHRSGGVLDYAVLHNANQTTQNTTVSASIMEN |
| | | QMRSQITSDHFPVSFVKKRGGEASGCAGNRVRRSVGSSL |
| | | SCINLDWDVIRDKTKTKIESLKEHGPIKNKMSESPNKTVS |
| | | EEKAKQYLEEFHQTALEHPELSELKTVTGTNPVFAGANY |
| | | AAWAVNVAQVIDSETADNLEKTTAALSILPGIGSVMGIA |
| | | DGAVHHNTEEIVAQSIALSSLMVAQAIPLVGELVDIGFAA |
| | | YNFVESIINLFQVVHNSYNRPAYSPGHKTGAEGAGQAGG |
| | | DIQLTQSPASLAVSLGQRATISCKASQSVDYDGDSYLNW |
| | | YQQIPGQPPKLLIYDASNLVSGIPPRFSGSGSGTDFTLNIHP |
| | | VEKVDAATYHCQQSTEDPWTFGGGTKLEIKGAPQSGSEG |
| | | ASQGAGQPGGQVQLQQSGAELVRPGSSVKISCKASGYAF |
| | | SSYWMNWVKQRPGQGLEWIGQIWPGDGDTNYNGKFKG |
| | | KATLTADESSSTAYMQLSSLASEDSAVYFCARRETTTVG |
| | | RYYYAMDYWGQGTTVTVSS |
| 83 | Human IL2(22- | APTSSSTKKTQLQLEHLLLDLQMILNGINNYKNPKLTRML |
| | 153, C125A | TFKFYMPKKATELKHLQCLEEELKPLEEVLNLAQSKNFH |
| | mutant) | LRPRDLISNINVIVLELKGSETTFMCEYADETATIVEFLNR |
| | | WITFAQSIISTLT |
| 84 | VCID1799 | NLENYTVATWNLQGSSAINESKWNINVRQLLTGPQAAGI |
| | (HpCDT-furin- | LMVQEAGSLPSTAVHTRRMVQPEGVGFPIDEYVWNLGT |
| | DTB403-linker- | RSRPNNVYIYYSRLDVGANRVNLAIIARRMASEVFVINSS |
| | hIL2) | SSVLTSRPAIGIRIDDDAFFSIHALSSGGADSLSLIQNIHTFF |
| | | NTEGRRHINWMAVGDFNRAPGRMQEALDSEPGLRNATL |
| | | IVAPTEPTHRSGGVLDYAVLHNANQTTQNTTVSASIMEN |
| | | QMRSQITSDHFPVSFVKKRAEAAAKAPAPQPAPAEAAAK |
| | | ACAGNRVRRSVGSSLSCINLDWDVIRDKTKTKIESLKEHG |
| | | PIKNKMSESPNKTVSEEKAKQYLEEFHQTALEHPELSELK |
| | | TVTGTNPVFAGANYAAWAVNVAQVIDSETADNLEKTTA |
| | | ALSILPGIGSVMGIADGAVHHNTEEIVAQSIALSSLMVAQ |
| | | AIPLVGELVDIGFAAYNFVESIINLFQVVHNSYNRPGAEG |
| | | AGQAGGAPTSSSTKKTQLQLEHLLLDLQMILNGINNYKN |
| | | PKLTRMLTFKFYMPKKATELKHLQCLEEELKPLEEVLNL |
| | | AQSKNFHLRPRDLISNINVIVLELKGSETTFMCEYADETA |
| | | TIVEFLNRWITFAQSIISTLT |
| 85 | Human IL4 (25- | HKCDITLQEIIKTLNSLTEQKTLCTELTVTDIFAASKNTTE |
| | 253) | KETFCRAATVLRQFYSHHEKDTRCLGATAQQFHRHKQLI |
| | | RFLKRLDRNLWGLAGLNSCPVKEANQSTLENFLERLKTI |
| | | MREKYSKCSS |
| 86 | VCID1801 | NLENYTVATWNLQGSSAINESKWNINVRQLLTGPQAAGI |
| | (HpCDT-furin- | LMVQEAGSLPSTAVHTRRMVQPEGVGFPIDEYVWNLGT |
| | DTB403-linker- | RSRPNNVYIYYSRLDVGANRVNLAIIARRMASEVFVINSS |
| | hIL4) | SSVLTSRPAIGIRIDDDAFFSIHALSSGGADSLSLIQNIHTFF |
| | | NTEGRRHINWMAVGDFNRAPGRMQEALDSEPGLRNATL |
| | | IVAPTEPTHRSGGVLDYAVLHNANQTTQNTTVSASIMEN |
| | | QMRSQITSDHFPVSFVKKRAEAAAKAPAPQPAPAEAAAK |
| | | ACAGNRVRRSVGSSLSCINLDWDVIRDKTKTKIESLKEHG |
| | | PIKNKMSESPNKTVSEEKAKQYLEEFHQTALEHPELSELK |
| | | TVTGTNPVFAGANYAAWAVNVAQVIDSETADNLEKTTA |
| | | ALSILPGIGSVMGIADGAVHHNTEEIVAQSIALSSLMVAQ |
| | | AIPLVGELVDIGFAAYNFVESIINLFQVVHNSYNRPGAEG |
| | | AGQAGGHKCDITLQEIIKTLNSLTEQKTLCTELTVTDIFAA |
| | | SKNTTEKETFCRAATVLRQFYSHHEKDTRCLGATAQQFH |
| | | RHKQLIRFLKRLDRNLWGLAGLNSCPVKEANQSTLENFL |
| | | ERLKTIMREKYSKCSSGGEGSGWSHPQFEK |
| 87 | VCID1800 | NLENYTVATWNLQGSSAINESKWNINVRQLLTGPQAAGI |
| | (HpCDT-furin- | LMVQEAGSLPSTAVHTRRMVQPEGVGFPIDEYVWNLGT |
| | DTB403-linker- | RSRPNNVYIYYSRLDVGANRVNLAIIARRMASEVFVINSS |
| | hIL4-linker- | SSVLTSRPAIGIRIDDDAFFSIHALSSGGADSLSLIQNIHTFF |
| | hIL2) | NTEGRRHINWMAVGDFNRAPGRMQEALDSEPGLRNATL |
| | | IVAPTEPTHRSGGVLDYAVLHNANQTTQNTTVSASIMFN |
| | | QMRSQITSDHFPVSFVKKRAEAAAKAPAPQPAPAEAAAK |
| | | ACAGNRVRRSVGSSLSCINLDWDVIRDKTKTKIESLKEHG |
| | | PIKNKMSESPNKTVSEEKAKQYLEEFHQTALEHPELSELK |
| | | TVTGTNPVFAGANYAAWAVNVAQVIDSETADNLEKTTA |
| | | ALSILPGIGSVMGIADGAVHHNTEEIVAQSIALSSLMVAQ |
| | | AIPLVGELVDIGFAAYNFVESIINLFQVVHNSYNRPGAEG |
| | | AGQAGGHKCDITLQEIIKTLNSLTEQKTLCTELTVTDIFAA |
| | | SKNTTEKETFCRAATVLRQFYSHHEKDTRCLGATAQQFH |
| | | RHKQLIRFLKRLDRNLWGLAGLNSCPVKEANQSTLENFL |
| | | ERLKTIMREKYSKCSSGGAQPSEAQATESQGGQPGGAPT |
| | | SSSTKKTQLQLEHLLLDLQMILNGINNYKNPKLTRMLTFK |
| | | FYMPKKATELKHLQCLEEELKPLEEVLNLAQSKNFHLRP |
| | | RDLISNINVIVLELKGSETTFMCEYADETATIVEFLNRWIT |
| | | FAQSIISTLT |
| 88 | VCID1572 | NLSDFKVATWNLQGSSAVNESKWNINVRQLLSGEQGADI |
| | (HdCDT-furin- | LMVQEAGSLPSSAVRTSRVIQHGGTPIEEYTWNLGTRSRP |
| | DTB-linker- | NMVYIYYSRLDVGANRVNLAIVSRRQADEAFIVHSDSSV |
| | hIL4-linker- | LQSRPAVGIRIGTDVFFTVHALATGGSDAVSLIRNIFTTFN |
| | hIL2) | SSSSPPERRVYSWMVVGDFNRAPANLEVALRQEPAVSEN |
| | | TIIIAPTEPTHRSGNILDYAILHDAHLPRREQARERIGASLM |
| | | LNQLRSQITSDHFPVSFVRDRGGEASGCAGNRVRRSVGSS |
| | | LSCINLDWDVIRDKTKTKIESLKEHGPIKNKMSESPNKTV |
| | | SEEKAKQYLEEFHQTALEHPELSELKTVTGTNPVFAGAN |
| | | YAAWAVNVAQVIDSETADNLEKTTAALSILPGIGSVMGI |
| | | ADGAVHHNTEEIVAQSIALSSLMVAQAIPLVGEDVDIGFA |
| | | AYNFVESIINLFQVVHNSYNRPAYSPGHKTHGGGGQGGG |
| | | GEGGGSGHKCDITLQEIIKTLNSLTEQKTLCTELTVTDIFA |
| | | ASKNTTEKETFCRAATVLRQFYSHHEKDTRCLGATAQQF |
| | | HRHKQLIRFLKRLDRNLWGLAGLNSCPVKEANQSTLENF |
| | | LETLKTIMREKYSKCSSGGAQPSEAQATESQGGQPGGAP |
| | | TSSSTKKTQLQLEHLLLDLQMILNGINNYKNPKLTRMLTF |
| | | KFYMPKKATELKHLQCLEEELKPLEEVLNLAQSKNFHLR |
| | | PRDLISNINVIVLELKGSETTFMCEYADETATIVEFLNRWI |
| | | TFAQSIISTLT |
| 89 | VCID1573 | NLSDFKVATWNLQGSSAVNESKWNINVRQLLSGEQGADI |
| | (HdCDT-furin- | LMVQEAGSLPSSAVRTSRVIQHGGTPIEEYTWNLGTRSRP |
| | DTB-linker- | NMVYIYYSRLDVGANRVNLAIVSRRQADEAFIVHSDSSV |
| | hIL4) | LQSRPAVGIRIGTDVFFTVHALATGGSDAVSLIRNIFTTFN |
| | | SSSSPPERRVYSWMVVGDFNRAPANLEVALRQEPAVSEN |
| | | TIIIAPTEPTHRSGNILDYAILHDAHLPRREQARERIGASLM |
| | | LNQLRSQITSDHFPVSFVRDRGGEASGCAGNRVRRSVGSS |
| | | LSCINLDWDVIRDKTKTKIESLKEHGPIKNKMSESPNKTV |
| | | SEEKAKQYLEEFHQTALEHPELSELKTVTGTNPVFAGAN |
| | | YAAWAVNVAQVIDSETADNLEKTTAALSILPGIGSVMGI |
| | | ADGAVHHNTEEIVAQSIALSSLMVAQAIPLVGELVDIGFA |
| | | AYNFVESIINLFQVVHNSYNRPAYSPGHKTHGGGGQGGG |
| | | GEGGGSGHKCDITLQEIIKTLNSLTEQKTLCTELTVTDIFA |
| | | ASKNTTEKETFCRAATVLRQFYSHHEKDTRCLGATAQQF |
| | | HRHKQLIRFLKRLDRNLWGLAGLNSCPVKEANQSTLENF |
| | | LERLKTIMREKYSKCSS |
| 90 | VCID1574 | NLSDFKVATWNLQGSSAVNESKWNINVRQLLSGEQGADI |
| | (HdCDT-furin- | LMVQEAGSLPSSAVRTSRVIQHGGTPIEEYTWNLGTRSRP |
| | DTB-linker- | NMVYIYYSRLDVGANRVNLAIVSRRQADEAFIVHSDSSV |
| | hIL4-linker- | LQSRPAVGIRIGTDVFFTVHALATGGSDAVSLIRNIFTTFN |
| | hIL2) | SSSSPPERRVYSWMVVGDFNRAPANLEVALRQEPAVSEN |
| | | TIIIAPTEPTHRSGNILDYAILHDAHLPRREQARERIGASLM |
| | | LNQLRSQITSDHFPVSFVRDRGGEASGCAGNRVRRSVGSS |
| | | LSCINLDWDVIRDKTKTKIESLKEHGPIKNKMSESPNKTV |
| | | SEEKAKQYLEEFHQTALEHPELSELKTVTGTNPVFAGAN |
| | | YAAWAVNVAQVIDSETADNLEKTTAALSILPGIGSVMGI |
| | | ADGAVHHNTEEIVAQSIALSSLMVAQAIPLVGELVDIGFA |
| | | AYNFVESIINLFQVVHNSYNRPAYSPGHKTHGGGGQGGG |
| | | GEGGGSGKQHKCDITLQEIIKTLNSLTEQKTLCTELTVTDI |
| | | FAASKNTTEKETFCRAATVLRQFYSHHEKDTRCLGATAQ |
| | | QFHRHKQLIRFLKRLDRNLWGLAGLNSCPVKEANQSTLE |
| | | NFLERLKTIMREKYSKCSSQEGGAQPSEAQATESQGGQP |
| | | GGAPTSSSTKKTQLQLEHLLLDLQMILNGINNYKNPKLTR |
| | | MLTFKFYMPKKATELKHLQCLEEELKPLEEVLNLAQSKN |
| | | FHLRPRDLISNINVIVLELKGSETTFMCEYADETATIVEFL |
| | | NRWITFAQSIISTLT |
| 91 | VCID1219 | NLSDFKVATWNLQGSSAVNESKWNINVRQLLSGEQGADI |
| | (HdCDT-furin- | LMVQEAGSLPSSAVRTSRVIQHGGTPIEEYTWNLGTRSRP |
| | DTB-linker- | NMVYIYYSRLDVGANRVNLAIVSRRQADEAFIVHSDSSV |
| | hIL2) | LQSRPAVGIRIGTDVFFTVHALATGGSDAVSLIRNIFTTFN |
| | | SSSSPPERRVYSWMVVGDFNRAPANLEVALRQEPAVSEN |
| | | TIIIAPTEPTHRSGNILDYAILHDAHLPRREQARERIGASLM |
| | | LNQLRSQITSDHFPVSFVRDRGGEASGCAGNRVRRSVGSS |
| | | LSCINLDWDVIRDKTKTKIESLKEHGPIKNKMSESPNKTV |
| | | SEEKAKQYLEEFHQTALEHPELSELKTVTGTNPVFAGAN |
| | | YAAWAVNVAQVIDSETADNLEKTTAALSILPGIGSVMGI |
| | | ADGAVHHNTEEIVAQSIALSSLMVAQAIPLVGELVDIGFA |
| | | AYNFVESIINLFQVVHNSYNRPAYSPGHKTHGGAQPSEA |
| | | QATESQGGQPGGAPTSSSTKKTQLQLEHLLLDLQMILNGI |
| | | NNYKNPKLTRMLTFKFYMPKKATELKHLQCLEEELKPLE |
| | | EVLNLAQSKNFHLRPRDLISNINVIVLELKGSETTFMCEY |
| | | ADETATIVEFLNRWITFAQSIISTLT |
| 92 | Anti-CLDN18.2 | DIVMTQSPDSLAVSLGERATINCKSSQSLLNSGNLKNYLT |
| | single chain | WYQQKPGQPPKLLIYWASTRKSGVPDRFSGSGSGTDFTL |
| | antibody | TISSLQAEDVAVYYCQNDYSYPLTFGGGTKVEIKGAPQS |
| | (osemitamab, | GSEGASQGAGQPGGQVQLVQSGAEVKKPGASVKVSCKA |
| | VL-linker-VH) | SGYTFTGYNMNWVRQAPGQGLEWMGNIDPYYGGTSYN |
| | | QKFKGRVTMTIDKSTSTVYMELSSLRSEDTAVYYCARMY |
| | | HGNAFDYWGQGTTVTVSS |
| 93 | VCID1418 | NLSDFKVATWNLQGSSAVNESKWNINVRQLLSGEQGADI |
| | (HdCDT-furin- | LMVQEAGSLPSSAVRTSRVIQHGGTPIEEYTWNLGTRSRP |
| | DTB-linker- | NMVYIYYSRLDVGANRVNLAIVSRRQADEAFIVHSDSSV |
| | antiCLDN18.20 | LQSRPAVGIRIGTDVFFTVHALATGGSDAVSLIRNIFTTFN |
| | se-sc) | SSSSPPERRVYSWMVVGDFNRAPANLEVALRQEPAVSEN |
| | | TIIIAPTEPTHRSGNILDYAILHDAHLPRREQARERIGASLM |
| | | LNQLRSQITSDHFPVSFVRDRGGEASGCAGNRVRRSVGSS |
| | | LSCINLDWDVIRDKTKTKIESLKEHGPIKNKMSESPNKTV |
| | | SEEKAKQYLEEFHQTALEHPELSELKTVTGTNPVFAGAN |
| | | YAAWAVNVAQVIDSETADNLEKTTAALSILPGIGSVMGI |
| | | ADGAVHHNTEEIVAQSIALSSLMVAQAIPLVGELVDIGFA |
| | | AYNFVESIINLFQVVHNSYNRPAYSPGHKTHGGGGEGGG |
| | | PDIVMTQSPDSLAVSLGERATINCKSSQSLLNSGNLKNYL |
| | | TWYQQKPGQPPKLLIYWASTRKSGVPDRFSGSGSGTDFT |
| | | LTISSLQAEDVAVYYCQNDYSYPLTFGGGTKVEIKGAPQS |
| | | GSEGASQGAGQPGGQVQLVQSGAEVKKPGASVKVSCKA |
| | | SGYTFTGYNMNWVRQAPGQGLEWMGNIDPYYGGTSYN |
| | | QKFKGRVTMTIDKSTSTVYMELSSLRSEDTAVYYCARMY |
| | | HGNAFDYWGQGTTVTVSS |
| 94 | Anti-CLDN18.2 | QVQLQQPGAELVRPGASVKLSCKASGYTFTSYWINWVK |
| | single chain | QRPGQGLEWIGNIYPSDSYTNYNQKFKDKATLTVDKSSS |
| | antibody | TAYMQLSSPTSEDSAVYYCTRSWRGNSFDYWGQGTTLT |
| | (zolbetuximab, | VSSGAPQSGSEGASQGAGQPGGDIVMTQSPSSLTVTAGE |
| | VH-linker-VL) | KVTMSCKSSQSLLNSGNQKNYLTWYQQKPGQPPKLLIY |
| | | WASTRESGVPDRFTGSGSGTDFTLTISSVQAEDLAVYYCQ |
| | | NDYSYPFTFGSGTKLEIK |
| 95 | VCID1419 | NLSDFKVATWNLQGSSAVNESKWNINVRQLLSGEQGADI |
| | (HdCDT-furin- | LMVQEAGSLPSSAVRTSRVIQHGGTPIEEYTWNLGTRSRP |
| | DTB-linker- | NMVYIYYSRLDVGANRVNLAIVSRRQADEAFIVHSDSSV |
| | antiCLDN18.2zol- | LQSRPAVGIRIGTDVFFTVHALATGGSDAVSLIRNIFTTFN |
| | sc | SSSSPPERRVYSWMVVGDFNRAPANLEVALRQEPAVSEN |
| | | TIIIAPTEPTHRSGNILDYAILHDAHLPRREQARERIGASLM |
| | | LNQLRSQITSDHFPVSFVRDRGGEASGCAGNRVRRSVGSS |
| | | LSCINLDWDVIRDKTKTKIESLKEHGPIKNKMSESPNKTV |
| | | SEEKAKQYLEEFHQTALEHPELSELKTVTGTNPVFAGAN |
| | | YAAWAVNVAQVIDSETADNLEKTTAALSILPGIGSVMGI |
| | | ADGAVHHNTEEIVAQSIALSSLMVAQAIPLVGELVDIGFA |
| | | AYNFVESIINLFQVVHNSYNRPAYSPGHKTHGGGGEGGG |
| | | PQVQLQQPGAELVRPGASVKLSCKASGYTFTSYWINWV |
| | | KQRPGQGLEWIGNIYPSDSYTNYNQKFKDKATLTVDKSS |
| | | STAYMQLSSPTSEDSAVYYCTRSWRGNSFDYWGQGTTL |
| | | TVSSGAPQSGSEGASQGAGQPGGDIVMTQSPSSLTVTAGE |
| | | KVTMSCKSSQSLLNSGNQKNYLTWYQQKPGQPPKLLIY |
| | | WASTRESGVPDRFTGSGSGTDFTLTISSVQAEDLAVYYCQ |
| | | NDYSYPFTFGSGTKLEIK |
| 96 | VCID1793 | NLENYTVATWNLQGSSAINESKWNINVRQLLTGPQAAGI |
| | (HpCDT-furin- | LMVQEAGSLPSTAVHTRRMVQPEGVGFPIDEYVWNLGT |
| | DTB403-linker- | RSRPNNVYIYYSRLDVGANRVNLAIIARRMASEVFVINSS |
| | antiCLDN18.20se- | SSVLTSRPAIGIRIDDDAFFSIHALSSGGADSLSLIQNIHTFF |
| | sc) | NTEGRRHINWMAVGDFNRAPGRMQEALDSEPGLRNATL |
| | | IVAPTEPTHRSGGVLDYAVLHNANQTTQNTTVSASIMEN |
| | | QMRSQITSDHFPVSFVKKRAEAAAKAPAPQPAPAEAAAK |
| | | ACAGNRVRRSVGSSLSCINLDWDVIRDKTKTKIESLKEHG |
| | | PIKNKMSESPNKTVSEEKAKQYLEEFHQTALEHPELSELK |
| | | TVTGTNPVFAGANYAAWAVNVAQVIDSETADNLEKTTA |
| | | ALSILPGIGSVMGIADGAVHHNTEEIVAQSIALSSLMVAQ |
| | | AIPLVGELVDIGFAAYNFVESIINLFQVVHNSYNRPGAEG |
| | | AGQAGGDIVMTQSPDSLAVSLGERATINCKSSQSLLNSGN |
| | | LKNYLTWYQQKPGQPPKLLIYWASTRKSGVPDRFSGSGS |
| | | GTDFTLTISSLQAEDVAVYYCQNDYSYPLTFGGGTKVEIK |
| | | GAPQSGSEGASQGAGQPGGQVQLVQSGAEVKKPGASVK |
| | | VSCKASGYTFTGYNMNWVRQAPGQGLEWMGNIDPYYG |
| | | GTSYNQKFKGRVTMTIDKSTSTVYMELSSLRSEDTAVYY |
| | | CARMYHGNAFDYWGQGTTVTVSS |
| 97 | VCID1794 | NLENYTVATWNLQGSSAINESKWNINVRQLLTGPQAAGI |
| | (HpCDT-furin- | LMVQEAGSLPSTAVHTRRMVQPEGVGFPIDEYVWNLGT |
| | DTB403-linker- | RSRPNNVYIYYSRLDVGANRVNLAIIARRMASEVFVINSS |
| | antiCLDN18.2zol- | SSVLTSRPAIGIRIDDDAFFSIHALSSGGADSLSLIQNIHTFF |
| | sc) | NTEGRRHINWMAVGDFNRAPGRMQEALDSEPGLRNATL |
| | | IVAPTEPTHRSGGVLDYAVLHNANQTTQNTTVSASIMEN |
| | | QMRSQITSDHFPVSFVKKRAEAAAKAPAPQPAPAEAAAK |
| | | ACAGNRVRRSVGSSLSCINLDWDVIRDKTKTKIESLKEHG |
| | | PIKNKMSESPNKTVSEEKAKQYLEEFHQTALEHPELSELK |
| | | TVTGTNPVFAGANYAAWAVNVAQVIDSETADNLEKTTA |
| | | ALSILPGIGSVMGIADGAVHHNTEEIVAQSIALSSLMVAQ |
| | | AIPLVGELVDIGFAAYNFVESIINLFQVVHNSYNRPGAEG |
| | | AGQAGGDIVMTQSPSSLTVTAGEKVTMSCKSSQSLLNSG |
| | | NQKNYLTWYQQKPGQPPKLLIYWASTRESGVPDRFTGSG |
| | | SGTDFTLTISSVQAEDLAVYYCQNDYSYPFTFGSGTKLEI |
| | | KGAPQSGSEGASQGAGQPGGQVQLQQPGAELVRPGASV |
| | | KLSCKASGYTFTSYWINWVKQRPGQGLEWIGNIYPSDSY |
| | | TNYNQKFKDKATLTVDKSSSTAYMQLSSPTSEDSAVYYC |
| | | TRSWRGNSFDYWGQGTTLTVSS |
| 98 | Anti-HER2 | VDNKFNKEMRNAYWEIALLPNLNNQQKRAFIRSLYDDPS |
| | affibody | QSANLLAEAKKLNDAQAPK |
| 99 | VCID1896 | NLENYTVATWNLQGSSAINESKWNINVRQLLTGPQAAGI |
| | (HpCDT-furin- | LMVQEAGSLPSTAVHTRRMVQPEGVGFPIDEYVWNLGT |
| | DTB403- | RSRPNNVYIYYSRLDVGANRVNLAIIARRMASEVFVINSS |
| | antiHER 2aff) | SSVLTSRPAIGIRIDDDAFFSIHALSSGGADSLSLIQNIHTFF |
| | | NTEGRRHINWMAVGDFNRAPGRMQEALDSEPGLRNATL |
| | | IVAPTEPTHRSGGVLDYAVLHNANQTTQNTTVSASIMEN |
| | | QMRSQITSDHFPVSFVKKRAEAAAKAPAPQPAPAEAAAK |
| | | ACAGNRVRRSVGSSLSCINLDWDVIRDKTKTKIESLKEHG |
| | | PIKNKMSESPNKTVSEEKAKQYLEEFHQTALEHPELSELK |
| | | TVTGTNPVFAGANYAAWAVNVAQVIDSETADNLEKTTA |
| | | ALSILPGIGSVMGIADGAVHHNTEEIVAQSIALSSLMVAQ |
| | | AIPLVGELVDIGFAAYNFVESIINLFQVVHNSYNRPGAEG |
| | | AGQAGGVDNKFNKEMRNAYWEIALLPNLNNQQKRAFIR |
| | | SLYDDPSQSANLLAEAKKLNDAQAPK |
| 100 | Anti-HER2 | GSDLGKKLLEAARAGQDDEVRILMANGADVNAKDEYGL |
| | DARPin | TPLYLATAHGHLEIVEVLLKNGADVNAVDAIGFTPLHLA |
| | | AFIGHLEIAEVLLKHGADVNAQDKFGKTAFDISIGNGNED |
| | | LAEILQKLN |
| 101 | VCID1898 | NLENYTVATWNLQGSSAINESKWNINVRQLLTGPQAAGI |
| | (HpCDT-furin- | LMVQEAGSLPSTAVHTRRMVQPEGVGFPIDEYVWNLGT |
| | DTB403-linker- | RSRPNNVYIYYSRLDVGANRVNLAIIARRMASEVFVINSS |
| | antiHER2dar) | SSVLTSRPAIGIRIDDDAFFSIHALSSGGADSLSLIQNIHTFF |
| | | NTEGRRHINWMAVGDFNRAPGRMQEALDSEPGLRNATL |
| | | IVAPTEPTHRSGGVLDYAVLHNANQTTQNTTVSASIMEN |
| | | QMRSQITSDHFPVSFVKKRAEAAAKAPAPQPAPAEAAAK |
| | | ACAGNRVRRSVGSSLSCINLDWDVIRDKTKTKIESLKEHG |
| | | PIKNKMSESPNKTVSEEKAKQYLEEFHQTALEHPELSELK |
| | | TVTGTNPVFAGANYAAWAVNVAQVIDSETADNLEKTTA |
| | | ALSILPGIGSVMGIADGAVHHNTEEIVAQSIALSSLMVAQ |
| | | AIPLVGELVDIGFAAYNFVESIINLFQVVHNSYNRPGAEG |
| | | AGQAGGGSDLGKKLLEAARAGQDDEVRILMANGADVN |
| | | AKDEYGLTPLYLATAHGHLEIVEVLLKNGADVNAVDAIG |
| | | FTPLHLAAFIGHLEIAEVLLKHGADVNAQDKFGKTAFDISI |
| | | GNGNEDLAEILQKLN |
| 102 | Anti-HER2 | GSDLGKKLLEAARAGQDDEVRILMANGADVNAHDFYGI |
| | DARPin | TPLHLAANFGHLEIVEVLLKHGADVNAFDYDNTPLHLAA |
| | | DAGHLEIVEVLLKYGADVNASDRDGHTPLHLAAREGHL |
| | | EIVEVLLKNGADVNAQDKFGKTPFDLAIDNGNEDIAEVL |
| | | QKAAKLN |
| 103 | VCID1899 | NLENYTVATWNLQGSSAINESKWNINVRQLLTGPQAAGI |
| | (HpCDT-furin- | LMVQEAGSLPSTAVHTRRMVQPEGVGFPIDEYVWNLGT |
| | DTB403-linker- | RSRPNNVYIYYSRLDVGANRVNLAIIARRMASEVFVINSS |
| | antiHER2dar) | SSVLTSRPAIGIRIDDDAFFSIHALSSGGADSLSLIQNIHTFF |
| | | NTEGRRHINWMAVGDFNRAPGRMQEALDSEPGLRNATL |
| | | IVAPTEPTHRSGGVLDYAVLHNANQTTQNTTVSASIMEN |
| | | QMRSQITSDHFPVSFVKKRAEAAAKAPAPQPAPAEAAAK |
| | | ACAGNRVRRSVGSSLSCINLDWDVIRDKTKTKIESLKEHG |
| | | PIKNKMSESPNKTVSEEKAKQYLEEFHQTALEHPELSELK |
| | | TVTGTNPVFAGANYAAWAVNVAQVIDSETADNLEKTTA |
| | | ALSILPGIGSVMGIADGAVHHNTEEIVAQSIALSSLMVAQ |
| | | AIPLVGELVDIGFAAYNFVESIINLFQVVHNSYNRPGAEG |
| | | AGQAGGGSDLGKKLLEAARAGQDDEVRILMANGADVN |
| | | AHDFYGITPLHLAANFGHLEIVEVLLKHGADVNAFDYDN |
| | | TPLHLAADAGHLEIVEVLLKYGADVNASDRDGHTPLHLA |
| | | AREGHLEIVEVLLKNGADVNAQDKFGKTPFDLAIDNGNE |
| | | DIAEVLQKAAKLN |
| 104 | Anti-MSLN | QVELVQSGAEVKKPGESLKISCKGSGYSFTSYWIGWVRQ |
| | single chain | APGKGLEWMGIIDPGDSRTRYSPSFQGQVTISADKSISTA |
| | (anetumab, VH- | YLQWSSLKASDTAMYYCARGQLYGGTYMDGWGQGTL |
| | linker-VL) | VTVSSGAPQSGSEGASQGAGQPGGDIALTQPASVSGSPGQ |
| | | SITISCTGTSSDIGGYNSVSWYQQHPGKAPKLMIYGVNNR |
| | | PSGVSNRFSGSKSGNTASLTISGLQAEDEADYYCSSYDIES |
| | | ATPVFGGGTKLTVL |
| 105 | VCID1862 | NLENYTVATWNLQGSSAINESKWNINVRQLLTGPQAAGI |
| | (HpCDT-furin- | LMVQEAGSLPSTAVHTRRMVQPEGVGFPIDEYVWNLGT |
| | DTB403-linker- | RSRPNNVYIYYSRLDVGANRVNLAIIARRMASEVFVINSS |
| | antiMSLNsc) | SSVLTSRPAIGIRIDDDAFFSIHALSSGGADSLSLIQNIHTFF |
| | | NTEGRRHINWMAVGDFNRAPGRMQEALDSEPGLRNATL |
| | | IVAPTEPTHRSGGVLDYAVLHNANQTTQNTTVSASIMEN |
| | | QMRSQITSDHFPVSFVKKRAEAAAKAPAPQPAPAEAAAK |
| | | ACAGNRVRRSVGSSLSCINLDWDVIRDKTKTKIESLKEHG |
| | | PIKNKMSESPNKTVSEEKAKQYLEEFHQTALEHPELSELK |
| | | TVTGTNPVFAGANYAAWAVNVAQVIDSETADNLEKTTA |
| | | ALSILPGIGSVMGIADGAVHHNTEEIVAQSIALSSLMVAQ |
| | | AIPLVGELVDIGFAAYNFVESIINLFQVVHNSYNRPGAEG |
| | | AGQAGGQVELVQSGAEVKKPGESLKISCKGSGYSFTSYW |
| | | IGWVRQAPGKGLEWMGIIDPGDSRTRYSPSFQGQVTISAD |
| | | KSISTAYLQWSSLKASDTAMYYCARGQLYGGTYMDGW |
| | | GQGTLVTVSSGAPQSGSEGASQGAGQPGGDIALTQPASV |
| | | SGSPGQSITISCTGTSSDIGGYNSVSWYQQHPGKAPKLMI |
| | | YGVNNRPSGVSNRFSGSKSGNTASLTISGLQAEDEADYY |
| | | CSSYDIESATPVFGGGTKLTVL |
| 106 | Anti-GPC3 | DVVMTQSPLSLPVTPGEPASISCRSSQSLVHSNRNTYLHW |
| | single chain | YLQKPGQSPQLLIYKVSNRFSGVPDRFSGSGSGTDFTLKIS |
| | (codrituzumab, | RVEAEDVGVYYCSQNTHVPPTFGQGTKLEIKGAPQSGSE |
| | VL-linker-VH) | GASQGAGQPGGQVQLVQSGAEVKKPGASVKVSCKASGY |
| | | TFTDYEMHWVRQAPGQGLEWMGALDPKTGDTAYSQKF |
| | | KGRVTLTADKSTSTAYMELSSLTSEDTAVYYCTRFYSYT |
| | | YWGQGTLVTVSS |
| 107 | VCID1861 | NLENYTVATWNLQGSSAINESKWNINVRQLLTGPQAAGI |
| | (HpCDT-furin- | LMVQEAGSLPSTAVHTRRMVQPEGVGFPIDEYVWNLGT |
| | DTB403-linker- | RSRPNNVYIYYSRLDVGANRVNLAIIARRMASEVFVINSS |
| | antiGPC3sc) | SSVLTSRPAIGIRIDDDAFFSIHALSSGGADSLSLIQNIHTFF |
| | | NTEGRRHINWMAVGDFNRAPGRMQEALDSEPGLRNATL |
| | | IVAPTEPTHRSGGVLDYAVLHNANQTTQNTTVSASIMEN |
| | | QMRSQITSDHFPVSFVKKRAEAAAKAPAPQPAPAEAAAK |
| | | ACAGNRVRRSVGSSLSCINLDWDVIRDKTKTKIESLKEHG |
| | | PIKNKMSESPNKTVSEEKAKQYLEEFHQTALEHPELSELK |
| | | TVTGTNPVFAGANYAAWAVNVAQVIDSETADNLEKTTA |
| | | ALSILPGIGSVMGIADGAVHHNTEEIVAQSIALSSLMVAQ |
| | | AIPLVGELVDIGFAAYNFVESIINLFQVVHNSYNRPGAEG |
| | | AGQAGGDVVMTQSPLSLPVTPGEPASISCRSSQSLVHSNR |
| | | NTYLHWYLQKPGQSPQLLIYKVSNRFSGVPDRFSGSGSG |
| | | TDFTLKISRVEAEDVGVYYCSQNTHVPPTFGQGTKLEIKG |
| | | APQSGSEGASQGAGQPGGQVQLVQSGAEVKKPGASVKV |
| | | SCKASGYTFTDYEMHWVRQAPGQGLEWMGALDPKTGD |
| | | TAYSQKFKGRVTLTADKSTSTAYMELSSLTSEDTAVYYC |
| | | TRFYSYTYWGQGTLVTVSS |
| 108 | Anti-EGFR | DILLTQSPVILSVSPGERVSFSCRASQSIGTNIHWYQQRTN |
| | single chain | GSPRLLIKYASESISGIPSRFSGSGSGTDFTLSINSVESEDIA |
| | (cetuximab, | DYYCQQNNNWPTTFGAGTKLELKGAPQSGSEGASQGAG |
| | VL-linker-VH) | QPGGQVQLKQSGPGLVQPSQSLSITCTVSGFSLTNYGVH |
| | | WVRQSPGKGLEWLGVIWSGGNTDYNTPFTSRLSINKDNS |
| | | KSQVFFKMNSLQSNDTAIYYCARALTYYDYEFAYWGQG |
| | | TLVTVSA |
| 109 | VCID1795 | NLENYTVATWNLQGSSAINESKWNINVRQLLTGPQAAGI |
| | (HpCDT-furin- | LMVQEAGSLPSTAVHTRRMVQPEGVGFPIDEYVWNLGT |
| | DTB403-linker- | RSRPNNVYIYYSRLDVGANRVNLAIIARRMASEVFVINSS |
| | antiEGFRsc) | SSVLTSRPAIGIRIDDDAFFSIHALSSGGADSLSLIQNIHTFF |
| | | NTEGRRHINWMAVGDFNRAPGRMQEALDSEPGLRNATL |
| | | IVAPTEPTHRSGGVLDYAVLHNANQTTQNTTVSASIMEN |
| | | QMRSQITSDHFPVSFVKKRAEAAAKAPAPQPAPAEAAAK |
| | | ACAGNRVRRSVGSSLSCINLDWDVIRDKTKTKIESLKEHG |
| | | PIKNKMSESPNKTVSEEKAKQYLEEFHQTALEHPELSELK |
| | | TVTGTNPVFAGANYAAWAVNVAQVIDSETADNLEKTTA |
| | | ALSILPGIGSVMGIADGAVHHNTEEIVAQSIALSSLMVAQ |
| | | AIPLVGELVDIGFAAYNFVESIINLFQVVHNSYNRPGAEG |
| | | AGQAGGDILLTQSPVILSVSPGERVSFSCRASQSIGTNIHW |
| | | YQQRTNGSPRLLIKYASESISGIPSRFSGSGSGTDFTLSINS |
| | | VESEDIADYYCQQNNNWPTTFGAGTKLELKGAPQSGSEG |
| | | ASQGAGQPGGQVQLKQSGPGLVQPSQSLSITCTVSGFSLT |
| | | NYGVHWVRQSPGKGLEWLGVIWSGGNTDYNTPFTSRLSI |
| | | NKDNSKSQVFFKMNSLQSNDTAIYYCARALTYYDYEFA |
| | | YWGQGTLVTVSA |
| 110 | Anti-EGFR | QVQLQESGGGLVQPGGSLRLSCAASGRTFSSYAMGWFR |
| | nanobody | QAPGKQREFVAAIRWSGGYTYYTDSVKGRFTISRDNAKT |
| | | TVYLQMNSLKPEDTAVYYCAATYLSSDYSRYALPQRPLD |
| | | YDYWGQGTQVTVSS |
| 111 | VCID1906 | NLENYTVATWNLQGSSAINESKWNINVRQLLTGPQAAGI |
| | (HpCDT-furin- | LMVQEAGSLPSTAVHTRRMVQPEGVGFPIDEYVWNLGT |
| | DTB403-linker- | RSRPNNVYIYYSRLDVGANRVNLAIIARRMASEVFVINSS |
| | antiEGFRnan) | SSVLTSRPAIGIRIDDDAFFSIHALSSGGADSLSLIQNIHTFF |
| | | NTEGRRHINWMAVGDFNRAPGRMQEALDSEPGLRNATL |
| | | IVAPTEPTHRSGGVLDYAVLHNANQTTQNTTVSASIMEN |
| | | QMRSQITSDHFPVSFVKKRAEAAAKAPAPQPAPAEAAAK |
| | | ACAGNRVRRSVGSSLSCINLDWDVIRDKTKTKIESLKEHG |
| | | PIKNKMSESPNKTVSEEKAKQYLEEFHQTALEHPELSELK |
| | | TVTGTNPVFAGANYAAWAVNVAQVIDSETADNLEKTTA |
| | | ALSILPGIGSVMGIADGAVHHNTEEIVAQSIALSSLMVAQ |
| | | AIPLVGELVDIGFAAYNFVESIINLFQVVHNSYNRPGAEG |
| | | AGQAGGQVQLQESGGGLVQPGGSLRLSCAASGRTFSSYA |
| | | MGWFRQAPGKQREFVAAIRWSGGYTYYTDSVKGRFTIS |
| | | RDNAKTTVYLQMNSLKPEDTAVYYCAATYLSSDYSRYA |
| | | LPQRPLDYDYWGQGTQVTVSS |
| 112 | Anti-EGFR | QVQLQESGGGSVQAGGSLKLSCAASGRSFSTYAMGWFR |
| | nanobody | QAPGQDREFVATISWTDSTDYADSVKGRFTISRDNAKNT |
| | | GYLQMNSLKPEDTAVYYCAADRWASSRRNVDYDYWGQ |
| | | GTQVTVSSHGS |
| 113 | VCID1907 | NLENYTVATWNLQGSSAINESKWNINVRQLLTGPQAAGI |
| | (HpCDT-furin- | LMVQEAGSLPSTAVHTRRMVQPEGVGFPIDEYVWNLGT |
| | DTB403- | RSRPNNVYIYYSRLDVGANRVNLAIIARRMASEVFVINSS |
| | antiEGFRnan) | SSVLTSRPAIGIRIDDDAFFSIHALSSGGADSLSLIQNIHTFF |
| | | NTEGRRHINWMAVGDFNRAPGRMQEALDSEPGLRNATL |
| | | IVAPTEPTHRSGGVLDYAVLHNANQTTQNTTVSASIMFN |
| | | QMRSQITSDHFPVSFVKKRAEAAAKAPAPQPAPAEAAAK |
| | | ACAGNRVRRSVGSSLSCINLDWDVIRDKTKTKIESLKEHG |
| | | PIKNKMSESPNKTVSEEKAKQYLEEFHQTALEHPELSELK |
| | | TVTGTNPVFAGANYAAWAVNVAQVIDSETADNLEKTTA |
| | | ALSILPGIGSVMGIADGAVHHNTEEIVAQSIALSSLMVAQ |
| | | AIPLVGELVDIGFAAYNFVESIINLFQVVHNSYNRPGAEG |
| | | AGQAGGQVQLQESGGGSVQAGGSLKLSCAASGRSFSTY |
| | | AMGWFRQAPGQDREFVATISWTDSTDYADSVKGRFTISR |
| | | DNAKNTGYLQMNSLKPEDTAVYYCAADRWASSRRNVD |
| | | YDYWGQGTQVTVSSHGS |
| 114 | Anti-TROP2 | QVQLVQSGAEVKKPGASVKVSCKASGYTFTTAGMQWV |
| | single chain | RQAPGQGLEWMGWINTHSGVPKYAEDFKGRVTISADTS |
| | (datopotamab, | TSTAYLQLSSLKSEDTAVYYCARSGFGSSYWYFDVWGQ |
| | VH-linker-VL) | GTLVTVSSGAPQSGSEGASQGAGQPGGDIQMTQSPSSLSA |
| | | SVGDRVTITCKASQDVSTAVAWYQQKPGKAPKLLIYSAS |
| | | YRYTGVPSRFSGSGSGTDFTLTISSLQPEDFAVYYCQQHYI |
| | | TPLTFGQGTKLEIK |
| 115 | VCID1880 | NLENYTVATWNLQGSSAINESKWNINVRQLLTGPQAAGI |
| | (HpCDT-furin- | LMVQEAGSLPSTAVHTRRMVQPEGVGFPIDEYVWNLGT |
| | DTB403- | RSRPNNVYIYYSRLDVGANRVNLAIIARRMASEVFVINSS |
| | antiTROP2dat- | SSVLTSRPAIGIRIDDDAFFSIHALSSGGADSLSLIQNIHTFF |
| | sc) | NTEGRRHINWMAVGDFNRAPGRMQEALDSEPGLRNATL |
| | | IVAPTEPTHRSGGVLDYAVLHNANQTTQNTTVSASIMEN |
| | | QMRSQITSDHFPVSFVKKRAEAAAKAPAPQPAPAEAAAK |
| | | ACAGNRVRRSVGSSLSCINLDWDVIRDKTKTKIESLKEHG |
| | | PIKNKMSESPNKTVSEEKAKQYLEEFHQTALEHPELSELK |
| | | TVTGTNPVFAGANYAAWAVNVAQVIDSETADNLEKTTA |
| | | ALSILPGIGSVMGIADGAVHHNTEEIVAQSIALSSLMVAQ |
| | | AIPLVGELVDIGFAAYNFVESIINLFQVVHNSYNRPGAEG |
| | | AGQAGGQVQLVQSGAEVKKPGASVKVSCKASGYTFTTA |
| | | GMQWVRQAPGQGLEWMGWINTHSGVPKYAEDFKGRVT |
| | | ISADTSTSTAYLQLSSLKSEDTAVYYCARSGFGSSYWYFD |
| | | VWGQGTLVTVSSGAPQSGSEGASQGAGQPGGDIQMTQS |
| | | PSSLSASVGDRVTITCKASQDVSTAVAWYQQKPGKAPKL |
| | | LIYSASYRYTGVPSRFSGSGSGTDFTLTISSLQPEDFAVYY |
| | | CQQHYITPLTFGQGTKLEIK |
| 116 | VCID1791 | NLENYTVATWNLQGSSAINESKWNINVRQLLTGPQAAGI |
| | (HpCDT-furin- | LMVQEAGSLPSTAVHTRRMVQPEGVGFPIDEYVWNLGT |
| | DTB403-linker- | RSRPNNVYIYYSRLDVGANRVNLAIIARRMASEVFVINSS |
| | hGMCSF- | SSVLTSRPAIGIRIDDDAFFSIHALSSGGADSLSLIQNIHTFF |
| | linker-hIL3) | NTEGRRHINWMAVGDFNRAPGRMQEALDSEPGLRNATL |
| | | IVAPTEPTHRSGGVLDYAVLHNANQTTQNTTVSASIMFN |
| | | QMRSQITSDHFPVSFVKKRAEAAAKAPAPQPAPAEAAAK |
| | | ACAGNRVRRSVGSSLSCINLDWDVIRDKTKTKIESLKEHG |
| | | PIKNKMSESPNKTVSEEKAKQYLEEFHQTALEHPELSELK |
| | | TVTGTNPVFAGANYAAWAVNVAQVIDSETADNLEKTTA |
| | | ALSILPGIGSVMGIADGAVHHNTEEIVAQSIALSSLMVAQ |
| | | AIPLVGELVDIGFAAYNFVESIINLFQVVHNSYNRPGAEG |
| | | AGQAGGAPARSPSPSTQPWEHVNAIQEARRLLNLSRDTA |
| | | AEMNETVEVISEMFDLQEPTCLQTRLELYKQGLRGSLTK |
| | | LKGPLTMMASHYKQHCPPTPETSCATQIITFESFKENLKD |
| | | FLLVIPFDCWEPVQEAPGEAAPAEATAKQAAQEAVEKAP |
| | | QAEQEAKHQAQEAVEKAEPAEKAAKHAGQGSWVNCSN |
| | | MIDEIITHLKQPPLPLLDFNNLNGEDQDILMENNLRRPNLE |
| | | AFNRAVKSLQNASAIESILKNLLPCLPLATAAPTRHPIHIK |
| | | DGDWNEFRRKLTFYLWTLENAQAQ |
| 117 | VCID1792 | NLENYTVATWNLQGSSAINESKWNINVRQLLTGPQAAGI |
| | (HpCDT-linker- | LMVQEAGSLPSTAVHTRRMVQPEGVGFPIDEYVWNLGT |
| | furin-DTB403- | RSRPNNVYIYYSRLDVGANRVNLAIIARRMASEVFVINSS |
| | linker- | SSVLTSRPAIGIRIDDDAFFSIHALSSGGADSLSLIQNIHTFF |
| | hGMCSF- | NTEGRRHINWMAVGDFNRAPGRMQEALDSEPGLRNATL |
| | linker-hIL3) | IVAPTEPTHRSGGVLDYAVLHNANQTTQNTTVSASIMEN |
| | | QMRSQITSDHFPVSFVKKRAPGEAAPAEATAKQAAQEAV |
| | | EKAPAPQPAPAEQEAKHQAQEAVEKAPQPEPAPAEKAAK |
| | | HAGQGQAPGEACAGNRVRRSVGSSLSCINLDWDVIRDKT |
| | | KTKIESLKEHGPIKNKMSESPNKTVSEEKAKQYLEEFHQT |
| | | ALEHPELSELKTVTGTNPVFAGANYAAWAVNVAQVIDSE |
| | | TADNLEKTTAALSILPGIGSVMGIADGAVHHNTEEIVAQSI |
| | | ALSSLMVAQAIPLVGELVDIGFAAYNFVESIINLFQVVHN |
| | | SYNRPGAEGAGQAGGAPARSPSPSTQPWEHVNAIQEARR |
| | | LLNLSRDTAAEMNETVEVISEMFDLQEPTCLQTRLELYK |
| | | QGLRGSLTKLKGPLTMMASHYKQHCPPTPETSCATQIITF |
| | | ESFKENLKDFLLVIPFDCWEPVQEGPAPEPAPAPQPAPAP |
| | | AGSWVNCSNMIDEIITHLKQPPLPLLDFNNLNGEDQDILM |
| | | ENNLRRPNLEAFNRAVKSLQNASAIESILKNLLPCLPLAT |
| | | AAPTRHPIHIKDGDWNEFRRKLTFYLWTLENAQAQ |
| 118 | Anti-HER2 | DIQMTQSPSSLSASVGDRVTITCRASQDVNTAVAWYQQK |
| | single chain | PGKAPKLLIYSASFLYSGVPSRFSGSRSGTDFTLTISSLQPE |
| | (trastuzumab, | DFATYYCQQHYTTPPTFGQGTKVEIKGAPQSGSEGASQG |
| | VL-linker-VH) | AGQPGGEVQLVESGGGLVQPGGSLRLSCAASGFNIKDTYI |
| | | HWVRQAPGKGLEWVARIYPTNGYTRYADSVKGRFTISA |
| | | DTSKNTAYLQMNSLRAEDTAVYYCSRWGGDGFYAMDY |
| | | WGQGTLVTVSS |
| 119 | VCID1797 | NLENYTVATWNLQGSSAINESKWNINVRQLLTGPQAAGI |
| | (HpCDT-furin- | LMVQEAGSLPSTAVHTRRMVQPEGVGFPIDEYVWNLGT |
| | DTB403- | RSRPNNVYIYYSRLDVGANRVNLAIIARRMASEVFVINSS |
| | antiHER2tra-sc) | SSVLTSRPAIGIRIDDDAFFSIHALSSGGADSLSLIQNIHTFF |
| | | NTEGRRHINWMAVGDFNRAPGRMQEALDSEPGLRNATL |
| | | IVAPTEPTHRSGGVLDYAVLHNANQTTQNTTVSASIMEN |
| | | QMRSQITSDHFPVSFVKKRAEAAAKAPAPQPAPAEAAAK |
| | | ACAGNRVRRSVGSSLSCINLDWDVIRDKTKTKIESLKEHG |
| | | PIKNKMSESPNKTVSEEKAKQYLEEFHQTALEHPELSELK |
| | | TVTGTNPVFAGANYAAWAVNVAQVIDSETADNLEKTTA |
| | | ALSILPGIGSVMGIADGAVHHNTEEIVAQSIALSSLMVAQ |
| | | AIPLVGELVDIGFAAYNFVESIINLFQVVHNSYNRPGAEG |
| | | AGQAGGDIQMTQSPSSLSASVGDRVTITCRASQDVNTAV |
| | | AWYQQKPGKAPKLLIYSASFLYSGVPSRFSGSRSGTDFTL |
| | | TISSLQPEDFATYYCQQHYTTPPTFGQGTKVEIKGAPQSG |
| | | SEGASQGAGQPGGEVQLVESGGGLVQPGGSLRLSCAASG |
| | | FNIKDTYIHWVRQAPGKGLEWVARIYPTNGYTRYADSVK |
| | | GRFTISADTSKNTAYLQMNSLRAEDTAVYYCSRWGGDG |
| | | FYAMDYWGQGTLVTVSS |
| 120 | VCID1950 | NLENYTVATWNLQGSSAINESKWNINVRQLLTGPQAAGI |
| | (HpCDT-furin- | LMVQEAGSLPSTAVHTRRMVQPEGVGFPIDEYVWNLGT |
| | DTB403-linker- | RSRPNNVYIYYSRLDVGANRVNLAIIARRMASEVFVINSS |
| | hGMCSF- | SSVLTSRPAIGIRIDDDAFFSIHALSSGGADSLSLIQNIHTFF |
| | linker-hIL3) | NTEGRRHINWMAVGDFNRAPGRMQEALDSEPGLRNATL |
| | | IVAPTEPTHRSGGVLDYAVLHNANQTTQNTTVSASIMFN |
| | | QMRSQITSDHFPVSFVKKRAEAAAKAPAPQPAPAEAAAK |
| | | ACAGNRVRRSVGSSLSCINLDWDVIRDKTKTKIESLKEHG |
| | | PIKNKMSESPNKTVSEEKAKQYLEEFHQTALEHPELSELK |
| | | TVTGTNPVFAGANYAAWAVNVAQVIDSETADNLEKTTA |
| | | ALSILPGIGSVMGIADGAVHHNTEEIVAQSIALSSLMVAQ |
| | | AIPLVGELVDIGFAAYNFVESIINLFQVVHNSYNRPGAEG |
| | | AGQAGGAPARSPSPSTQPWEHVNAIQEARRLLNLSRDTA |
| | | AEMNETVEVISEMFDLQEPTCLQTRLELYKQGLRGSLTK |
| | | LKGPLTMMASHYKQHCPPTPETSCATQIITFESFKENLKD |
| | | FLLVIPFDCWEPVQEGGGGSGGGGSGGGSAPMTQTTPLK |
| | | TSWVNCSNMIDEIITHLKQPPLPLLDFNNLNGEDQDILME |
| | | NNLRRPNLEAFNRAVKSLQNASAIESILKNLLPCLPLATA |
| | | APTRHPIHIKDGDWNEFRRKLTFYLKTLENAQAQ |
| 121 | VCID1951 | NLENYTVATWNLQGSSAINESKWNINVRQLLTGPQAAGI |
| | (HpCDT-furin- | LMVQEAGSLPSTAVHTRRMVQPEGVGFPIDEYVWNLGT |
| | DTB403-linker- | RSRPNNVYIYYSRLDVGANRVNLAIIARRMASEVFVINSS |
| | hGMCSF- | SSVLTSRPAIGIRIDDDAFFSIHALSSGGADSLSLIQNIHTFF |
| | linker-hIL3) | NTEGRRHINWMAVGDFNRAPGRMQEALDSEPGLRNATL |
| | | IVAPTEPTHRSGGVLDYAVLHNANQTTQNTTVSASIMFN |
| | | QMRSQITSDHFPVSFVKKRAEAAAKAPAPQPAPAEAAAK |
| | | ACAGNRVRRSVGSSLSCINLDWDVIRDKTKTKIESLKEHG |
| | | PIKNKMSESPNKTVSEEKAKQYLEEFHQTALEHPELSELK |
| | | TVTGTNPVFAGANYAAWAVNVAQVIDSETADNLEKTTA |
| | | ALSILPGIGSVMGIADGAVHHNTEEIVAQSIALSSLMVAQ |
| | | AIPLVGELVDIGFAAYNFVESIINLFQVVHNSYNRPGAEG |
| | | AGQAGGAPARSPSPSTQPWEHVNAIQEARRLLNLSRDTA |
| | | AEMNETVEVISEMFDLQEPTCLQTRLELYKQGLRGSLTK |
| | | LKGPLTMMASHYKQHCPPTPETSCATQIITFESFKENLKD |
| | | FLLVIPFDCWEPVQEGGGGSGGGGSGGGSAPMTQTTPLK |
| | | TSWVNCSNMIDEIITHLKQPPLPLLDFNNLNGEDQDILME |
| | | NNLRRPNLEAFNRAVKSLQNASAIESILKNLLPCLPLATA |
| | | APTRHPIHIKDGDWNEFRRKLTFYLWTLENAQAQ |
| 122 | VCID1952 | NLENYTVATWNLQGSSAINESKWNINVRQLLTGPQAAGI |
| | (HpCDT-furin- | LMVQEAGSLPSTAVHTRRMVQPEGVGFPIDEYVWNLGT |
| | DTB403-linker- | RSRPNNVYIYYSRLDVGANRVNLAIIARRMASEVFVINSS |
| | hGMCSF- | SSVLTSRPAIGIRIDDDAFFSIHALSSGGADSLSLIQNIHTFF |
| | linker-hIL3) | NTEGRRHINWMAVGDFNRAPGRMQEALDSEPGLRNATL |
| | | IVAPTEPTHRSGGVLDYAVLHNANQTTQNTTVSASIMEN |
| | | QMRSQITSDHFPVSFVKKRAEAAAKAPAPQPAPAEAAAK |
| | | ACAGNRVRRSVGSSLSCINLDWDVIRDKTKTKIESLKEHG |
| | | PIKNKMSESPNKTVSEEKAKQYLEEFHQTALEHPELSELK |
| | | TVTGTNPVFAGANYAAWAVNVAQVIDSETADNLEKTTA |
| | | ALSILPGIGSVMGIADGAVHHNTEEIVAQSIALSSLMVAQ |
| | | AIPLVGELVDIGFAAYNFVESIINLFQVVHNSYNRPGAEG |
| | | AGQAGGAPARSPSPSTQPWEHVNAIQEARRLLNLSRDTA |
| | | AEMNETVEVISEMFDLQEPTCLQTRLELYKQGLRGSLTK |
| | | LKGPLTMMASHYKQHCPPTPETSCATQIITFESFKENLKD |
| | | FLLVIPFDCWEPVQEGGGGGGGGSGGGSSWVNCSNMID |
| | | EIITHLKQPPLPLLDFNNLNGEDQDILMENNLRRPNLEAFN |
| | | RAVKSLQNASAIESILKNLLPCLPLATAAPTRHPIHIKDGD |
| | | WNEFRRKLTFYLKTLENAQAQ |
| 123 | VCID1953 | NLENYTVATWNLQGSSAINESKWNINVRQLLTGPQAAGI |
| | (HpCDT-furin- | LMVQEAGSLPSTAVHTRRMVQPEGVGFPIDEYVWNLGT |
| | DTB403-linker- | RSRPNNVYIYYSRLDVGANRVNLAIIARRMASEVFVINSS |
| | hGMCSF- | SSVLTSRPAIGIRIDDDAFFSIHALSSGGADSLSLIQNIHTFF |
| | linker-hIL3) | NTEGRRHINWMAVGDFNRAPGRMQEALDSEPGLRNATL |
| | | IVAPTEPTHRSGGVLDYAVLHNANQTTQNTTVSASIMFN |
| | | QMRSQITSDHFPVSFVKKRAEAAAKAPAPQPAPAEAAAK |
| | | ACAGNRVRRSVGSSLSCINLDWDVIRDKTKTKIESLKEHG |
| | | PIKNKMSESPNKTVSEEKAKQYLEEFHQTALEHPELSELK |
| | | TVTGTNPVFAGANYAAWAVNVAQVIDSETADNLEKTTA |
| | | ALSILPGIGSVMGIADGAVHHNTEEIVAQSIALSSLMVAQ |
| | | AIPLVGELVDIGFAAYNFVESIINLFQVVHNSYNRPGAEG |
| | | AGQAGGAPARSPSPSTQPWEHVNAIQEARRLLNLSRDTA |
| | | AEMNETVEVISEMFDLQEPTCLQTRLELYKQGLRGSLTK |
| | | LKGPLTMMASHYKQHCPPTPETSCATQIITFESFKENLKD |
| | | FLLVIPFDCWEPVQEGGGGSGGGGSGGGSSWVNCSNMID |
| | | EIITHLKQPPLPLLDFNNLNGEDQDILMENNLRRPNLEAFN |
| | | RAVKSLQNASAIESILKNLLPCLPLATAAPTRHPIHIKDGD |
| | | WNEFRRKLTFYLWTLENAQAQ |
| 124 | Linker | GGGGSGGGGSGGGS |
| | (Between | |
| | GMCSF and | |
| | IL3, 1950- | |
| | 1953) | |

### VII. Examples

The following Examples merely illustrate various aspects and embodiments of the inventions of this disclosure, and should not be construed to limit the disclosure to the embodiments represented in the Examples.

### Example 1

A fusion molecule was produced comprising a CDT-derived domain, a DTB-derived translocation domain, and one or more targeting moieties that bind a cancer-related surface antigen or receptor. For production purposes, an N-terminal tag was included in the construct. Molecule VCID0719 (SEQ ID NO:56) comprises, from N-terminus to C-terminus: a tag, a CDT-derived domain, a furin-cleavable linker, a DTB-derived translocation domain, and a GM-CSF-derived targeting moiety. A second molecule was prepared in which the N-terminal tag was cleaved from the purified molecule (VCID0719 N-terminal tag removed; SEQ ID NO:57).

The ability of these molecules to kill cancer cells was tested using an in vitro cytotoxicity assay using either U-937 cells or TF-1a cells. Cells from AML cancer cell lines U-937 or TF-1a were plated in flat bottom 96-well plates, 5000 cells per well in 100 μl RPMI-1640 complete media. Drugs were serially diluted 1:6 in RPMI-1640 complete media starting at 1000 nM. 100 μl of drug dilution was added to duplicate wells containing cells for a final starting concentration of 500 nM. Cells were incubated for 5 days at 37° C. and 5% CO₂. After 5 days, Promega CellTiter-Glo was used to assess ATP levels by luminescence, as an assessment of relative cell viability in each well. Signal was normalized to vehicle control wells. Cell viability was plotted as percent live cells, normalized to vehicle control set at 100%. Average+/−SD was plotted.

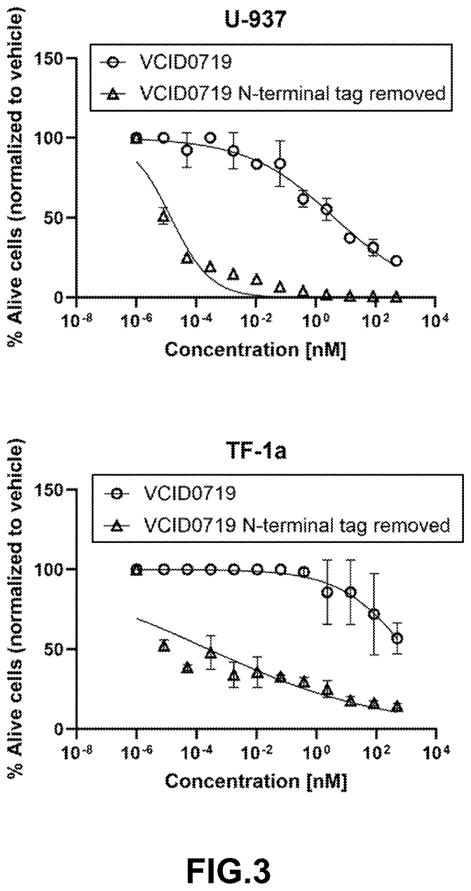

Results are shown in FIG. 3. In both tested cell types, killing activity was dramatically higher for the molecule with the N-terminal tag removed. IC50 values were calculated from non-linear fitted data. The IC50 for VCID0719 having an N-terminal tag was 0.4 nM for U-937 cells and 2.5 nM for TF-1a cells. The IC50 for VCID0719 with the N-terminal tag removed was <8.3 fM for both U-937 and TF-1a cells.

### Example 2

In vitro cytotoxicity assays were carried out using three molecules with different targeting moieties. Molecule VCID0230 (SEQ ID NO:31) comprises, from N-terminus to C-terminus, a CDT-derived domain, a furin-cleavable linker, a DTB-derived translocation domain, and a GM-CSF-derived targeting moiety. Molecule VCID0904 (SEQ ID NO:42) comprises, from N-terminus to C-terminus, a CDT-derived domain, a furin-cleavable linker, a DTB-derived translocation domain, a GM-CSF-derived targeting moiety, and an IL3-derived targeting moiety. VCID0231 (SEQ ID NO:38) comprises, from N-terminus to C-terminus, a CDT-derived domain, a furin-cleavable linker, a DTB-derived translocation domain, and an anti-CD33-derived targeting moiety. Each molecule was tested at different concentrations using three or four different AML-related cell lines: U-937, TF-1a, MOLM-13, and (for VCID0230 and VCID0904) MV4-11.

Cells from AML cancer cell lines U-937, TF-1a, MOLM-13, or MV4-11, were plated in flat bottom 96-well plates, 5000 cells per well in 100 μl RPMI-1640 complete media. VCID0230 and VCID0904 were serially diluted 1:10 in RPMI-1640 complete media starting at 200 nM. 100 μl of drug dilution was added to duplicate wells containing cells for a final starting concentration of 100 nM. VCID0231 was serially diluted 1:10 in RPMI-1640 complete media starting at 1000 nM. 100 μl of drug dilution was added to duplicate wells containing cells for a final starting concentration of 500 nM. After 5 days, Promega CellTiter-Glo was used to assess ATP levels by luminescence, as an assessment of relative cell viability in each well. Signal was normalized to vehicle control wells. Cell viability was plotted as percent live cells, normalized to vehicle control set at 100%. Average+/−SD was plotted.

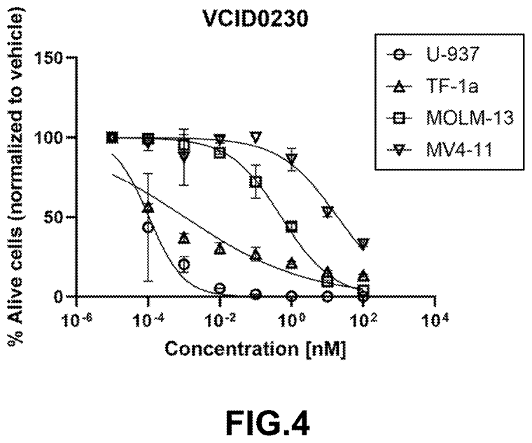

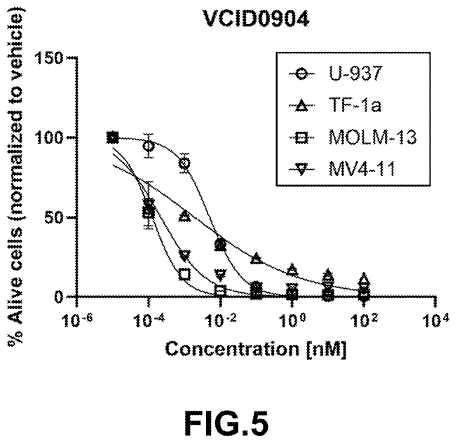

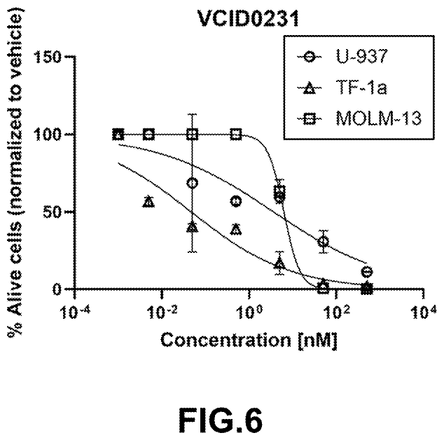

Results are shown in FIG. 4 (VCID0230), FIG. 5 (VCID0904), and FIG. 6 (VCID0231). For all drugs tested, each cell line tested showed greater than 50% killing at one or more of the concentrations tested. IC50 values were calculated from non-linear fitted data. The IC50 for VCID0230 was 0.05 pM for U-937 cells, 0.3 pM for TF-1a cells, 0.4 nM for MOLM-13 cells, and 10 nM for MV4-11 cells. The IC50 for VCID0904 was 4 pM for U-937 cells, 0.6 pM for TF-1a cells, 0.1 pM for MOLM-13 cells, and 0.1 pM for MV4-11 cells. The IC50 for VCID0231 was 17.6 nM for U-937 cells, 1.3 nM for TF-1a cells, and 8 nM for MOLM-13 cells.

### Example 3

In vitro cytotoxicity assays were carried out for additional molecules. Each molecule comprises, from N-terminus to C-terminus, a CDT-derived domain, a furin-cleavable linker, a DTB-derived translocation domain, and one or more targeting moieties. The CDT-derived domain and the targeting moieties were derived from various sources, as noted in the table below. Cells from AML cancer cell lines U-937, TF-1a, or MOLM-13 were plated in flat bottom 96-well plates, 2500 cells per well in 50 μl RPMI-1640 complete media. Drugs were serially diluted 1:100 in RPMI-1640 complete media starting at 200 nM and added 1:1 to cells for a starting concentration of 100 nM. Cells were incubated for 5 days at 37° C. and 5% CO₂. After 5 days, Promega CellTiter-Glo was used to assess ATP levels by luminescence, as an assessment of relative cell viability in each well. Signal was normalized to vehicle control wells. Results for drug concentrations of 1 nM and 10 pM are summarized in Table 2. For CDT, Hd indicates Haemophilus ducreyi, Hp indicates Haemophilus parasuis, Aa indicates Aggregatibacter actinomycetemcomitans, Ec indicates Escherichia coli, and Se indicates Salmonella enterica. For killing activity, ++ indicates <50% live cells, + indicates 50-80% live cells, and − indicates >80% live cells. N/D: not done. All of the molecules tested showed cytotoxic activity with respect to one or more of the cell lines tested, regardless of which CDT-derived domain was used.

TABLE 2

| | Targeting | | SEQ ID | Killing activity at 1 nM | | | Killing activity at 10 pM | | |
|---|---|---|---|---|---|---|---|---|---|
| | Moiety | CDT | NO. | U937 | TF1a | MOLM13 | U937 | TF1a | MOLM13 |
| VCID1300 | GM-CSF + | Hd | 43 | ++ | N/D | ++ | + | N/D | ++ |
| | IL3 | | | | | | | | |
| VCID1302 | GM-CSF + | Hd | 44 | ++ | N/D | ++ | ++ | N/D | ++ |
| | IL3 | | | | | | | | |
| VCID1303 | GM-CSF + | Hd | 45 | ++ | N/D | ++ | ++ | N/D | ++ |
| | IL3 | | | | | | | | |
| VCID1333 | GM-CSF + | Aa | 46 | ++ | N/D | ++ | + | N/D | ++ |
| | IL3 | | | | | | | | |
| VCID1334 | GM-CSF + | Aa | 47 | ++ | N/D | ++ | + | N/D | ++ |
| | IL3 | | | | | | | | |
| VCID1337 | GM-CSF + | Ec | 48 | ++ | N/D | ++ | + | N/D | ++ |
| | IL3 | | | | | | | | |
| VCID1338 | GM-CSF + | Ed | 49 | ++ | N/D | ++ | ++ | N/D | ++ |
| | IL3 | | | | | | | | |
| VCID1339 | GM-CSF + | Hp | 50 | ++ | N/D | ++ | ++ | N/D | ++ |
| | IL3 | | | | | | | | |
| VCID1340 | GM-CSF + | Hp | 51 | ++ | N/D | ++ | ++ | N/D | ++ |
| | IL3 | | | | | | | | |
| VCID1341 | GM-CSF | Hd | 32 | ++ | N/D | ++ | ++ | N/D | ++ |
| VCID1342 | GM-CSF | Hd | 33 | ++ | N/D | ++ | ++ | N/D | ++ |
| VCID1225 | GM-CSF | Aa | 34 | ++ | ++ | N/D | ++ | ++ | N/D |
| VCID1228 | GM-CSF | Ec | 35 | ++ | ++ | N/D | ++ | ++ | N/D |
| VCID1230 | GM-CSF | Hp | 36 | ++ | ++ | N/D | ++ | ++ | N/D |
| VCID1232 | GM-CSF | Se | 37 | ++ | + | N/D | ++ | − | N/D |

### Example 4

Molecule VCID0230 was tested in an in vivo study using a human tumor xenograft model in mice. Molecule VCID0230 (SEQ ID NO:31) comprises, from N-terminus to C-terminus, an HdCDT-derived domain, a furin-cleavable linker, a DTB-derived translocation domain, and a GM-CSF-derived targeting moiety.

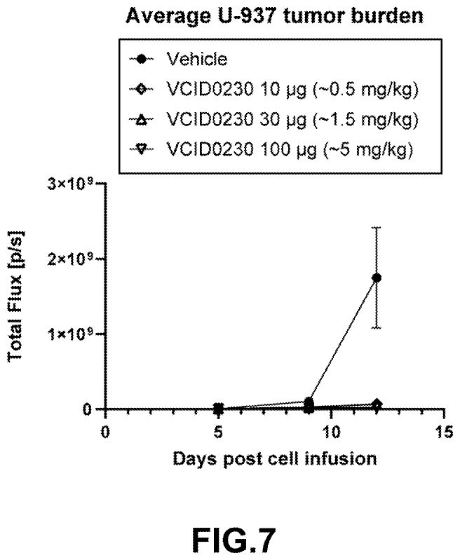

300,000 luciferase-expressing cells from AML cancer cell line U-937 were infused by tail vein injection (100 μl volume per injection) into female non-obese diabetic (NOD) severe combined immunodeficiency (scid) IL2 receptor gamma chain null mutation (gamma) mice (NSG mice) 8-12 weeks old. These mice are immunodeficient, lacking mature T, B, and NK cells, which allow for development of cancers from human tumor cells. Mice were treated starting 5 days after cell infusion to allow for engraftment of cells. VCID0230 or vehicle control (PBS) was dosed intraperitoneally (IP) once daily for 5 days at one of three doses: 10 μg, 30 μg, or 100 μg per mouse per day. These doses are approximately 0.5 mg/kg, 1.5 mg/kg, and 5 mg/kg, respectively. Tumor burden was monitored through an in vivo imaging system for luciferase signal twice a week. N=5 mice per condition. Average+/−SEM for tumor burden (i.e., luciferase signal) was plotted over time. Results are shown in FIG. 7. Average tumor burden for the VCID0230 treated mice in all dosage groups was notably lower than for vehicle-treated mice.

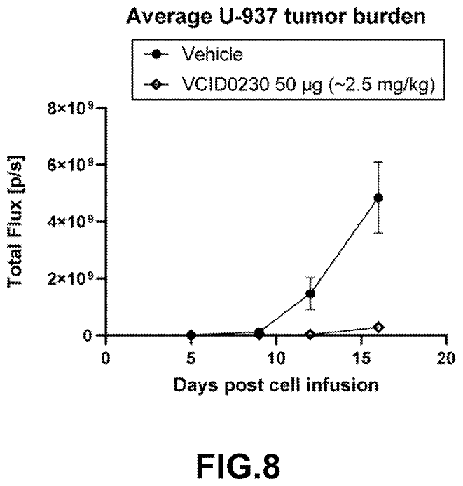

A similar experiment was carried out as described above, but treating with 50 μg of VCID0230 per mouse per day, which is approximately 2.5 mg/kg, or vehicle control. N=4 mice per condition. Results are shown in FIG. 8. Average tumor burden for the VCID0230 treated mice was again notably lower than for vehicle-treated mice.

### Example 5

Molecule VCID0230 was tested in additional in vivo studies using human tumor xenograft models in mice created with several different human tumor cell lines. Molecule VCID0230 (SEQ ID NO:31) comprises, from N-terminus to C-terminus, an HdCDT-derived domain, a furin-cleavable linker, a DTB-derived translocation domain, and a GM-CSF-derived targeting moiety.

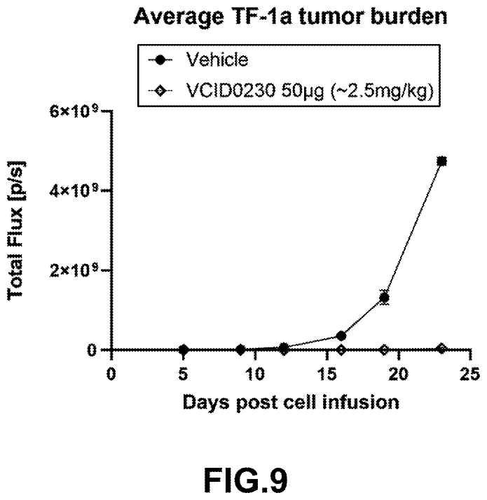

750,000 luciferase-expressing cells from AML cancer cell line TF-1a were infused by tail vein injection (100 μl volume per injection) into female NSG mice 8-12 weeks old. Mice were treated starting 5 days after cell infusion to allow for engraftment of cells. VCID0230 or vehicle control (PBS) was dosed IP once daily for 5 days at 50 μg per mouse per day, which is approximately 2.5 mg/kg. Tumor burden was monitored through an in vivo imaging system for luciferase signal twice a week. N=4 mice per condition. Average+/−SEM for tumor burden (i.e., luciferase signal) was plotted over time. Results are shown in FIG. 9. Average tumor burden for the VCID0230-treated mice was notably lower than for vehicle-treated mice.

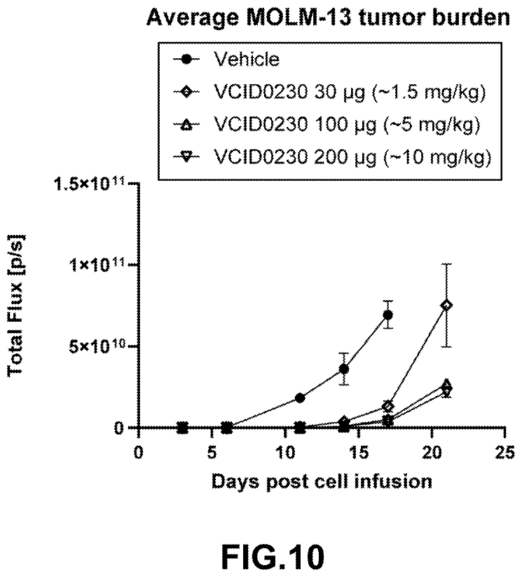

200,000 luciferase-expressing cells from AML cancer cell line MOLM-13 were infused by tail vein injection (100 μl volume per injection) into female NSG mice 8-12 weeks old. Mice were treated starting 1 day after cell infusion to allow for engraftment of cells. VCID0230 or vehicle control (PBS) was dosed IP once daily for 5 days at one of three doses: 30 μg, 100 μg, or 200 μg per mouse per day, which is approximately 1.5 mg/kg, 5 mg/kg, and 10 mg/kg, respectively. Tumor burden was monitored through an in vivo imaging system for luciferase signal twice a week. N=4 mice per condition. Results are shown in FIG. 10. Tumor burden is significantly delayed in mice in all VCID0230-dosage groups as compared to vehicle-treated mice.

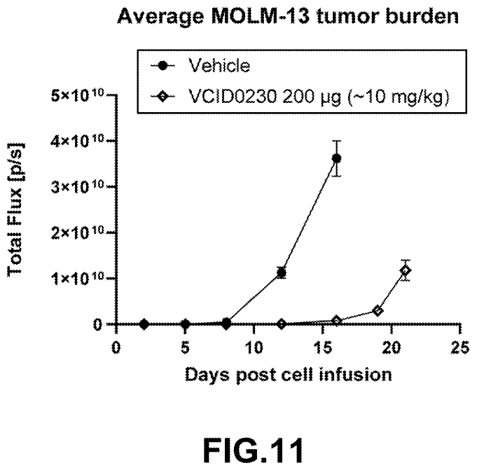

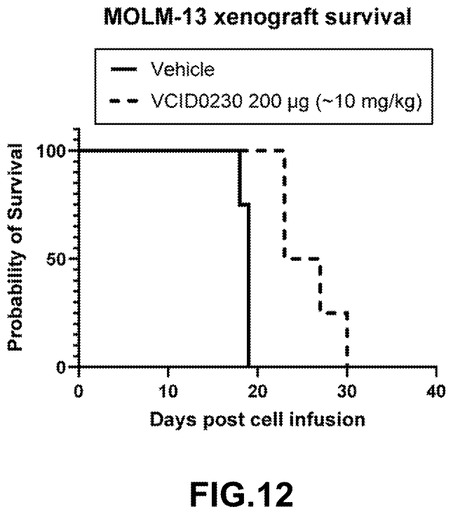

A similar experiment was carried out as described above with the mice receiving 200 μg of VCID0230 per mouse per day, which is approximately 10 mg/kg, or vehicle control. N=4 mice per condition. Results are shown in FIG. 11. Average tumor burden for the VCID0230-treated mice was notably lower than for vehicle-treated mice. The condition of all experimental mice was monitored against a set of standard euthanasia criteria, including specific guidelines regarding impaired mobility, impaired physiological function, weight loss, decreased food or water intake, and the like. Survival of treated and vehicle-treated control mice was recorded and is shown in FIG. 12, taking into consideration both euthanized animals and those found dead in cage. Compared to vehicle-treated mice, median survival of mice treated with VCID0230 increased from 19 days to 25 days post cell infusion.

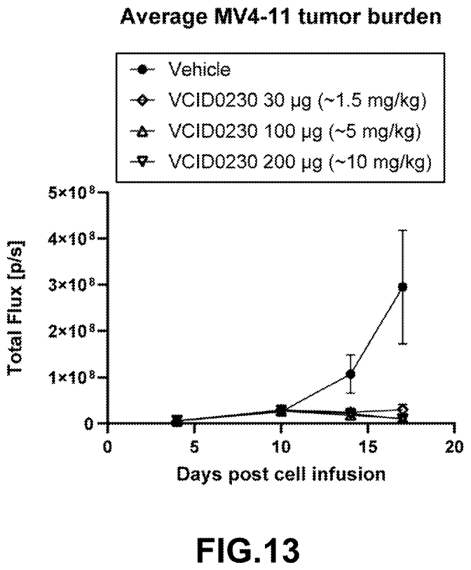

750,000 luciferase-expressing cells from AML cancer cell line MV4-11 were infused by tail vein injection (100 μl volume per injection) into female NSG mice 8-12 weeks old. Mice were treated starting 10 days after cell infusion to allow for engraftment of cells. VCID0230 or vehicle control (PBS) was dosed IP once daily for 5 days at one of three doses: 30 μg, 100 μg, or 200 μg per mouse per day, which is approximately 1.5 mg/kg, 5 mg/kg, and 10 mg/kg, respectively. Tumor burden was monitored through an in vivo imaging system for luciferase signal twice a week. N=4 mice per condition. Results are shown in FIG. 13. Average tumor burden for the VCID0230-treated mice in all dosage groups was notably lower than for vehicle-treated mice.

As can be observed in FIGS. 7-13, one or more tested doses of VCID0230 provided control of tumor burden in every cell line tested in these in vivo models.

### Example 6

Molecule VCID0904 was tested in in vivo studies using human tumor xenograft models in mice created with several different human cell lines. Molecule VCID0904 (SEQ ID NO:42) comprises, from N-terminus to C-terminus, an HdCDT-derived domain, a furin-cleavable linker, a DTB-derived translocation domain, a GM-CSF-derived targeting moiety, and an IL3-derived targeting moiety.

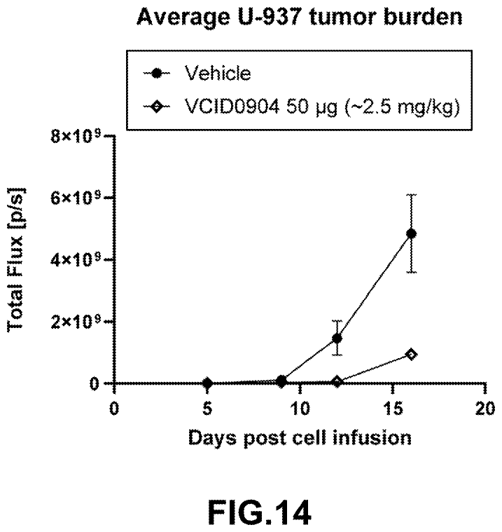

300,000 luciferase-expressing cells from AML cancer cell line U-937 were infused by tail vein injection (100 μl volume per injection) into female NSG mice 8-12 weeks old. Mice were treated starting 5 days after cell infusion to allow for engraftment of cells. VCID0904 or vehicle control (PBS) was dosed IP once daily for 5 days at 50 μg per mouse per day, which is approximately 2.5 mg/kg. Tumor burden was monitored through an in vivo imaging system for luciferase signal twice a week. N=4 mice per condition. Average+/−SEM for tumor burden (i.e., luciferase signal) was plotted over time. Results are shown in FIG. 14. Average tumor burden for the VCID0904-treated mice was notably lower than for vehicle-treated mice.

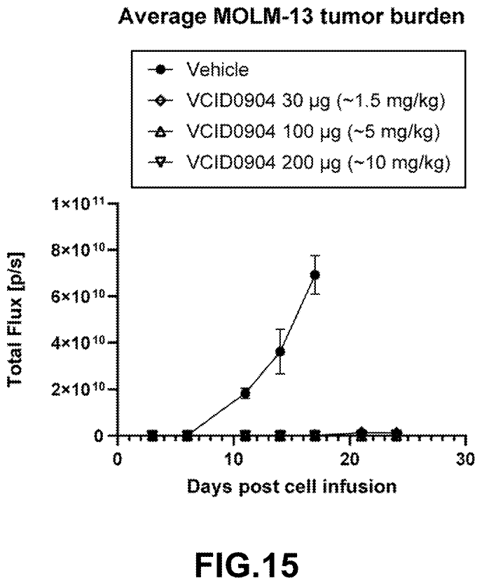

200,000 luciferase-expressing cells from AML cancer cell line MOLM-13 were infused by tail vein injection (100 μl volume per injection) into female NSG mice 8-12 weeks old. Mice were treated starting 1 day after cell infusion to allow for engraftment of cells. VCID0904 or vehicle control (PBS) was dosed IP once daily for 5 days at one of three doses: 30 μg, 100 μg, or 200 μg per mouse per day, which is approximately 1.5 mg/kg, 5 mg/kg, and 10 mg/kg, respectively. Tumor burden was monitored through an in vivo imaging system for luciferase signal twice a week. N=4 mice per condition. Results are shown in FIG. 15. Average tumor burden for the VCID0904-treated mice in all dosage groups was notably lower than for vehicle-treated mice.

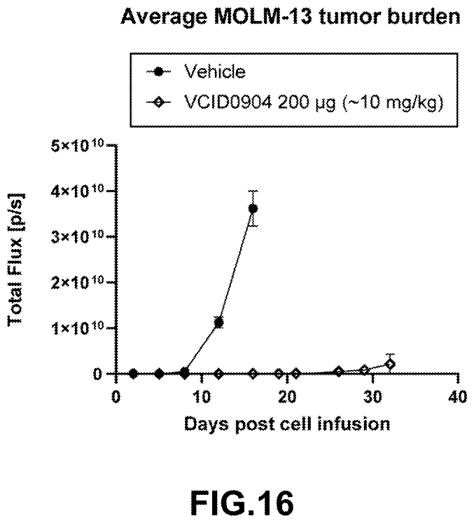

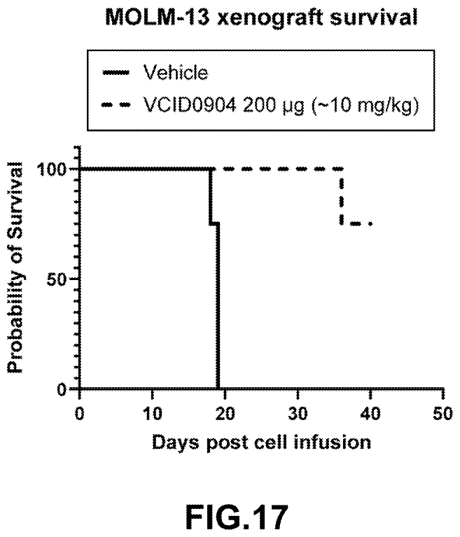

A similar experiment was carried out as described above with the mice receiving 200 μg of VCID0904 per mouse per day, which is approximately 10 mg/kg, or vehicle control. N=4 mice per condition. Results are shown in FIG. 16. Average tumor burden for the treated mice was notably lower than for untreated mice. Survival of treated and control mice was recorded and is shown in FIG. 17, taking into consideration both euthanized animals and those found dead in cage. Compared to vehicle-treated mice, median survival of mice treated with VCID0904 increased from 19 days to more than 40 days post cell infusion.

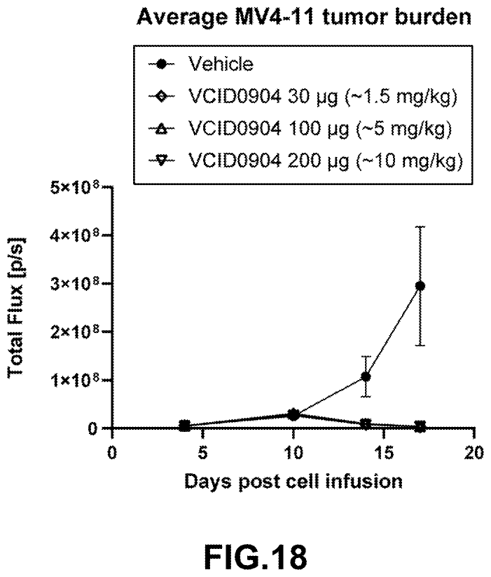

750,000 luciferase-expressing cells from AML cancer cell line MV4-11 were infused by tail vein injection (100 μl volume per injection) into female NSG mice 8-12 weeks old. Mice were treated starting 10 days after cell infusion to allow for engraftment of cells. VCID0904 or vehicle control (PBS) was dosed IP once daily for 5 days at one of three doses: 30 μg, 100 μg, or 200 μg per mouse per day, which is approximately 1.5 mg/kg, 5 mg/kg, and 10 mg/kg, respectively. Tumor burden was monitored through an in vivo imaging system for luciferase signal twice a week. N=4 mice per condition. Results are shown in FIG. 18. Average tumor burden for the VCID0904-treated mice in all dosage groups was notably lower than for vehicle-treated mice.

### Example 7

Molecule VCID0231 was tested in in vivo studies using a human tumor xenograft model in mice. Molecule VCID0231 (SEQ ID NO:38) comprises, from N-terminus to C-terminus, an HdCDT-derived domain, a furin-cleavable linker, a DTB-derived translocation domain, and an anti-CD33-derived targeting moiety.

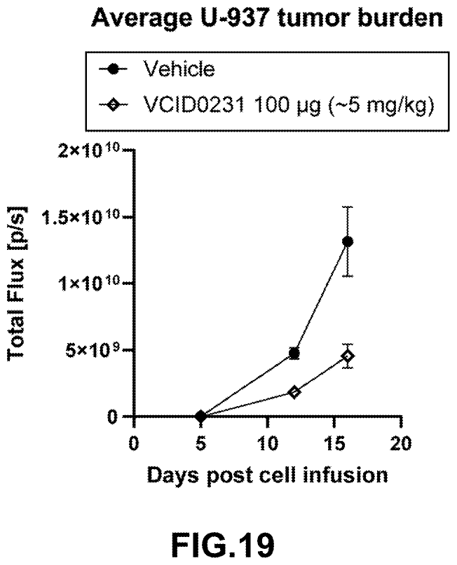

300,000 luciferase-expressing cells from AML cancer cell line U-937 were infused by tail vein injection (100 μl volume per injection) into female NSG mice 8-12 weeks old. Mice were treated starting 5 days after cell infusion to allow for engraftment of cells. VCID0231 or vehicle control (PBS) was dosed IP once daily for 5 days at 100 μg per mouse per day, which is approximately 5 mg/kg. Tumor burden was monitored through an in vivo imaging system for luciferase signal twice a week. N=5 mice per condition. Average+/−SEM for tumor burden (i.e., luciferase signal) was plotted over time. Results are shown in FIG. 19. Average tumor burden for the VCID0231-treated mice was notably lower than for vehicle-treated mice.

### Example 8

Several molecules were created and tested in an in vitro cytotoxicity assay to elucidate the importance of different components.

The first experiment uses molecules VCID0230 and VCID0539. Molecule VCID0230 (SEQ ID NO:31) comprises, from N-terminus to C-terminus, an HdCDT-derived domain, a furin-cleavable linker, a DTB-derived translocation domain, and a GM-CSF-derived targeting moiety. Molecule VCID0539 (SEQ ID NO:52) is similar to VCID0230, but incorporates a linker with reduced furin cleavage between the CDT-derived domain and the DTB-derived translocation domain.

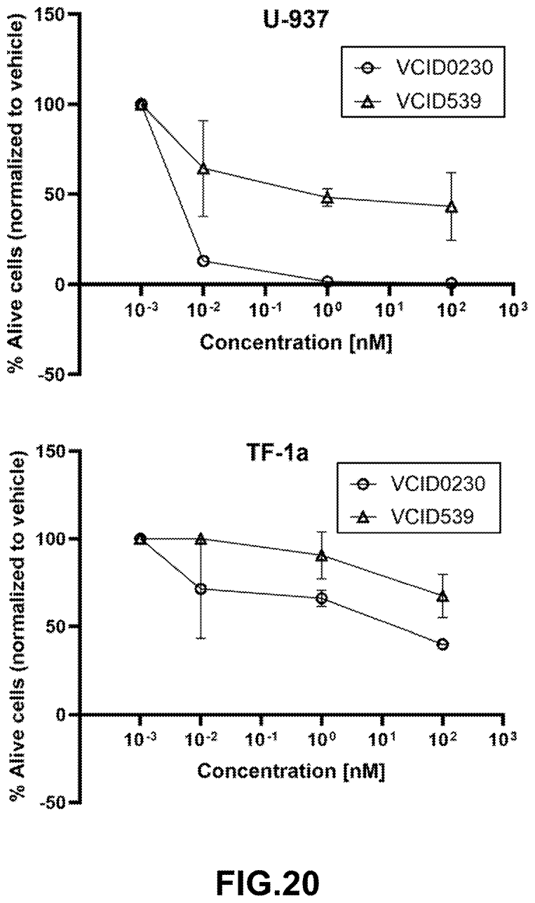

Cells from AML cancer cell line U-937 or TF-1a were plated in flat bottom 96-well plates, 2500 cells per well in 50 μl RPMI-1640 complete media. Drugs were serially diluted 1:100 in RPMI-1640 complete media starting at 200 nM. 50 μl of drug dilution was added to wells containing cells for a final starting concentration of 100 nM. Cells were incubated for 5 days at 37° C. and 5% CO₂. After 5 days, Promega CellTiter-Glo was used to assess ATP levels by luminescence, as an assessment of relative cell viability in each well. Signal was normalized to vehicle control wells. Cell viability was plotted as percent live cells, normalized to vehicle control set at 100%. Average+/−SD was plotted. Results are shown in FIG. 20. VCID0539, having a linker with reduced furin cleavage between the CDT-derived domain and the DTB-derived translocation domain, showed markedly less cell killing activity than VCID0230.

A similar experiment was conducted using molecules VCID0230 and VCID1172. Molecule VCID0230 (SEQ ID NO:31) comprises, from N-terminus to C-terminus, an HdCDT-derived domain, a furin-cleavable linker, a DTB-derived translocation domain, and a GM-CSF-derived targeting moiety. Molecule VCID1172 (SEQ ID NO:54) is similar to VCID0230, but does not include a DTB-derived translocation domain.

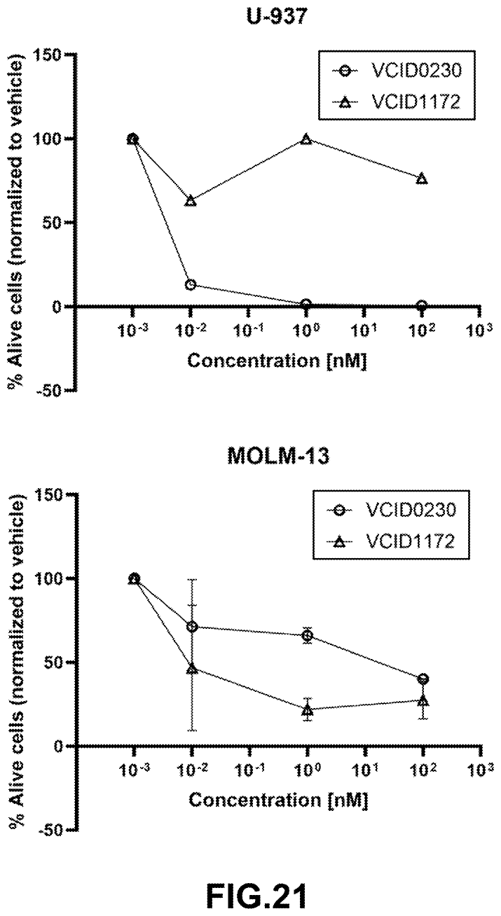

Cells from AML cancer cell line U-937 or MOLM-13 were plated in flat bottom 96-well plates, 2500 cells per well in 50 μl RPMI-1640 complete media. Drugs were serially diluted 1:100 in RPMI-1640 complete media starting at 200 nM. 50 μl of drug dilution was added to wells containing cells for a final starting concentration of 100 nM. Cells were incubated for 5 days at 37° C. and 5% CO₂. After 5 days, Promega CellTiter-Glo was used to assess ATP levels by luminescence, as an assessment of relative cell viability in each well. Signal was normalized to vehicle control wells. Cell viability was plotted as percent live cells, normalized to vehicle control set at 100%. Average+/−SD was plotted. Results are shown in FIG. 21. VCID1172, lacking a DTB-derived translocation domain, showed markedly less cell killing activity than VCID0230.

A third experiment was conducted using molecules VCID0230 and VCID1170. Molecule VCID0230 (SEQ ID NO:31) comprises, from N-terminus to C-terminus, an HdCDT-derived domain, a furin-cleavable linker, a DTB-derived translocation domain, and a GM-CSF-derived targeting moiety. Molecule VCID1170 (SEQ ID NO:55) is similar to VCID0230, but does not include either a furin-cleavable linker or a DTB-derived translocation domain.

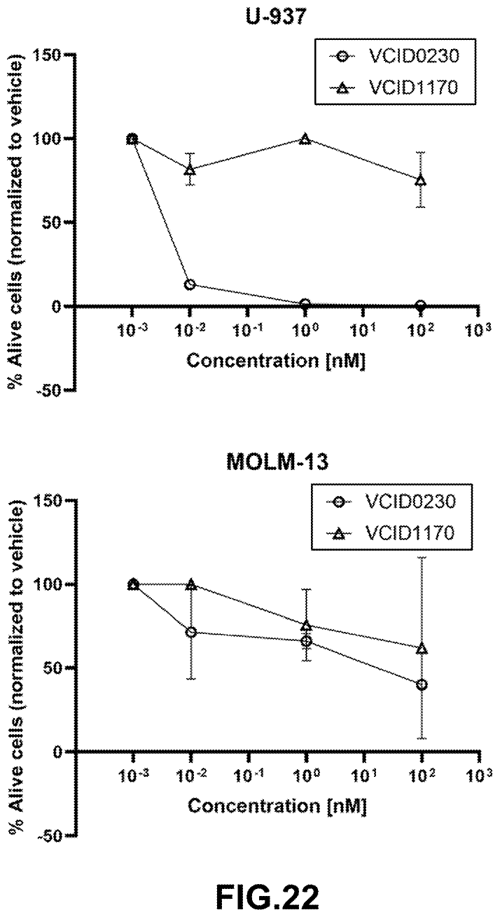

Cells from AML cancer cell line U-937 or MOLM-13 were plated in flat bottom 96-well plates, 2500 cells per well in 50 μl RPMI-1640 complete media. Drugs were serially diluted 1:100 in RPMI-1640 complete media starting at 200 nM. 50 μl of drug dilution was added to wells containing cells for a final starting concentration of 100 nM. Cells were incubated for 5 days at 37° C. and 5% CO₂. After 5 days, Promega CellTiter-Glo was used to assess ATP levels by luminescence, as an assessment of relative cell viability in each well. Signal was normalized to vehicle control wells. Cell viability was plotted as percent live cells, normalized to vehicle control set at 100%. Average+/−SD was plotted. Results are shown in FIG. 22. VCID1170, lacking both a furin-cleavable linker and a DTB-derived translocation domain, showed markedly less cell killing activity than VCID0230.

### Example 9

In vitro cytotoxicity assays were carried out using two further molecules. Molecule VCID1726 (SEQ ID NO:61) comprises, from N-terminus to C-terminus, an HpCDT-derived domain, a furin-cleavable linker, a DTB-derived translocation domain, a GM-CSF-derived targeting moiety, and an IL3-derived targeting moiety. Molecule VCID1728 (SEQ ID NO:62) comprises, from N-terminus to C-terminus, an HpCDT-derived domain, a furin-cleavable linker, a DTB-derived translocation domain which is a truncated version of DTB that retains translocation function (DTB403), a GM-CSF-derived targeting moiety, and an IL3-derived targeting moiety. Each molecule was tested at different concentrations using four different AML-related cell lines: U-937, TF-1a, MOLM-13, and MV4-11.

Cells from AML cancer cell lines U-937, TF-1a, MOLM-13, or MV4-11, were plated in flat bottom 96-well plates, 5000 cells per well in 100 μl RPMI-1640 complete media or 2500 cells in 50 μl RPMI-1640 complete media. VCID1726 and VCID1728 were serially diluted RPMI-1640 complete media then added 1:1 by volume into wells containing cells. After 5 days, cells were pipetted to mix, then 50 μl of cell suspension was transferred to white-sided 96 well flat bottom plates. Promega CellTiter-Glo Luminescent Cell Viability Assay® was added to cell suspension and mixed. After ten minutes' incubation in the dark, luciferase signal was read out with a luminometer. Luciferase signal was normalized to vehicle control wells. Cell viability was plotted as percent live cells, normalized to vehicle control set at 100%. Average+/−SD was plotted.

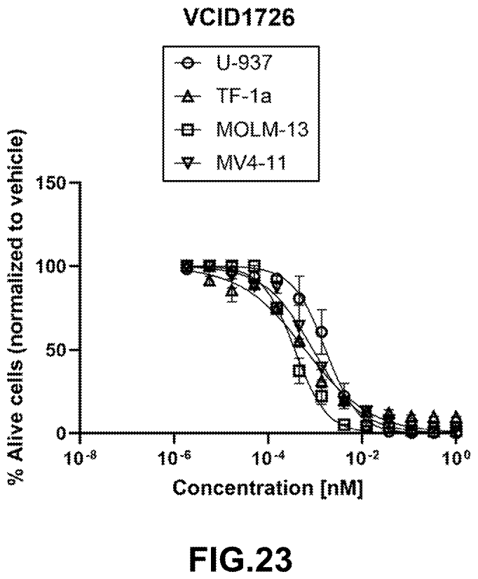

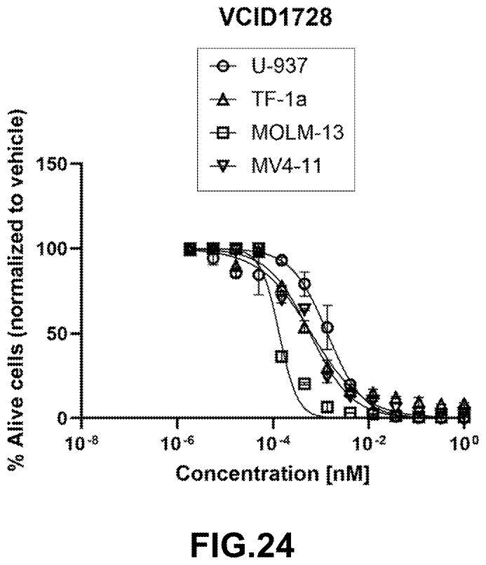

Results are shown in FIG. 23 (VCID1726) and FIG. 24 (VCID1728). For both tested molecules, each cell line tested showed greater than 50% killing at one or more of the concentrations tested. IC₅₀ values were calculated from non-linear fitted data. The IC50 for VCID1726 was 1.66 pM for U-937 cells, 0.62 pM for TF-1a cells, 0.37 pM for MOLM-13 cells, and 0.90 pM for MV4-11 cells. The IC₅₀ for VCID1728 was 1.38 pM for U-937 cells, 0.64 pM for TF-1a cells, 0.14 pM for MOLM-13 cells, and 0.60 pM for MV4-11 cells.

### Example 10

Molecule VCID1726 was tested in an in vivo study using a human tumor xenograft model in mice. Molecule VCID1726 (SEQ ID NO:61) comprises, from N-terminus to C-terminus, an HpCDT-derived domain, a furin-cleavable linker, a DTB-derived translocation domain, a GM-CSF-derived targeting moiety, and an IL3-derived targeting moiety.

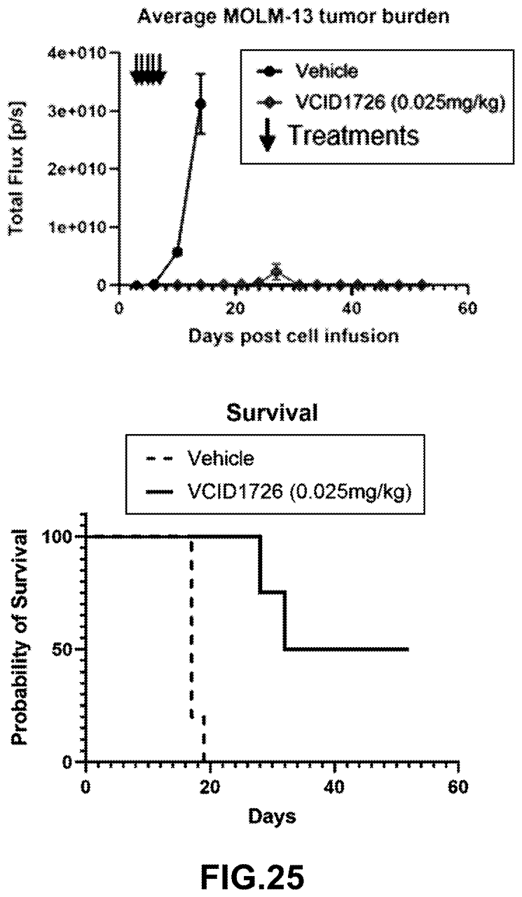

200,000 luciferase-expressing cells from AML cancer cell line MOLM-13 were infused by tail vein injection (100 μl volume per injection) into female NSG mice 8-12 weeks old. These mice are immunodeficient, lacking mature T, B, and NK cells, which allow for development of cancers from human tumor cells. Mice were treated starting 3 days after cell infusion to allow for engraftment of cells. VCID1726 or vehicle control (HBS) was dosed intraperitoneally (IP) once daily for 5 days at 0.025 mg/kg per mouse per day. Tumor burden was monitored through an in vivo imaging system for luciferase signal twice a week. N=4 mice per condition. Average+/−SEM for tumor burden (i.e., luciferase signal) was plotted over time. Results are shown in FIG. 25. The top panel shows average tumor burden. The tumor burden was controlled in the VCID1726-treated mice as compared to vehicle-treated mice. The bottom panel shows survival of VCID1726-treated mice as compared to vehicle-treated mice, taking into consideration both euthanized animals and those found dead in cage. VCID1726-treated mice have improved survival over vehicle-treated mice.

### Example 11

Molecule VCID1728 was tested in an in vivo study using a human tumor xenograft model in mice. Molecule VCID1728 (SEQ ID NO:62) comprises, from N-terminus to C-terminus, an HpCDT-derived domain, a furin-cleavable linker, a DTB-derived translocation domain, a GM-CSF-derived targeting moiety, and an IL3-derived targeting moiety.

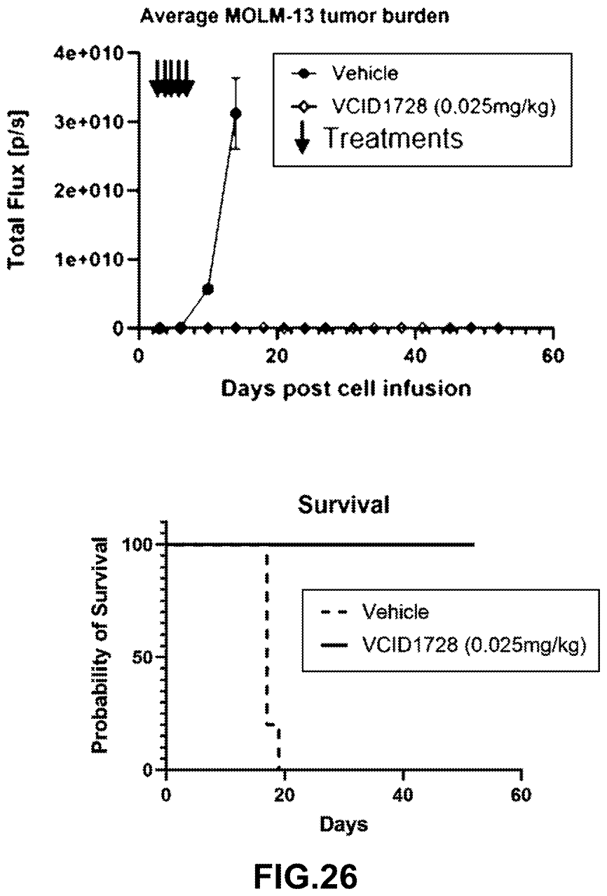

200,000 luciferase-expressing cells from AML cancer cell line MOLM-13 were infused by tail vein injection (100 μl volume per injection) into female NSG mice 8-12 weeks old. These mice are immunodeficient, lacking mature T, B, and NK cells, which allow for development of cancers from human tumor cells. Mice were treated starting 3 days after cell infusion to allow for engraftment of cells. VCID1728 or vehicle control (HBS) was dosed intraperitoneally (IP) once daily for 5 days at 0.025 mg/kg per mouse per day. Tumor burden was monitored through an in vivo imaging system for luciferase signal twice a week. N=4 mice per condition. Average+/−SEM for tumor burden (i.e., luciferase signal) was plotted over time. Results are shown in FIG. 26. The top panel shows average tumor burden. The tumor burden was controlled in the VCID1728-treated mice as compared to vehicle-treated mice. The bottom panel shows survival of VCID1728-treated mice as compared to vehicle-treated mice, taking into consideration both euthanized animals and those found dead in cage. VCID1728-treated mice all survived out to at least 50 days, as compared to vehicle-treated mice who did not survive past 20 days.

### Example 12

In vitro cytotoxicity assays were carried out using an additional molecule with a different targeting moiety. Molecule VCID1694 (SEQ ID NO:80) comprises, from N-terminus to C-terminus, an HpCDT-derived domain, a furin-cleavable linker, a DTB-derived translocation domain, and an anti-BCMA-derived targeting moiety. Different concentrations of VCID1694 were tested using two different lymphoma-related cell lines: Ramos and RPMI-8226.

Cells from lymphoma cancer cell lines Ramos or RPMI-8226 were plated in flat bottom 96-well plates, 5000 cells per well in 100 μl RPMI-1640 complete media. VCID1694 was serially diluted in RPMI-1640 complete media then added 1:1 by volume into wells containing cells. After 5 days, cells were pipetted to mix, then 50 μl of cell suspension was transferred to white-sided 96 well flat bottom plates. Promega CellTiter-Glo Luminescent Cell Viability Assay® was added to cell suspension and mixed. After ten minutes' incubation in the dark, luciferase signal was read out with a luminometer. Luciferase signal was normalized to vehicle control wells. Cell viability was plotted as percent live cells, normalized to vehicle control set at 100%. Average+/−SD was plotted.

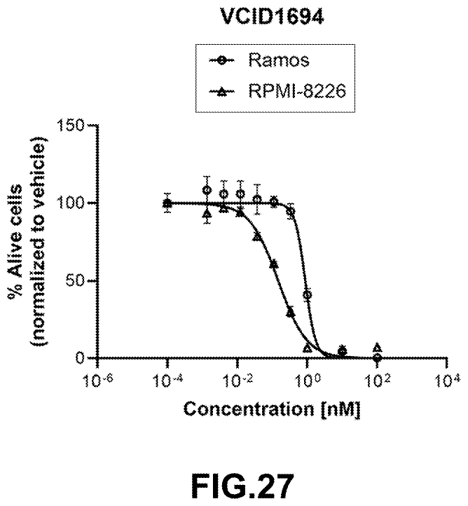

Results are shown in FIG. 27. For both cell lines tested, the drug showed greater than 50% killing at one or more of the concentrations tested. IC₅₀ values were calculated from non-linear fitted data. The IC50 for Ramos cells was 0.88 nM. The IC₅₀ for RPMI-8226 cells was 0.15 nM.

### Example 13

Molecule VCID1694 was tested in an in vivo study using a human tumor xenograft model in mice. Molecule VCID1694 (SEQ ID NO:80) comprises, from N-terminus to C-terminus, an HpCDT-derived domain, a furin-cleavable linker, a DTB-derived translocation domain, and an anti-BCMA-derived targeting moiety.

750,000 luciferase-expressing cells from lymphoma cancer cell line Ramos were infused by tail vein injection (100 μl volume per injection) into female NSG mice 8-12 weeks old. These mice are immunodeficient, lacking mature T, B, and NK cells, which allow for development of cancers from human tumor cells. Mice were treated starting 5 days after cell infusion to allow for engraftment of cells. VCID1694 or vehicle control (PBS) was dosed intraperitoneally (IP) once daily for 5 days at 1 mg/kg or 5 mg/kg per mouse per day. Tumor burden was monitored through an in vivo imaging system for luciferase signal twice a week. N=5 mice per condition. Average+/−SEM for tumor burden (i.e., luciferase signal) was plotted over time.

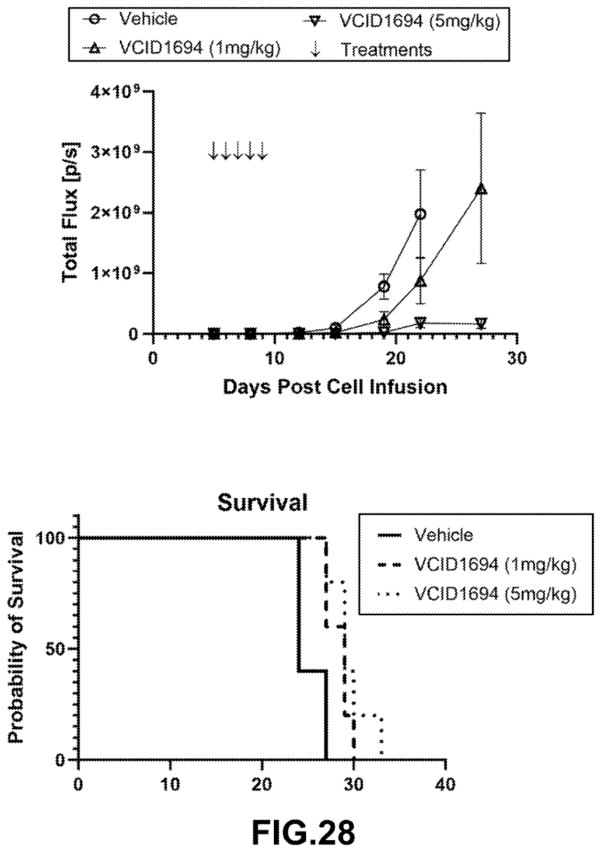

Results are shown in FIG. 28. The top panel shows average tumor burden. The tumor burden was reduced in the VCID1694-treated mice as compared to vehicle-treated mice, with greater reduction in tumor burden observed with the 5 mg/kg dosing than with the 1 mg/kg dosing. The bottom panel shows survival of VCID1694-treated mice as compared to vehicle-treated mice, taking into consideration both euthanized animals and those found dead in cage. VCID1694-treated mice showed improved survival as compared to vehicle-treated mice, with a greater increase in survival observed with the 5 mg/kg dosing than with the 1 mg/kg dosing.

### Example 14

In vitro cytotoxicity assays were carried out using an additional molecule with a different targeting moiety. VCID1700 (SEQ ID NO:82) comprises, from N-terminus to C-terminus, an HpCDT-derived domain, a furin-cleavable linker, a DTB-derived translocation domain, and an anti-CD19-derived targeting moiety. Different concentrations of VCID1700 were tested using two different lymphoma-related cell lines: Ramos and Daudi.

Cells from lymphoma cancer cell lines Ramos or Daudi were plated in flat bottom 96-well plates, 5000 cells per well in 100 μl RPMI-1640 complete media. VCID1700 was serially diluted in RPMI-1640 complete media then added 1:1 by volume into wells containing cells. After 5 days, cells were pipetted to mix, then 50 μl of cell suspension was transferred to white-sided 96 well flat bottom plates. Promega CellTiter-Glo Luminescent Cell Viability Assay® was added to cell suspension and mixed. After ten minutes' incubation in the dark, luciferase signal was read out with a luminometer. Luciferase signal was normalized to vehicle control wells. Cell viability was plotted as percent live cells, normalized to vehicle control set at 100%. Average+/−SD was plotted.

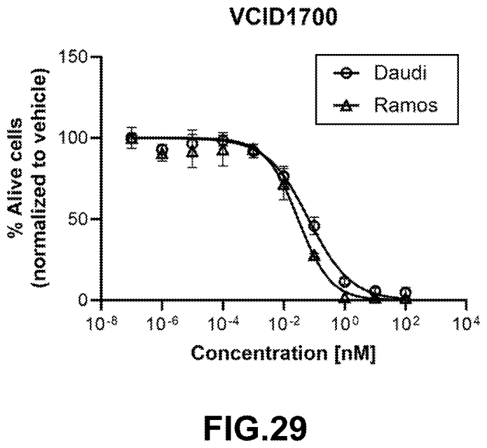

Results for VCID1700 are shown in FIG. 29. For both cell lines tested, the drug showed greater than 50% killing at one or more of the concentrations tested. IC₅₀ values were calculated from non-linear fitted data. The IC50 for Ramos cells was 0.03 nM. The IC₅₀ for Daudi cells was 0.07 nM.

### Example 15

Molecule VCID1700 was tested in an in vivo study using a human tumor xenograft model in mice. Molecule VCID1700 (SEQ ID NO:82) comprises, from N-terminus to C-terminus, an HpCDT-derived domain, a furin-cleavable linker, a DTB-derived translocation domain, and an anti-CD19-derived targeting moiety.

750,000 luciferase-expressing cells from lymphoma cancer cell line Ramos were infused by tail vein injection (100 μl volume per injection) into female NSG mice 8-12 weeks old. These mice are immunodeficient, lacking mature T, B, and NK cells, which allow for development of cancers from human tumor cells. Mice were treated starting 5 days after cell infusion to allow for engraftment of cells. VCID1700 or vehicle control (PBS) was dosed intraperitoneally (IP) once daily for 5 days at 1 mg/kg or 5 mg/kg per mouse per day. Tumor burden was monitored through an in vivo imaging system for luciferase signal twice a week. N=5 mice per condition. Average+/−SEM for tumor burden (i.e., luciferase signal) was plotted over time.

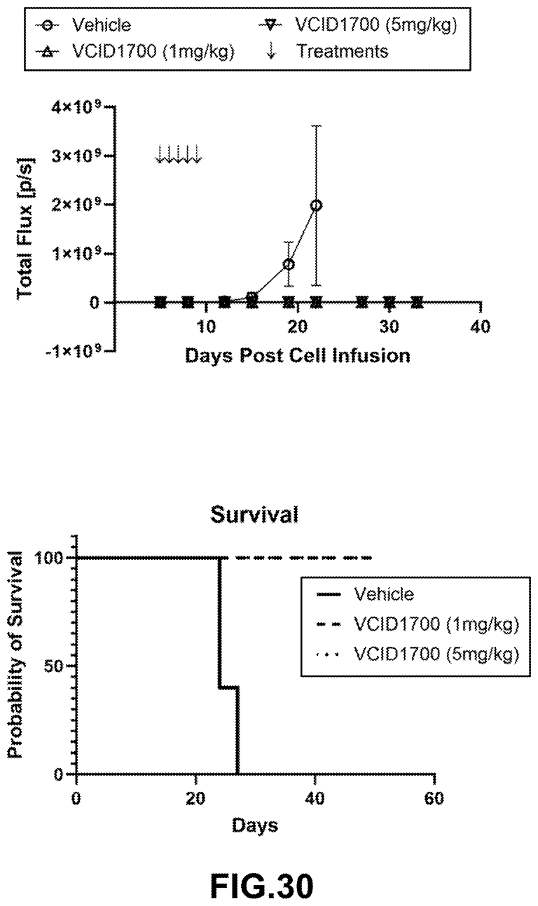

Results are shown in FIG. 30. The top panel shows average tumor burden. The tumor burden was well controlled in the VCID1700-treated mice as compared to vehicle-treated mice at both dose levels. The bottom panel shows survival of VCID1700-treated mice as compared to vehicle-treated mice, taking into consideration both euthanized animals and those found dead in cage. VCID1700-treated mice showed improved survival as compared to vehicle-treated mice, with all VCID1700-treated mice surviving past 32 days, while no vehicle-treated mice survived past 28 days.

### Example 16

In vitro cytotoxicity assays were carried out using three additional molecules. Molecule VCID1572 (SEQ ID NO:88) comprises, from N-terminus to C-terminus, an HdCDT-derived domain, a furin-cleavable linker, a DTB-derived translocation domain, an IL4-derived targeting moiety, and an IL2-derived targeting moiety. VCID1573 (SEQ ID NO:89) comprises, from N-terminus to C-terminus, an HdCDT-derived domain, a furin-cleavable linker, a DTB-derived translocation domain, and an IL4-derived targeting moiety. VCID1219 (SEQ ID NO:91) comprises, from N-terminus to C-terminus, an HdCDT-derived domain, a furin-cleavable linker, a DTB-derived translocation domain, and an IL2-derived targeting moiety. Different concentrations of each drug were tested using cells from large cell lymphoma cancer cell line SU-DHL-1.

Cells from lymphoma cancer cell line SU-DHL-1 were plated in flat bottom 96-well plates, 5000 cells per well in 100 μl RPMI-1640 complete media. VCID1572, VCID1573, or VCID1219 was serially diluted in RPMI-1640 complete media then added 1:1 by volume into wells containing cells. After 5 days, cells were pipetted to mix, then 50 μl of cell suspension was transferred to white-sided 96 well flat bottom plates. Promega CellTiter-Glo Luminescent Cell Viability Assay® was added to cell suspension and mixed. After ten minutes' incubation in the dark, luciferase signal was read out with a luminometer. Luciferase signal was normalized to vehicle control wells. Cell viability was plotted as percent live cells, normalized to vehicle control set at 100%. Average+/−SD was plotted.

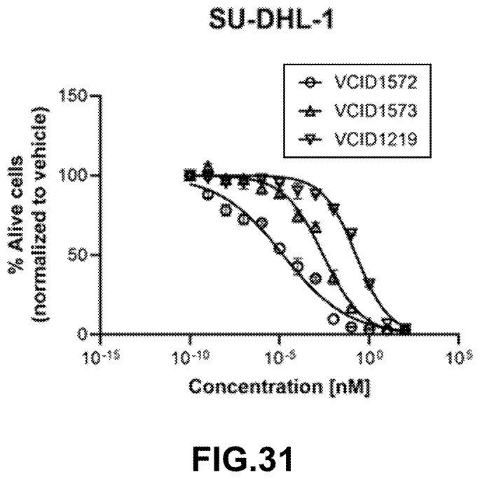

Results are shown in FIG. 31. For all drugs tested, the drug showed greater than 50% killing at one or more of the concentrations tested. IC₅₀ values were calculated from non-linear fitted data. The IC₅₀ for VCID1572 was 0.02 pM. The IC₅₀ for VCID1573 was 2.72 pM. The IC₅₀ for VCID1219 was 183.90 pM. Thus, while all three molecules have strong cytotoxic activity, it appears that the molecule comprising both an IL2-derived targeting moiety and an IL4-derived targeting moiety has improved potency to a degree that indicates a synergistic effect from the combination of these two targeting moieties.

### Example 17

In vitro cytotoxicity assays were carried out using two additional molecules, to confirm the effect of having two targeting moieties in the context of an HpCDT-derived domain. Molecule VCID1800 (SEQ ID NO:87) comprises, from N-terminus to C-terminus, an HpCDT-derived domain, a furin-cleavable linker, a DTB-derived translocation domain, an IL4-derived targeting moiety, and an IL2-derived targeting moiety. VCID1801 (SEQ ID NO:86) comprises, from N-terminus to C-terminus, an HpCDT-derived domain, a furin-cleavable linker, a DTB-derived translocation domain, and an IL4-derived targeting moiety. Different concentrations of each drug were tested using cells from large cell lymphoma cancer cell line SU-DHL-1.

Cells from lymphoma cancer cell line SU-DHL-1 were plated in flat bottom 96-well plates, 5000 cells per well in 100 μl RPMI-1640 complete media. VCID1800 or VCID1801 was serially diluted in RPMI-1640 complete media then added 1:1 by volume into wells containing cells. After 5 days, cells were pipetted to mix, then 50 μl of cell suspension was transferred to white-sided 96 well flat bottom plates. Promega CellTiter-Glo Luminescent Cell Viability Assay® was added to cell suspension and mixed. After ten minutes' incubation in the dark, luciferase signal was read out with a luminometer. Luciferase signal was normalized to vehicle control wells. Cell viability was plotted as percent live cells, normalized to vehicle control set at 100%. Average+/−SD was plotted.

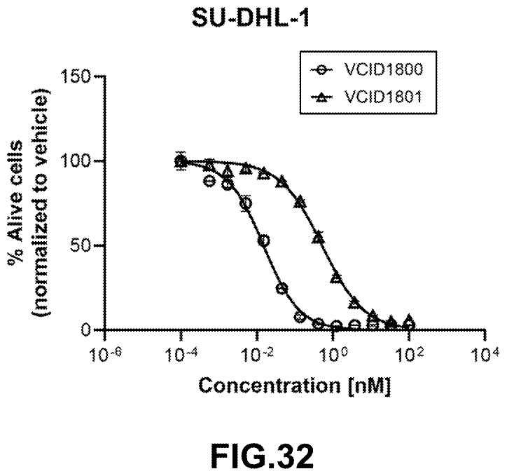

Results are shown in FIG. 32. For both drugs tested, the drug showed greater than 50% killing at one or more of the concentrations tested. IC₅₀ values were calculated from non-linear fitted data. The IC₅₀ for VCID1800 was 0.015 nM. The IC₅₀ for VCID1801 was 0.52 nM. Again, while both molecules have strong cytotoxic activity, the molecule with both an IL2-derived targeting moiety and an IL4-derived targeting moiety shows greater potency than the molecule with an IL4-derived targeting moiety alone.

### Example 18

In vitro cytotoxicity assays were carried out using four molecules with targeting moieties directed to CLDN18.2. Molecule VCID1418 (SEQ ID NO:93) comprises, from N-terminus to C-terminus, an HdCDT-derived domain, a furin-cleavable linker, a DTB-derived translocation domain, and an anti-CLDN18.2-derived targeting moiety. Molecule VCID1419 (SEQ ID NO:95) comprises, from N-terminus to C-terminus, an HdCDT-derived domain, a furin-cleavable linker, a DTB-derived translocation domain, and an anti-CLDN18.2-derived targeting moiety. VCID1418 and VCID1419 use two different targeting moieties. The VCID1418 targeting moiety is derived from an osemitamab scFv and the VCID1419 targeting moiety is derived from a zolbetuximab scFv. Molecule VCID1793 (SEQ ID NO:96) comprises, from N-terminus to C-terminus, an HpCDT-derived domain, a furin-cleavable linker, a DTB-derived translocation domain, and an anti-CLDN18.2-derived targeting moiety. Molecule VCID1794 (SEQ ID NO:97) comprises, from N-terminus to C-terminus, an HpCDT-derived domain, a furin-cleavable linker, a DTB-derived translocation domain, and an anti-CLDN18.2-derived targeting moiety. VCID1793 and VCID1794 are similar to VCID1418 and VCID1419, respectively, but incorporate the DTB403 translocation domain. Different concentrations of each drug were tested using cells from cancer cell line HEK293 engineered to express CLDN18.2 (HEK293-CLDN18.2 cells).

HEK293-CLDN18.2 cells were plated in flat bottom 96-well plates, 10,000 cells per well in 100 μl RPMI-1640 complete media. VCID1418, VCID1419, VCID1793, or VCID1794 was serially diluted in RPMI-1640 complete media then added 1:1 by volume into wells containing cells. Drug dilutions were added to the wells containing cells slowly and carefully to avoid cells detaching from the plate. Plates were immediately placed inside a Revvity, Inc. Incucyte® SX5 cell culture incubator at 37° C. and 5% CO₂ for live cell imaging. Images were taken at 10× with the phase channel every 12 hours for 3-5 days. Images were analyzed for % confluence in the well based on phase imaging. Average+/−SD was plotted.

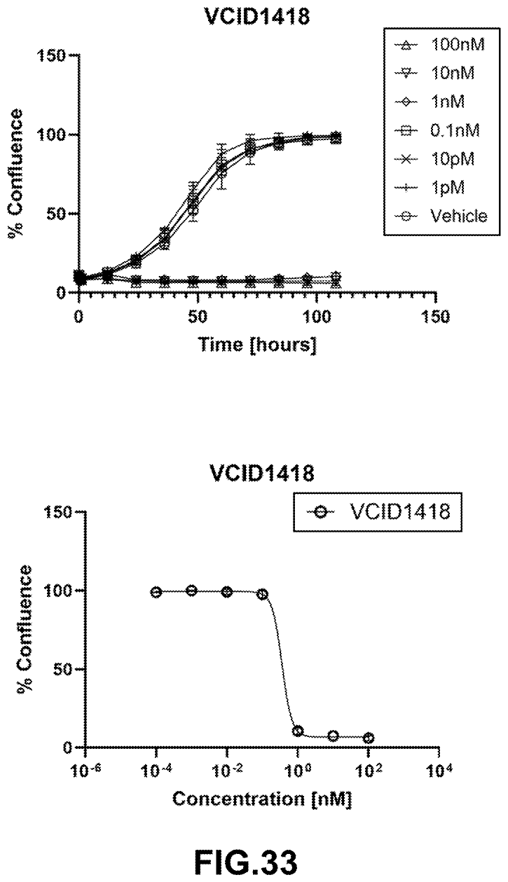

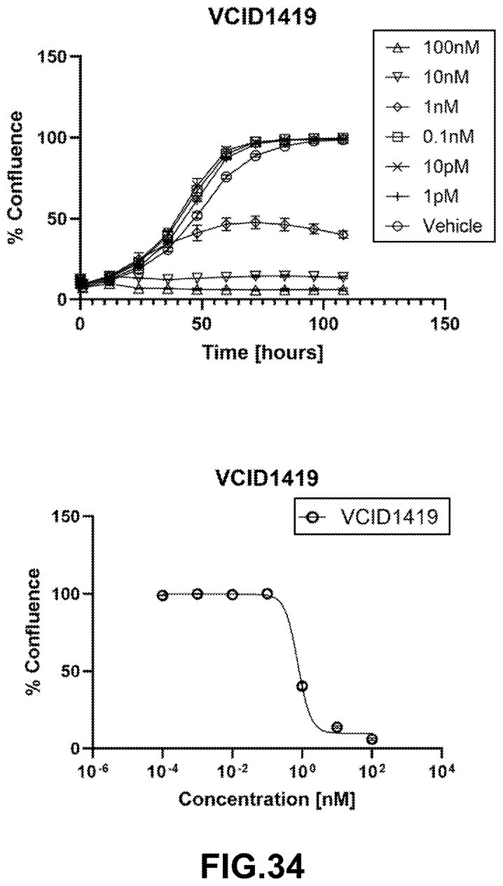

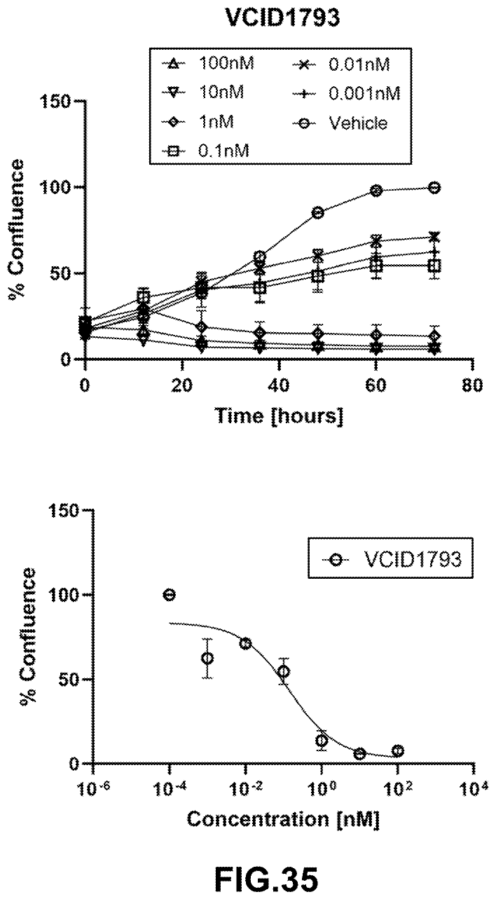

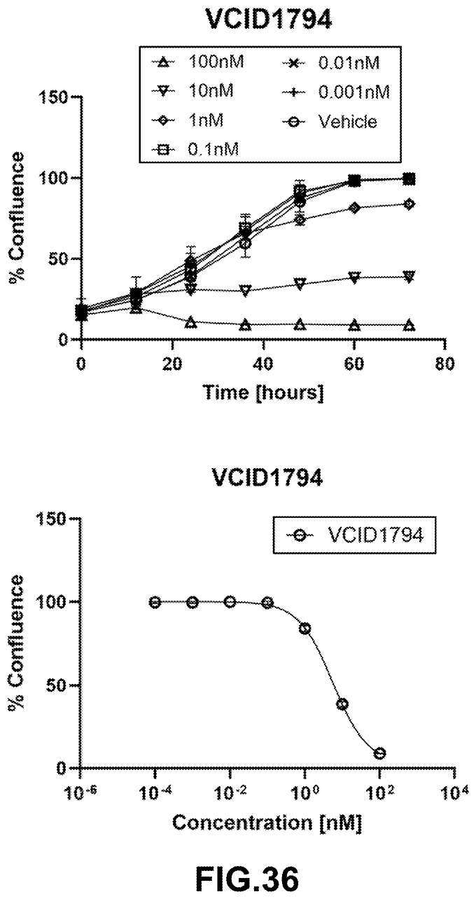

Results for VCID1418 are shown in FIG. 33. Top panel shows % confluence over time. The dose curve at 72 hours was plotted, and is shown in the bottom panel. The IC₅₀ for VCID1418 was 0.36 nM. Results for VCID1419 are shown in FIG. 34. Top panel shows % confluence over time. The dose curve at 72 hours was plotted, and is shown in the bottom panel. The IC₅₀ for VCID1419 was 0.75 nM. Results for VCID1793 are shown in FIG. 35. Top panel shows % confluence over time. The dose curve at 72 hours was plotted, and is shown in the bottom panel. The IC₅₀ for VCID1793 was 0.14 nM. Results for VCID1794 are shown in FIG. 36. Top panel shows % confluence over time. The dose curve at 72 hours was plotted, and is shown in the bottom panel. The IC₅₀ for VCID1794 was 0.75 nM. All molecules tested showed strong cytotoxic activity.

### Example 19

In vitro cytotoxicity assays were carried out using three molecules with targeting moieties directed to HER2. Molecule VCID1896 (SEQ ID NO:99) comprises, from N-terminus to C-terminus, an HpCDT-derived domain, a furin-cleavable linker, a DTB-derived translocation domain, and an anti-HER2-derived targeting moiety. Molecule VCID1898 (SEQ ID NO:101) comprises, from N-terminus to C-terminus, an HpCDT-derived domain, a furin-cleavable linker, a DTB-derived translocation domain, and an anti-HER2-derived targeting moiety. Molecule VCID1899 (SEQ ID NO: 103) comprises, from N-terminus to C-terminus, an HpCDT-derived domain, a furin-cleavable linker, a DTB-derived translocation domain, and an anti-HER2-derived targeting moiety. These three molecules are similar, but use targeting moieties derived from different anti-HER2 antibodies. Different concentrations of each drug were tested using cells from cancer cell line HEK293 engineered to express HER2 (HEK293-HER2 cells).

HEK293-HER2 cells were plated in flat bottom 96-well plates, 10,000 cells per well in 100 μl RPMI-1640 complete media. VCID1896, VCID1898, or VCID1899 was serially diluted in RPMI-1640 complete media then added 1:1 by volume into wells containing cells. Drug dilutions were added to the wells containing cells slowly and carefully to avoid cells detaching from the plate. Plates were immediately placed inside a Revvity, Inc. Incucyte® SX5 cell culture incubator at 37° C. and 5% CO₂ for live cell imaging. Images were taken at 10× with the phase channel every 12 hours for 3-5 days. Images were analyzed for % confluence in the well based on phase imaging. Average+/−SD was plotted.

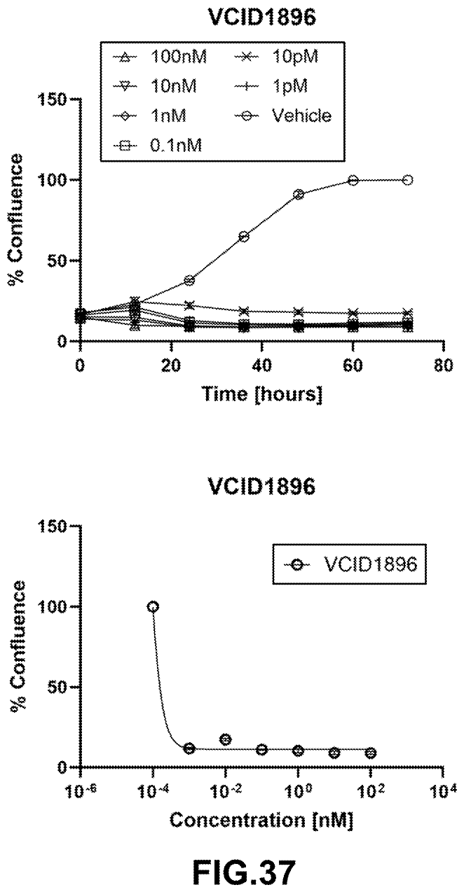

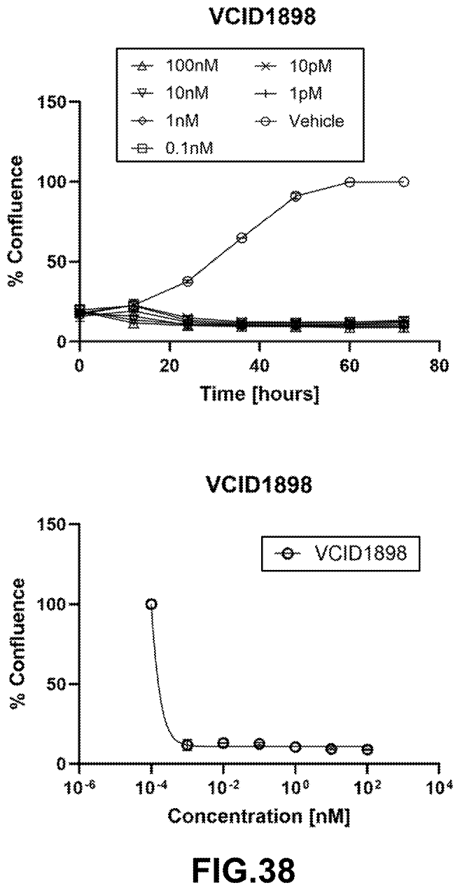

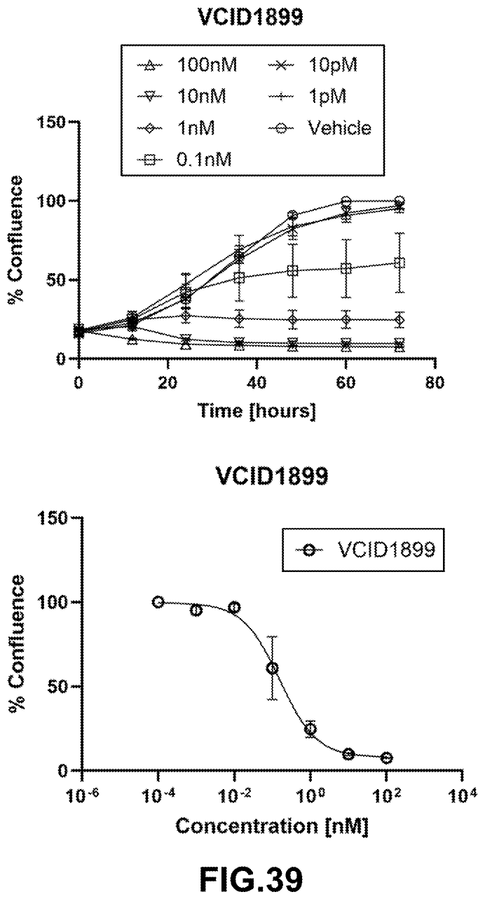

Results for VCID1896 are shown in FIG. 37. Top panel shows % confluence over time. The dose curve at 72 hours was plotted, and is shown in the bottom panel. The IC₅₀ for VCID1896 was <1 pM. Results for VCID1898 are shown in FIG. 38. Top panel shows % confluence over time. The dose curve at 72 hours was plotted, and is shown in the bottom panel. The IC₅₀ for VCID1898 was <1 pM. Results for VCID1899 are shown in FIG. 39. Top panel shows % confluence over time. The dose curve at 72 hours was plotted, and is shown in the bottom panel. The IC₅₀ for VCID1899 was <1 pM. All molecules tested showed strong cytotoxic activity.

### Example 20

In vitro cytotoxicity assays were carried out using a molecule with a targeting moiety directed to MSLN. Molecule VCID1862 (SEQ ID NO:105) comprises, from N-terminus to C-terminus, an HpCDT-derived domain, a furin-cleavable linker, a DTB-derived translocation domain, and an anti-MSLN-derived targeting moiety. Different concentrations of drug were tested using cells from cancer cell line HEK293 engineered to express MSLN (HEK293-MSLN cells).

HEK293-MSLN cells were plated in flat bottom 96-well plates, 10,000 cells per well in 100 μl RPMI-1640 complete media. VCID1862 was serially diluted in RPMI-1640 complete media then added 1:1 by volume into wells containing cells. Drug dilutions were added to the wells containing cells slowly and carefully to avoid cells detaching from the plate. Plates were immediately placed inside a Revvity, Inc. Incucyte® SX5 cell culture incubator at 37° C. and 5% CO₂ for live cell imaging. Images were taken at 10× with the phase channel every 12 hours for 3-5 days. Images were analyzed for % confluence in the well based on phase imaging. Average+/−SD was plotted.

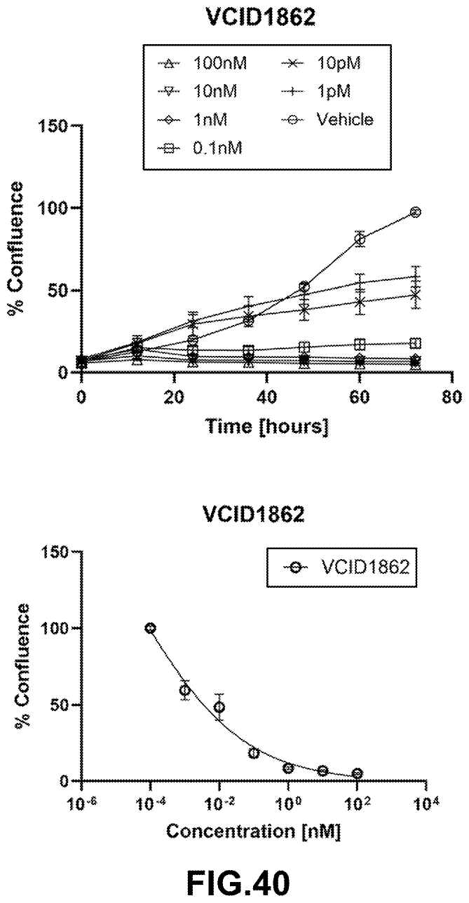

Results for VCID1862 are shown in FIG. 40. Top panel shows % confluence over time. The dose curve at 72 hours was plotted, and is shown in the bottom panel. The IC₅₀ for VCID1862 was 0.04 pM.

### Example 21

In vitro cytotoxicity assays were carried out using a molecule with a targeting moiety directed to GPC3. Molecule VCID1861 (SEQ ID NO: 107) comprises, from N-terminus to C-terminus, an HpCDT-derived domain, a furin-cleavable linker, a DTB-derived translocation domain, and an anti-GPC3-derived targeting moiety. Different concentrations of drug were tested using cells from cancer cell line HEK293 engineered to express GPC3 (HEK293-GPC3 cells).

HEK293-GPC3 cells were plated in flat bottom 96-well plates, 10,000 cells per well in 100 μl RPMI-1640 complete media. VCID1861 was serially diluted in RPMI-1640 complete media then added 1:1 by volume into wells containing cells. Drug dilutions were added to the wells containing cells slowly and carefully to avoid cells detaching from the plate. Plates were immediately placed inside a Revvity, Inc. Incucyte® SX5 cell culture incubator at 37° C. and 5% CO₂ for live cell imaging. Images were taken at 10× with the phase channel every 12 hours for 3-5 days. Images were analyzed for % confluence in the well based on phase imaging. Average+/−SD was plotted.

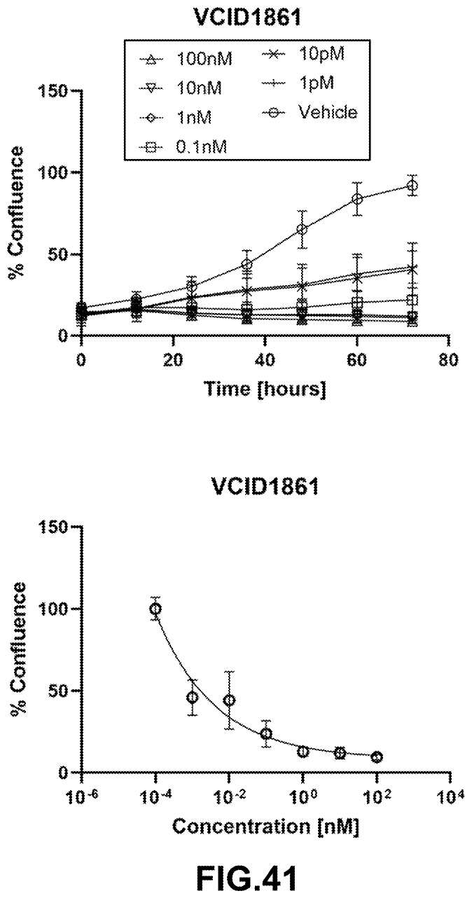

Results for VCID1861 are shown in FIG. 41. Top panel shows % confluence over time. The dose curve at 72 hours was plotted, and is shown in the bottom panel. The IC₅₀ for VCID1861 was <1 pM.

### Example 22

In vitro cytotoxicity assays were carried out using three molecules with targeting moieties directed to EGFR. Molecule VCID1795 (SEQ ID NO:109) comprises, from N-terminus to C-terminus, an HpCDT-derived domain, a furin-cleavable linker, a DTB-derived translocation domain, and an anti-EGFR-derived targeting moiety. VCID1906 (SEQ ID NO:111) comprises, from N-terminus to C-terminus, an HpCDT-derived domain, a furin-cleavable linker, a DTB-derived translocation domain, and an anti-EGFR-derived targeting moiety. VCID1907 (SEQ ID NO:113) comprises, from N-terminus to C-terminus, an HpCDT-derived domain, a furin-cleavable linker, a DTB-derived translocation domain, and an anti-EGFR-derived targeting moiety. These three molecules each comprise a targeting moiety derived from a different anti-EGFR antibody. Different concentrations of drug were tested using cells from cancer cell line HEK293 engineered to express EGFR (HEK293-EGFR cells).

HEK293-EGFR cells were plated in flat bottom 96-well plates, 10,000 cells per well in 100 μl RPMI-1640 complete media. VCID1795, VCID1906, or VCID1907 was serially diluted in RPMI-1640 complete media then added 1:1 by volume into wells containing cells. Drug dilutions were added to the wells containing cells slowly and carefully to avoid cells detaching from the plate. Plates were immediately placed inside a Revvity, Inc. Incucyte® SX5 cell culture incubator at 37° C. and 5% CO₂ for live cell imaging. Images were taken at 10× with the phase channel every 12 hours for 3-5 days. Images were analyzed for % confluence in the well based on phase imaging. Average+/−SD was plotted.

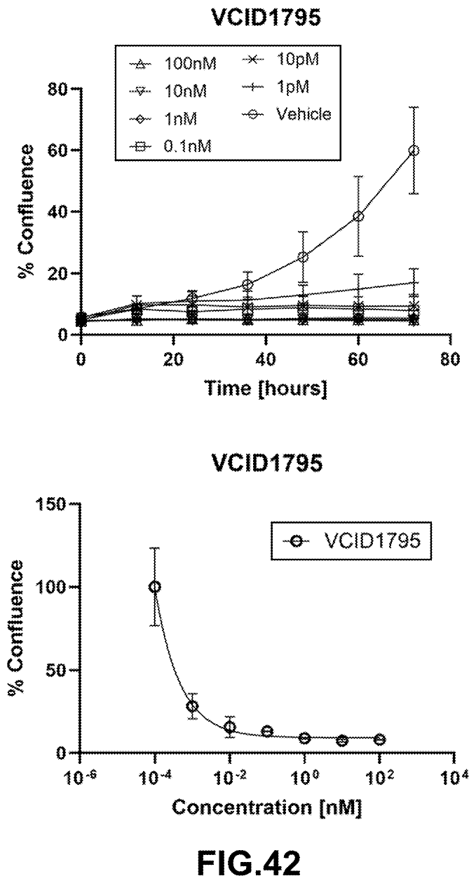

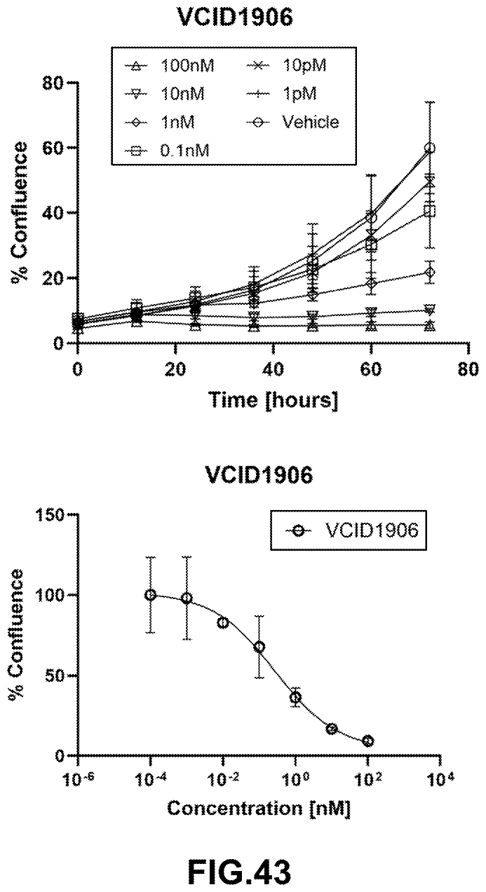

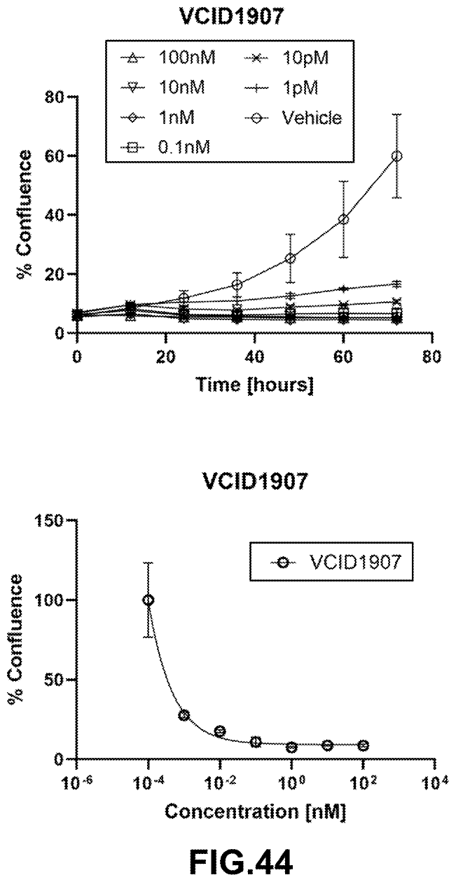

Results for VCID1795 are shown in FIG. 42. Top panel shows % confluence over time. The dose curve at 72 hours was plotted, and is shown in the bottom panel. The IC₅₀ for VCID1795 was <1 pM. Results for VCID1906 are shown in FIG. 43. Top panel shows % confluence over time. The dose curve at 72 hours was plotted, and is shown in the bottom panel. The IC₅₀ for VCID1906 was 0.27 nM. Results for VCID1907 are shown in FIG. 44. Top panel shows % confluence over time. The dose curve at 72 hours was plotted, and is shown in the bottom panel. The IC₅₀ for VCID1907 was <1 pM. All three molecules tested show strong cytotoxic activity.

### Example 23

In vitro cytotoxicity assays were carried out using a molecule with a targeting moiety directed to TROP2. Molecule VCID1880 (SEQ ID NO:115) comprises, from N-terminus to C-terminus, an HpCDT-derived domain, a furin-cleavable linker, a DTB-derived translocation domain, and an anti-TROP2-derived targeting moiety. Different concentrations of drug were tested using cells from cancer cell line HEK293 engineered to express TROP2 (HEK293-TROP2 cells).

HEK293-TROP2 cells were plated in flat bottom 96-well plates, 10,000 cells per well in 100 μl RPMI-1640 complete media. VCID1880 was serially diluted in RPMI-1640 complete media then added 1:1 by volume into wells containing cells. Drug dilutions were added to the wells containing cells slowly and carefully to avoid cells detaching from the plate. Plates were immediately placed inside a Revvity, Inc. Incucyte® SX5 cell culture incubator at 37° C. and 5% CO₂ for live cell imaging. Images were taken at 10× with the phase channel every 12 hours for 3-5 days. Images were analyzed for % confluence in the well based on phase imaging. Average+/−SD was plotted.

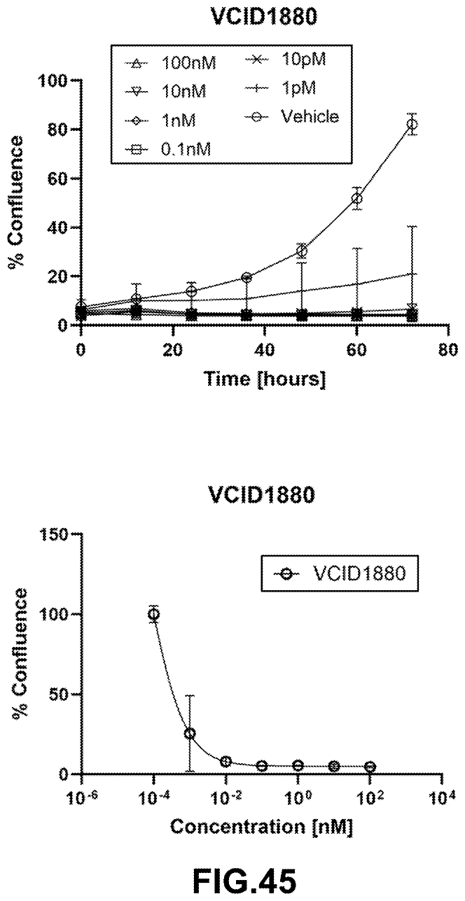

Results for VCID1880 are shown in FIG. 45. Top panel shows % confluence over time. The dose curve at 72 hours was plotted, and is shown in the bottom panel. The IC₅₀ for VCID1861 was <1 pM.

### Example 24

Molecule VCID1793 was tested in an in vivo study using a human tumor xenograft model in mice. Molecule VCID1793 (SEQ ID NO:96) comprises, from N-terminus to C-terminus, an HpCDT-derived domain, a furin-cleavable linker, a DTB-derived translocation domain, and an anti-CLDN18.2-derived targeting moiety.

Cells were passaged 2 days prior to cell transplantation to ensure cells were in growth phase. For cell preparation, cells were counted four times to obtain an average cell count, washed twice in sterile PBS, and resuspended in 1:1 growth factor reduced (GFR) phenol red-free Matrigel: sterile PBS and kept on ice. 750,000 HEK293-CLND18.2 cells were transplanted by subcutaneous injection (100 μl volume per injection) into female NSG mice 8-12 weeks old. These mice are immunodeficient, lacking mature T, B, and NK cells, which allow for development of cancers from human tumor cells. Mice were treated starting 3 days after cell infusion to allow for engraftment of cells, when estimated average tumor size was 85 mm³. VCID1793 or vehicle control (PBS) was dosed intraperitoneally (IP) once daily for 2 weeks with a 5 days on, 2 days off cycle. N=8 mice per group. Tumor size was monitored at least twice weekly by caliper measurements. Estimated tumor volume was calculated by V=0.5×L×W², where V is estimated tumor volume, L is length of longest tumor dimension, and W is length of the dimension perpendicular to L. Average+/−SEM was plotted over time.

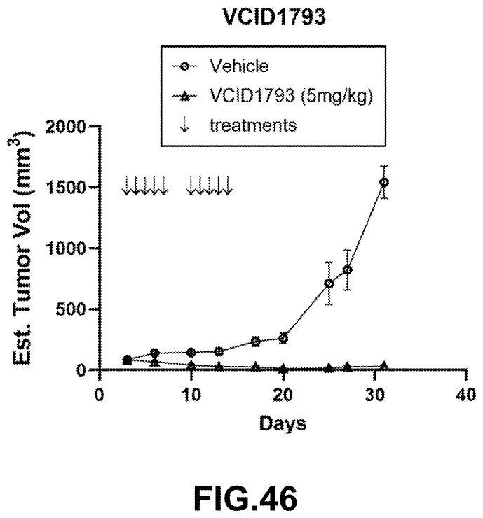

Results for VCID1793 are shown in FIG. 46. The tumor burden was well controlled in the VCID1793-treated mice as compared to vehicle-treated mice.

### Example 25

Molecules VCID1797, VCID1896, and VCID1898 were tested in an in vivo study using a human tumor xenograft model in mice. Molecule VCID1797 (SEQ ID NO: 119) comprises, from N-terminus to C-terminus, an HpCDT-derived domain, a furin-cleavable linker, a DTB-derived translocation domain, and an anti-HER2-derived targeting moiety. Molecule VCID1896 (SEQ ID NO:99) comprises, from N-terminus to C-terminus, an HpCDT-derived domain, a furin-cleavable linker, a DTB-derived translocation domain, and an anti-HER2-derived targeting moiety. Molecule VCID1898 (SEQ ID NO:101) comprises, from N-terminus to C-terminus, an HpCDT-derived domain, a furin-cleavable linker, a DTB-derived translocation domain, and an anti-HER2-derived targeting moiety. Each of these molecules comprises a targeting moiety derived from a different anti-HER2 antibody.

Cells were passaged 2 days prior to cell transplantation to ensure cells were in growth phase. For cell preparation, cells were counted four times to obtain an average cell count, washed twice in sterile PBS, and resuspended in 1:1 growth factor reduced (GFR) phenol red-free Matrigel: sterile PBS and kept on ice. 750,000 HEK293-HER2 cells were transplanted by subcutaneous injection (100 μl volume per injection) into female NSG mice 8-12 weeks old. These mice are immunodeficient, lacking mature T, B, and NK cells, which allow for development of cancers from human tumor cells. Mice were treated starting 3 days after cell infusion to allow for engraftment of cells, when estimated average tumor size was 85 mm³. VCID1797, VCID1896, VCID1898, or vehicle control (PBS) was dosed intraperitoneally (IP) once daily for 2 weeks with a 5 days on, 2 days off cycle. N=7-8 mice per group. Tumor size was monitored at least twice weekly by caliper measurements. Estimated tumor volume was calculated by V=0.5×L×W², where V is estimated tumor volume, L is length of longest tumor dimension, and W is length of the dimension perpendicular to L. Average+/−SEM was plotted over time.

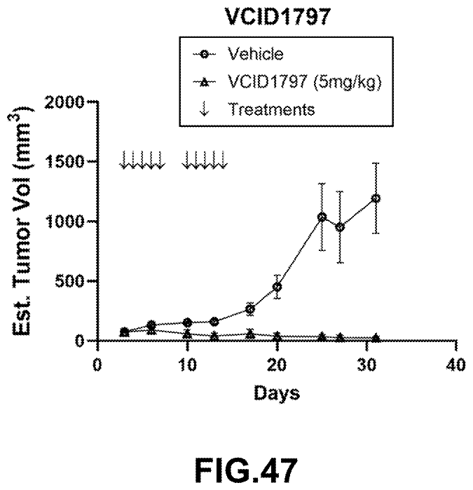

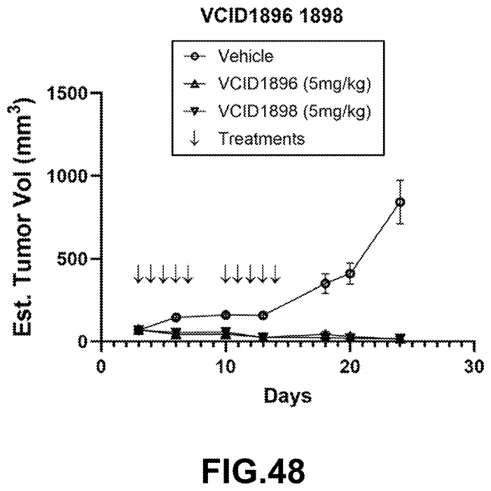

Results for VCID1797 are shown in FIG. 47. Results for VCID1896 and VCID1898 are shown in FIG. 48. For all three drugs tested, the tumor burden was well controlled in the drug-treated mice as compared to vehicle-treated mice.

### Example 26

Molecule VCID1862 was tested in an in vivo study using a human tumor xenograft model in mice. Molecule VCID1862 (SEQ ID NO: 105) comprises, from N-terminus to C-terminus, an HpCDT-derived domain, a furin-cleavable linker, a DTB-derived translocation domain, and an anti-MSLN-derived targeting moiety.

Cells were passaged 2 days prior to cell transplantation to ensure cells were in growth phase. For cell preparation, cells were counted four times to obtain an average cell count, washed twice in sterile PBS, and resuspended in 1:1 growth factor reduced (GFR) phenol red-free Matrigel: sterile PBS and kept on ice. 750,000 HEK293-MSLN cells were transplanted by subcutaneous injection (100 μl volume per injection) into female NSG mice 8-12 weeks old. These mice are immunodeficient, lacking mature T, B, and NK cells, which allow for development of cancers from human tumor cells. Mice were treated starting 3 days after cell infusion to allow for engraftment of cells, when estimated average tumor size was 85 mm³. VCID1862 or vehicle control (PBS) was dosed intraperitoneally (IP) once daily for 2 weeks with a 5 days on, 2 days off cycle. N=8 mice per group. Tumor size was monitored at least twice weekly by caliper measurements. Estimated tumor volume was calculated by V=0.5×L×W², where V is estimated tumor volume, L is length of longest tumor dimension, and W is length of the dimension perpendicular to L. Average+/−SEM was plotted over time.

Results for VCID1862 are shown in FIG. 48. For all three drugs tested, the tumor burden was greatly reduced in the drug-treated mice as compared to vehicle-treated mice.

### Example 27

In vitro cytotoxicity assays were carried out to test certain molecules for off-target cytotoxicity. Molecule VCID1793 (SEQ ID NO:96) comprises, from N-terminus to C-terminus, an HpCDT-derived domain, a furin-cleavable linker, a DTB-derived translocation domain, and an anti-CLDN18.2-derived targeting moiety. Molecule VCID1797 (SEQ ID NO: 119) comprises, from N-terminus to C-terminus, an HpCDT-derived domain, a furin-cleavable linker, a DTB-derived translocation domain, and an anti-HER2-derived targeting moiety. Molecule VCID1896 (SEQ ID NO:99) comprises, from N-terminus to C-terminus, an HpCDT-derived domain, a furin-cleavable linker, a DTB-derived translocation domain, and an anti-HER2-derived targeting moiety. Molecule VCID1862 (SEQ ID NO:105) comprises, from N-terminus to C-terminus, an HpCDT-derived domain, a furin-cleavable linker, a DTB-derived translocation domain, and an anti-MSLN-derived targeting moiety. Molecule VCID1861 (SEQ ID NO:107) comprises, from N-terminus to C-terminus, an HpCDT-derived domain, a furin-cleavable linker, a DTB-derived translocation domain, and an anti-GPC3-derived targeting moiety. Different concentrations of drug were tested using cells from cancer cell line HEK293, which does not express high levels of the antigens targeted by these molecules (i.e., CLND18.2, HER2, MSLN, or GPC3).

HEK293 cells were plated in flat bottom 96-well plates, 10,000 cells per well in 100 μl RPMI-1640 complete media. VCID1880 was serially diluted in RPMI-1640 complete media then added 1:1 by volume into wells containing cells. Drug dilutions were added to the wells containing cells slowly and carefully to avoid cells detaching from the plate. Plates were immediately placed inside a Revvity, Inc. Incucyte® SX5 cell culture incubator at 37° C. and 5% CO₂ for live cell imaging. Images were taken at 10× with the phase channel every 12 hours for 3-5 days. Images were analyzed for % confluence in the well based on phase imaging. Average+/−SD was plotted.

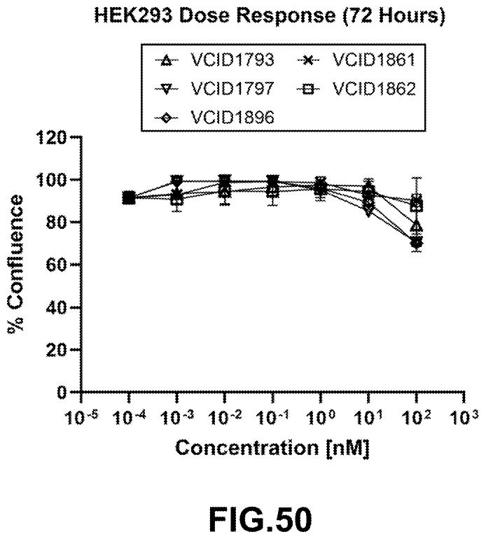

Results are shown in FIG. 50. The dose curve at 72 hours is shown. None of the tested drugs showed significant cytotoxicity for the tested HEK293 cells. In each case, the IC₅₀ was calculated to be >100 nM.

### Example 28

In vitro cytotoxicity assays were carried out to compare different variants of a given targeting moiety in a molecule of the present disclosure. Molecule VCID1950 (SEQ ID NO:120) comprises, from N-terminus to C-terminus, an HpCDT-derived domain, a furin-cleavable linker, a DTB-derived translocation domain, and an IL3-derived targeting moiety. Molecule VCID1951 (SEQ ID NO:121) comprises, from N-terminus to C-terminus, an HpCDT-derived domain, a furin-cleavable linker, a DTB-derived translocation domain, and an IL3-derived targeting moiety. Molecule VCID1952 (SEQ ID NO:122) comprises, from N-terminus to C-terminus, an HpCDT-derived domain, a furin-cleavable linker, a DTB-derived translocation domain, and an IL3-derived targeting moiety. Molecule VCID1953 (SEQ ID NO:123) comprises, from N-terminus to C-terminus, an HpCDT-derived domain, a furin-cleavable linker, a DTB-derived translocation domain, and an IL3-derived targeting moiety. Different concentrations of drug were tested using cells from AML cancer cell lines MOLM-13 or MV4-11.

MOLM-13 or MV4-11 cells were plated in flat bottom 96-well plates, 5,000 cells per well in 100 μl RPMI-1640 complete media. VCID1950, VCID1951, VCID1952, or VCID1953 was serially diluted in RPMI-1640 complete media then added 1:1 by volume into wells containing cells. After 5 days, cells were pipetted to mix, then 50 μl of cell suspension was transferred to white-sided 96 well flat bottom plates. Promega CellTiter-Glo Luminescent Cell Viability Assay® was added to cell suspension and mixed. After ten minutes' incubation in the dark, luciferase signal was read out with a luminometer. Luciferase signal was normalized to vehicle control wells. Cell viability was plotted as percent live cells, normalized to vehicle control set at 100%. Average+/−SD was plotted.

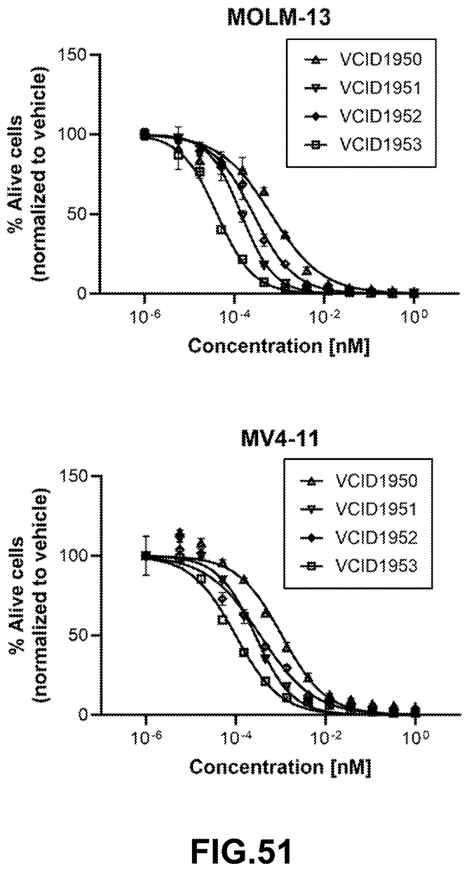

Results are shown in FIG. 51. Top panel shows results using MOLM-13 cells. Bottom panel shows results using MV4-11 cells. For both cell lines and all drugs tested, the drug showed greater than 50% killing at one or more of the concentrations tested. IC₅₀ values were calculated from non-linear fitted data. The IC₅₀ values for MOLM-13 cells were calculated to be: 0.66 pM for VCID1950, 0.14 pM for VCID1951, 0.27 pM for VCID1952, 0.04 pM for VCID1953, and 0.10 pM for VCID1728. The IC₅₀ values for MV4-11 cells were calculated to be: 1.06 pM for VCID1950, 0.27 pM for VCID1951, 0.35 pM for VCID1952, 0.10 pM for VCID1953, and 0.27 pM for VCID1728.

### Example 29

In vitro cytotoxicity assays were carried out using a molecule of the present disclosure with an additional cell line. Molecule VCID1728 (SEQ ID NO:62) comprises, from N-terminus to C-terminus, an HpCDT-derived domain, a furin-cleavable linker, a DTB-derived translocation domain which is a truncated version of DTB that retains translocation function (DTB403), a GM-CSF-derived targeting moiety, and an IL3-derived targeting moiety. VCID1728 was tested at different concentrations using cancer cell line SKM-1, which is derived from a patient suffering from myelodysplastic syndrome (MDS).

Cells from MDS cancer cell line SKM-1 were plated in flat bottom 96-well plates, 5000 cells per well in 100 μl RPMI-1640 complete media. VCID1728 was serially diluted in RPMI-1640 complete media then added 1:1 by volume into wells containing cells. After 5 days, cells were pipetted to mix, then 50 μl of cell suspension was transferred to white-sided 96 well flat bottom plates. Promega CellTiter-Glo Luminescent Cell Viability Assay® was added to cell suspension and mixed. After ten minutes' incubation in the dark, luciferase signal was read out with a luminometer. Luciferase signal was normalized to vehicle control wells. Cell viability was plotted as percent live cells, normalized to vehicle control set at 100%. Average+/−SD was plotted.

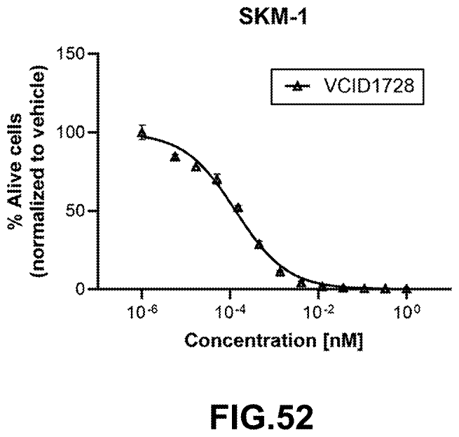

Results are shown in FIG. 52. VCID1728 showed greater than 50% killing at multiple concentrations tested. IC₅₀ values were calculated from non-linear fitted data. The IC50 for VCID1728 was 0.13 pM for SKM-1 cells.

### VIII. Other Embodiments

All publications, patent applications, and patents mentioned in this specification are herein incorporated by reference.

While certain embodiments of the invention have been illustrated and described, it will be appreciated that various changes can be made therein without departing from the spirit and scope of the invention. Although the invention has been described in connection with specific embodiments, it should be understood that the invention as claimed should not be unduly limited to such specific embodiments. Indeed, various modifications of the specific embodiments described that are obvious to those skilled in the fields of medicine, immunology, pharmacology, or related fields are intended to be within the scope of the invention.

Accordingly, the following numbered paragraphs describing specific embodiments are provided for clarity, but should not be construed to limit the claims.
1. A molecule comprising a toxin domain, a translocation domain, and one or more targeting moieties that bind an antigen or receptor present on the surface of a cancer cell; wherein the toxin domain has DNase activity; the translocation domain is capable of translocating the molecule or a fragment thereof from within the endosome, across the endosomal membrane, and into the cytosol of a cell; and the toxin domain is linked to the translocation domain by a linker containing a protease cleavage site.
2. The molecule of numbered paragraph 1, wherein the toxin domain is capable of translocating across a cell nuclear membrane.
3. The molecule of numbered paragraph 1, wherein the toxin domain induces G2/M cell cycle arrest in mammalian cells.
4. The molecule of numbered paragraph 1, wherein the toxin domain is derived from cytolethal distending toxin (CDT).
5. The molecule of numbered paragraph 4, wherein the toxin domain is derived from CDT domain b (CdtB).
6. The molecule of numbered paragraph 4 or 5, wherein the CDT-derived toxin domain is derived from a Haemophilus species, an Escherichia species, an Aggregatibacter species, or a Salmonella species CDT.
7. The molecule of numbered paragraph 6, wherein the CDT-derived toxin domain is derived from a Haemophilus ducreyi CDT.
8. The molecule of numbered paragraph 6, wherein the CDT-derived toxin domain is derived from a Haemophilus parasuis CDT.
9. The molecule of numbered paragraph 6, wherein the CDT-derived toxin domain is derived from an Escherichia coli CDT.
10. The molecule of numbered paragraph 6, wherein the CDT-derived toxin domain is derived from an Aggregatibacter actinomycetemcomitans CDT.
11. The molecule of numbered paragraph 6, wherein the CDT-derived toxin domain is derived from a Salmonella enterica CDT.
12. The molecule of any one of numbered paragraphs 4-11, wherein the CDT-derived toxin domain is a functional fragment of CDT having DNase activity.
13. The molecule of numbered paragraph 5, wherein the CDT-derived toxin domain comprises the amino acid sequence set forth as any one of SEQ ID NOs:1-5.
14. The molecule of numbered paragraph 5, wherein the CDT-derived toxin domain comprises an amino acid sequence having 75%, 80%, 85%, 90%, 95%, 97%, or 99% identity with any one of SEQ ID NOs:1-5.
15. The molecule of numbered paragraph 5, wherein the CDT-derived toxin domain is a functional fragment or functional variant of the amino acid sequence set forth as any one of SEQ ID NOs:1-5, wherein a functional fragment or functional variant is a fragment or variant having DNase activity.
16. The molecule of any of numbered paragraphs 1-15, wherein the translocation domain is derived from diphtheria toxin domain B (DTB).
17. The molecule of numbered paragraph 16, wherein the DTB-derived translocation domain is a functional fragment of DTB capable of mediating the translocation of the molecule or a fragment thereof from within the endosome, across the endosomal membrane, and into the cell cytosol.
18. The molecule of numbered paragraph 16, wherein the translocation domain comprises the amino acid sequence set forth as SEQ ID NO:10 or SEQ ID NO:63.
19. The molecule of numbered paragraph 16, wherein the translocation domain comprises a functional fragment or functional variant of the amino acid sequence set forth as SEQ ID NO:10 or SEQ ID NO:63, wherein the functional fragment or functional variant is a fragment or variant that is capable of mediating the translocation of the molecule or a fragment thereof from within the endosome, across the endosomal membrane, and into the cell cytosol.
20. The molecule of numbered paragraph 19, wherein the translocation domain comprises an amino acid sequence having 75%, 80%, 85%, 90%, 95%, 97%, or 99% identity with SEQ ID NO:10 or SEQ ID NO:63.
21. The molecule of any of numbered paragraphs 1-20, wherein the linker containing a protease cleavage site comprises an amino acid sequence cleaved by a protease present in endosomes.
22. The molecule of numbered paragraph 21, wherein the linker containing a protease cleavage site comprises a furin cleavage site.
23. The molecule of numbered paragraph 22, wherein the linker containing a protease cleavage site comprises a cysteine-bounded furin cleavage site.
24. The molecule of any of numbered paragraphs 21-23, wherein the linker containing a protease cleavage site comprises an amino acid sequence selected from SEQ ID NOs:6, 7, and 8.
25. The molecule of any of numbered paragraphs 1-24, wherein the one or more targeting moieties bind an antigen or receptor present on the surface of a cancer cell and wherein the antigen or receptor is capable of internalizing the bound molecule or a fragment thereof.
26. The molecule of numbered paragraph 25, wherein the one or more targeting moieties are derived from an antibody, a cytokine, or a receptor binding protein or peptide.
27. The molecule of numbered paragraph 26, wherein at least one targeting moiety is derived from a cytokine.
28. The molecule of numbered paragraph 27, wherein the at least one targeting moiety is derived from GM-CSF.
29. The molecule of numbered paragraph 28, wherein the at least one targeting moiety comprises the amino acid sequence set forth as SEQ ID NO:11.
30. The molecule of numbered paragraph 29, wherein the at least one targeting moiety comprises a functional fragment or functional variant of the amino acid sequence set forth as SEQ ID NO:11, wherein the functional fragment or functional variant is capable of binding an antigen present on the surface of a cancer cell.
31. The molecule of numbered paragraph 30, wherein the at least one targeting moiety comprises an amino acid sequence having 75%, 80%, 85%, 90%, 95%, 97%, or 99% identity with SEQ ID NO:11.
32. The molecule of numbered paragraph 28, wherein at least one targeting moiety is derived from IL3.
33. The molecule of numbered paragraph 32, wherein the at least one targeting moiety comprises the amino acid sequence set forth as any one of SEQ ID NOs:12-15.
34. The molecule of numbered paragraph 32, wherein the at least one targeting moiety comprises a functional fragment or functional variant of the amino acid sequence set forth as any one of SEQ ID NOs:12-15, wherein the functional fragment or functional variant is capable of binding an antigen present on the surface of a cancer cell.
35. The molecule of numbered paragraph 34, wherein the at least one targeting moiety comprises an amino acid sequence having 75%, 80%, 85%, 90%, 95%, 97%, or 99% identity with any one of SEQ ID NOs:12-15.
36. The molecule of numbered paragraph 28, wherein at least one targeting moiety is derived from IL2.
37. The molecule of numbered paragraph 36, wherein the at least one targeting moiety comprises the amino acid sequence set forth as SEQ ID NO:83.
38. The molecule of numbered paragraph 36, wherein the at least one targeting moiety comprises a functional fragment or functional variant of the amino acid sequence set forth as SEQ ID NO:83, wherein the functional fragment or functional variant is capable of binding an antigen present on the surface of a cancer cell.
39. The molecule of numbered paragraph 38, wherein the at least one targeting moiety comprises an amino acid sequence having 75%, 80%, 85%, 90%, 95%, 97%, or 99% identity with SEQ ID NO:83.
40. The molecule of numbered paragraph 28, wherein at least one targeting moiety is derived from IL4.
41. The molecule of numbered paragraph 40, wherein the at least one targeting moiety comprises the amino acid sequence set forth as SEQ ID NO:84.
42. The molecule of numbered paragraph 40, wherein the at least one targeting moiety comprises a functional fragment or functional variant of the amino acid sequence set forth as SEQ ID NO:84, wherein the functional fragment or functional variant is capable of binding an antigen present on the surface of a cancer cell.
43. The molecule of numbered paragraph 40, wherein the at least one targeting moiety comprises an amino acid sequence having 75%, 80%, 85%, 90%, 95%, 97%, or 99% identity with SEQ ID NO:84.
44. The molecule of numbered paragraph 26, wherein at least one targeting moiety is derived from an antibody.
45. The molecule of numbered paragraph 44, wherein at least one targeting moiety is derived from an anti-CD33 antibody.
46. The molecule of numbered paragraph 45, wherein the at least one targeting moiety is derived from gemtuzumab.
47. The molecule of numbered paragraph 45, wherein the at least one targeting moiety comprises the amino acid sequence set forth as SEQ ID NO:16.
48. The molecule of numbered paragraph 45, wherein the at least one targeting moiety comprises a functional fragment or functional variant of the amino acid sequence set forth as SEQ ID NO:16, wherein the functional fragment or functional variant is capable of binding CD33.
49. The molecule of numbered paragraph 48, wherein the at least one targeting moiety comprises an amino acid sequence having 75%, 80%, 85%, 90%, 95%, 97%, or 99% identity with SEQ ID NO:16.
50. The molecule of numbered paragraph 45, wherein the at least one targeting moiety comprises an antigen-binding fragment of an anti-CD33 antibody.
51. The molecule of numbered paragraph 49, wherein the at least one targeting moiety comprises an antigen-binding fragment of gemtuzumab.
52. The molecule of numbered paragraph 45, wherein the at least one targeting moiety is derived from an scFv derived from gemtuzumab.
53. The molecule of numbered paragraph 44, wherein at least one targeting moiety is derived from an anti-B-cell maturation antigen (BCMA) antibody.
54. The molecule of numbered paragraph 53, wherein the at least one targeting moiety comprises the amino acid sequence set forth as SEQ ID NO:79.
55. The molecule of numbered paragraph 53, wherein the at least one targeting moiety comprises a functional fragment or functional variant of the amino acid sequence set forth as SEQ ID NO:79, wherein the functional fragment or functional variant is capable of binding BCMA.
56. The molecule of numbered paragraph 55, wherein the at least one targeting moiety comprises an amino acid sequence having 75%, 80%, 85%, 90%, 95%, 97%, or 99% identity with SEQ ID NO:79.
57. The molecule of numbered paragraph 53, wherein the at least one targeting moiety comprises an antigen-binding fragment of an anti-BCMA antibody.
58. The molecule of numbered paragraph 53, wherein the at least one targeting moiety is derived from an scFv.
59. The molecule of numbered paragraph 44, wherein at least one targeting moiety is derived from an anti-CD19 antibody.
60. The molecule of numbered paragraph 59, wherein the at least one targeting moiety comprises the amino acid sequence set forth as SEQ ID NO:81.
61. The molecule of numbered paragraph 59, wherein the at least one targeting moiety comprises a functional fragment or functional variant of the amino acid sequence set forth as SEQ ID NO:81, wherein the functional fragment or functional variant is capable of binding CD19.
62. The molecule of numbered paragraph 61, wherein the at least one targeting moiety comprises an amino acid sequence having 75%, 80%, 85%, 90%, 95%, 97%, or 99% identity with SEQ ID NO:81.
63. The molecule of numbered paragraph 59, wherein the at least one targeting moiety comprises an antigen-binding fragment of an anti-CD19 antibody.
64. The molecule of numbered paragraph 59, wherein the at least one targeting moiety is derived from an scFv.
65. The molecule of numbered paragraph 44, wherein at least one targeting moiety is derived from an anti-claudin18.2 (CLDN18.2) antibody.
66. The molecule of numbered paragraph 65, wherein the at least one targeting moiety is derived from osemitamab.
67. The molecule of numbered paragraph 65, wherein the at least one targeting moiety is derived from zolbetuximab.
68. The molecule of numbered paragraph 65, wherein the at least one targeting moiety comprises the amino acid sequence set forth as SEQ ID NO:92 or 94.
69. The molecule of numbered paragraph 65, wherein the at least one targeting moiety comprises a functional fragment or functional variant of the amino acid sequence set forth as SEQ ID NO:92 or 94, wherein the functional fragment or functional variant is capable of binding CLDN18.2.
70. The molecule of numbered paragraph 69, wherein the at least one targeting moiety comprises an amino acid sequence having 75%, 80%, 85%, 90%, 95%, 97%, or 99% identity with SEQ ID NO:92 or 94.
71. The molecule of numbered paragraph 65, wherein the at least one targeting moiety comprises an antigen-binding fragment of an anti-CLDN18.2 antibody.
72. The molecule of numbered paragraph 65, wherein the at least one targeting moiety comprises an antigen-binding fragment of osemitamab or zolbetuximab.
73. The molecule of numbered paragraph 65, wherein the at least one targeting moiety is derived from an scFv derived from osemitamab or zolbetuximab.
74. The molecule of numbered paragraph 44, wherein at least one targeting moiety is derived from an anti-human epidermal growth factor receptor 2 (HER2) antibody.
75. The molecule of numbered paragraph 74, wherein the at least one targeting moiety is derived from trastuzumab.
76. The molecule of numbered paragraph 74, wherein the at least one targeting moiety comprises the amino acid sequence set forth as any one of SEQ ID NOs:98, 100, 102, and 118.
77. The molecule of numbered paragraph 74, wherein the at least one targeting moiety comprises a functional fragment or functional variant of the amino acid sequence set forth as any one of SEQ ID NOs:98, 100, 102, and 118, wherein the functional fragment or functional variant is capable of binding HER2.
78. The molecule of numbered paragraph 77, wherein the at least one targeting moiety comprises an amino acid sequence having 75%, 80%, 85%, 90%, 95%, 97%, or 99% identity with any one of SEQ ID NOs:98, 100, 102, and 118.
79. The molecule of numbered paragraph 74, wherein the at least one targeting moiety comprises an antigen-binding fragment of an anti-HER2 antibody.
80. The molecule of numbered paragraph 74, wherein the at least one targeting moiety comprises an antigen-binding fragment of trastuzumab.
81. The molecule of numbered paragraph 74, wherein the at least one targeting moiety is derived from an scFv, an affibody, or a DARPin.
82. The molecule of numbered paragraph 44, wherein at least one targeting moiety is derived from an anti-mesothelin (MSLN) antibody.
83. The molecule of numbered paragraph 82, wherein the at least one targeting moiety is derived from anetumab.
84. The molecule of numbered paragraph 82, wherein the at least one targeting moiety comprises the amino acid sequence set forth as SEQ ID NO: 104.
85. The molecule of numbered paragraph 82, wherein the at least one targeting moiety comprises a functional fragment or functional variant of the amino acid sequence set forth as SEQ ID NO:104, wherein the functional fragment or functional variant is capable of binding MSLN.
86. The molecule of numbered paragraph 85, wherein the at least one targeting moiety comprises an amino acid sequence having 75%, 80%, 85%, 90%, 95%, 97%, or 99% identity with SEQ ID NO:104.
87. The molecule of numbered paragraph 82, wherein the at least one targeting moiety comprises an antigen-binding fragment of an anti-MSLN antibody.
88. The molecule of numbered paragraph 82, wherein the at least one targeting moiety comprises an antigen-binding fragment of anetumab.
89. The molecule of numbered paragraph 82, wherein the at least one targeting moiety is derived from an scFv derived from anetumab.
90. The molecule of numbered paragraph 44, wherein at least one targeting moiety is derived from an anti-glypican-3 (GPC3) antibody.
91. The molecule of numbered paragraph 90, wherein the at least one targeting moiety is derived from codrituzumab.
92. The molecule of numbered paragraph 90, wherein the at least one targeting moiety comprises the amino acid sequence set forth as SEQ ID NO: 106.
93. The molecule of numbered paragraph 90, wherein the at least one targeting moiety comprises a functional fragment or functional variant of the amino acid sequence set forth as SEQ ID NO:106, wherein the functional fragment or functional variant is capable of binding GPC3.
94. The molecule of numbered paragraph 93, wherein the at least one targeting moiety comprises an amino acid sequence having 75%, 80%, 85%, 90%, 95%, 97%, or 99% identity with SEQ ID NO:106.
95. The molecule of numbered paragraph 90, wherein the at least one targeting moiety comprises an antigen-binding fragment of an anti-GPC3 antibody.
96. The molecule of numbered paragraph 90, wherein the at least one targeting moiety comprises an antigen-binding fragment of codrituzumab.
97. The molecule of numbered paragraph 90, wherein the at least one targeting moiety is derived from an scFv derived from codrituzumab.
98. The molecule of numbered paragraph 44, wherein at least one targeting moiety is derived from an anti-epidermal growth factor receptor (EGFR) antibody.
99. The molecule of numbered paragraph 98, wherein the at least one targeting moiety is derived from cetuximab.
100. The molecule of numbered paragraph 98, wherein the at least one targeting moiety comprises the amino acid sequence set forth as any one of SEQ ID NOs:108, 110, or 112.
101. The molecule of numbered paragraph 98, wherein the at least one targeting moiety comprises a functional fragment or functional variant of the amino acid sequence set forth as any one of SEQ ID NOs:108, 110, or 112, wherein the functional fragment or functional variant is capable of binding EGFR.
102. The molecule of numbered paragraph 101, wherein the at least one targeting moiety comprises an amino acid sequence having 75%, 80%, 85%, 90%, 95%, 97%, or 99% identity with any one of SEQ ID NOs:108, 110, or 112.
103. The molecule of numbered paragraph 98, wherein the at least one targeting moiety comprises an antigen-binding fragment of an anti-EGFR antibody.
104. The molecule of numbered paragraph 98, wherein the at least one targeting moiety comprises an antigen-binding fragment of cetuximab.
105. The molecule of numbered paragraph 98, wherein the at least one targeting moiety is derived from an scFv or a nanobody.
106. The molecule of numbered paragraph 44, wherein at least one targeting moiety is derived from an anti-trophoblast cell surface antigen 2 (TROP2) antibody.
107. The molecule of numbered paragraph 106, wherein the at least one targeting moiety is derived from datopotamab.
108. The molecule of numbered paragraph 106, wherein the at least one targeting moiety comprises the amino acid sequence set forth as SEQ ID NO: 114.
109. The molecule of numbered paragraph 106, wherein the at least one targeting moiety comprises a functional fragment or functional variant of the amino acid sequence set forth as SEQ ID NO:114, wherein the functional fragment or functional variant is capable of binding TROP2.
110. The molecule of numbered paragraph 109, wherein the at least one targeting moiety comprises an amino acid sequence having 75%, 80%, 85%, 90%, 95%, 97%, or 99% identity with SEQ ID NOs:101.
111. The molecule of numbered paragraph 106, wherein the at least one targeting moiety comprises an antigen-binding fragment of an anti-TROP2 antibody.
112. The molecule of numbered paragraph 106, wherein the at least one targeting moiety comprises an antigen-binding fragment of datopotamab.
113. The molecule of numbered paragraph 106, wherein the at least one targeting moiety is derived from an scFv derived from datopotamab.
114. The molecule of numbered paragraph 1, wherein the molecule comprises two or more targeting moieties.
115. The molecule of numbered paragraph 114, wherein the molecule comprises two targeting moieties.
116. The molecule of numbered paragraph 115, wherein the two targeting moieties bind the same cancer-related surface antigen or receptor.
117. The molecule of numbered paragraph 115, wherein the two targeting moieties bind different cancer-related surface antigens or receptors.
118. The molecule of numbered paragraph 115, wherein one of the two targeting moieties is derived from GM-CSF and one of the two targeting moieties is derived from IL3.
119. The molecule of numbered paragraph 118, wherein the GM-CSF-derived targeting moiety comprises the sequence set forth as SEQ ID NO:11 or a functional fragment or functional variant thereof and the IL3-derived targeting moiety comprises the sequence set forth as any one of SEQ ID NO:12-15 or a functional fragment or functional variant thereof.
120. The molecule of numbered paragraph 115, wherein one of the two targeting moieties is derived from IL2 and one of the two targeting moieties is derived from IL4.
121. The molecule of numbered paragraph 120, wherein the IL2-derived targeting moiety comprises the sequence set forth as SEQ ID NO:83 or a functional fragment or functional variant thereof and the IL4-derived targeting moiety comprises the sequence set forth as SEQ ID NO:85 or a functional fragment or functional variant thereof.
122. The molecule of numbered paragraph 1, wherein the translocation domain is linked to the one or more targeting moieties by a linker.
123. The molecule of numbered paragraph 122, wherein the linker is a non-cleavable linker.
124. The molecule of numbered paragraph 123, wherein the linker comprises the sequence set forth as any one of SEQ ID NOs:9, 17-30, 58-60, 65-78, and 124.
125. The molecule of numbered paragraph 114, wherein the two or more targeting moieties are linked by a linker.
126. The molecule of numbered paragraph 125, wherein the linker is a non-cleavable linker.
127. The molecule of numbered paragraph 125, wherein the two or more targeting moieties are linked by linkers which are independently selected from any one of SEQ ID NOs: 9, 17-30, 58-60, 65-78, and 124.
128. The molecule of numbered paragraph 1, wherein the molecule comprises a CDT-derived domain linked to a furin-cleavable linker, linked to a DTB-derived domain, linked to a GM-CSF-derived targeting moiety.
129. The molecule of numbered paragraph 128, comprising the sequence set forth as any one of SEQ ID NOs:31-37, and 53-57.
130. The molecule of numbered paragraph 1, wherein the molecule comprises a CDT-derived domain linked to a furin-cleavable linker, linked to a DTB-derived domain, linked to an anti-CD33-derived targeting domain.
131. The molecule of numbered paragraph 130, comprising the sequence set forth as SEQ ID NO:38.
132. The molecule of numbered paragraph 1, wherein the molecule comprises a CDT-derived domain linked to a furin-cleavable linker, linked to a DTB-derived domain, linked to an IL3-derived targeting domain.
133. The molecule of numbered paragraph 132, comprising the sequence set forth as any one of SEQ ID NOs:39-41.
134. The molecule of numbered paragraph 1, wherein the molecule comprises a CDT-derived domain linked to a furin-cleavable linker, linked to a DTB-derived domain, linked to a GM-CSF-derived targeting domain, linked to an IL3-derived targeting domain.
135. The molecule of numbered paragraph 134, comprising the sequence set forth as any one of SEQ ID NOs:42-51, 61, 62, 116-117, and 120-123.
136. The molecule of numbered paragraph 1, wherein the molecule comprises a CDT-derived domain linked to a furin-cleavable linker, linked to a DTB-derived domain, linked to an anti-BCMA-derived targeting domain.
137. The molecule of numbered paragraph 136, comprising the sequence set forth as SEQ ID NO:80.
138. The molecule of numbered paragraph 1, wherein the molecule comprises a CDT-derived domain linked to a furin-cleavable linker, linked to a DTB-derived domain, linked to an anti-CD19-derived targeting domain.
139. The molecule of numbered paragraph 138, comprising the sequence set forth as SEQ ID NO:82.
140. The molecule of numbered paragraph 1, wherein the molecule comprises a CDT-derived domain linked to a furin-cleavable linker, linked to a DTB-derived domain, linked to an IL2-derived targeting domain.
141. The molecule of numbered paragraph 140, comprising the sequence set forth as SEQ ID NO:84 or 91.
142. The molecule of numbered paragraph 1, wherein the molecule comprises a CDT-derived domain linked to a furin-cleavable linker, linked to a DTB-derived domain, linked to an IL4-derived targeting domain.
143. The molecule of numbered paragraph 142, comprising the sequence set forth as SEQ ID NO:86 or 89.
144. The molecule of numbered paragraph 1, wherein the molecule comprises a CDT-derived domain linked to a furin-cleavable linker, linked to a DTB-derived domain, linked to an IL4-derived targeting domain, linked to an IL2-derived targeting domain.
145. The molecule of numbered paragraph 144, comprising the sequence set forth as any one of SEQ ID NOs:87-88, 90.
146. The molecule of numbered paragraph 1, wherein the molecule comprises a CDT-derived domain linked to a furin-cleavable linker, linked to a DTB-derived domain, linked to an anti-CLDN18.2-derived targeting domain.
147. The molecule of numbered paragraph 146, comprising the sequence set forth as any one of SEQ ID NOs:93 and 95-97.
148. The molecule of numbered paragraph 1, wherein the molecule comprises a CDT-derived domain linked to a furin-cleavable linker, linked to a DTB-derived domain, linked to an anti-HER2-derived targeting domain.
149. The molecule of numbered paragraph 148, comprising the sequence set forth as any one of SEQ ID NOs:99, 101, 103 and 119.
150. The molecule of numbered paragraph 1, wherein the molecule comprises a CDT-derived domain linked to a furin-cleavable linker, linked to a DTB-derived domain, linked to an anti-MSLN-derived targeting domain.
151. The molecule of numbered paragraph 150, comprising the sequence set forth as SEQ ID NO:105.
152. The molecule of numbered paragraph 1, wherein the molecule comprises a CDT-derived domain linked to a furin-cleavable linker, linked to a DTB-derived domain, linked to an anti-GPC3-derived targeting domain.
153. The molecule of numbered paragraph 152, comprising the sequence set forth as SEQ ID NO:107.
154. The molecule of numbered paragraph 1, wherein the molecule comprises a CDT-derived domain linked to a furin-cleavable linker, linked to a DTB-derived domain, linked to an anti-EGFR-derived targeting domain.
155. The molecule of cl numbered paragraph aim 154, comprising the sequence set forth as any one of SEQ ID NOs:109, 111, and 113.
156. The molecule of numbered paragraph 1, wherein the molecule comprises a CDT-derived domain linked to a furin-cleavable linker, linked to a DTB-derived domain, linked to an anti-TROP2-derived targeting domain.
157. The molecule of numbered paragraph 156, comprising the sequence set forth as SEQ ID NO:115.
158. An isolated DNA sequence encoding the molecule of any one of numbered paragraphs 1-157.
159. A vector comprising the DNA sequence of numbered paragraph 158.
160. A host cell comprising the vector of numbered paragraph 159.
161. A method of producing the molecule of any one of numbered paragraphs 1-157, comprising culturing the host cell of numbered paragraph 160 and isolating the molecule.
162. A composition comprising the molecule of any one of numbered paragraphs 1-157 and a pharmaceutically acceptable excipient.
163. A method of treating cancer in a subject comprising administering an effective amount of the molecule of any one of numbered paragraphs 1-157 or the composition of numbered paragraph 162 to the subject.
164. The method of numbered paragraph 163, wherein the cancer is a hematological cancer.
165. The method of numbered paragraph 164, wherein the cancer is a leukemia, lymphoma, plasma cell neoplasm, myelodysplastic syndrome (MDS), myeloproliferative neoplasm (MPN), histiocytic or dendritic cell neoplasm, or a mast cell neoplasm.
166. The method of numbered paragraph 164, wherein the cancer is an acute leukemia, a chronic leukemia, or another leukemia.
167. The method of numbered paragraph 164, wherein the cancer is acute myeloid leukemia (AML), acute promyelocytic leukemia, acute lymphoblastic leukemia (ALL), chronic myeloid leukemia (CMIL), chronic lymphocytic leukemia (CLL), chronic myelomonocytic leukemia (CMML), juvenile myelomonocytic leukemia (JMML), hairy cell leukemia, prolymphocytic leukemia, large granular lymphocytic leukemia (LGL) including T-cell LGL and NK-cell LGL, Hodgkin lymphoma, non-Hodgkin lymphoma including B-cell lymphoma and T-cell/NK-cell lymphoma, cutaneous T cell lymphoma (CTCL), adult T cell leukemia/lymphoma (ATLL), multiple myeloma (MM), monoclonal gammopathy of undetermined significance (MGUS), plasmacytoma, Waldenstrom macroglobulinemia, light chain (AL) amyloidosis, polycythemia vera (PV), essential thrombocythemia (ET), primary myelofibrosis (PMF), Langerhans cell histiocytosis (LCH), histiocytic sarcoma, follicular dendritic cell sarcoma, interdigitating dendritic cell sarcoma, systemic mastocytosis, mast cell leukemia, or blastic plasmacytoid dendritic cell neoplasm (BPDCN).
168. The method of numbered paragraph 163 wherein the cancer is a non-hematological cancer.
169. The method of numbered paragraph 168, wherein the cancer is a carcinoma, a sarcoma, a melanoma, a neuroendocrine tumor, a central nervous system tumor, a germ cell tumor, or an embryonal tumor.
170. The method of numbered paragraph 168, wherein the cancer is a thoracic cancer, such as a lung cancer (including small cell lung cancer and non-small cell lung cancer) or a mesothelioma; a gastrointestinal cancer, such as a pancreatic, colorectal, gastric, gastroesophageal, hepatic, or biliary tract cancer; a genitourinary cancer, such as a prostate, renal, bladder/urothelial, or testicular cancer; a gynecologic cancer, such as an ovarian, cervical, endometrial, or uterine cancer; a breast cancer; a head and neck cancer; an esophageal cancer; a thyroid cancer; a skin cancer; or another epithelial cancer.
171. The method of numbered paragraph 168, wherein the cancer is a sarcoma, such as a soft tissue sarcoma, osteosarcoma, or chondrosarcoma; a melanoma, such as a cutaneous melanoma or an uveal melanoma; a central nervous system tumor, such as a glioblastoma, astrocytoma, or medulloblastoma; a neuroendocrine tumor, such as a carcinoid tumor or a pancreatic neuroendocrine tumor; or a germ cell tumor such as a testicular germ cell tumor or an ovarian germ cell tumor.
172. The method of numbered paragraph 168, wherein the cancer is an adenocarcinoma, squamous cell carcinoma, small cell carcinoma, or large cell carcinoma.
173. The use of the molecule of any one of numbered paragraphs 1-157 or the composition of numbered paragraph 162 for the treatment of cancer in a subject.
174. The molecule of any one of numbered paragraphs 1-157 or the composition of numbered paragraph 162 for use in the manufacture of a medicament for treating cancer.

What is claimed is:

1. A molecule comprising a toxin domain, a translocation domain, and one or more targeting moieties that bind an antigen or receptor present on the surface of a cancer cell; wherein the toxin domain has DNase activity; the translocation domain is capable of translocating the molecule or a fragment thereof from within the endosome, across the endosomal membrane, and into the cytosol of a cell; and the toxin domain is linked to the translocation domain by a linker containing a protease cleavage site.

2-3. (canceled)

4. The molecule of claim 1, wherein the toxin domain is derived from cytolethal distending toxin (CDT).

5. The molecule of claim 4, wherein the toxin domain is derived from CDT domain b (CdtB).

6. The molecule of claim 4, wherein the CDT-derived toxin domain is derived from a Haemophilus species, an Escherichia species, an Aggregatibacter species, or a Salmonella species CDT.

7-12. (canceled)

13. The molecule of claim 5, wherein the CDT-derived toxin domain comprises the amino acid sequence set forth as any one of SEQ ID NOs:1-5.

14. (canceled)

15. The molecule of claim 5, wherein the CDT-derived toxin domain is a functional fragment or functional variant of the amino acid sequence set forth as any one of SEQ ID NOs:1-5, wherein a functional fragment or functional variant is a fragment or variant having DNase activity.

16. The molecule of claim 1, wherein the translocation domain is derived from diphtheria toxin domain B (DTB).

17. (canceled)

18. The molecule of claim 16, wherein the translocation domain comprises the amino acid sequence set forth as SEQ ID NO:10 or SEQ ID NO:63, or a functional fragment or functional variant of the amino acid sequence set forth as SEQ ID NO:10 or SEQ ID NO:63, wherein the functional fragment or functional variant is a fragment or variant that is capable of mediating the translocation of the molecule or a fragment thereof from within the endosome, across the endosomal membrane, and into the cell cytosol.

19-20. (canceled)

21. The molecule of claim 1, wherein the linker containing a protease cleavage site comprises an amino acid sequence cleaved by a protease present in endosomes.

22. The molecule of claim 21, wherein the linker containing a protease cleavage site comprises a furin cleavage site.

23. (canceled)

24. The molecule of claim 21, wherein the linker containing a protease cleavage site comprises an amino acid sequence selected from SEQ ID NOs:6, 7, and 8.

25. The molecule of claim 1, wherein the one or more targeting moieties bind an antigen or receptor present on the surface of a cancer cell and wherein the antigen or receptor is capable of internalizing the bound molecule or a fragment thereof.

26. The molecule of claim 25, wherein the one or more targeting moieties are derived from an antibody, a cytokine, or a receptor binding protein or peptide.

27. (canceled)

28. The molecule of claim 26, wherein the at least one targeting moiety is derived from GM-CSF.

29. The molecule of claim 28, wherein the at least one targeting moiety comprises the amino acid sequence set forth as SEQ ID NO:11 or a functional fragment or functional variant of the amino acid sequence set forth as SEQ ID NO:11, wherein the functional fragment or functional variant is capable of binding an antigen present on the surface of a cancer cell.

30-31. (canceled)

32. The molecule of claim 28, wherein at least one targeting moiety is derived from IL3.

33. The molecule of claim 32, wherein the at least one targeting moiety comprises the amino acid sequence set forth as any one of SEQ ID NOs:12-15, or a functional fragment or functional variant of the amino acid sequence set forth as any one of SEQ ID NOs:12-15, wherein the functional fragment or functional variant is capable of binding an antigen present on the surface of a cancer cell.

34-35. (canceled)

36. The molecule of claim 28, wherein at least one targeting moiety is derived from IL2.

37. The molecule of claim 36, wherein the at least one targeting moiety comprises the amino acid sequence set forth as SEQ ID NO:83, or a functional fragment or functional variant of the amino acid sequence set forth as SEQ ID NO:83, wherein the functional fragment or functional variant is capable of binding an antigen present on the surface of a cancer cell.

38-39. (canceled)

40. The molecule of claim 28, wherein at least one targeting moiety is derived from IL4.

41. The molecule of claim 40, wherein the at least one targeting moiety comprises the amino acid sequence set forth as SEQ ID NO:84, or a functional fragment or functional variant of the amino acid sequence set forth as SEQ ID NO:84, wherein the functional fragment or functional variant is capable of binding an antigen present on the surface of a cancer cell.

42-44. (canceled)

45. The molecule of claim 26, wherein at least one targeting moiety is derived from an anti-CD33 antibody.

46. (canceled)

47. The molecule of claim 45, wherein the at least one targeting moiety comprises the amino acid sequence set forth as SEQ ID NO:16, or a functional fragment or functional variant of the amino acid sequence set forth as SEQ ID NO:16, wherein the functional fragment or functional variant is capable of binding CD33.

48-52. (canceled)

53. The molecule of claim 44, wherein at least one targeting moiety is derived from an anti-B-cell maturation antigen (BCMA) antibody.

54. The molecule of claim 53, wherein the at least one targeting moiety comprises the amino acid sequence set forth as SEQ ID NO:79, or a functional fragment or functional variant of the amino acid sequence set forth as SEQ ID NO:79, wherein the functional fragment or functional variant is capable of binding BCMA.

55-58. (canceled)

59. The molecule of claim 44, wherein at least one targeting moiety is derived from an anti-CD19 antibody.

60. The molecule of claim 59, wherein the at least one targeting moiety comprises the amino acid sequence set forth as SEQ ID NO:81, or a functional fragment or functional variant of the amino acid sequence set forth as SEQ ID NO:81, wherein the functional fragment or functional variant is capable of binding CD19.

61-64. (canceled)

65. The molecule of claim 44, wherein at least one targeting moiety is derived from an anti-claudin18.2 (CLDN18.2) antibody.

66-67. (canceled)

68. The molecule of claim 65, wherein the at least one targeting moiety comprises the amino acid sequence set forth as SEQ ID NO:92 or 94, or a functional fragment or functional variant of the amino acid sequence set forth as SEQ ID NO:92 or 94, wherein the functional fragment or functional variant is capable of binding CLDN18.2.

69-73. (canceled)

74. The molecule of claim 44, wherein at least one targeting moiety is derived from an anti-human epidermal growth factor receptor 2 (HER2) antibody.

75. (canceled)

76. The molecule of claim 74, wherein the at least one targeting moiety comprises the amino acid sequence set forth as any one of SEQ ID NOs:98, 100, 102, and 118, or a functional fragment or functional variant of the amino acid sequence set forth as any one of SEQ ID NOs:98, 100, 102, and 118, wherein the functional fragment or functional variant is capable of binding HER2.

77-81. (canceled)

82. The molecule of claim 44, wherein at least one targeting moiety is derived from an anti-mesothelin (MSLN) antibody.

83. (canceled)

84. The molecule of claim 82, wherein the at least one targeting moiety comprises the amino acid sequence set forth as SEQ ID NO:104, or a functional fragment or functional variant of the amino acid sequence set forth as SEQ ID NO:104, wherein the functional fragment or functional variant is capable of binding MSLN.

85-89. (canceled)

90. The molecule of claim 44, wherein at least one targeting moiety is derived from an anti-glypican-3 (GPC3) antibody.

91. (canceled)

92. The molecule of claim 90, wherein the at least one targeting moiety comprises the amino acid sequence set forth as SEQ ID NO:106, or a functional fragment or functional variant of the amino acid sequence set forth as SEQ ID NO:106, wherein the functional fragment or functional variant is capable of binding GPC3.

93-97. (canceled)

98. The molecule of claim 44, wherein at least one targeting moiety is derived from an anti-epidermal growth factor receptor (EGFR) antibody.

99. (canceled)

100. The molecule of claim 98, wherein the at least one targeting moiety comprises the amino acid sequence set forth as any one of SEQ ID NOs:108, 110, or 112, or a functional fragment or functional variant of the amino acid sequence set forth as any one of SEQ ID NOs:108, 110, or 112, wherein the functional fragment or functional variant is capable of binding EGFR.

101-105. (canceled)

106. The molecule of claim 44, wherein at least one targeting moiety is derived from an anti-trophoblast cell surface antigen 2 (TROP2) antibody.

107. (canceled)

108. The molecule of claim 106, wherein the at least one targeting moiety comprises the amino acid sequence set forth as SEQ ID NO:114, or a functional fragment or functional variant of the amino acid sequence set forth as SEQ ID NO:114, wherein the functional fragment or functional variant is capable of binding TROP2.

109-113. (canceled)

114. The molecule of claim 1, wherein the molecule comprises two or more targeting moieties.

115. The molecule of claim 114, wherein the molecule comprises two targeting moieties.

116-117. (canceled)

118. The molecule of claim 115, wherein one of the two targeting moieties is derived from GM-CSF and one of the two targeting moieties is derived from IL3.

119. The molecule of claim 118, wherein the GM-CSF-derived targeting moiety comprises the sequence set forth as SEQ ID NO:11 or a functional fragment or functional variant thereof and the IL3-derived targeting moiety comprises the sequence set forth as any one of SEQ ID NO:12-15 or a functional fragment or functional variant thereof.

120. The molecule of claim 115, wherein one of the two targeting moieties is derived from IL2 and one of the two targeting moieties is derived from IL4.

121. The molecule of claim 120, wherein the IL2-derived targeting moiety comprises the sequence set forth as SEQ ID NO:83 or a functional fragment or functional variant thereof and the IL4-derived targeting moiety comprises the sequence set forth as SEQ ID NO:85 or a functional fragment or functional variant thereof.

122. The molecule of claim 1, wherein the translocation domain is linked to the one or more targeting moieties by a linker.

123-124. (canceled)

125. The molecule of claim 114, wherein the two or more targeting moieties are linked by a linker.

126-127. (canceled)

128. The molecule of claim 1, wherein the molecule comprises a CDT-derived domain linked to a furin-cleavable linker, linked to a DTB-derived domain, linked to a GM-CSF-derived targeting moiety.

129. The molecule of claim 128, comprising the sequence set forth as any one of SEQ ID NOs:31-37, and 53-57.

130. The molecule of claim 1, wherein the molecule comprises a CDT-derived domain linked to a furin-cleavable linker, linked to a DTB-derived domain, linked to an anti-CD33-derived targeting domain.

131. The molecule of claim 130, comprising the sequence set forth as SEQ ID NO:38.

132. The molecule of claim 1, wherein the molecule comprises a CDT-derived domain linked to a furin-cleavable linker, linked to a DTB-derived domain, linked to an IL3-derived targeting domain.

133. The molecule of claim 132, comprising the sequence set forth as any one of SEQ ID NOs:39-41.

134. The molecule of claim 1, wherein the molecule comprises a CDT-derived domain linked to a furin-cleavable linker, linked to a DTB-derived domain, linked to a GM-CSF-derived targeting domain, linked to an IL3-derived targeting domain.

135. The molecule of claim 134, comprising the sequence set forth as any one of SEQ ID NOs:42-51, 61, 62, 116-117, and 120-123.

136. The molecule of claim 1, wherein the molecule comprises a CDT-derived domain linked to a furin-cleavable linker, linked to a DTB-derived domain, linked to an anti-BCMA-derived targeting domain.

137. The molecule of claim 136, comprising the sequence set forth as SEQ ID NO:80.

138. The molecule of claim 1, wherein the molecule comprises a CDT-derived domain linked to a furin-cleavable linker, linked to a DTB-derived domain, linked to an anti-CD19-derived targeting domain.

139. The molecule of claim 138, comprising the sequence set forth as SEQ ID NO:82.

140. The molecule of claim 1, wherein the molecule comprises a CDT-derived domain linked to a furin-cleavable linker, linked to a DTB-derived domain, linked to an IL2-derived targeting domain.

141. The molecule of claim 140, comprising the sequence set forth as SEQ ID NO:84 or 91.

142. The molecule of claim 1, wherein the molecule comprises a CDT-derived domain linked to a furin-cleavable linker, linked to a DTB-derived domain, linked to an IL4-derived targeting domain.

143. The molecule of claim 142, comprising the sequence set forth as SEQ ID NO:86 or 89.

144. The molecule of claim 1, wherein the molecule comprises a CDT-derived domain linked to a furin-cleavable linker, linked to a DTB-derived domain, linked to an IL4-derived targeting domain, linked to an IL2-derived targeting domain.

145. The molecule of claim 144, comprising the sequence set forth as any one of SEQ ID NOs:87-88, 90.

146. The molecule of claim 1, wherein the molecule comprises a CDT-derived domain linked to a furin-cleavable linker, linked to a DTB-derived domain, linked to an anti-CLDN18.2-derived targeting domain.

147. The molecule of claim 146, comprising the sequence set forth as any one of SEQ ID NOs:93 and 95-97.

148. The molecule of claim 1, wherein the molecule comprises a CDT-derived domain linked to a furin-cleavable linker, linked to a DTB-derived domain, linked to an anti-HER2-derived targeting domain.

149. The molecule of claim 148, comprising the sequence set forth as any one of SEQ ID NOs:99, 101, 103 and 119.

150. The molecule of claim 1, wherein the molecule comprises a CDT-derived domain linked to a furin-cleavable linker, linked to a DTB-derived domain, linked to an anti-MSLN-derived targeting domain.

151. The molecule of claim 150, comprising the sequence set forth as SEQ ID NO:105.

152. The molecule of claim 1, wherein the molecule comprises a CDT-derived domain linked to a furin-cleavable linker, linked to a DTB-derived domain, linked to an anti-GPC3-derived targeting domain.

153. The molecule of claim 152, comprising the sequence set forth as SEQ ID NO:107.

154. The molecule of claim 1, wherein the molecule comprises a CDT-derived domain linked to a furin-cleavable linker, linked to a DTB-derived domain, linked to an anti-EGFR-derived targeting domain.

155. The molecule of claim 154, comprising the sequence set forth as any one of SEQ ID NOs:109, 111, and 113.

156. The molecule of claim 1, wherein the molecule comprises a CDT-derived domain linked to a furin-cleavable linker, linked to a DTB-derived domain, linked to an anti-TROP2-derived targeting domain.

157. The molecule of claim 156, comprising the sequence set forth as SEQ ID NO:115.

158. An isolated DNA sequence encoding the molecule of claim 1.

159. A vector comprising the DNA sequence of claim 158.

160. A host cell comprising the vector of claim 159.

161. A method of producing the molecule of claim 1, comprising culturing the host cell of claim 160 and isolating the molecule.

162. A composition comprising the molecule of claim 1 and a pharmaceutically acceptable excipient.

163. A method of treating cancer in a subject comprising administering an effective amount of the molecule of claim 1 or the composition of claim 162 to the subject.

164. The method of claim 163, wherein the cancer is a hematological cancer.

165. The method of claim 164, wherein the cancer is a leukemia, lymphoma, plasma cell neoplasm, myelodysplastic syndrome (MDS), myeloproliferative neoplasm (MPN), histiocytic or dendritic cell neoplasm, or a mast cell neoplasm.

166-167. (canceled)

168. The method of claim 163 wherein the cancer is a non-hematological cancer.

169. The method of claim 168, wherein the cancer is a carcinoma, a sarcoma, a melanoma, a neuroendocrine tumor, a central nervous system tumor, a germ cell tumor, or an embryonal tumor.

170. The method of claim 168, wherein the cancer is a thoracic cancer, such as a lung cancer (including small cell lung cancer and non-small cell lung cancer) or a mesothelioma; a gastrointestinal cancer, such as a pancreatic, colorectal, gastric, gastroesophageal, hepatic, or biliary tract cancer; a genitourinary cancer, such as a prostate, renal, bladder/urothelial, or testicular cancer; a gynecologic cancer, such as an ovarian, cervical, endometrial, or uterine cancer; a breast cancer; a head and neck cancer; an esophageal cancer; a thyroid cancer; a skin cancer; or another epithelial cancer.

171-174. (canceled)
